SEQUENCE LISTING (109 sequences)

SEQ ID NO: 1 - DNA, 8069 bp, Artificial Sequence
  Description of Artificial Sequence Synthetic polynucleotide
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatgtcgacgttaacgctagtgatatcaactttgtatagaaaagttgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctccgttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggttgatatccagcacagtggcggccgctcgagtctagagggcccgcggttcgaaggtaagcctatccctaaccctctcctcggtctcgattctacgcgtaccggttagtaatgagtttggaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 2 - DNA, 22460 bp, Artificial Sequence
  Description of Artificial Sequence Synthetic polynucleotide
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatgtcgacgttaacgctagtgatatcaactttgtatagaaaagttgaacgagaaacgtaaaatgatataaatatcaatatattaaattagattttgcataaaaaacagactacataatactgtaaaacacaacatatccagtcactatgggacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaagcttaccatgccttcgcaagccctcatttcaccaggcccccggcttggggcgccttccttccccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaggtgcctgcccttctaggaatgggggacagggggaggggaggagctagggaaagaaaacctggagtttgtgccagggtttttgggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcctgggacacagtagatagacacacttaaaaaaaaaaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggacggatcgggagatctcccgatcccctatggtgcactctcagtacaatcttgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaagcttaccatgctattaacttgttcaaaaaagtatcaggagttgtcaaggcagagaagagagtgtttgcaaaagggggaaagtagtttgctgcctctttaagactaggactgagagaaagaagaggagagagaaagaaagggagagaagtttgagccccaggcttaagcctttccaaaaaataataataacaatcatcggcggcggcaggatcggccagaggaggagggaagcgctttttttgatcctgattccagtttgcctctctctttttttcccccaaattattcttcgcctgattttcctcgcggagccctgcgctcccgacacccccgcccgcctcccctcctcctctccccccgcccgcgggccccccaaagtcccggccgggccgagggtcggcggccgccggcgggccgggcccgcgcacagcgcccgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgagggccggacagcgaactggaggggggagaaattttcaaagaaaaacgagggaaatgggaggggtgcaaaagaggagagtaagaaacagcatggagaaaacccggtacgctcaaaaagaaaaaggaaaaaaaaaaatcccatcacccacagcaaatgacagctgcaaaagagaacaccaatcccatccacactcacgcaaaaaccgcgatgccgacaagaaaacttttatgagagagatcctggacttctttttgggggactatttttgtacagagaaaacctggggagggtggggagggcgggggaatggaccttgtatagatctggaggaaagaaagctacgaaaaactttttaaaagttctagtggtacggtaggagctttgcaggaagtttgcaaaagtctttaccaataatatttagagctagtctccaagcgacgaaaaaaatgttttaatatttgcaagcaacttttgtacagtatttatcgagataaacatggcaatcaaaatgtccattgtttataagctgagaatttgccaatatttttcaaggagaggcttcttgctgaattttgattctgcagctgaaatttaggacagttgcaaacgtgaaaagaagaaaattattcaaatttggacattttaattgtttaaaaattgtacaaaaggaaaaaattagaataagtactggcgaaccatctctgtggtcttgtttaaaaagggcaaaagttttagactgtactaaattttataacttactgttaaaagcaaaaatggccatgcaggttgacaccgttggtaatttataatagcttttgttcgatcccaactttccattttgttcagataaaaaaaaccatgaaattactgtgtttgaaatattttcttatggtttgtaatatttctgtaaatttattgtgatattttaaggttttcccccctttattttccgtagttgtattttaaaagattcggctctgtattatttgaatcagtctgccgagaatccatgtatatatttgaactaatatcatccttataacaggtacattttcaacttaagtttttactccattatgcacagtttgagataaataaatttttgaaatatggacactgaaaaaaaaaaaaaaaaaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaagcttaccatggttgtcatgggggaggtggtggcgcttggtggccactggcggccgaggtagaggcagtggcgcttgagttggtcgggggcagcggcagatttgaggcttaagcaacttcttccggggaagagtgccagtgcagccactgttacaattcaagatcttgatctatatccatagattggaatattggtgggccagcaatcctcagacgcctcacttaggacaaatgaggaaactgaggcttggtgaagttacgaaacttgtccaaaatcacacaacttgtaaagggcacagccaagattcagagccaggctgtaaaaattaaaatgaacaaattacggcaaagttttaggagaaagaaggatgtttatgttccagaggccagtcgtccacatcagtggcagacagatgaagaaggcgttcgcaccggaaaatgtagcttcccggttaagtaccttggccatgtagaagttgatgaatcaagaggaatgcacatctgtgaagatgctgtaaaaagattgaaagctgaaaggaagttcttcaaaggcttctttggaaaaactggaaagaaagcagttaaagcagttctgtgggtctcagcagatggactcagagttgtggatgaaaaaactaaggacctcatagttgaccagacgatagagaaagtttctttctgtgccccagacaggaactttgatagagccttttcttacatatgccgtgatggcaccactcgtcgctggatctgtcactgcttcatggctgtcaaggacacaggtgaaaggttgagccatgcagtaggctgtgcttttgcagcctgtttagagcgcaagcagaagcgggagaaggaatgtggagtgactgctacttttgatgctagtcggaccacttttacaagagaaggatcattccgtgtcacaacagccactgaacaagcagaaagagaggagatcatgaaacaaatgcaagatgccaagaaagctgaaacagataagatagtcgttggttcatcagttgcccctggcaacactgccccatccccatcctctcccacctctcctacttctgatgccacgacctctctggagatgaacaatcctcatgccatcccacgccggcatgctccaattgaacagcttgctcgccaaggctctttccgaggttttcctgctcttagccagaagatgtcaccctttaaacgccaactatccctacgcatcaatgagttgccttccactatgcagaggaagactgatttccccattaaaaatgcagtgccagaagtagaaggggaggcagagagcatcagctccctgtgctcacagatcaccaatgccttcagcacacctgaggaccccttctcatctgctccgatgaccaaaccagtgacagtggtggcaccacaatctcctaccttccaagctaatggcactgactcagccttccatgtgcttgctaagccagcccatactgctctagcacccgtagcaatgcctgtgcgtgaaaccaacccttgggcccatgcccctgatgctgctaacaaggaaattgcagccacatgttcggggaccgagtggggtcaatcttctggtgctgcctctccaggtctcttccaggccggtcatagacgtactccctctgaggccgaccgatggttagaagaggtgtctaagagcgtccgggctcagcagccccaggcctcagctgctcctctgcagccagttctccagcctcctccacccactgccatctcccagccagcatcacctttccaagggaatgcattcctcacctctcagcctgtgccagtgggtgtggtcccagccctgcaaccagcctttgtccctgcccagtcctatcctgtggccaatggaatgccctatccagcccctaatgtgcctgtggtgggcatcactccctcccagatggtggccaacgtatttggcactgcaggccaccctcaggctgcccatccccatcagtcacccagcctggtcaggcagcagacattccctcactacgaggcaagcagtgctaccaccagtcccttctttaagcctcctgctcagcacctcaacggttctgcagctttcaatggtgtagatgatggcaggttggcctcagcagacaggcatacagaggttcctacaggcacctgcccagtggatccttttgaagcccagtgggctgcattagaaaataagtccaagcagcgtactaatccctcccctaccaaccctttctccagtgacttacagaagacgtttgaaattgaactttaagcaatcattatggctatgtatcttgtccataccagacagggagcagggggtagcggtcaaaggagcaaaacagactttgtctcctgattagtactcttttcactaatcccaaaggtcccaaggaacaagtccaggcccagagtactgtgaggggtgattttgaaagacatgggaaaaagcattcctagagaaaagctgccttgcaattaggctaaagaagtcaaggaaatgttgctttctgtactccctcttcccttacccccttacaaatctctggcaacagagaggcaaagtatctgaacaagaatctatattccaagcacatttactgaaatgtaaaacacaacaggaagcaaagcaatctccctttgtttttcaggccattcacctgcctcctgtcagtagtggcctgtattagagatcaagaagagtggtttgtgctcaggctggggaacagagaggcacgctatgctgccagaattcccaggagggcatatcagcaactgcccagcagagctatattttgggggagaagttgagcttccattttgagtaacagaataaatattatatatatcaaaagccaaaatctttatttttatgcatttagaatattttaaatagttctcagatattaagaagttgtatgagttgtaagtaatcttgccaaaggtaaaggggctagttgtaagaaattgtacataagattgatttatcattgatgcctactgaaataaaaagaggaaaggctggaagctgcagacaggatccctagcttgttttctgtcagtcattcattgtaagtagcacattgcaacaacaatcatgcttatgaccaatacagtcactaggttgtagttttttttaaataaaggaaaagcagtattgtcctggttttaaacctatgatggaattctaatgtcattattttaatggaatcaatcgaaatatgctctatagagaatatatcttttatatattgctgcagtttccttatgttaatcctttaacactaaggtaacatgacataatcataccatagaagggaacacaggttaccatattggtttgtaatatgggtcttggtgggttttgttttatcctttaaattttgttcccatgagttttgtggggatggggattctggttttattagctttgtgtgtgtcctcttcccccaaacccccttttggtgagaacatccccttgacagttgcagcctcttgacctcggataacaataagagagctcatctcatttttacttttgaacgttggccttacaatcaaatgtaagttatatatatttgtactgatgaaaatttataatctgctttaacaaaaataaatgttcatggtagaagcttttaaaaaaaaaaaaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaagcttaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctcgtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattgatatgtctagattagataaaagtaaagtgattaacagcgcattagagctgcatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagcggatcccgggaattcagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccccatagtgactggatatgttgtgttttacagtattatgtagtctgttttttatgcaaaatctaatttaatatattgatatttatatcattttacgtttctcgttcagctttcttgtacaaagtggttgatatccagcacagtggcggccgctcgagtctagagggcccgcggttcgaaggtaagcctatccctaaccctctcctcggtctcgattctacgcgtaccggttagtaatgagtttggaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 3 - DNA, 1751 bp, Artificial Sequence
  Description of Artificial Sequence Synthetic polynucleotide
gtcgaccagtggatcctggaggcttgctgaaggctgtatgctgatcgggtgtaaactgagcttggttttggccactgactgaccaagctcattacacccgatcaggacacaaggcctgttactagcactcacatggaacaaatggcccagatcctggaggcttgctgaaggctgtatgctgataccaggcaggataaggccagttttggccactgactgactggccttactgcctggtatcaggacacaaggcctgttactagcactcacatggaacaaatggcccagatcctggaggcttgctgaaggctgtatgctgtgaccaggatgaccaatccatgttttggccactgactgacatggattgcatcctggtcacaggacacaaggcctgttactagcactcacatggaacaaatggcccagatcctggaggcttgctgaaggctgtatgctgatagcttggtccaacctgttagttttggccactgactgactaacaggtgaccaagctatcaggacacaaggcctgttactagcactcacatggaacaaatggcccagatctccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacggtgctcgcttcggcagcacgtcgtgctagggttcttgggttttctcgcaacagcaggttctgcaatgggcgcggcgtccctgaccgtgtcggctcagtcccggactttactggccgggatagtgcagcaacagcaacagctgttggacgtggtcaagagacaacaagaactgttgcgactgaccgtctggggaacgaaaaacctccaggcaagagtcactgctatagagaagtacctacaggaccaggcgcggctaaattcatggggatgtctagacctagagcggacttcggtccgctttttccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacggtgctcgcttcggcagcacgtcggtcgctctgcggagaggctggcagattgagccctgggaggttctctccagcactagcaggtagagcctgggtgttccctgctagactctcaccagtgcttggccggcactgggcagacggctccacgcttgcttgcttaaagacctcttaataaagctgctctagacctagagcggacttcggtccgcttttttacgtactcgag

SEQ ID NO: 4 - DNA, 64 bp, Artificial Sequence
  Description of Artificial Sequence Synthetic oligonucleotide
tgctgataccaggcaggataaggccagttttggccactgactgactggccttactgcctggtat

SEQ ID NO: 5 - DNA, 64 bp, Artificial Sequence
  Description of Artificial Sequence Synthetic oligonucleotide
cctgataccaggcagtaaggccagtcagtcagtggccaaaactggccttatcctgcctggtatc

SEQ ID NO: 6 - DNA, 64 bp, Artificial Sequence
  Description of Artificial Sequence Synthetic oligonucleotide
tgctgtgaccaggatgaccaatccatgttttggccactgactgacatggattgcatcctggtca

SEQ ID NO: 7 - DNA, 64 bp, Artificial Sequence
  Description of Artificial Sequence Synthetic oligonucleotide
cctgtgaccaggatgcaatccatgtcagtcagtggccaaaacatggattggtcatcctggtcac

SEQ ID NO: 8 - DNA, 64 bp, Artificial Sequence
  Description of Artificial Sequence Synthetic oligonucleotide
tgctgatcgggtgtaaactgagcttggttttggccactgactgaccaagctcattacacccgat

SEQ ID NO: 9 - DNA, 64 bp, Artificial Sequence
  Description of Artificial Sequence Synthetic oligonucleotide
cctgatcgggtgtaatgagcttggtcagtcagtggccaaaaccaagctcagtttacacccgatc

SEQ ID NO: 10 - DNA, 64 bp, Artificial Sequence
  Description of Artificial Sequence Synthetic oligonucleotide
tgctgatagcttggtccaacctgttagttttggccactgactgactaacaggtgaccaagctat

SEQ ID NO: 11 - DNA, 64 bp, Artificial Sequence
  Description of Artificial Sequence Synthetic oligonucleotide
cctgatagcttggtcacctgttagtcagtcagtggccaaaactaacaggttggaccaagctatc

SEQ ID NO: 12 - DNA, 160 bp, Artificial Sequence
  Description of Artificial Sequence Synthetic polynucleotide
gtcgctctgcggagaggctggcagattgagccctgggaggttctctccagcactagcaggtagagcctgggtgttccctgctagactctcaccagtgcttggccggcactgggcagacggctccacgcttgcttgcttaaagacctcttaataaagctgc

SEQ ID NO: 13 - DNA, 247 bp, Artificial Sequence
  Description of Artificial Sequence Synthetic polynucleotide
tgctagggttcttgggttttctcgcaacagcaggttctgcaatgggcgcggcgtccctgaccgtgtcggctcagtcccggactttactggccgggatagtgcagcaacagcaacagctgttggacgtggtcaagagacaacaagaactgttgcgactgaccgtctggggaacgaaaaacctccaggcaagagtcactgctatagagaagtacctacaggaccaggcgcggctaaattcatggggatg

SEQ ID NO: 14 - DNA, 25 bp, Artificial Sequence
  Description of Combined DNA/RNA Molecule Synthetic oligonucleotide
gugcucgcuucggcagcacgtcgac

SEQ ID NO: 15 - RNA, 26 nt, Artificial Sequence
  Description of Artificial Sequence Synthetic olgionucleotide
ucuagagcggacuucgguccgcuuuu

SEQ ID NO: 16 - DNA, 25 bp, Artificial Sequence
  Description of Combined DNA/RNA Molecule Synthetic oligonucleotide
gugcucgcuucggcagcacgtcgac

SEQ ID NO: 17 - RNA, 26 nt, Artificial Sequence
  Description of Artificial Sequence Synthetic oligonucleotide
ucuagagcggacuucgguccgcuuuu

SEQ ID NO: 18 - DNA, 606 bp, Homo sapiens
atgagccggagggagggaagtctggaagacccccagactgattcctcagtctcacttcttccccacttggaggccaagatccgtcagacacacagccttgcgcacctcctcaccaaatacgctgagcagctgctccaggaatatgtgcagctccagggagaccccttcgggctgcccagcttctcgccgccgcggctgccggtggccggcctgagcgccccggctccgagccacgcggggctgccagtgcacgagcggctgcggctggacgcggcggcgctggccgcgctgcccccgctgctggacgcagtgtgtcgccgccaggccgagctgaacccgcgcgcgccgcgcctgctgcgccgcctggaggacgcggcgcgccaggcccgggccctgggcgccgccgtggaggccttgctggccgcgctgggcgccgccaaccgcgggccccgggccgagccccccgccgccaccgcctcagccgcctccgccaccggggtcttccccgccaaggtgctggggctccgcgtttgcggcctctaccgcgagtggctgagccgcaccgagggcgacctgggccagctgctgcccgggggctcggcctga

SEQ ID NO: 19 - DNA, 603 bp, Homo sapiens
atggctttcacagagcattcaccgctgacccctcaccgtcgggacctctgtagccgctctatctggctagcaaggaagattcgttcagacctgactgctcttacggaatcctatgtgaagcatcagggcctgaacaagaacatcaacctggactctgcggatgggatgccagtggcaagcactgatcagtggagtgagctgaccgaggcagagcgactccaagagaaccttcaagcttatcgtaccttccatgttttgttggccaggctcttagaagaccagcaggtgcattttaccccaaccgaaggtgacttccatcaagctatacatacccttcttctccaagtcgctgcctttgcataccagatagaggagttaatgatactcctggaatacaagatcccccgcaatgaggctgatgggatgcctattaatgttggagatggtggtctctttgagaagaagctgtggggcctaaaggtgctgcaggagctttcacagtggacagtaaggtccatccatgaccttcgtttcatttcttctcatcagactgggatcccagcacgtgggagccattatattgctaacaacaagaaaatgtag

SEQ ID NO: 20 - DNA, 990 bp, Homo sapiens
atgttgacgttgcagacttggctagtgcaagccttgtttattttcctcaccactgaatctacaggtgaacttctagatccatgtggttatatcagtcctgaatctccagttgtacaacttcattctaatttcactgcagtttgtgtgctaaaggaaaaatgtatggattattttcatgtaaatgctaattacattgtctggaaaacaaaccattttactattcctaaggagcaatatactatcataaacagaacagcatccagtgtcacctttacagatatagcttcattaaatattcagctcacttgcaacattcttacattcggacagcttgaacagaatgtttatggaatcacaataatttcaggcttgcctccagaaaaacctaaaaatttgagttgcattgtgaacgaggggaagaaaatgaggtgtgagtgggatggtggaagggaaacacacttggagacaaacttcactttaaaatctgaatgggcaacacacaagtttgctgattgcaaagcaaaacgtgacacccccacctcatgcactgttgattattctactgtgtattttgtcaacattgaagtctgggtagaagcagagaatgcccttgggaaggttacatcagatcatatcaattttgatcctgtatataaagtgaagcccaatccgccacataatttatcagtgatcaactcagaggaactgtctagtatcttaaaattgacatggaccaacccaagtattaagagtgttataatactaaaatataacattcaatataggaccaaagatgcctcaacttggagccagattcctcctgaagacacagcatccacccgatcttcattcactgtccaagaccttaaaccttttacagaatatgtgtttaggattcgctgtatgaaggaagatggtaagggatactggagtgactggagtgaagaagcaagtgggatcacctatgaagataacattgcctccttttga

SEQ ID NO: 21 - DNA, 639 bp, Homo sapiens
atgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtag

SEQ ID NO: 22 - DNA, 756 bp, Homo sapiens
atggctatgagttcttttttgatcaactcaaactatgtcgaccccaagttccctccatgcgaggaatattcacagagcgattacctacccagcgaccactcgcccgggtactacgccggcggccagaggcgagagagcagcttccagccggaggcgggcttcgggcggcgcgcggcgtgcaccgtgcagcgctacgcggcctgccgggaccctgggcccccgccgcctccgccaccacccccgccgcccccgccaccgcccggtctgtcccctcgggctcctgcgccgccacccgccggggccctcctcccggagcccggccagcgctgcgaggcggtcagcagcagccccccgccgcctccctgcgcccagaaccccctgcaccccagcccgtcccactccgcgtgcaaagagcccgtcgtctacccctggatgcgcaaagttcacgtgagcacggtaaaccccaattacgccggcggggagcccaagcgctctcggaccgcctacacgcgccagcaggtcttggagctggagaaggaatttcactacaaccgctacctgacacggcgccggagggtggagatcgcccacgcgctctgcctctccgagcgccagatcaagatctggttccagaaccggcgcatgaagtggaaaaaagaccacaagttgcccaacaccaagatccgctcgggtggtgcggcaggctcagccggagggccccctggccggcccaatggaggcccccgcgcgctctag

SEQ ID NO: 23 - DNA, 1407 bp, Homo sapiens
atgctggccgtcggctgcgcgctgctggctgccctgctggccgcgccgggagcggcgctggccccaaggcgctgccctgcgcaggaggtggcgagaggcgtgctgaccagtctgccaggagacagcgtgactctgacctgcccgggggtagagccggaagacaatgccactgttcactgggtgctcaggaagccggctgcaggctcccaccccagcagatgggctggcatgggaaggaggctgctgctgaggtcggtgcagctccacgactctggaaactattcatgctaccgggccggccgcccagctgggactgtgcacttgctggtggatgttccccccgaggagccccagctctcctgcttccggaagagccccctcagcaatgttgtttgtgagtggggtcctcggagcaccccatccctgacgacaaaggctgtgctcttggtgaggaagtttcagaacagtccggccgaagacttccaggagccgtgccagtattcccaggagtcccagaagttctcctgccagttagcagtcccggagggagacagctctttctacatagtgtccatgtgcgtcgccagtagtgtcgggagcaagttcagcaaaactcaaacctttcagggttgtggaatcttgcagcctgatccgcctgccaacatcacagtcactgccgtggccagaaacccccgctggctcagtgtcacctggcaagacccccactcctggaactcatctttctacagactacggtttgagctcagatatcgggctgaacggtcaaagacattcacaacatggatggtcaaggacctccagcatcactgtgtcatccacgacgcctggagcggcctgaggcacgtggtgcagcttcgtgcccaggaggagttcgggcaaggcgagtggagcgagtggagcccggaggccatgggcacgccttggacagaatccaggagtcctccagctgagaacgaggtgtccacccccatgcaggcacttactactaataaagacgatgataatattctcttcagagattctgcaaatgcgacaagcctcccagtgcaagattcttcttcagtaccactgcccacattcctggttgctggagggagcctggccttcggaacgctcctctgcattgccattgttctgaggttcaagaagacgtggaagctgcgggctctgaaggaaggcaagacaagcatgcatccgccgtactctttggggcagctggtcccggagaggcctcgacccaccccagtgcttgttcctctcatctccccaccggtgtcccccagcagcctggggtctgacaatacctcgagccacaaccgaccagatgccagggacccacggagcccttatgacatcagcaatacagactacttcttccccagatag

SEQ ID NO: 24 - DNA, 600 bp, Rattus norvegicus
atgaactgtgtttgtcgcctggtcctggtggtgctgagcctctggccagatagagtcgttgcccctgggccaccagctggctcccctcgagtgtcttcagaccctcgtgcagatctggatagcgctgtcctcttgaccaggtccctcctggcagacacacggcaactagctgcacagatgagagacaaattcccagctgatggagaccacaatctggactccctacctaccttggccatgagcgctgggacactgggatctttgcagcttcctggagtgctgacaaggcttcgagtagacttaatgtcctacttccgacatgtacagtggttgcgccgggcagctggtccttccctaaagactctggagccagagctgggtgccctgcaagcccgactggaacggctacttcgtcgcttacagctcttgatgtctcgcctagccttgccccaggcagccccggaccaacctgcggtccctctgggccctcctgcctcggcctggggaagcatccgggcagctcatgccatcctaggagggctgcacctgaccttggactgggccgtgcggggcctgctgttgttaaagactcggctgtaa

SEQ ID NO: 25 - DNA, 609 bp, Homo sapiens
atgaaggtcttggcggcaggagttgtgcccctgctgttggttctgcactggaaacatggggcggggagccccctccccatcacccctgtcaacgccacctgtgccatacgccacccatgtcacaacaacctcatgaaccagatcaggagccaactggcacagctcaatggcagtgccaatgccctctttattctctattacacagcccagggggagccgttccccaacaacctggacaagctatgtggccccaacgtgacggacttcccgcccttccacgccaacggcacggagaaggccaagctggtggagctgtaccgcatagtcgtgtaccttggcacctccctgggcaacatcacccgggaccagaagatcctcaaccccagtgccctcagcctccacagcaagctcaacgccaccgccgacatcctgcgaggcctccttagcaacgtgctgtgccgcctgtgcagcaagtaccacgtgggccatgtggacgtgacctacggccctgacacctcgggtaaggatgtcttccagaagaagaagctgggctgtcaactcctggggaagtataagcagatcatcgccgtgttggcccaggccttctag

SEQ ID NO: 26 - DNA, 3294 bp, Homo sapiens
atgatggatatttacgtatgtttgaaacgaccatcctggatggtggacaataaaagaatgaggactgcttcaaatttccagtggctgttatcaacatttattcttctatatctaatgaatcaagtaaatagccagaaaaagggggctcctcatgatttgaagtgtgtaactaacaatttgcaagtgtggaactgttcttggaaagcaccctctggaacaggccgtggtactgattatgaagtttgcattgaaaacaggtcccgttcttgttatcagttggagaaaaccagtattaaaattccagctctttcacatggtgattatgaaataacaataaattctctacatgattttggaagttctacaagtaaattcacactaaatgaacaaaacgtttccttaattccagatactccagagatcttgaatttgtctgctgatttctcaacctctacattatacctaaagtggaacgacaggggttcagtttttccacaccgctcaaatgttatctgggaaattaaagttctacgtaaagagagtatggagctcgtaaaattagtgacccacaacacaactctgaatggcaaagatacacttcatcactggagttgggcctcagatatgcccttggaatgtgccattcattttgtggaaattagatgctacattgacaatcttcatttttctggtctcgaagagtggagtgactggagccctgtgaagaacatttcttggatacctgattctcagactaaggtttttcctcaagataaagtgatacttgtaggctcagacataacattttgttgtgtgagtcaagaaaaagtgttatcagcactgattggccatacaaactgccccttgatccatcttgatggggaaaatgttgcaatcaagattcgtaatatttctgtttctgcaagtagtggaacaaatgtagtttttacaaccgaagataacatatttggaaccgttatttttgctggatatccaccagatactcctcaacaactgaattgtgagacacatgatttaaaagaaattatatgtagttggaatccaggaagggtgacagcgttggtgggcccacgtgctacaagctacactttagttgaaagtttttcaggaaaatatgttagacttaaaagagctgaagcacctacaaacgaaagctatcaattattatttcaaatgcttccaaatcaagaaatatataattttactttgaatgctcacaatccgctgggtcgatcacaatcaacaattttagttaatataactgaaaaagtttatccccatactcctacttcattcaaagtgaaggatattaattcaacagctgttaaactttcttggcatttaccaggcaactttgcaaagattaattttttatgtgaaattgaaattaagaaatctaattcagtacaagagcagcggaatgtcacaatcaaaggagtagaaaattcaagttatcttgttgctctggacaagttaaatccatacactctatatacttttcggattcgttgttctactgaaactttctggaaatggagcaaatggagcaataaaaaacaacatttaacaacagaagccagtccttcaaaggggcctgatacttggagagagtggagttctgatggaaaaaatttaataatctattggaagcctttacccattaatgaagctaatggaaaaatactttcctacaatgtatcgtgttcatcagatgaggaaacacagtccctttctgaaatccctgatcctcagcacaaagcagagatacgacttgataagaatgactacatcatcagcgtagtggctaaaaattctgtgggctcatcaccaccttccaaaatagcgagtatggaaattccaaatgatgatctcaaaatagaacaagttgttgggatgggaaaggggattctcctcacctggcattacgaccccaacatgacttgcgactacgtcattaagtggtgtaactcgtctcggtcggaaccatgccttatggactggagaaaagttccctcaaacagcactgaaactgtaatagaatctgatgagtttcgaccaggtataagatataattttttcctgtatggatgcagaaatcaaggatatcaattattacgctccatgattggatatatagaagaattggctcccattgttgcaccaaattttactgttgaggatacttctgcagattcgatattagtaaaatgggaagacattcctgtggaagaacttagaggctttttaagaggatatttgttttactttggaaaaggagaaagagacacatctaagatgagggttttagaatcaggtcgttctgacataaaagttaagaatattactgacatatcccagaagacactgagaattgctgatcttcaaggtaaaacaagttaccacctggtcttgcgagcctatacagatggtggagtgggcccggagaagagtatgtatgtggtgacaaaggaaaattctgtgggattaattattgccattctcatcccagtggcagtggctgtcattgttggagtggtgacaagtatcctttgctatcggaaacgagaatggattaaagaaaccttctaccctgatattccaaatccagaaaactgtaaagcattacagtttcaaaagagtgtctgtgagggaagcagtgctcttaaaacattggaaatgaatccttgtaccccaaataatgttgaggttctggaaactcgatcagcatttcctaaaatagaagatacagaaataatttccccagtagctgagcgtcctgaagatcgctctgatgcagagcctgaaaaccatgtggttgtgtcctattgtccacccatcattgaggaagaaataccaaacccagccgcagatgaagctggagggactgcacaggttatttacattgatgttcagtcgatgtatcagcctcaagcaaaaccagaagaagaacaagaaaatgaccctgtaggaggggcaggctataagccacagatgcacctccccattaattctactgtggaagatatagctgcagaagaggacttagataaaactgcgggttacagacctcaggccaatgtaaatacatggaatttagtgtctccagactctcctagatccatagacagcaacagtgagattgtctcatttggaagtccatgctccattaattcccgacaatttttgattcctcctaaagatgaagactctcctaaatctaatggaggagggtggtcctttacaaacttttttcagaacaaaccaaacgattaa

SEQ ID NO: 27 - DNA, 2310 bp, Homo sapiens
atggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatcttgagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggaggcgtcactttcacttgggtggagaaggacatcagcggtaagacccagatccagtccgtggaaccatacacaaagcagcagctgaacaacatgtcatttgctgaaatcatcatgggctataagatcatggatgctaccaatatcctggtgtctccactggtctatctctatcctgacattcccaaggaggaggcattcggaaagtattgtcggccagagagccaggagcatcctgaagctgacccaggcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatgtga

SEQ ID NO: 28 - DNA, 1956 bp, Homo sapiens
atgaacaaattacggcaaagttttaggagaaagaaggatgtttatgttccagaggccagtcgtccacatcagtggcagacagatgaagaaggcgttcgcaccggaaaatgtagcttcccggttaagtaccttggccatgtagaagttgatgaatcaagaggaatgcacatctgtgaagatgctgtaaaaagattgaaagctgaaaggaagttcttcaaaggcttctttggaaaaactggaaagaaagcagttaaagcagttctgtgggtctcagcagatggactcagagttgtggatgaaaaaactaaggacctcatagttgaccagacgatagagaaagtttctttctgtgccccagacaggaactttgatagagccttttcttacatatgccgtgatggcaccactcgtcgctggatctgtcactgcttcatggctgtcaaggacacaggtgaaaggttgagccatgcagtaggctgtgcttttgcagcctgtttagagcgcaagcagaagcgggagaaggaatgtggagtgactgctacttttgatgctagtcggaccacttttacaagagaaggatcattccgtgtcacaacagccactgaacaagcagaaagagaggagatcatgaaacaaatgcaagatgccaagaaagctgaaacagataagatagtcgttggttcatcagttgcccctggcaacactgccccatccccatcctctcccacctctcctacttctgatgccacgacctctctggagatgaacaatcctcatgccatcccacgccggcatgctccaattgaacagcttgctcgccaaggctctttccgaggttttcctgctcttagccagaagatgtcaccctttaaacgccaactatccctacgcatcaatgagttgccttccactatgcagaggaagactgatttccccattaaaaatgcagtgccagaagtagaaggggaggcagagagcatcagctccctgtgctcacagatcaccaatgccttcagcacacctgaggaccccttctcatctgctccgatgaccaaaccagtgacagtggtggcaccacaatctcctaccttccaagctaatggcactgactcagccttccatgtgcttgctaagccagcccatactgctctagcacccgtagcaatgcctgtgcgtgaaaccaacccttgggcccatgcccctgatgctgctaacaaggaaattgcagccacatgttcggggaccgagtggggtcaatcttctggtgctgcctctccaggtctcttccaggccggtcatagacgtactccctctgaggccgaccgatggttagaagaggtgtctaagagcgtccgggctcagcagccccaggcctcagctgctcctctgcagccagttctccagcctcctccacccactgccatctcccagccagcatcacctttccaagggaatgcattcctcacctctcagcctgtgccagtgggtgtggtcccagccctgcaaccagcctttgtccctgcccagtcctatcctgtggccaatggaatgccctatccagcccctaatgtgcctgtggtgggcatcactccctcccagatggtggccaacgtatttggcactgcaggccaccctcaggctgcccatccccatcagtcacccagcctggtcaggcagcagacattccctcactacgaggcaagcagtgctaccaccagtcccttctttaagcctcctgctcagcacctcaacggttctgcagctttcaatggtgtagatgatggcaggttggcctcagcagacaggcatacagaggttcctacaggcacctgcccagtggatccttttgaagcccagtgggctgcattagaaaataagtccaagcagcgtactaatccctcccctaccaaccctttctccagtgacttacagaagacgtttgaaattgaactttaa

SEQ ID NO: 29 - DNA, 1812 bp, Homo sapiens
atgaacaaattacggcaaagttttaggagaaagaaggatgtttatgttccagaggccagtcgtccacatcagtggcagacagatgaagaaggcgttcgcaccggaaaatgtagcttcccggttaagtaccttggccatgtagaagttgatgaatcaagaggaatgcacatctgtgaagatgctgtaaaaagattgaaagctgaaaggaagttcttcaaaggcttctttggaaaaactggaaagaaagcagttaaagcagttctgtgggtctcagcagatggactcagagttgtggatgaaaaaactaaggacctcatagttgaccagacgatagagaaagtttctttctgtgccccagacaggaactttgatagagccttttcttacatatgccgtgatggcaccactcgtcgctggatctgtcactgcttcatggctgtcaaggacacaggtgaaaggttgagccatgcagtaggctgtgcttttgcagcctgtttagagcgcaagcagaagcgggagaaggaatgtggagtgactgctacttttgatgctagtcggaccacttttacaagagaaggatcattccgtgtcacaacagccactgaacaagcagaaagagaggagatcatgaaacaaatgcaagatgccaagaaagctgaaacagataagatagtcgttggttcatcagttgcccctggcaacactgccccatccccatcctctcccacctctcctacttctgatgccacgacctctctggagatgaacaatcctcatgccatcccacgccggcatgctccaattgaacagcttgctcgccaaggctctttccgaggttttcctgctcttagccagaagatgtcaccctttaaacgccaactatccctacgcatcaatgagttgccttccactatgcagaggaagactgatttccccattaaaaatgcagtgccagaagtagaaggggaggcagagagcatcagctccctgtgctcacagatcaccaatgccttcagcacacctgaggaccccttctcatctgctccgatgaccaaaccagtgacagtggtggcaccacaatctcctaccttccaagggaccgagtggggtcaatcttctggtgctgcctctccaggtctcttccaggccggtcatagacgtactccctctgaggccgaccgatggttagaagaggtgtctaagagcgtccgggctcagcagccccaggcctcagctgctcctctgcagccagttctccagcctcctccacccactgccatctcccagccagcatcacctttccaagggaatgcattcctcacctctcagcctgtgccagtgggtgtggtcccagccctgcaaccagcctttgtccctgcccagtcctatcctgtggccaatggaatgccctatccagcccctaatgtgcctgtggtgggcatcactccctcccagatggtggccaacgtatttggcactgcaggccaccctcaggctgcccatccccatcagtcacccagcctggtcaggcagcagacattccctcactacgaggcaagcagtgctaccaccagtcccttctttaagcctcctgctcagcacctcaacggttctgcagctttcaatggtgtagatgatggcaggttggcctcagcagacaggcatacagaggttcctacaggcacctgcccagtggatccttttgaagcccagtgggctgcattagaaaataagtccaagcagcgtactaatccctcccctaccaaccctttctccagtgacttacagaagacgtttgaaattgaactttaa

SEQ ID NO: 30 - DNA, 1923 bp, Homo sapiens
atgaacaaattacggcaaagttttaggagaaagaaggatgtttatgttccagaggccagtcgtccacatcagtggcagacagatgaagaaggcgttcgcaccggaaaatgtagcttcccggttaagtaccttggccatgtagaagttgatgaatcaagaggaatgcacatctgtgaagatgctgtaaaaagattgaaagctactggaaagaaagcagttaaagcagttctgtgggtctcagcagatggactcagagttgtggatgaaaaaactaaggacctcatagttgaccagacgatagagaaagtttctttctgtgccccagacaggaactttgatagagccttttcttacatatgccgtgatggcaccactcgtcgctggatctgtcactgcttcatggctgtcaaggacacaggtgaaaggttgagccatgcagtaggctgtgcttttgcagcctgtttagagcgcaagcagaagcgggagaaggaatgtggagtgactgctacttttgatgctagtcggaccacttttacaagagaaggatcattccgtgtcacaacagccactgaacaagcagaaagagaggagatcatgaaacaaatgcaagatgccaagaaagctgaaacagataagatagtcgttggttcatcagttgcccctggcaacactgccccatccccatcctctcccacctctcctacttctgatgccacgacctctctggagatgaacaatcctcatgccatcccacgccggcatgctccaattgaacagcttgctcgccaaggctctttccgaggttttcctgctcttagccagaagatgtcaccctttaaacgccaactatccctacgcatcaatgagttgccttccactatgcagaggaagactgatttccccattaaaaatgcagtgccagaagtagaaggggaggcagagagcatcagctccctgtgctcacagatcaccaatgccttcagcacacctgaggaccccttctcatctgctccgatgaccaaaccagtgacagtggtggcaccacaatctcctaccttccaagctaatggcactgactcagccttccatgtgcttgctaagccagcccatactgctctagcacccgtagcaatgcctgtgcgtgaaaccaacccttgggcccatgcccctgatgctgctaacaaggaaattgcagccacatgttcggggaccgagtggggtcaatcttctggtgctgcctctccaggtctcttccaggccggtcatagacgtactccctctgaggccgaccgatggttagaagaggtgtctaagagcgtccgggctcagcagccccaggcctcagctgctcctctgcagccagttctccagcctcctccacccactgccatctcccagccagcatcacctttccaagggaatgcattcctcacctctcagcctgtgccagtgggtgtggtcccagccctgcaaccagcctttgtccctgcccagtcctatcctgtggccaatggaatgccctatccagcccctaatgtgcctgtggtgggcatcactccctcccagatggtggccaacgtatttggcactgcaggccaccctcaggctgcccatccccatcagtcacccagcctggtcaggcagcagacattccctcactacgaggcaagcagtgctaccaccagtcccttctttaagcctcctgctcagcacctcaacggttctgcagctttcaatggtgtagatgatggcaggttggcctcagcagacaggcatacagaggttcctacaggcacctgcccagtggatccttttgaagcccagtgggctgcattagaaaataagtccaagcagcgtactaatccctcccctaccaaccctttctccagtgacttacagaagacgtttgaaattgaactttaa

SEQ ID NO: 31 - DNA, 1779 bp, Homo sapiens
atgaacaaattacggcaaagttttaggagaaagaaggatgtttatgttccagaggccagtcgtccacatcagtggcagacagatgaagaaggcgttcgcaccggaaaatgtagcttcccggttaagtaccttggccatgtagaagttgatgaatcaagaggaatgcacatctgtgaagatgctgtaaaaagattgaaagctactggaaagaaagcagttaaagcagttctgtgggtctcagcagatggactcagagttgtggatgaaaaaactaaggacctcatagttgaccagacgatagagaaagtttctttctgtgccccagacaggaactttgatagagccttttcttacatatgccgtgatggcaccactcgtcgctggatctgtcactgcttcatggctgtcaaggacacaggtgaaaggttgagccatgcagtaggctgtgcttttgcagcctgtttagagcgcaagcagaagcgggagaaggaatgtggagtgactgctacttttgatgctagtcggaccacttttacaagagaaggatcattccgtgtcacaacagccactgaacaagcagaaagagaggagatcatgaaacaaatgcaagatgccaagaaagctgaaacagataagatagtcgttggttcatcagttgcccctggcaacactgccccatccccatcctctcccacctctcctacttctgatgccacgacctctctggagatgaacaatcctcatgccatcccacgccggcatgctccaattgaacagcttgctcgccaaggctctttccgaggttttcctgctcttagccagaagatgtcaccctttaaacgccaactatccctacgcatcaatgagttgccttccactatgcagaggaagactgatttccccattaaaaatgcagtgccagaagtagaaggggaggcagagagcatcagctccctgtgctcacagatcaccaatgccttcagcacacctgaggaccccttctcatctgctccgatgaccaaaccagtgacagtggtggcaccacaatctcctaccttccaagggaccgagtggggtcaatcttctggtgctgcctctccaggtctcttccaggccggtcatagacgtactccctctgaggccgaccgatggttagaagaggtgtctaagagcgtccgggctcagcagccccaggcctcagctgctcctctgcagccagttctccagcctcctccacccactgccatctcccagccagcatcacctttccaagggaatgcattcctcacctctcagcctgtgccagtgggtgtggtcccagccctgcaaccagcctttgtccctgcccagtcctatcctgtggccaatggaatgccctatccagcccctaatgtgcctgtggtgggcatcactccctcccagatggtggccaacgtatttggcactgcaggccaccctcaggctgcccatccccatcagtcacccagcctggtcaggcagcagacattccctcactacgaggcaagcagtgctaccaccagtcccttctttaagcctcctgctcagcacctcaacggttctgcagctttcaatggtgtagatgatggcaggttggcctcagcagacaggcatacagaggttcctacaggcacctgcccagtggatccttttgaagcccagtgggctgcattagaaaataagtccaagcagcgtactaatccctcccctaccaaccctttctccagtgacttacagaagacgtttgaaattgaactttaa

SEQ ID NO: 32 - DNA, 1830 bp, Mus musculus
atgtcccgcagcgcggcggccagcggcggaccccggaggcctgagcggcacctgcccccagccccctgtggggccccggggcccccagaaacctgcaggacggagccagacggggcgggcaccatgaacaagttacggcagagcctgcggcggaggaagccagcctacgtgcccgaggcgtcgcgcccgcaccagtggcaggcagacgaggacgcggtgcggaagggcacgtgcagcttcccggtcaggtacctgggtcacgtggaggtagaggagtcccggggaatgcacgtgtgtgaagatgcggtgaagaagctgaaggcgatgggccgaaagtccgtgaagtctgtcctgtgggtgtcagccgatgggctccgagtggtggacgacaaaaccaaggatcttctggtcgaccagaccatcgaaaaggtctccttttgtgctcctgaccgcaacctggacaaggctttctcctatatctgtcgtgacgggactacccgccgctggatctgccactgttttctggcactgaaggactccggcgagaggctgagccacgctgtgggctgtgcttttgccgcctgcctggagcgaaaacagcgacgggagaaggaatgtggggtcacggccgccttcgatgccagccgcaccagcttcgcccgcgagggctccttccgcctgtctgggggtgggcggcctgctgagcgagaggccccggacaagaagaaagcagaggcagcagctgcccccactgtggctcctggccctgcccagcctgggcacgtgtccccgacaccagccaccacatcccctggtgagaagggtgaggcaggcacccctgtggctgcaggcaccactgcggccgccatcccccggcgccatgcacccctggagcagctggttcgccagggctccttccgtgggttcccagcactcagccagaagaactcgcctttcaaacggcagctgagcctacggctgaatgagctgccatccacgctgcagcgccgcactgacttccaggtgaagggcacagtgcctgagatggagcctcctggtgccggcgacagtgacagcatcaacgctctgtgcacacagatcagttcatcttttgccagtgctggagcgccagcaccagggccaccacctgccacaacagggacttctgcctggggtgagccctccgtgccccctgcagctgccttccagcctgggcacaagcggacaccttcagaggctgagcgatggctggaggaggtgtcacaggtggccaaggcccagcagcagcagcagcagcaacagcaacagcagcagcagcagcagcagcaacagcagcaagcagcctcagtggccccagtgcccaccatgcctcctgccctgcagcctttccccgcccccgtggggccctttgacgctgcacctgcccaagtggccgtgttcctgccacccccacacatgcagcccccttttgtgcccgcctacccgggcttgggctacccaccgatgccccgggtgcccgtggtgggcatcacaccctcacagatggtggcaaacgccttctgctcagccgcccagctccagcctcagcctgccactctgcttgggaaagctggggccttcccgccccctgccatacccagtgcccctgggagccaggcccgccctcgccccaatggggccccctggccccctgagccagcgcctgccccagctccagagttggacccctttgaggcccagtgggcggcattagaaggcaaagccactgtagagaaaccctccaaccccttttctggcgacctgcaaaagacattcgagattgaactgtag

SEQ ID NO: 33 - DNA, 918 bp, Homo sapiens
atgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccttcctccatggatctgcttattcaggacagccctgattcttccaccagtcccaaaggcaaacaacccacttctgcagagaagagtgtcgcaaaaaaggaagacaaggtcccggtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctttactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgccttggaagctgctggggaaggccttaatgtaatacagcagaccactaggtattttagtactccacaaaccatggatttattcctaaactactccatgaacatgcaacctgaagacgtgtga

SEQ ID NO: 34 - DNA, 759 bp, Homo sapiens
atgggggtactgctcacacagaggacgctgctcagtctggtccttgcactcctgtttccaagcatggcgagcatggcggctataggcagctgctcgaaagagtaccgcgtgctccttggccagctccagaagcagacagatctcatgcaggacaccagcagactcctggacccctatatacgtatccaaggcctggatgttcctaaactgagagagcactgcagggagcgccccggggccttccccagtgaggagaccctgagggggctgggcaggcggggcttcctgcagaccctcaatgccacactgggctgcgtcctgcacagactggccgacttagagcagcgcctccccaaggcccaggatttggagaggtctgggctgaacatcgaggacttggagaagctgcagatggcgaggccgaacatcctcgggctcaggaacaacatctactgcatggcccagctgctggacaactcagacacggctgagcccacgaaggctggccggggggcctctcagccgcccacccccacccctgcctcggatgcttttcagcgcaagctggagggctgcaggttcctgcatggctaccatcgcttcatgcactcagtggggcgggtcttcagcaagtggggggagagcccgaaccggagccggagacacagcccccaccaggccctgaggaagggggtgcgcaggaccagaccctccaggaaaggcaagagactcatgaccaggggacagctgccccggtag

SEQ ID NO: 35 - DNA, 2940 bp, Homo sapiens
atggctctatttgcagtctttcagacaacattcttcttaacattgctgtccttgaggacttaccagagtgaagtcttggctgaacgtttaccattgactcctgtatcacttaaagtttccaccaattctacgcgtcagagtttgcacttacaatggactgtccacaaccttccttatcatcaggaattgaaaatggtatttcagatccagatcagtaggattgaaacatccaatgtcatctgggtggggaattacagcaccactgtgaagtggaaccaggttctgcattggagctgggaatctgagctccctttggaatgtgccacacactttgtaagaataaagagtttggtggacgatgccaagttccctgagccaaatttctggagcaactggagttcctgggaggaagtcagtgtacaagattctactggacaggatatattgttcgttttccctaaagataagctggtggaagaaggcaccaatgttaccatttgttacgtttctaggaacattcaaaataatgtatcctgttatttggaagggaaacagattcatggagaacaacttgatccacatgtaactgcattcaacttgaatagtgtgcctttcattaggaataaagggacaaatatctattgtgaggcaagtcaaggaaatgtcagtgaaggcatgaaaggcatcgttctttttgtctcaaaagtacttgaggagcccaaggacttttcttgtgaaaccgaggacttcaagactttgcactgtacttgggatcctgggacggacactgccttggggtggtctaaacaaccttcccaaagctacactttatttgaatcattttctggggaaaagaaactttgtacacacaaaaactggtgtaattggcaaataactcaagactcacaagaaacctataacttcacactcatagctgaaaattacttaaggaagagaagtgtcaatatcctttttaacctgactcatcgagtttatttaatgaatccttttagtgtcaactttgaaaatgtaaatgccacaaatgccatcatgacctggaaggtgcactccataaggaataatttcacatatttgtgtcagattgaactccatggtgaaggaaaaatgatgcaatacaatgtttccatcaaggtgaacggtgagtacttcttaagtgaactggaacctgccacagagtacatggcgcgagtacggtgtgctgatgccagccacttctggaaatggagtgaatggagtggtcagaacttcaccacacttgaagctgctccctcagaggcccctgatgtctggagaattgtgagcttggagccaggaaatcatactgtgaccttattctggaagccattatcaaaactgcatgccaatggaaagatcctgttctataatgtagttgtagaaaacctagacaaaccatccagttcagagctccattccattccagcaccagccaacagcacaaaactaatccttgacaggtgttcctaccaaatctgcgtcatagccaacaacagtgtgggtgcttctcctgcttctgtaatagtcatctctgcagaccccgaaaacaaagaggttgaggaagaaagaattgcaggcacagagggtggattctctctgtcttggaaaccccaacctggagatgttataggctatgttgtggactggtgtgaccatacccaggatgtgctcggtgatttccagtggaagaatgtaggtcccaataccacaagcacagtcattagcacagatgcttttaggccaggagttcgatatgacttcagaatttatgggttatctacaaaaaggattgcttgtttattagagaaaaaaacaggatactctcaggaacttgctccttcagacaaccctcacgtgctggtggatacattgacatcccactccttcactctgagttggaaagattactctactgaatctcaacctggttttatacaagggtaccatgtctatctgaaatccaaggcgaggcagtgccacccacgatttgaaaaggcagttctttcagatggttcagaatgttgcaaatacaaaattgacaacccggaagaaaaggcattgattgtggacaacctaaagccagaatccttctatgagtttttcatcactccattcactagtgctggtgaaggccccagtgctacgttcacgaaggtcacgactccggatgaacactcctcgatgctgattcatatcctactgcccatggttttctgcgtcttgctcatcatggtcatgtgctacttgaaaagtcagtggatcaaggagacctgttatcctgacatccctgacccttacaagagcagcatcctgtcattaataaaattcaaggagaaccctcacctaataataatgaatgtcagtgactgtatcccagatgctattgaagttgtaagcaagccagaagggacaaagatacagttcctaggcactaggaagtcactcacagaaaccgagttgactaagcctaactacctttatctccttccaacagaaaagaatcactctggccctggcccctgcatctgttttgagaacttgacctataaccaggcagcttctgactctggctcttgtggccatgttccagtatccccaaaagccccaagtatgctgggactaatgacctcacctgaaaatgtactaaaggcactagaaaaaaactacatgaactccctgggagaaatcccagctggagaaacaagtttgaattatgtgtcccagttggcttcacccatgtttggagacaaggacagtctcccaacaaacccagtagaggcaccacactgttcagagtataaaatgcaaatggcagtctccctgcgtcttgccttgcctcccccgaccgagaatagcagcctctcctcaattacccttttagatccaggtgaacactactgctaa

SEQ ID NO: 36 - DNA, 798 bp, Homo sapiens
atgcacttctacagactattccttggggccacacgtaggttcttgaatcccgaatggaaaggggagattgataactggtgtgtttatgttcttacaagtcttctgccttttaaaatccagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactga

SEQ ID NO: 37 - DNA, 1083 bp, Homo sapiens
atggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactga

SEQ ID NO: 38 - DNA, 953 bp, Homo sapiens
tgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtga

SEQ ID NO: 39 - DNA, 621 bp, Homo sapiens
atgtcggggcccgggacggccgcggtagcgctgctcccggcggtcctgctggccttgctggcgccctgggcgggccgagggggcgccgccgcacccactgcacccaacggcacgctggaggccgagctggagcgccgctgggagagcctggtggcgctctcgttggcgcgcctgccggtggcagcgcagcccaaggaggcggccgtccagagcggcgccggcgactacctgctgggcatcaagcggctgcggcggctctactgcaacgtgggcatcggcttccacctccaggcgctccccgacggccgcatcggcggcgcgcacgcggacacccgcgacagcctgctggagctctcgcccgtggagcggggcgtggtgagcatcttcggcgtggccagccggttcttcgtggccatgagcagcaagggcaagctctatggctcgcccttcttcaccgatgagtgcacgttcaaggagattctccttcccaacaactacaacgcctacgagtcctacaagtaccccggcatgttcatcgccctgagcaagaatgggaagaccaagaaggggaaccgagtgtcgcccaccatgaaggtcacccacttcctccccaggctgtga

SEQ ID NO: 40 - DNA, 1443 bp, Homo sapiens
atggaggtggcgccggagcagccgcgctggatggcgcacccggccgtgctgaatgcgcagcaccccgactcacaccacccgggcctggcgcacaactacatggaacccgcgcagctgctgcctccagacgaggtggacgtcttcttcaatcacctcgactcgcagggcaacccctactatgccaaccccgctcacgcgcgggcgcgcgtctcctacagccccgcgcacgcccgcctgaccggaggccagatgtgccgcccacacttgttgcacagcccgggtttgccctggctggacgggggcaaagcagccctctctgccgctgcggcccaccaccacaacccctggaccgtgagccccttctccaagacgccactgcacccctcagctgctggaggccctggaggcccactctctgtgtacccaggggctgggggtgggagcgggggaggcagcgggagctcagtggcctccctcacccctacagcagcccactctggctcccaccttttcggcttcccacccacgccacccaaagaagtgtctcctgaccctagcaccacgggggctgcgtctccagcctcatcttccgcggggggtagtgcagcccgaggagaggacaaggacggcgtcaagtaccaggtgtcactgacggagagcatgaagatggaaagtggcagtcccctgcgcccaggcctagctactatgggcacccagcctgctacacaccaccccatccccacctacccctcctatgtgccggcggctgcccacgactacagcagcggactcttccaccccggaggcttcctggggggaccggcctccagcttcacccctaagcagcgcagcaaggctcgttcctgttcagaaggccgggagtgtgtcaactgtggggccacagccacccctctctggcggcgggacggcaccggccactacctgtgcaatgcctgtggcctctaccacaagatgaatgggcagaaccgaccactcatcaagcccaagcgaagactgtcggccgccagaagagccggcacctgttgtgcaaattgtcagacgacaaccaccaccttatggcgccgaaacgccaacggggaccctgtctgcaacgcctgtggcctctactacaagctgcacaatgttaacaggccactgaccatgaagaaggaagggatccagactcggaaccggaagatgtccaacaagtccaagaagagcaagaaaggggcggagtgcttcgaggagctgtcaaagtgcatgcaggagaagtcatcccccttcagtgcagctgccctggctggacacatggcacctgtgggccacctcccgcccttcagccactccggacacatcctgcccactccgacgcccatccacccctcctccagcctctccttcggccacccccacccgtccagcatggtgaccgccatgggctag

SEQ ID NO: 41 - DNA, 1335 bp, Homo sapiens
atggaggtgacggcggaccagccgcgctgggtgagccaccaccaccccgccgtgctcaacgggcagcacccggacacgcaccacccgggcctcagccactcctacatggacgcggcgcagtacccgctgccggaggaggtggatgtgctttttaacatcgacggtcaaggcaaccacgtcccgccctactacggaaactcggtcagggccacggtgcagaggtaccctccgacccaccacgggagccaggtgtgccgcccgcctctgcttcatggatccctaccctggctggacggcggcaaagccctgggcagccaccacaccgcctccccctggaatctcagccccttctccaagacgtccatccaccacggctccccggggcccctctccgtctaccccccggcctcgtcctcctccttgtcggggggccacgccagcccgcacctcttcaccttcccgcccaccccgccgaaggacgtctccccggacccatcgctgtccaccccaggctcggccggctcggcccggcaggacgagaaagagtgcctcaagtaccaggtgcccctgcccgacagcatgaagctggagtcgtcccactcccgtggcagcatgaccgccctgggtggagcctcctcgtcgacccaccaccccatcaccacctacccgccctacgtgcccgagtacagctccggactcttcccccccagcagcctgctgggcggctcccccaccggcttcggatgcaagtccaggcccaaggcccggtccagcacagaaggcagggagtgtgtgaactgtggggcaacctcgaccccactgtggcggcgagatggcacgggacactacctgtgcaacgcctgcgggctctatcacaaaatgaacggacagaaccggcccctcattaagcccaagcgaaggctgtctgcagccaggagagcagggacgtcctgtgcgaactgtcagaccaccacaaccacactctggaggaggaatgccaatggggaccctgtctgcaatgcctgtgggctctactacaagcttcacaatattaacagacccctgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagcctgtcctttggaccacaccacccctccagcatggtcaccgccatgggttag

SEQ ID NO: 42 - DNA, 1329 bp, Homo sapiens
atgtatcagagcttggccatggccgccaaccacgggccgccccccggtgcctacgaggcgggcggccccggcgccttcatgcacggcgcgggcgccgcgtcctcgccagtctacgtgcccacaccgcgggtgccctcctccgtgctgggcctgtcctacctccagggcggaggcgcgggctctgcgtccggaggcgcctcgggcggcagctccggtggggccgcgtctggtgcggggcccgggacccagcagggcagcccgggatggagccaggcgggagccgacggagccgcttacaccccgccgccggtgtcgccgcgcttctccttcccggggaccaccgggtccctggcggccgccgccgccgctgccgcggcccgggaagctgcggcctacagcagtggcggcggagcggcgggtgcgggcctggcgggccgcgagcagtacgggcgcgccggcttcgcgggctcctactccagcccctacccggcttacatggccgacgtgggcgcgtcctgggccgcagccgccgccgcctccgccggccccttcgacagcccggtcctgcacagcctgcccggccgggccaacccggccgcccgacaccccaatctcgatatgtttgacgacttctcagaaggcagagagtgtgtcaactgtggggctatgtccaccccgctctggaggcgagatgggacgggtcactatctgtgcaacgcctgcggcctctaccacaagatgaacggcatcaaccggccgctcatcaagcctcagcgccggctgtccgcctcccgccgagtgggcctctcctgtgccaactgccagaccaccaccaccacgctgtggcgccgcaatgcggagggcgagcctgtgtgcaatgcctgcggcctctacatgaagctccacggggtccccaggcctcttgcaatgcggaaagaggggatccaaaccagaaaacggaagcccaagaacctgaataaatctaagacaccagcagctccttcaggcagtgagagccttcctcccgccagcggtgcttccagcaactccagcaacgccaccaccagcagcagcgaggagatgcgtcccatcaagacggagcctggcctgtcatctcactacgggcacagcagctccgtgtcccagacgttctcagtcagtgcgatgtctggccatgggccctccatccaccctgtcctctcggccctgaagctctccccacaaggctatgcgtctcccgtcagccagtctccacagaccagctccaagcaggactcttggaacagcctggtcttggccgacagtcacggggacataatcactgcgtaa

SEQ ID NO: 43 - DNA, 1194 bp, Homo sapiens
atgtaccagagcctggcgctggccgcgagcccccgccaggccgcctacgccgactcgggctccttcctgcacgctccgggcgccggctctccgatgtttgtgccgccggcgcgcgtcccctcgatgctgtcctacctgtccgggtgtgagccgagcccgcagccccccgagctcgctgcgcgccccggctgggcgcagacagccaccgcggattcgtcggccttcggcccgggcagtccgcaccccccagccgcgcacccgcccggggccaccgccttccctttcgcgcacagcccctcggggcccggcagcggcggcagcgcggggggccgagacggcagtgcctaccagggcgcgctgttgcctcgagaacagttcgcggccccgcttgggcggccggtggggacctcgtactccgccacctacccggcctacgtgagccccgacgtggcccagtcctggactgccgggcccttcgatggcagcgtcctgcacggcctcccaggccgcaggcccaccttcgtgtccgacttcttggaggagttcccgggtgagggtcgtgagtgtgtcaactgcggggccctgtccacaccgctgtggcgccgagatggcaccggccactacctgtgcaatgcctgcggcctctaccacaagatgaatggcgtcaaccggccgctcgttcggcctcagaagcgcctgtcctcgtcccgccgcgccggcctctgctgcaccaactgccacacgaccaacaccacgctgtggcggcggaactcggagggggagcccgtgtgcaatgcctgcggcctctacatgaagctgcacggggtgccgcggcctctggctatgaagaaagaaagcatccagacacggaagcggaagccaaagaccatcgccaaggccaggggctcctcaggatccacaaggaatgcctcggcctccccatctgctgtcgccagcactgacagctcagcagccacttccaaagccaagcccagcctggcgtccccagtgtgccctgggcccagcatggccccccaggcctctggccaggaggatgactctcttgcccccggccacttggagttcaagttcgagcctgaggactttgccttcccctccacggccccgagcccccaggctggcctcaggggggctctgcgccaagaggcctggtgtgcgctggccttggcctag

SEQ ID NO: 44 - DNA, 1788 bp, Homo sapiens
atggccttgactgacggcggctggtgcttgccgaagcgcttcggggccgcgggtgcggacgccagcgactccagagcctttccagcgcgggagccctccacgccgccttcccccatctcttcctcgtcctcctcctgctcccggggcggagagcggggccccggcggcgccagcaactgcgggacgcctcagctcgacacggaggcggcggccggacccccggcccgctcgctgctgctcagttcctacgcttcgcatcccttcggggctccccacggaccttcggcgcctggggtcgcgggccccgggggcaacctgtcgagctgggaggacttgctgctgttcactgacctcgaccaagccgcgaccgccagcaagctgctgtggtccagccgcggcgccaagctgagccccttcgcacccgagcagccggaggagatgtaccagaccctcgccgctctctccagccagggtccggccgcctacgacggcgcgcccggcggcttcgtgcactctgcggccgcggcggcagcagccgcggcggcggccagctccccggtctacgtgcccaccacccgcgtgggttccatgctgcccggcctaccgtaccacctgcaggggtcgggcagtgggccagccaaccacgcgggcggcgcgggcgcgcaccccggctggcctcaggcctcggccgacagccctccatacggcagcggaggcggcgcggctggcggcggggccgcggggcctggcggcgctggctcagccgcggcgcacgtctcggcgcgcttcccctactctcccagcccgcccatggccaacggcgccgcgcgggagccgggaggctacgcggcggcgggcagtgggggcgcgggaggcgtgagcggcggcggcagtagcctggcggccatgggcggccgcgagccccagtacagctcgctgtcggccgcgcggccgctgaacgggacgtaccaccaccaccaccaccaccaccaccaccatccgagcccctactcgccctacgtgggggcgccactgacgcctgcctggcccgccggacccttcgagaccccggtgctgcacagcctgcagagccgcgccggagccccgctcccggtgccccggggtcccagtgcagacctgctggaggacctgtccgagagccgcgagtgcgtgaactgcggctccatccagacgccgctgtggcggcgggacggcaccggccactacctgtgcaacgcctgcgggctctacagcaagatgaacggcctcagccggcccctcatcaagccgcagaagcgcgtgccttcatcacggcggcttggattgtcctgtgccaactgtcacaccacaactaccaccttatggcgcagaaacgccgagggtgaacccgtgtgcaatgcttgtggactctacatgaaactccatggggtgcccagaccacttgctatgaaaaaagagggaattcaaaccaggaaacgaaaacctaagaacataaataaatcaaagacttgctctggtaatagcaataattccattcccatgactccaacttccacctcttctaactcagatgattgcagcaaaaatacttcccccacaacacaacctacagcctcaggggcgggtgccccggtgatgactggtgcgggagagagcaccaatcccgagaacagcgagctcaagtattcgggtcaagatgggctctacataggcgtcagtctcgcctcgccggccgaagtcacgtcctccgtgcgaccggattcctggtgcgccctggccctggcctga

SEQ ID NO: 45 - DNA, 1674 bp, Homo sapiens
atggtgtccaagctcacgtcgctccagcaagaactcctgagcgccctgctgagctccggggtcaccaaggaggtgctggttcaggccttggaggagttgctgccatccccgaacttcggggtgaagctggagacgctgcccctgtcccctggcagcggggccgagcccgacaccaagccggtcttccatactctcaccaacggccacgccaagggccgcttgtccggcgacgagggctccgaggacggcgacgactatgacacacctcccatcctcaaggagctgcaggcgctcaacaccgaggaggcggcggagcagcgggcggaggtggaccggatgctcagtgaggacccttggagggctgctaaaatgatcaagggttacatgcagcaacacaacatcccccagagggaggtggtcgatgtcaccggcctgaaccagtcgcacctctcccagcatctcaacaagggcacccctatgaagacccagaagcgtgccgctctgtacacctggtacgtcagaaagcaacgagagatcctccgacaattcaaccagacagtccagagttctggaaatatgacagacaaaagcagtcaggatcagctgctgtttctctttccagagttcagtcaacagagccatgggcctgggcagtccgatgatgcctgctctgagcccaccaacaagaagatgcgccgcaaccggttcaaatgggggcccgcgtcccagcaaatcttgtaccaggcctacgatcggcaaaagaaccccagcaaggaagagagagaggccttagtggaggaatgcaacagggcagaatgtttgcagcgaggggtgtccccctccaaagcccacggcctgggctccaacttggtcactgaggtccgtgtctacaactggtttgcaaaccgcaggaaggaggaggcattccggcaaaagctggccatggacgcctatagctccaaccagactcacagcctgaaccctctgctctcccacggctccccccaccaccagcccagctcctctcctccaaacaagctgtcaggagtgcgctacagccagcagggaaacaatgagatcacttcctcctcaacaatcagtcaccatggcaacagcgccatggtgaccagccagtcggttttacagcaagtctccccagccagcctggacccaggccacaatctcctctcacctgatggtaaaatgatctcagtctcaggaggaggtttgcccccagtcagcaccttgacgaatatccacagcctctcccaccataatccccagcaatctcaaaacctcatcatgacacccctctctggagtcatggcaattgcacaaagcctcaacacctcccaagcacagagtgtccctgtcatcaacagtgtggccggcagcctggcagccctgcagcccgtccagttctcccagcagctgcacagccctcaccagcagcccctcatgcagcagagcccaggcagccacatggcccagcagcccttcatggcagctgtgactcagctgcagaactcacacatgtacgcacacaagcaggaacccccccagtattcccacacctcccggtttccatctgcaatggtggtcacagataccagcagcatcagtacactcaccaacatgtcttcaagtaaacagtgtcctctacaagcctggtga

SEQ ID NO: 46 - DNA, 1887 bp, Rattus norvegicus
atggtttctaagttgagccagctgcagacggagctcctggctgctctgctcgagtcgggcctgagcaaagaggctctgatccaggctctgggggagcccgggccctacctgatggttggagatggtcccctggacaagggggagtcctgcggtgggactcgaggggacctgaccgagctgcccaatggcctgggggagacgcgtggctcggaagatgacacggatgacgatggggaagacttcgcgccacccattctgaaagagctggagaacctcagcccagaggaggcagcccaccagaaagccgtggtggagtcacttcttcaggaggacccatggcgcgtggcaaagatggtcaagtcgtacctgcagcaacacaacatcccccagcgggaggtggtggacactacgggtctcaaccagtcccacctgtcccagcacctcaacaagggcacccccatgaagacgcagaagcgggccgcgctgtacacctggtacgtccgcaagcagcgagaggtggctcagcaattcacccacgcggggcagggcggactgattgaagagcccacaggtgatgagctgccaaccaaaaaggggcggaggaaccggttcaagtggggccccgcatcccagcagatcctgttccaggcttacgagaggcagaagaaccccagcaaggaagagcgagagaccttggtggaggagtgcaatagggcggagtgcatccagagaggggtgtcaccatcgcaggcccaggggctaggctccaaccttgtcaccgaggtgcgtgtctacaactggtttgccaaccggcgcaaggaagaagcctttcggcataagctggccatggacacgtataacgggcctccacccgggccaggccccggccctgcgctacctgcccacagttccccgggcctgcccacaaccaccctctctcccagtaaggtccacggtgtgcggtatggacagtctgcaaccagcgaggcagctgaggtgccctccagcagcggaggtcccttagtcacagtgtctgcggccttacaccaagtgtcccccacaggcttggagcccagcagcctgctgagcaccgaggccaagctggtctcagccacggggggtcccctgcctcccgtcagcaccctgacagcactgcacagcttggagcagacgtctccaggtctcaaccagcagccgcagaaccttatcatggcctcgctgcctggggtcatgaccatcggcccaggggagcccgcctccctgggtcccacgttcactaacacgggtgcctctaccctggtcattggtctggcctccacacaggcacagagcgtgccagtcatcaacagcatggggagcagcctgaccaccctgcagccggtccagttttcccagccactgcacccttcctatcagcagcctctcatgccccctgtacagagccacgtggcccagagtcccttcatggcaaccatggcccagctgcagagcccccacgccctgtacagccacaagcctgaggtggcccagtacacgcatacaagcctgcttccgcagaccatgctgatcacagacaccaacctcagcacccttgccagcctcacgcccaccaagcaggtcttcacctcagacacagaggcctccagtgagcctgggcttcatgagccgtcgtctccagccacaaccattcacatccccagccaggacccgtcaaacatccagcacctgcagcctgctcaccggctcagcaccagtcccacagtgtcctccagcagcctggtgttgtaccagagttctgactccaacgggcacagccacctgctgccatccaaccacggtgtcatcgagacttttatctccacccagatggcctcctcctcccagtaa

SEQ ID NO: 47 - DNA, 1407 bp, Mus musculus
atgttagggactgtgaagatggaagggcatgagagcaacgactggaacagctactacgcggacacgcaggaggcctactcctctgtccctgtcagcaacatgaactccggcctgggctctatgaactccatgaacacctacatgaccatgaacaccatgaccacgagcggcaacatgaccccggcttccttcaacatgtcctacgccaacacgggcttaggggccggcctgagtcccggtgctgtggctggcatgccaggggcctctgcaggcgccatgaacagcatgactgcggcgggcgtcacggccatgggtacggcgctgagcccgggaggcatgggctccatgggcgcgcagcccgtcacctccatgaacggcctgggtccctacgccgccgccatgaacccgtgcatgagtcccatggcgtacgcgccgtccaacctgggccgcagccgcgcggggggcggcggcgacgccaagacattcaagcgcagctaccctcacgccaagccgccttactcctacatctcgctcatcacgatggccatccagcaggcgcccagcaagatgctcacgctgagcgagatctaccagtggatcatggacctcttcccctattaccgccagaaccagcagcgctggcagaactccatccgccactcgctgtccttcaacgattgtttcgtcaaggtggcacgatccccagacaagccaggcaagggctcctactggacgctgcacccggactccggcaacatgttcgagaacggctgctacttgcgccgccaaaagcgcttcaagtgtgagaagcagccgggggccggaggtgggagtgggggcggcggctccaaagggggcccagaaagtcgcaaggacccctcaggcccggggaaccccagcgccgagtcaccccttcattggggtgtgcacggaaaggctagccagctagagggcgcgccggcccccgggcccgccgccagcccccagactctggaccacagcggggccacggcgacagggggcgcttcggagttgaagtctccagcgtcttcatctgcgccccccataagctccgggccaggggcgctggcatctgtacccccctctcacccggctcacggcctggcaccccacgaatctcagctgcatctgaaaggggatccccactactcctttaatcaccccttctccatcaacaacctcatgtcctcctccgagcaacagcacaagctggacttcaaggcatacgagcaggcgctgcagtactctccttatggcgctaccttgcccgccagtctgccccttggcagcgcctcagtggccacgaggagccccatcgagccctcagccctggagccagcctactaccaaggtgtgtattccagacccgtgctaaatacttcctag

SEQ ID NO: 48 - DNA, 1062 bp, Mus musculus
atgctgggctcagtgaagatggaggctcatgacctggccgagtggagctactacccggaggcgggcgaggtgtattctccagtgaatcctgtgcccaccatggcccctctcaactcctacatgaccttgaacccactcagctctccctaccctcccggagggcttcaggcctccccactgcctacaggacccctggcacccccagcccccactgcgcccttggggcccaccttcccaagcttgggcactggtggcagcaccggaggcagtgcttccgggtatgtagccccagggcccgggcttgtacatggaaaagagatggcaaaggggtaccggcggccactggcccacgccaaaccaccatattcctacatctctctcataaccatggctattcagcaggctccaggcaagatgctgaccctgagtgaaatctaccaatggatcatggacctcttcccgtactaccgggagaaccagcaacgttggcagaactccatccggcattcgctgtccttcaatgactgcttcgtcaaggtggcacgctccccagacaagccaggcaaaggctcctactgggccttgcatcccagctctgggaacatgtttgagaacggatgctatctccgccggcagaagcgcttcaagctggaggagaaggcaaagaaaggaaacagcgccacatcggccagcaggaatggtactgcggggtcagccacctctgccaccactacagctgccactgcagtcacctccccggctcagccccagcctacgccatctgagcccgaggcccagagtggggatgatgtggggggtctggactgcgcctcacctccttcgtccacaccttatttcagcggcctggagctcccgggggaactaaagttggatgcgccctataacttcaaccaccctttctctatcaacaacctgatgtcagaacagacatcgacaccttccaaactggatgtggggtttgggggctacggggctgagagtggggagcctggagtctactaccagagcctctattcccgctctctgcttaatgcatcctag

SEQ ID NO: 49 - DNA, 1380 bp, Mus musculus
atgctgggagccgtgaagatggaagggctcgagccatccgactggagcagctactacgcggagcccgagggctactcttccgtgagcaacatgaacgccggcctggggatgaatggcatgaacacatacatgagcatgtccgcggctgccatgggcggcggttccggcaacatgagcgcgggctccatgaacatgtcatcctatgtgggcgctggaatgagcccgtcgctagctggcatgtccccgggcgccggcgccatggcgggcatgagcggctcagccggggcggccggcgtggcgggcatgggacctcacctgagtccgagtctgagcccgctcgggggacaggcggccggggccatgggtggccttgccccctacgccaacatgaactcgatgagccccatgtacgggcaggccggcctgagccgcgctcgggaccccaagacataccgacgcagctacacacacgccaaacctccctactcgtacatctcgctcatcaccatggccatccagcagagccccaacaagatgctgacgctgagcgagatctatcagtggatcatggacctcttccctttctaccggcagaaccagcagcgctggcagaactccatccgccactctctctccttcaacgactgctttctcaaggtgccccgctcgccagacaagcctggcaagggctccttctggaccctgcacccagactcgggcaacatgttcgagaacggctgctacctgcgccgccagaagcgcttcaagtgtgagaagcaactggcactgaaggaagccgcgggtgcggccagtagcggaggcaagaagaccgctcctgggtcccaggcctctcaggctcagctcggggaggccgcgggctcggcctccgagactccggcgggcaccgagtccccccattccagcgcttctccgtgtcaggagcacaagcgaggtggcctaagcgagctaaagggagcacctgcctctgcgctgagtcctcccgagccggcgccctcgcctgggcagcagcagcaggctgcagcccacctgctgggcccacctcaccacccaggcctgccaccagaggcccacctgaagcccgagcaccattacgccttcaaccaccccttctctatcaacaacctcatgtcgtccgagcagcaacatcaccacagccaccaccaccatcagccccacaaaatggacctcaaggcctacgaacaggtcatgcactacccagggggctatggttcccccatgccaggcagcttggccatgggcccagtcacgaacaaagcgggcctggatgcctcgcccctggctgcagacacttcctactaccaaggagtgtactccaggcctattatgaactcatcctaa

SEQ ID NO: 50 - DNA, 1053 bp, Homo sapiens
atgctgggctcagtgaagatggaggcccatgacctggccgagtggagctactacccggaggcgggcgaggtctactcgccggtgaccccagtgcccaccatggcccccctcaactcctacatgaccctgaatcctctaagctctccctatccccctggggggctccctgcctccccactgccctcaggacccctggcacccccagcacctgcagcccccctggggcccactttcccaggcctgggtgtcagcggtggcagcagcagctccgggtacggggccccgggtcctgggctggtgcacgggaaggagatgccgaaggggtatcggcggcccctggcacacgccaagccaccgtattcctatatctcactcatcaccatggccatccagcaggcgccgggcaagatgctgaccttgagtgaaatctaccagtggatcatggacctcttcccttactaccgggagaatcagcagcgctggcagaactccattcgccactcgctgtctttcaacgactgcttcgtcaaggtggcgcgttccccagacaagcctggcaagggctcctactgggccctacaccccagctcagggaacatgtttgagaatggctgctacctgcgccgccagaaacgcttcaagctggaggagaaggtgaaaaaagggggcagcggggctgccaccaccaccaggaacgggacagggtctgctgcctcgaccaccacccccgcggccacagtcacctccccgccccagcccccgcctccagcccctgagcctgaggcccagggcggggaagatgtgggggctctggactgtggctcacccgcttcctccacaccctatttcactggcctggagctcccaggggagctgaagctggacgcgccctacaacttcaaccaccctttctccatcaacaacctaatgtcagaacagacaccagcacctcccaaactggacgtggggtttgggggctacggggctgaaggtggggagcctggagtctactaccagggcctctattcccgctctttgcttaatgcatcctag

SEQ ID NO: 51 - DNA, 1422 bp, Homo sapiens
atgttaggaactgtgaagatggaagggcatgaaaccagcgactggaacagctactacgcagacacgcaggaggcctactcctcggtcccggtcagcaacatgaactcaggcctgggctccatgaactccatgaacacctacatgaccatgaacaccatgactacgagcggcaacatgaccccggcgtccttcaacatgtcctatgccaacccggccttaggggccggcctgagtcccggcgcagtagccggcatgccggggggctcggcgggcgccatgaacagcatgactgcggccggcgtgacggccatgggtacggcgctgagcccgagcggcatgggcgccatgggtgcgcagcaggcggcctccatgatgaatggcctgggcccctacgcggccgccatgaacccgtgcatgagccccatggcgtacgcgccgtccaacctgggccgcagccgcgcgggcggcggcggcgacgccaagacgttcaagcgcagttacccgcacgccaagccgccctactcgtacatctcgctcatcaccatggccatccagcgggcgcccagcaagatgctcacgctgagcgagatctaccagtggatcatggacctcttcccctattaccggcagaaccagcagcgctggcagaactccatccgccactcgctgtccttcaatgactgcttcgtcaaggtggcacgctccccggacaagccgggcaagggctcctactggacgctgcacccggactccggcaacatgttcgagaacggctgctacttgcgccgccagaagcgcttcaagtgcgagaagcagccgggggccggcggcgggggcgggagcggaagcgggggcagcggcgccaagggcggccctgagagccgcaaggacccctctggcgcctctaaccccagcgccgactcgcccctccatcggggtgtgcacgggaagaccggccagctagagggcgcgccggccccgggcccggccgccagcccccagactctggaccacagtggggcgacggcgacagggggcgcctcggagttgaagactccagcctcctcaactgcgccccccataagctccgggcccggggcgctggcctctgtgcccgcctctcacccggcacacggcttggcaccccacgagtcccagctgcacctgaaaggggacccccactactccttcaaccacccgttctccatcaacaacctcatgtcctcctcggagcagcagcataagctggacttcaaggcatacgaacaggcactgcaatactcgccttacggctctacgttgcccgccagcctgcctctaggcagcgcctcggtgaccaccaggagccccatcgagccctcagccctggagccggcgtactaccaaggtgtgtattccagacccgtcctaaacacttcctag

SEQ ID NO: 52 - DNA, 1425 bp, Mus musculus
atgcgactctctaaaacccttgccggcatggatatggccgactacagcgctgccctggacccagcctacaccaccctggagtttgaaaatgtgcaggtgttgaccatgggcaatgacacgtccccatctgaaggtgccaacctcaattcatccaacagcctgggcgtcagtgccctgtgcgccatctgtggcgaccgggccaccggcaaacactacggagcctcgagctgtgacggctgcaaggggttcttcaggaggagcgtgaggaagaaccacatgtactcctgcaggtttagccgacaatgtgtggtagacaaagataagaggaaccagtgtcgttactgcaggcttaagaagtgcttccgggctggcatgaagaaggaagctgtccaaaatgagcgggaccggatcagcacgcggaggtcaagctacgaggacagcagcctgccctccatcaacgcgctcctgcaggcagaggttctgtcccagcagatcacctctcccatctctgggatcaatggcgacattcgggcaaagaagattgccaacatcacagacgtgtgtgagtctatgaaggagcagctgctggtcctggtcgagtgggccaagtacatcccggccttctgcgaactccttctggatgaccaggtggcgctgctcagggcccacgccggtgagcatctgctgcttggagccaccaagaggtccatggtgtttaaggacgtgctgctcctaggcaatgactacatcgtccctcggcactgtccagagctagcggagatgagccgtgtgtccatccgcatcctcgatgagctggtcctgcccttccaagagctgcagattgatgacaatgaatatgcctgcctcaaagccatcatcttctttgatccagatgccaaggggctgagtgacccgggcaagatcaagcggctgcggtcacaggtgcaagtgagcctggaggattacatcaacgaccggcagtacgactctcggggccgctttggagagctgctgctgctgttgcccacgctgcagagcatcacctggcagatgatcgaacagatccagttcatcaagctcttcggcatggccaagattgacaacctgctgcaggagatgcttctcggagggtctgccagtgatgcaccccacacccaccaccccctgcaccctcacctgatgcaagaacacatgggcaccaatgtcattgttgctaacacgatgccctctcacctcagcaatggacagatgtgtgagtggccccgacccagggggcaggcagccactcccgagactccacagccatcaccaccaagtggctcgggatctgaatcctacaagctcctgccaggagccatcaccaccatcgtcaagcctccctctgccattccccagccaacgatcaccaagcaagaagccatctag

SEQ ID NO: 53 - DNA, 1398 bp, Rattus norvegicus
atggacatggctgactacagtgctgccttggacccagcctacaccaccctggagtttgaaaatgtgcaggtgttgaccatgggcaatgacacatccccatctgaaggtgccaacctcaactcatccaacagcctgggtgtcagtgccctgtgtgccatctgtggcgatcgggccactggcaaacactacggagcctcaagctgtgacggctgcaagggattcttcaggaggagcgtgaggaagaaccacatgtactcctgcaggtttagcaggcagtgcgtggtagacaaagataagaggaaccagtgtcgttactgcaggctcaagaagtgcttccgggctggcatgaagaaagaagccgtccaaaatgagcgggatcggatcagcacgcggaggtcaagctacgaggacagcagcctaccctccattaatgcgctcctgcaggcagaggtcctgtctcagcagatcacctcccccatctctgggatcaatggcgacattcgggccaagaagattgccaacatcacggatgtgtgtgagtctatgaaggagcagctgctggttctggtcgaatgggccaagtacatcccggccttctgtgaacttcttctggatgaccaggtggcgctgctcagagcccacgctggtgagcacctgctgcttggagccaccaagaggtccatggtgttcaaggatgtgctgctcctaggcaatgactacatcgtccctcggcactgtccagagctagcagagatgagccgtgtgtccattcgcatcctcgatgagctggtcttgcccttccaagagctgcagatcgatgataatgaatacgcctgcctcaaagccatcatcttctttgacccagatgccaaggggctgagtgacccaggcaagatcaagcggctgcggtcacaggtgcaggtgagcctggaggattacatcaacgaccggcagtatgactctcggggtcgttttggagagctgctgctgctcctgcccactctgcagagcattacctggcagatgatcgagcagatccagttcatcaagctctttggcatggccaagattgacaacctgctgcaggagatgctgcttggagggtctgccagtgacgcgccccacgcccaccaccccctgcaccctcacctgatgcaagaacacatgggcaccaatgtcatagttgccaacacgatgccctctcacctcagcaatggacagatgtgtgagtggccccggcccagggggcaggcagccacccctgagactccacagccatcaccaccaagtggctctggatctgaatcctacaagctcctgccaggagccatcaccaccatcgtcaagcctccctctgccatcccccagccaacgatcaccaagcaggaagccatctag

SEQ ID NO: 54 - DNA, 1398 bp, Mus musculus
atgaacgcacagctgaccatggaggcgatcggcgagctgcacggggtgagccatgagccggtgcccgcccctgctgacctgctgggcggcagccctcacgcgcgcagctccgtgggacaccgcggcagccacctgcctcccgcgcacccgcgttccatgggcatggcgtccctgctggacggcggcagcggaggcagcgattaccaccaccaccaccgcgcccctgagcacagcttggctggccccctgcaccccaccatgaccatggcctgtgaaactcccccaggtatgagcatgcccaccacctacactaccttaacccctctgcagccgctgccgcccatctccaccgtgtccgacaagttccctcaccatcatcaccaccaccatcaccaccaccacccacaccaccaccagcgcctggcgggcaacgtgagcggtagtttcacacttatgcgggatgagcgcgggctggcctctatgaataacctctataccccctaccacaaggacgtggctggcatgggccagagcctctcgcccctctctggctccggtctgggcagcattcacaactcccagcaaggacttccccactatgctcatcccggcgcggctatgcccaccgacaagatgctcaccccaaatggctttgaagcccaccaccctgccatgctcggtcgccacggggagcagcacctcacgcccacctcggccggcatggtacccatcaacggccttcctccgcaccatcctcatgcccacctgaatgcccagggccacggacagctcctgggcacagcccgagagcccaacccttcggtgaccggcgcgcaggtcagcaatggaagtaattcagggcagatggaagagatcaataccaaagaggtggcgcagcgtatcaccaccgagctcaaacgttacagcatcccacaggccatcttcgcgcagagggtgctctgccgttcccaggggaccctttcggacctgctgcgaaaccccaagccctggagcaaactcaagtcgggtcgggagaccttccggaggatgtggaagtggctgcaggagccggagttccagcgcatgtcggcgctccgcttagcagcctgcaaacggaaagagcaagaacatgggaaggacagaggcaacacccccaaaaagcccaggctggtcttcacagacgtccaacgtcgaactctacatgcaatattcaaggaaaataagcgtccgtccaaagaattacaaatcaccatctcccagcagctggggttggagctgagcactgtcagcaacttcttcatgaatgccagaaggaggagtctggacaagtggcaggacgagggcggctccaactcaggcagttcatcgtcctcatcgagcacttgtaccaaagcatga

SEQ ID NO: 55 - DNA, 1002 bp, Xenopus laevis
atggagaagtccaagaatttcaggattgacgctctcctggcgatagatccccccaaggctcagacctccccattggctctggtcacctcgctgtcctcctcgtctctctccgggagccccccgtccgagcacactgacagcctcaggactgactccccctcccctccaaggacttgtggactggtccctaaaccaggtttcctgagcagccaccagcaccccccaaacatgatgtcattgcacccccaggctgctccagggatcccccctcaggccctgtatggacacccgatgtacagctacttggcagcggggcagcacccagctctgtcctacccctactcccagatgcagagcagccaccacccccaccccatggaccccatcaagatcagcgctggcaccttccaactggaccagtggctcagagcctccactgccggcatgatgctgcccaaaatggcagactttaactcccaggcccaatccaacctgctgggaaagtgcagaagaccaaggacagcgtttaccagtcagcagctgttggaactggagcaccaattcaagctgaacaagtacctctccaggccgaaacgctttgaagtggccacttccctgatgctcactgagacgcaggtgaagatctggttccagaacaggcgcatgaaatggaagaggagtaagaaagccaaggagcaggcggcgcaggactcagcagagaaacagcagagggcaggcaagggcagcagcgaggagaagtgctcggatgagctgcaggaagagaagaaatcctaccatctccatcccaggggggagcccatcaaagggaacggccgcctgcagcccagagactatacagacagcgaagaggacgaggaggaggacagggaagaggaggaagaggaagatcacagaggggaggggaagcggttttaccatcattcttctgactgcacatccgaggaagaggagaacagccacaataagcagagcggccactga

SEQ ID NO: 56 - DNA, 1215 bp, Mus musculus
atggaaaaatccaaaaatttccgcatcgacgccctgctggccgtggatcccccgcgagccgcctccacgcagagcgcgcctctggccttggtcacttccctcgcgactacagtatctggtcccggccgcggcggcagcggcggcggggggaccagtagcggggcgagccgtagctgcagtcccgcatcctcggaggccactgcagcgcccggtgaccggctgagagctgagagcccgtcgcccccacgcttgctggctgcacactgcgcgctgctgcccaagcccggattcctgggcgccggaggaggcggcggcgcggcgggtgggccgggcactccccaccaccacgcgcaccctggtgcagcagccgccgcggctgccgctgccgctgccgcggctgccggtggcctggcactggggctgcacccggggggcgcacagggcggcgcgggcctccctgcacaggcggctctctatggacacccggtctacagttattcggcagcagctgcagcggccgcgctagctggccagcacccggcgctttcctactcataccctcaggtgcagggcgcgcaccctgcgcaccctgccgaccccatcaagctgggtgccagcaccttccaactggaccagtggctgcgcgcgtctactgcgggcatgatcctgcccaagatgccggacttcagctgtcaggcgcagtcgaacctcttggggaagtgccgaaggcctcgcacggccttcaccagccagcagctgttggagctggaacaccagttcaagctcaacaagtacctgtctcgacccaagcgttttgaggtggctacctcgctcatgctcaccgagactcaggtgaagatttggttccagaaccgccgaatgaaatggaaacgcagcaaaaaggccaaagagcaggctgcgcaggaggcggagaagcagaagggcggcggcgggggcaccggcaaaggcggcagtgaggagaagacggaagaggagctgatggggcctccggtttcgggggacaaggcaagcggccgtcgcctgcgggacttgcgggacagtgaccctgatgaggacgaggatgatgaagaagaggacaacttcccgtacagcaatggtgccggtgcccatgctgcctcatccgactgctcatctgaggacgactcgcctcctccaagactaggcgggcctggacaccaacctctgccccagtag

SEQ ID NO: 57 - DNA, 1215 bp, Homo sapiens; identical to SEQ ID NO: 56

SEQ ID NO: 58 - DNA, 846 bp, Homo sapiens
atgacttccaaggaggacggcaaggcggcgccgggggaggagcggcggcgcagcccgctggaccacctgcctccgcctgccaactccaacaagccactgacgccgttcagcatcgaggacatcctcaacaagccgtctgtgcggagaagttactcgctgtgcggggcggcgcacctgctggccgccgcggacaagcacgcgcagggcggcttgcccctggcgggccgcgcgctgctctcgcagacctcgccgctgtgcgcgctggaggagctcgccagcaagacgtttaaggggctggaggtcagcgttctgcaggcagccgaaggccgcgacggtatgaccatctttgggcagcggcagacccctaagaagcggcgaaagtcgcgcacggccttcaccaaccaccagatctatgaattggaaaagcgctttctataccagaagtacctgtcccccgccgatcgcgaccaaatcgcgcagcagctgggcctcaccaacgcgcaagtcatcacctggttccagaatcggcgcgctaagctcaagcgggacctggaggagatgaaggccgacgtagagtccgccaagaaactgggccccagcgggcagatggacatcgtggcgctggccgaactcgagcagaactcggaggccacagccggcggtggcggcggctgcggcagggccaagtcgaggcccggctctccggtcctccccccaggcgccccgaaggccccgggcgctggcgccctgcagctctcgcctgcctctccgctcacggaccagccggccagcagccaggactgctcggaggacgaggaagacgaagagatcgacgtggacgattga

SEQ ID NO: 59 - DNA, 1119 bp, Mus musculus
atgttggacggcatcaagatggaggagcacgccctgcgccccgggcccgccactctgggggtgctgctgggctccgactgcccgcatcccgccgtctgcgagggctgccagcggcccatctccgaccgcttcctgatgcgagtcaacgagtcgtcctggcacgaggagtgtttgcagtgcgcggcgtgtcagcaagccctcaccaccagctgctacttccgggatcggaaactgtactgcaaacaagactaccaacagctcttcgcggccaagtgcagcggctgcatggagaagatcgcccccaccgagttcgtgatgcgggcgctggagtgcgtgtaccacctgggctgcttctgctgctgcgtgtgtgaacggcagctacgcaagggcgacgaattcgtgctcaaggagggccagctgctgtgcaagggtgactacgagaaggagaaggacctgctcagctccgtgagccccgacgagtccgactccgtgaagagcgaggatgaagatggggacatgaagccggccaaggggcagggcagtcagagcaagggcagcggggatgacgggaaggacccgcggaggcccaagcgaccccggaccatcctcaccacgcagcagcgaagagccttcaaggcctccttcgaggtctcgtcgaagccttgccgaaaggtccgagagacactggcagctgagacgggcctcagtgtgcgcgtggtccaggtctggtttcagaaccaaagagcaaagatgaagaagctggcgcggcggcaccagcagcagcaggagcagcagaactcccagcggctgggccaggaggtcctgtccagccgcatggagggcatgatggcttcctacacgccgctggccccaccacagcagcagatcgtggccatggaacagagcccctacggcagcagcgaccccttccagcagggcctcacgccgccccaaatgccagggaacgactccatcttccatgacatcgacagcgatacctccttaaccagcctcagcgactgcttcctcggctcctcagacgtgggctccctgcaggcccgcgtggggaaccccatcgaccggctctactccatgcagagttcctacttcgcctcctga

SEQ ID NO: 60 - DNA, 714 bp, Homo sapiens
atgccagcccgccttgagacctgcatctccgacctcgactgcgccagcagcagcggcagtgacctatccggcttcctcaccgacgaggaagactgtgccagactccaacaggcagcctccgcttcggggccgcccgcgccggcccgcaggggcgcgcccaatatctcccgggcgtctgaggttccaggggcacaggacgacgagcaggagaggcggcggcgccgcggccggacgcgggtccgctccgaggcgctgctgcactcgctgcgcaggagccggcgcgtcaaggccaacgatcgcgagcgcaaccgcatgcacaacttgaacgcggccctggacgcactgcgcagcgtgctgccctcgttccccgacgacaccaagctcaccaaaatcgagacgctgcgcttcgcctacaactacatctgggctctggccgagacactgcgcctggcggatcaagggctgcccggaggcggtgcccgggagcgcctcctgccgccgcagtgcgtcccctgcctgcccggtcccccaagccccgccagcgacgcggagtcctggggctcaggtgccgccgccgcctccccgctctctgaccccagtagcccagccgcctccgaagacttcacctaccgccccggcgaccctgttttctccttcccaagcctgcccaaagacttgctccacacaacgccctgtttcattccttaccactag

SEQ ID NO: 61 - DNA, 819 bp, Homo sapiens
atgttcgtcaaatccgagaccttggagttgaaggaggaagaggacgtgttagtgctgctcggatcggcctcccccgccttggcggccctgaccccgctgtcatccagcgccgacgaagaagaggaggaggagccgggcgcgtcaggcggggcgcgtcggcagcgcggggctgaggccgggcagggggcgcggggcggcgtggctgcgggtgcggagggctgccggcccgcacggctgctgggtctggtacacgattgcaaacggcgcccttcccgggcgcgggccgtctcccgaggcgccaagacggccgagacggtgcagcgcatcaagaagacccgtagactgaaggccaacaaccgcgagcgaaaccgcatgcacaacctcaacgcggcactggacgcgctgcgcgaggtgctccccacgttccccgaggacgccaagctcaccaagatcgagaccctgcgcttcgcccacaactacatctgggcactcaccgagaccctgcgcctggcggatcactgcgggggcggcggcgggggcctgccgggggcgctcttctccgaggcagtgttgctgagcccgggaggcgccagcgccgccctgagcagcagcggagacagcccctcgcccgcctccacgtggagttgcaccaacagccccgcgccgtcctcctccgtgtcctccaattccacctccccctacagctgcactttatcgcccgccagcccggccgggtcagacatggactattggcagcccccacctcccgacaagcaccgctatgcacctcacctccccatagccagggattgtatctag

SEQ ID NO: 62 - DNA, 645 bp, Mus musculus
atgacgcctcaaccctcgggtgcgcccactgtccaagtgacccgtgagacggagcggtccttccccagagcctcggaagacgaagtgacctgccccacgtccgccccgcccagccccactcgcacacgggggaactgcgcagaggcggaagagggaggctgccgaggggccccgaggaagctccgggcacggcgcgggggacgcagccggcctaagagcgagttggcactgagcaagcagcgacggagtcggcgaaagaaggccaacgaccgcgagcgcaatcgaatgcacaacctcaactcggcactggacgccctgcgcggtgtcctgcccaccttcccagacgacgcgaagctcaccaagatcgagacgctgcgcttcgcccacaactacatctgggcgctgactcaaacgctgcgcatagcggaccacagcttgtacgcgctggagccgccggcgccgcactgcggggagctgggcagcccaggcggttcccccggggactgggggtccctctactccccagtctcccaggctggcagcctgagtcccgccgcgtcgctggaggagcgacccgggctgctgggggccaccttttccgcctgcttgagcccaggcagtctggctttctcagattttctgtga

SEQ ID NO: 63 - DNA, 711 bp, Homo sapiens
atggaaagctctgccaagatggagagcggcggcgccggccagcagccccagccgcagccccagcagcccttcctgccgcccgcagcctgtttctttgccacggccgcagccgcggcggccgcagccgccgcagcggcagcgcagagcgcgcagcagcagcagcagcagcagcagcagcagcagcaggcgccgcagctgagaccggcggccgacggccagccctcagggggcggtcacaagtcagcgcccaagcaagtcaagcgacagcgctcgtcttcgcccgaactgatgcgctgcaaacgccggctcaacttcagcggctttggctacagcctgccgcagcagcagccggccgccgtggcgcgccgcaacgagcgcgagcgcaaccgcgtcaagttggtcaacctgggctttgccacccttcgggagcacgtccccaacggcgcggccaacaagaagatgagtaaggtggagacactgcgctcggcggtcgagtacatccgcgcgctgcagcagctgctggacgagcatgacgcggtgagcgccgccttccaggcaggcgtcctgtcgcccaccatctcccccaactactccaacgacttgaactccatggccggctcgccggtctcatcctactcgtcggacgagggctcttacgacccgctcagccccgaggagcaggagcttctcgacttcaccaactggttctga

SEQ ID NO: 64 - DNA, 957 bp, Mus musculus
atggagcttctatcgccgccactccgggacatagacttgacaggccccgacggctctctctgctcctttgagacagcagacgacttctatgatgacccgtgtttcgactcaccagacctgcgcttttttgaggacctggacccgcgcctggtgcacatgggagccctcctgaaaccggaggagcacgcacacttccctactgcggtgcacccaggcccaggcgctcgtgaggatgagcatgtgcgcgcgcccagcgggcaccaccaggcgggtcgctgcttgctgtgggcctgcaaggcgtgcaagcgcaagaccaccaacgctgatcgccgcaaggccgccaccatgcgcgagcgccgccgcctgagcaaagtgaatgaggccttcgagacgctcaagcgctgcacgtccagcaacccgaaccagcggctacccaaggtggagatcctgcgcaacgccatccgctacatcgaaggtctgcaggctctgctgcgcgaccaggacgccgcgccccctggcgccgctgccttctacgcacctggaccgctgcccccaggccgtggcagcgagcactacagtggcgactcagatgcatccagcccgcgctccaactgctctgatggcatgatggattacagcggccccccaagcggcccccggcggcagaatggctacgacaccgcctactacagtgaggcggcgcgcgagtccaggccagggaagagtgcggctgtgtcgagcctcgactgcctgtccagcatagtggagcgcatctccacagacagccccgctgcgcctgcgctgcttttggcagatgcaccaccagagtcgcctccgggtccgccagagggggcatccctaagcgacacagaacagggaacccagaccccgtctcccgacgccgcccctcagtgtcctgcaggctcaaaccccaatgcgatttatcaggtgctttga

SEQ ID NO: 65 - DNA, 963 bp, Homo sapiens
atggagctactgtcgccaccgctccgcgacgtagacctgacggcccccgacggctctctctgctcctttgccacaacggacgacttctatgacgacccgtgtttcgactccccggacctgcgcttcttcgaagacctggacccgcgcctgatgcacgtgggcgcgctcctgaaacccgaagagcactcgcacttccccgcggcggtgcacccggccccgggcgcacgtgaggacgagcatgtgcgcgcgcccagcgggcaccaccaggcgggccgctgcctactgtgggcctgcaaggcgtgcaagcgcaagaccaccaacgccgaccgccgcaaggccgccaccatgcgcgagcggcgccgcctgagcaaagtaaatgaggcctttgagacactcaagcgctgcacgtcgagcaatccaaaccagcggttgcccaaggtggagatcctgcgcaacgccatccgctatatcgagggcctgcaggctctgctgcgcgaccaggacgccgcgccccctggcgccgcagccgccttctatgcgccgggcccgctgcccccgggccgcggcggcgagcactacagcggcgactccgacgcgtccagcccgcgctccaactgctccgacggcatgatggactacagcggccccccgagcggcgcccggcggcggaactgctacgaaggcgcctactacaacgaggcgcccagcgaacccaggcccgggaagagtgcggcggtgtcgagcctagactgcctgtccagcatcgtggagcgcatctccaccgagagccctgcggcgcccgccctcctgctggcggacgtgccttctgagtcgcctccgcgcaggcaagaggctgccgcccccagcgagggagagagcagcggcgaccccacccagtcaccggacgccgccccgcagtgccctgcgggtgcgaaccccaacccgatataccaggtgctctga

SEQ ID NO: 66 - DNA, 768 bp, Homo sapiens
atggacgtgatggatggctgccagttctcaccttctgagtacttctacgacggctcctgcataccgtcccccgagggtgaatttggggacgagtttgtgccgcgagtggctgccttcggagcgcacaaagcagagctgcagggctcagatgaggacgagcacgtgcgagcgcctaccggccaccaccaggctggtcactgcctcatgtgggcctgcaaagcctgcaagaggaagtccaccaccatggatcggcggaaggcagccactatgcgcgagcggaggcgcctgaagaaggtcaaccaggctttcgaaaccctcaagaggtgtaccacgaccaaccccaaccagaggctgcccaaggtggagatcctcaggaatgccatccgctacatcgagagcctgcaggagttgctgagagagcaggtggagaactactatagcctgccgggacagagctgctcggagcccaccagccccacctccaactgctctgatggcatgcccgaatgtaacagtcctgtctggtccagaaagagcagtacttttgacagcatctactgtcctgatgtatcaaatgtatatgccacagataaaaactccttatccagcttggattgcttatccaacatagtggaccggatcacctcctcagagcaacctgggttgcctctccaggatctggcttctctctctccagttgccagcaccgattcacagcctgcaactccaggggcttctagttccaggcttatctatcatgtgctatga

SEQ ID NO: 67 - DNA, 729 bp, Homo sapiens
atgatgatggacctttttgaaactggctcctatttcttctacttggatggggaaaatgttactctgcagccattagaagtggcagaaggctctcctttgtatccagggagtgatggtaccttgtccccctgccaggaccaaatgcccccggaagcggggagcgacagcagcggagaggaacatgtcctggcgcccccgggcctgcagcctccacactgccccggccagtgtctgatctgggcttgcaagacctgcaagagaaaatctgcccccactgaccggcgaaaagccgccaccctgcgcgaaaggaggaggctaaagaaaatcaacgaggccttcgaggcactgaagcggcgaactgtggccaaccccaaccagaggctgcccaaggtggagattctgcggagcgccatcagctatattgagcggctgcaggacctgctgcaccggctggatcagcaggagaagatgcaggagctgggggtggaccccttcagctacagacccaaacaagaaaatcttgagggtgcggatttcctgcgcacctgcagctcccagtggccaagtgtttccgatcattccagggggctcgtgataacggctaaggaaggaggagcaagtattgattcgtcagcctcgagtagccttcgatgcctttcttccatcgtggacagtatttcctcggaggaacgcaaactcccctgcgtggaggaagtggtggagaagtaa

SEQ ID NO: 68 - DNA, 1356 bp, Homo sapiens
atgccgaagaacaagaagcggaacactccccaccgcggtagcagtgctggcggcggcgggtcaggagcagccgcagcgacggcggcgacagcaggtggccagcatcgaaatgttcagccttttagtgatgaagatgcatcaattgaaacaatgagccattgcagtggttatagcgatccttccagttttgctgaagatggaccagaagtccttgatgaggaaggaactcaagaagacctagagtacaagttgaagggattaattgacctaaccctggataagagtgcgaagacaaggcaagcagctcttgaaggtattaaaaatgcactggcttcaaaaatgctgtatgaatttattctggaaaggagaatgactttaactgatagcattgaacgctgcctgaaaaaaggtaagagtgatgagcaacgtgcagctgcagcgttagcatctgttctttgtattcagctgggccctggaattgaaagtgaagagattttgaaaactcttggaccaatcctaaagaaaatcatttgtgatgggtcagctagtatgcaggctaggcaaacttgtgcaacttgctttggtgtttgctgttttattgccacagatgacattactgaactatactcaactctggaatgtttggaaaatatcttcactaaatcctatctcaaagagaaagacactactgttatttgcagcactcctaatacagtgcttcatatcagctctcttcttgcatggacactactgctgaccatatgcccaatcaatgaagtgaagaaaaagcttgagatgcatttccataagcttccaagcctcctctcttgtgatgatgtaaacatgagaatagctgctggtgaatctttggcacttctctttgaattggccagaggaatagagagtgactttttttatgaagacatggagtccttgacgcagatgcttagggccttggcaacagatggaaataaacaccgggccaaagtggacaagagaaagcagcggtcagttttcagagatgtcctgagggcagtggaggaacgggattttccaacagaaaccattaaatttggtcctgaacgcatgtatattgattgctgggtaaaaaaacacacctatgacacctttaaggaggttcttggatcagggatgcagtaccacttgcagtcaaatgaattccttcgaaatgtatttgaacttggacccccagtgatgcttgatgctgcaacgcttaaaacgatgaagatttctcgtttcgaaaggcatttatataactctgcagccttcaaagctcgaaccaaagctagaagcaaatgtcgagataagagagcagatgttggagaattcttctag

SEQ ID NO: 69 - DNA, 1524 bp, Rattus norvegicus
atggggcggaagaaaatacaaatcacacgcataatggatgaaaggaaccgacaggtcacttttacaaagagaaagtttggattaatgaagaaagcctatgaacttagtgtgctctgtgactgtgaaatagcactcatcattttcaacagctctaacaaactgtttcaatatgctagcactgatatggacaaagttcttctcaagtatacagaatataatgaacctcatgaaagcagaaccaactcggatattgttgaggctctgaacaagaaggaacacagagggtgcgacagcccagaccctgatacttcatatgtgctaactccacatacagaagaaaaatataaaaaaattaatgaggaatttgataatatgatgcggaatcataaaatcgcacctggtctgccacctcagaacttttcaatgtctgtcacagttccagtgaccagccccaatgctttgtcctacactaacccagggagttcactggtgtccccatctttggcagccagctcaacgttaacagattcaagcatgctctctccacctcaaaccacattacatagaaatgtgtctcctggagctcctcagagaccaccaagtactggcaatgcaggtgggatgttgagcactacagacctcacagtgccaaatggagctggaagcagtccagtggggaatggatttgtaaactcaagagcttctccaaatttgattggagctactggtgcaaatagcttaggcaaagtcatgcctacaaagtctccccctccaccaggtggtggtaatcttggaatgaacagtaggaaaccagatcttcgagttgtcatccccccttcaagcaagggcatgatgcctccactatcggaggaagaggaattggagttgaacacccaaaggatcagtagttctcaagccactcaacctcttgctaccccagtcgtgtctgtgacaaccccaagcttgcctccgcaaggacttgtgtactcagcaatgccgactgcctacaacactgattattcactgaccagcgctgacctgtcagcccttcaaggcttcaactcgccaggaatgctgtcgctgggacaggtgtcggcctggcagcagcaccacctaggacaagcagccctcagctctcttgttgctggagggcagttatctcagggttccaatttatccattaataccaaccaaaacatcagcatcaagtccgaaccgatttcacctcctcgggatcgtatgaccccatcgggcttccagcagcagcagcagcagcagcagcagcagcagccgccgccaccaccgcagccccagccacaacccccgcagccccagccccgacaggaaatggggcgctcccctgtggacagtctgagcagctctagtagctcctatgatggcagtgatcgggaggatccacggggcgacttccattctccaattgtgcttggccgacccccaaacactgaggacagagaaagcccttctgtaaagcgaatgaggatggacgcgtgggtgacctaa

SEQ ID NO: 70 - DNA, 675 bp, Homo sapiens
atggagctgtatgagacatccccctacttctaccaggaaccccgcttctatgatggggaaaactacctgcctgtccacctccagggcttcgaaccaccaggctacgagcggacggagctcaccctgagccccgaggccccagggccccttgaggacaaggggctggggacccccgagcactgtccaggccagtgcctgccgtgggcgtgtaaggtgtgtaagaggaagtcggtgtccgtggaccggcggcgggcggccacactgagggagaagcgcaggctcaagaaggtgaatgaggccttcgaggccctgaagagaagcaccctgctcaaccccaaccagcggctgcccaaggtggagatcctgcgcagtgccatccagtacatcgagcgcctccaggccctgctcagctccctcaaccaggaggagcgtgacctccgctaccggggcgggggcgggccccagccaggggtgcccagcgaatgcagctctcacagcgcctcctgcagtccagagtggggcagtgcactggagttcagcgccaacccaggggatcatctgctcacggctgaccctacagatgcccacaacctgcactccctcacctccatcgtggacagcatcacagtggaagatgtgtctgtggccttcccagatgaaaccatgcccaactga

SEQ ID NO: 71 - DNA, 822 bp, Homo sapiens
atgtcgctgaccaacacaaagacggggttttcggtcaaggacatcttagacctgccggacaccaacgatgaggagggctctgtggccgaaggtccggaggaagagaacgaggggcccgagccagccaagagggccgggccgctggggcagggcgccctggacgcggtgcagagcctgcccctgaagaaccccttctacgacagcagcgacaacccgtacacgcgctggctggccagcaccgagggccttcagtactccctgcacggtctggctgccggggcgccccctcaggactcaagctccaagtccccggagccctcggccgacgagtcaccggacaatgacaaggagaccccgggcggcgggggggacgccggcaagaagcgaaagcggcgagtgcttttctccaaggcgcagacctacgagctggagcggcgctttcggcagcagcggtacctgtcggcgcccgagcgcgaacacctggccagcctcatccgcctcacgcccacgcaggtcaagatctggttccagaaccaccgctacaagatgaagcgcgcccgggccgagaaaggtatggaggtgacgcccctgccctcgccgcgccgggtggccgtgcccgtcttggtcagggacggcaaaccatgtcacgcgctcaaagcccaggacctggcagccgccaccttccaggcgggcattcccttttctgcctacagcgcgcagtcgctgcagcacatgcagtacaacgcccagtacagctcggccagcaccccccagtacccgacagcacaccccctggtccaggcccagcagtggacttggtga

SEQ ID NO: 72 - DNA, 7668 bp, Homo sapiens
atgccgccgctcctggcgcccctgctctgcctggcgctgctgcccgcgctcgccgcacgaggcccgcgatgctcccagcccggtgagacctgcctgaatggcgggaagtgtgaagcggccaatggcacggaggcctgcgtctgtggcggggccttcgtgggcccgcgatgccaggaccccaacccgtgcctcagcaccccctgcaagaacgccgggacatgccacgtggtggaccgcagaggcgtggcagactatgcctgcagctgtgccctgggcttctctgggcccctctgcctgacacccctggacaatgcctgcctcaccaacccctgccgcaacgggggcacctgcgacctgctcacgctgacggagtacaagtgccgctgcccgcccggctggtcagggaaatcgtgccagcaggctgacccgtgcgcctccaacccctgcgccaacggtggccagtgcctgcccttcgaggcctcctacatctgccactgcccacccagcttccatggccccacctgccggcaggatgtcaacgagtgtggccagaagcccgggctttgccgccacggaggcacctgccacaacgaggtcggctcctaccgctgcgtctgccgcgccacccacactggccccaactgcgagcggccctacgtgccctgcagcccctcgccctgccagaacgggggcacctgccgccccacgggcgacgtcacccacgagtgtgcctgcctgccaggcttcaccggccagaactgtgaggaaaatatcgacgattgtccaggaaacaactgcaagaacgggggtgcctgtgtggacggcgtgaacacctacaactgccgctgcccgccagagtggacaggtcagtactgtaccgaggatgtggacgagtgccagctgatgccaaatgcctgccagaacggcgggacctgccacaacacccacggtggctacaactgcgtgtgtgtcaacggctggactggtgaggactgcagcgagaacattgatgactgtgccagcgccgcctgcttccacggcgccacctgccatgaccgtgtggcctccttctactgcgagtgtccccatggccgcacaggtctgctgtgccacctcaacgacgcatgcatcagcaacccctgtaacgagggctccaactgcgacaccaaccctgtcaatggcaaggccatctgcacctgcccctcggggtacacgggcccggcctgcagccaggacgtggatgagtgctcgctgggtgccaacccctgcgagcatgcgggcaagtgcatcaacacgctgggctccttcgagtgccagtgtctgcagggctacacgggcccccgatgcgagatcgacgtcaacgagtgcgtctcgaacccgtgccagaacgacgccacctgcctggaccagattggggagttccagtgcatctgcatgcccggctacgagggtgtgcactgcgaggtcaacacagacgagtgtgccagcagcccctgcctgcacaatggccgctgcctggacaagatcaatgagttccagtgcgagtgccccacgggcttcactgggcatctgtgccagtacgatgtggacgagtgtgccagcaccccctgcaagaatggtgccaagtgcctggacggacccaacacttacacctgtgtgtgcacggaagggtacacggggacgcactgcgaggtggacatcgatgagtgcgaccccgacccctgccactacggctcctgcaaggacggcgtcgccaccttcacctgcctctgccgcccaggctacacgggccaccactgcgagaccaacatcaacgagtgctccagccagccctgccgccacgggggcacctgccaggaccgcgacaacgcctacctctgcttctgcctgaaggggaccacaggacccaactgcgagatcaacctggatgactgtgccagcagcccctgcgactcgggcacctgtctggacaagatcgatggctacgagtgtgcctgtgagccgggctacacagggagcatgtgtaacatcaacatcgatgagtgtgcgggcaacccctgccacaacgggggcacctgcgaggacggcatcaatggcttcacctgccgctgccccgagggctaccacgaccccacctgcctgtctgaggtcaatgagtgcaacagcaacccctgcgtccacggggcctgccgggacagcctcaacgggtacaagtgcgactgtgaccctgggtggagtgggaccaactgtgacatcaacaacaatgagtgtgaatccaacccttgtgtcaacggcggcacctgcaaagacatgaccagtggctacgtgtgcacctgccgggagggcttcagcggtcccaactgccagaccaacatcaacgagtgtgcgtccaacccatgtctgaaccagggcacgtgtattgacgacgttgccgggtacaagtgcaactgcctgctgccctacacaggtgccacgtgtgaggtggtgctggccccgtgtgcccccagcccctgcagaaacggcggggagtgcaggcaatccgaggactatgagagcttctcctgtgtctgccccacgggctggcaagggcagacctgtgaggtcgacatcaacgagtgcgttctgagcccgtgccggcacggcgcatcctgccagaacacccacggcggctaccgctgccactgccaggccggctacagtgggcgcaactgcgagaccgacatcgacgactgccggcccaacccgtgtcacaacgggggctcctgcacagacggcatcaacacggccttctgcgactgcctgcccggcttccggggcactttctgtgaggaggacatcaacgagtgtgccagtgacccctgccgcaacggggccaactgcacggactgcgtggacagctacacgtgcacctgccccgcaggcttcagcgggatccactgtgagaacaacacgcctgactgcacagagagctcctgcttcaacggtggcacctgcgtggacggcatcaactcgttcacctgcctgtgtccacccggcttcacgggcagctactgccagcacgatgtcaatgagtgcgactcacagccctgcctgcatggcggcacctgtcaggacggctgcggctcctacaggtgcacctgcccccagggctacactggccccaactgccagaaccttgtgcactggtgtgactcctcgccctgcaagaacggcggcaaatgctggcagacccacacccagtaccgctgcgagtgccccagcggctggaccggcctttactgcgacgtgcccagcgtgtcctgtgaggtggctgcgcagcgacaaggtgttgacgttgcccgcctgtgccagcatggagggctctgtgtggacgcgggcaacacgcaccactgccgctgccaggcgggctacacaggcagctactgtgaggacctggtggacgagtgctcacccagcccctgccagaacggggccacctgcacggactacctgggcggctactcctgcaagtgcgtggccggctaccacggggtgaactgctctgaggagatcgacgagtgcctctcccacccctgccagaacgggggcacctgcctcgacctccccaacacctacaagtgctcctgcccacggggcactcagggtgtgcactgtgagatcaacgtggacgactgcaatccccccgttgaccccgtgtcccggagccccaagtgctttaacaacggcacctgcgtggaccaggtgggcggctacagctgcacctgcccgccgggcttcgtgggtgagcgctgtgagggggatgtcaacgagtgcctgtccaatccctgcgacgcccgtggcacccagaactgcgtgcagcgcgtcaatgacttccactgcgagtgccgtgctggtcacaccgggcgccgctgcgagtccgtcatcaatggctgcaaaggcaagccctgcaagaatgggggcacctgcgccgtggcctccaacaccgcccgcgggttcatctgcaagtgccctgcgggcttcgagggcgccacgtgtgagaatgacgctcgtacctgcggcagcctgcgctgcctcaacggcggcacatgcatctccggcccgcgcagccccacctgcctgtgcctgggccccttcacgggccccgaatgccagttcccggccagcagcccctgcctgggcggcaacccctgctacaaccaggggacctgtgagcccacatccgagagccccttctaccgttgcctgtgccccgccaaattcaacgggctcttgtgccacatcctggactacagcttcgggggtggggccgggcgcgacatccccccgccgctgatcgaggaggcgtgcgagctgcccgagtgccaggaggacgcgggcaacaaggtctgcagcctgcagtgcaacaaccacgcgtgcggctgggacggcggtgactgctccctcaacttcaatgacccctggaagaactgcacgcagtctctgcagtgctggaagtacttcagtgacggccactgtgacagccagtgcaactcagccggctgcctcttcgacggctttgactgccagcgtgcggaaggccagtgcaaccccctgtacgaccagtactgcaaggaccacttcagcgacgggcactgcgaccagggctgcaacagcgcggagtgcgagtgggacgggctggactgtgcggagcatgtacccgagaggctggcggccggcacgctggtggtggtggtgctgatgccgccggagcagctgcgcaacagctccttccacttcctgcgggagctcagccgcgtgctgcacaccaacgtggtcttcaagcgtgacgcacacggccagcagatgatcttcccctactacggccgcgaggaggagctgcgcaagcaccccatcaagcgtgccgccgagggctgggccgcacctgacgccctgctgggccaggtgaaggcctcgctgctccctggtggcagcgagggtgggcggcggcggagggagctggaccccatggacgtccgcggctccatcgtctacctggagattgacaaccggcagtgtgtgcaggcctcctcgcagtgcttccagagtgccaccgacgtggccgcattcctgggagcgctcgcctcgctgggcagcctcaacatcccctacaagatcgaggccgtgcagagtgagaccgtggagccgcccccgccggcgcagctgcacttcatgtacgtggcggcggccgcctttgtgcttctgttcttcgtgggctgcggggtgctgctgtcccgcaagcgccggcggcagcatggccagctctggttccctgagggcttcaaagtgtctgaggccagcaagaagaagcggcgggagcccctcggcgaggactccgtgggcctcaagcccctgaagaacgcttcagacggtgccctcatggacgacaaccagaatgagtggggggacgaggacctggagaccaagaagttccggttcgaggagcccgtggttctgcctgacctggacgaccagacagaccaccggcagtggactcagcagcacctggatgccgctgacctgcgcatgtctgccatggcccccacaccgccccagggtgaggttgacgccgactgcatggacgtcaatgtccgcgggcctgatggcttcaccccgctcatgatcgcctcctgcagcgggggcggcctggagacgggcaacagcgaggaagaggaggacgcgccggccgtcatctccgacttcatctaccagggcgccagcctgcacaaccagacagaccgcacgggcgagaccgccttgcacctggccgcccgctactcacgctctgatgccgccaagcgcctgctggaggccagcgcagatgccaacatccaggacaacatgggccgcaccccgctgcatgcggctgtgtctgccgacgcacaaggtgtcttccagatcctgatccggaaccgagccacagacctggatgcccgcatgcatgatggcacgacgccactgatcctggctgcccgcctggccgtggagggcatgctggaggacctcatcaactcacacgccgacgtcaacgccgtagatgacctgggcaagtccgccctgcactgggccgccgccgtgaacaatgtggatgccgcagttgtgctcctgaagaacggggctaacaaagatatgcagaacaacagggaggagacacccctgtttctggccgcccgggagggcagctacgagaccgccaaggtgctgctggaccactttgccaaccgggacatcacggatcatatggaccgcctgccgcgcgacatcgcacaggagcgcatgcatcacgacatcgtgaggctgctggacgagtacaacctggtgcgcagcccgcagctgcacggagccccgctggggggcacgcccaccctgtcgcccccgctctgctcgcccaacggctacctgggcagcctcaagcccggcgtgcagggcaagaaggtccgcaagcccagcagcaaaggcctggcctgtggaagcaaggaggccaaggacctcaaggcacggaggaagaagtcccaggacggcaagggctgcctgctggacagctccggcatgctctcgcccgtggactccctggagtcaccccatggctacctgtcagacgtggcctcgccgccactgctgccctccccgttccagcagtctccgtccgtgcccctcaaccacctgcctgggatgcccgacacccacctgggcatcgggcacctgaacgtggcggccaagcccgagatggcggcgctgggtgggggcggccggctggcctttgagactggcccacctcgtctctcccacctgcctgtggcctctggcaccagcaccgtcctgggctccagcagcggaggggccctgaatttcactgtgggcgggtccaccagtttgaatggtcaatgcgagtggctgtcccggctgcagagcggcatggtgccgaaccaatacaaccctctgcgggggagtgtggcaccaggccccctgagcacacaggccccctccctgcagcatggcatggtaggcccgctgcacagtagccttgctgccagcgccctgtcccagatgatgagctaccagggcctgcccagcacccggctggccacccagcctcacctggtgcagacccagcaggtgcagccacaaaacttacagatgcagcagcagaacctgcagccagcaaacatccagcagcagcaaagcctgcagccgccaccaccaccaccacagccgcaccttggcgtgagctcagcagccagcggccacctgggccggagcttcctgagtggagagccgagccaggcagacgtgcagccactgggccccagcagcctggcggtgcacactattctgccccaggagagccccgccctgcccacgtcgctgccatcctcgctggtcccacccgtgaccgcagcccagttcctgacgcccccctcgcagcacagctactcctcgcctgtggacaacacccccagccaccagctacaggtgcctgagcaccccttcctcaccccgtcccctgagtcccctgaccagtggtccagctcgtccccgcattccaacgtctccgactggtccgagggcgtctccagccctcccaccagcatgcagtcccagatcgcccgcattccggaggccttcaagtaa

SEQ ID NO: 73 - DNA, 6966 bp, Homo sapiens
atggggccgggggcccgtggccgccgccgccgccgtcgcccgatgtcgccgccaccgccaccgccacccgtgcgggcgctgcccctgctgctgctgctagcggggccgggggctgcagcccccccttgcctggacggaagcccgtgtgcaaatggaggtcgttgcacccagctgccctcccgggaggctgcctgcctgtgcccgcctggctgggtgggtgagcggtgtcagctggaggacccctgtcactcaggcccctgtgctggccgtggtgtctgccagagttcagtggtggctggcaccgcccgattctcatgccggtgcccccgtggcttccgaggccctgactgctccctgccagatccctgcctcagcagcccttgtgcccacggtgcccgctgctcagtggggcccgatggacgcttcctctgctcctgcccacctggctaccagggccgcagctgccgaagcgacgtggatgagtgccgggtgggtgagccctgccgccatggtggcacctgcctcaacacacctggctccttccgctgccagtgtccagctggctacacagggccactatgtgagaaccccgcggtgccctgtgcaccctcaccatgccgtaacgggggcacctgcaggcagagtggcgacctcacttacgactgtgcctgtcttcctgggtttgagggtcagaattgtgaagtgaacgtggacgactgtccaggacaccgatgtctcaatggggggacatgcgtggatggcgtcaacacctataactgccagtgccctcctgagtggacaggccagttctgcacggaggacgtggatgagtgtcagctgcagcccaacgcctgccacaatgggggtacctgcttcaacacgctgggtggccacagctgcgtgtgtgtcaatggctggacaggcgagagctgcagtcagaatatcgatgactgtgccacagccgtgtgcttccatggggccacctgccatgaccgcgtggcttctttctactgtgcctgccccatgggcaagactggcctcctgtgtcacctggatgacgcctgtgtcagcaacccctgccacgaggatgctatctgtgacacaaatccggtgaacggccgggccatttgcacctgtcctcccggcttcacgggtggggcatgtgaccaggatgtggacgagtgctctatcggcgccaacccctgcgagcacttgggcaggtgcgtgaacacgcagggctccttcctgtgccagtgcggtcgtggctacactggacctcgctgtgagaccgatgtcaacgagtgtctgtcggggccctgccgaaaccaggccacgtgcctcgaccgcataggccagttcacctgtatctgtatggcaggcttcacaggaacctattgcgaggtggacattgacgagtgtcagagtagcccctgtgtcaacggtggggtctgcaaggaccgagtcaatggcttcagctgcacctgcccctcgggcttcagcggctccacgtgtcagctggacgtggacgaatgcgccagcacgccctgcaggaatggcgccaaatgcgtggaccagcccgatggctacgagtgccgctgtgccgagggctttgagggcacgctgtgtgatcgcaacgtggacgactgctcccctgacccatgccaccatggtcgctgcgtggatggcatcgccagcttctcatgtgcctgtgctcctggctacacgggcacacgctgcgagagccaggtggacgaatgccgcagccagccctgccgccatggcggcaaatgcctagacctggtggacaagtacctctgccgctgcccttctgggaccacaggtgtgaactgcgaagtgaacattgacgactgtgccagcaacccctgcacctttggagtctgccgtgatggcatcaaccgctacgactgtgtctgccaacctggcttcacagggcccctttgtaacgtggagatcaatgagtgtgcttccagcccatgcggcgagggaggttcctgtgtggatggggaaaatggcttccgctgcctctgcccgcctggctccttgcccccactctgcctccccccgagccatccctgtgcccatgagccctgcagtcacggcatctgctatgatgcacctggcgggttccgctgtgtgtgtgagcctggctggagtggcccccgctgcagccagagcctggcccgagacgcctgtgagtcccagccgtgcagggccggtgggacatgcagcagcgatggaatgggtttccactgcacctgcccgcctggtgtccagggacgtcagtgtgaactcctctccccctgcaccccgaacccctgtgagcatgggggccgctgcgagtctgcccctggccagctgcctgtctgctcctgcccccagggctggcaaggcccacgatgccagcaggatgtggacgagtgtgctggccccgcaccctgtggccctcatggtatctgcaccaacctggcagggagtttcagctgcacctgccatggagggtacactggcccttcctgcgatcaggacatcaatgactgtgaccccaacccatgcctgaacggtggctcgtgccaagacggcgtgggctccttttcctgctcctgcctccctggtttcgccggcccacgatgcgcccgcgatgtggatgagtgcctgagcaacccctgcggcccgggcacctgtaccgaccacgtggcctccttcacctgcacctgcccgccaggctacggaggcttccactgcgaacaggacctgcccgactgcagccccagctcctgcttcaatggcgggacctgtgtggacggcgtgaactcgttcagctgcctgtgccgtcccggctacacaggagcccactgccaacatgaggcagacccctgcctctcgcggccctgcctacacgggggcgtctgcagcgccgcccaccctggcttccgctgcacctgcctcgagagcttcacgggcccgcagtgccagacgctggtggattggtgcagccgccagccttgtcaaaacgggggtcgctgcgtccagactggggcctattgcctttgtccccctggatggagcggacgcctctgtgacatccgaagcttgccctgcagggaggccgcagcccagatcggggtgcggctggagcagctgtgtcaggcgggtgggcagtgtgtggatgaagacagctcccactactgcgtgtgcccagagggccgtactggtagccactgtgagcaggaggtggacccctgcttggcccagccctgccagcatggggggacctgccgtggctatatggggggctacatgtgtgagtgtcttcctggctacaatggtgataactgtgaggacgacgtggacgagtgtgcctcccagccctgccagcacgggggttcatgcattgacctcgtggcccgctatctctgctcctgtcccccaggaacgctgggggtgctctgcgagattaatgaggatgactgcggcccaggcccaccgctggactcagggccccggtgcctacacaatggcacctgcgtggacctggtgggtggtttccgctgcacctgtcccccaggatacactggtttgcgctgcgaggcagacatcaatgagtgtcgctcaggtgcctgccacgcggcacacacccgggactgcctgcaggacccaggcggaggtttccgttgcctttgtcatgctggcttctcaggtcctcgctgtcagactgtcctgtctccctgcgagtcccagccatgccagcatggaggccagtgccgtcctagcccgggtcctgggggtgggctgaccttcacctgtcactgtgcccagccgttctggggtccgcgttgcgagcgggtggcgcgctcctgccgggagctgcagtgcccggtgggcgtcccatgccagcagacgccccgcgggccgcgctgcgcctgccccccagggttgtcgggaccctcctgccgcagcttcccggggtcgccgccgggggccagcaacgccagctgcgcggccgccccctgtctccacgggggctcctgccgccccgcgccgctcgcgcccttcttccgctgcgcttgcgcgcagggctggaccgggccgcgctgcgaggcgcccgccgcggcacccgaggtctcggaggagccgcggtgcccgcgcgccgcctgccaggccaagcgcggggaccagcgctgcgaccgcgagtgcaacagcccaggctgcggctgggacggcggcgactgctcgctgagcgtgggcgacccctggcggcaatgcgaggcgctgcagtgctggcgcctcttcaacaacagccgctgcgaccccgcctgcagctcgcccgcctgcctctacgacaacttcgactgccacgccggtggccgcgagcgcacttgcaacccggtgtacgagaagtactgcgccgaccactttgccgacggccgctgcgaccagggctgcaacacggaggagtgcggctgggatgggctggattgtgccagcgaggtgccggccctgctggcccgcggcgtgctggtgctcacagtgctgctgccgccagaggagctactgcgttccagcgccgactttctgcagcggctcagcgccatcctgcgcacctcgctgcgcttccgcctggacgcgcacggccaggccatggtcttcccttaccaccggcctagtcctggctccgaaccccgggcccgtcgggagctggcccccgaggtgatcggctcggtagtaatgctggagattgacaaccggctctgcctgcagtcgcctgagaatgatcactgcttccccgatgcccagagcgccgctgactacctgggagcgttgtcagcggtggagcgcctggacttcccgtacccactgcgggacgtgcggggggagccgctggagcctccagaacccagcgtcccgctgctgccactgctagtggcgggcgctgtcttgctgctggtcattctcgtcctgggtgtcatggtggcccggcgcaagcgcgagcacagcaccctctggttccctgagggcttctcactgcacaaggacgtggcctctggtcacaagggccggcgggaacccgtgggccaggacgcgctgggcatgaagaacatggccaagggtgagagcctgatgggggaggtggccacagactggatggacacagagtgcccagaggccaagcggctaaaggtagaggagccaggcatgggggctgaggaggctgtggattgccgtcagtggactcaacaccatctggttgctgctgacatccgcgtggcaccagccatggcactgacaccaccacagggcgacgcagatgctgatggcatggatgtcaatgtgcgtggcccagatggcttcaccccgctaatgctggcttccttctgtgggggggctctggagccaatgccaactgaagaggatgaggcagatgacacatcagctagcatcatctccgacctgatctgccagggggctcagcttggggcacggactgaccgtactggcgagactgctttgcacctggctgcccgttatgcccgtgctgatgcagccaagcggctgctggatgctggggcagacaccaatgcccaggaccactcaggccgcactcccctgcacacagctgtcacagccgatgcccagggtgtcttccagattctcatccgaaaccgctctacagacttggatgcccgcatggcagatggctcaacggcactgatcctggcggcccgcctggcagtagagggcatggtggaagagctcatcgccagccatgctgatgtcaatgctgtggatgagcttgggaaatcagccttacactgggctgcggctgtgaacaacgtggaagccactttggccctgctcaaaaatggagccaataaggacatgcaggatagcaaggaggagacccccctattcctggccgcccgcgagggcagctatgaggctgccaagctgctgttggaccactttgccaaccgtgagatcaccgaccacctggacaggctgccgcgggacgtagcccaggagagactgcaccaggacatcgtgcgcttgctggatcaacccagtgggccccgcagcccccccggtccccacggcctggggcctctgctctgtcctccaggggccttcctccctggcctcaaagcggcacagtcggggtccaagaagagcaggaggccccccgggaaggcggggctggggccgcaggggccccgggggcggggcaagaagctgacgctggcctgcccgggccccctggctgacagctcggtcacgctgtcgcccgtggactcgctggactccccgcggcctttcggtgggccccctgcttcccctggtggcttcccccttgaggggccctatgcagctgccactgccactgcagtgtctctggcacagcttggtggcccaggccgggcgggtctagggcgccagccccctggaggatgtgtactcagcctgggcctgctgaaccctgtggctgtgcccctcgattgggcccggctgcccccacctgcccctccaggcccctcgttcctgctgccactggcgccgggaccccagctgctcaacccagggacccccgtctccccgcaggagcggcccccgccttacctggcagtcccaggacatggcgaggagtacccggcggctggggcacacagcagccccccaaaggcccgcttcctgcgggttcccagtgagcacccttacctgaccccatcccccgaatcccctgagcactgggccagcccctcacctccctccctctcagactggtccgaatccacgcctagcccagccactgccactggggccatggccaccaccactggggcactgcctgcccagccacttcccttgtctgttcccagctcccttgctcaggcccagacccagctggggccccagccggaagttacccccaagaggcaagtgttggcctga

SEQ ID NO: 74 - DNA, 1797 bp, Homo sapiens
atgccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaatcactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccgggtcggtttactacaagccctcctcgcccccgacgcccaccaccccgggcttccaggtgcagcacagccccatgtgggacgacccgggatctctccacaacttccaccagaactacgtggccactacgcacatgatcgagcagaggaaaacgccagtctcccgcctctccctcttctcctttaagcaatcgccccctggcaccccggtgtctagttgccagatgcgcttcgacgggcccctgcacgtccccatgaacccggagcccgccggcagccaccacgtggtggacgggcagaccttcgctgtgcccaaccccattcgcaagcccgcgtccatgggcttcccgggcctgcagatcggccacgcgtctcagctgctcgacacgcaggtgccctcaccgccgtcgcggggctccccctccaacgaggggctgtgcgctgtgtgtggggacaacgcggcctgccaacactacggcgtgcgcacctgtgagggctgcaaaggcttctttaagcgcacagtgcaaaaaaatgcaaaatacgtgtgtttagcaaataaaaactgcccagtggacaagcgtcgccggaatcgctgtcagtactgccgatttcagaagtgcctggctgttgggatggtcaaagaagtggttcgcacagacagtttaaaaggccggagaggtcgtttgccctcgaaaccgaagagcccacaggagccctctcccccttcgcccccggtgagtctgatcagtgccctcgtcagggcccatgtcgactccaacccggctatgaccagcctggactattccaggttccaggcgaaccctgactatcaaatgagtggagatgacacccagcatatccagcaattctatgatctcctgactggctccatggagatcatccggggctgggcagagaagatccctggcttcgcagacctgcccaaagccgaccaagacctgctttttgaatcagctttcttagaactgtttgtccttcgattagcatacaggtccaacccagtggagggtaaactcatcttttgcaatggggtggtcttgcacaggttgcaatgcgttcgtggctttggggaatggattgattccattgttgaattctcctccaacttgcagaatatgaacatcgacatttctgccttctcctgcattgctgccctggctatggtcacagagagacacgggctcaaggaacccaagagagtggaagaactgcaaaacaagattgtaaattgtctcaaagaccacgtgactttcaacaatggggggttgaaccgccccaattatttgtccaaactgttggggaagctcccagaacttcgtaccctttgcacacaggggctacagcgcattttctacctgaaattggaagacttggtgccaccgccagcaataattgacaaacttttcctggacactttacctttctaa

SEQ ID NO: 75 - DNA, 1074 bp, Homo sapiens
atgcagagtgtgcagagcacgagcttttgtctccgaaagcagtgcctttgcctgaccttcctgcttctccatctcctgggacaggtcgctgcgactcagcgctgccctccccagtgcccgggccggtgccctgcgacgccgccgacctgcgcccccggggtgcgcgcggtgctggacggctgctcatgctgtctggtgtgtgcccgccagcgtggcgagagctgctcagatctggagccatgcgacgagagcagtggcctctactgtgatcgcagcgcggaccccagcaaccagactggcatctgcacggcggtagagggagataactgtgtgttcgatggggtcatctaccgcagtggagagaaatttcagccaagctgcaaattccagtgcacctgcagagatgggcagattggctgtgtgccccgctgtcagctggatgtgctactgcctgagcctaactgcccagctccaagaaaagttgaggtgcctggagagtgctgtgaaaagtggatctgtggcccagatgaggaggattcactgggaggccttacccttgcagcttacaggccagaagccaccctaggagtagaagtctctgactcaagtgtcaactgcattgaacagaccacagagtggacagcatgctccaagagctgtggtatggggttctccacccgggtcaccaataggaaccgtcaatgtgagatgctgaaacagactcggctctgcatggtgcggccctgtgaacaagagccagagcagccaacagataagaaaggaaaaaagtgtctccgcaccaagaagtcactcaaagccatccacctgcagttcaagaactgcaccagcctgcacacctacaagcccaggttctgtggggtctgcagtgatggccgctgctgcactccccacaataccaaaaccatccaggcagagtttcagtgctccccagggcaaatagtcaagaagccagtgatggtcattgggacctgcacctgtcacaccaactgtcctaagaacaatgaggccttcctccaggagctggagctgaagactaccagagggaaaatgtaa

SEQ ID NO: 76 - DNA, 768 bp, Homo sapiens
atgctgcggccacagcggcccggagacttgcagctcggggcctccctctacgagctggtgggctacaggcagccgccctcctcctcctcctcctccacctcctccacctcctccacttcctcctcctccacgacggcccccctcctccccaaggctgcgcgcgagaagccggaggcgccggccgagcctccaggccccgggcccgggtcaggcgcgcacccgggcggcagcgcccggccggacgccaaggaggagcagcagcagcagctgcggcgcaagatcaacagccgcgagcggaagcgcatgcaggacctgaacctggccatggacgccctgcgcgaggtcatcctgccctactcagcggcgcactgccagggcgcgcccggccgcaagctctccaagatagccacgctgctgctcgcccgcaactacatcctactgctgggcagctcgctgcaggagctgcgccgcgcgctgggcgagggcgccgggcccgccgcgccgcgcctgctgctggccgggctgcccctgctcgccgccgcgcccggctccgtgttgctggcgcccggcgccgtaggaccccccgacgcgctgcgccccgccaagtacctgtcgctggcgctggacgagccgccgtgcggccagttcgctctccccggcggcggcgcaggcggccccggcctctgcacctgcgccgtgtgcaagttcccgcacctggtcccggccagcctgggcctggccgccgtgcaggcgcaattctccaagtga

SEQ ID NO: 77 - DNA, 972 bp, Homo sapiens
atggactcggacgccagcctggtgtccagccgcccgtcgtcgccagagcccgatgacctttttctgccggcccggagtaagggcagcagcggcagcgccttcactgggggcaccgtgtcctcgtccaccccgagtgactgcccgccggagctgagcgccgagctgcgcggcgctatgggctctgcgggcgcgcatcctggggacaagctaggaggcagtggcttcaagtcatcctcgtccagcacctcgtcgtctacgtcgtcggcggctgcgtcgtccaccaagaaggacaagaagcaaatgacagagccggagctgcagcagctgcgtctcaagatcaacagccgcgagcgcaagcgcatgcacgacctcaacatcgccatggatggcctccgcgaggtcatgccgtacgcacacggcccttcggtgcgcaagctttccaagatcgccacgctgctgctggcgcgcaactacatcctcatgctcaccaactcgctggaggagatgaagcgactggtgagcgagatctacgggggccaccacgctggcttccacccgtcggcctgcggcggcctggcgcactccgcgcccctgcccgccgccaccgcgcacccggcagcagcagcgcacgccgcacatcaccccgcggtgcaccaccccatcctgccgcccgccgccgcagcggctgctgccgccgctgcagccgcggctgtgtccagcgcctctctgcccggatccgggctgccgtcggtcggctccatccgtccaccgcacggcctactcaagtctccgtctgctgccgcggccgccccgctggggggcgggggcggcggcagtggggcgagcgggggcttccagcactggggcggcatgccctgcccctgcagcatgtgccaggtgccgccgccgcaccaccacgtgtcggctatgggcgccggcagcctgccgcgcctcacctccgacgccaagtga

SEQ ID NO: 78 - DNA, 852 bp, Homo sapiens
atgaacggcgaggagcagtactacgcggccacgcagctttacaaggacccatgcgcgttccagcgaggcccggcgccggagttcagcgccagcccccctgcgtgcctgtacatgggccgccagcccccgccgccgccgccgcacccgttccctggcgccctgggcgcgctggagcagggcagccccccggacatctccccgtacgaggtgccccccctcgccgacgaccccgcggtggcgcaccttcaccaccacctcccggctcagctcgcgctcccccacccgcccgccgggcccttcccggagggagccgagccgggcgtcctggaggagcccaaccgcgtccagctgcctttcccatggatgaagtctaccaaagctcacgcgtggaaaggccagtgggcaggcggcgcctacgctgcggagccggaggagaacaagcggacgcgcacggcctacacgcgcgcacagctgctagagctggagaaggagttcctattcaacaagtacatctcacggccgcgccgggtggagctggctgtcatgttgaacttgaccgagagacacatcaagatctggttccaaaaccgccgcatgaagtggaaaaaggaggaggacaagaagcgcggcggcgggacagctgtcgggggtggcggggtcgcggagcctgagcaggactgcgccgtgacctccggcgaggagcttctggcgctgccgccgccgccgccccccggaggtgctgtgccgcccgctgcccccgttgccgcccgagagggccgcctgccgcctggccttagcgcgtcgccacagccctccagcgtcgcgcctcggcggccgcaggaaccacgatga

SEQ ID NO: 79 - DNA, 717 bp, Homo sapiens
atgagacagagcggcgcctcccagcccctgctgatcaacatgtacctgccagatcccgtcggagacggtctcttcaaggacgggaagaacccgagctgggggccgctgagccccgcggttcagaaaggcagcggacagatccagctgtggcagtttctgctggagctgctggctgaccgcgcgaacgccggctgcatcgcgtgggagggcggtcacggcgagttcaagctcacggacccggacgaggtggcgcggcggtggggcgagcgcaagagcaagcccaacatgaactacgacaagctgagccgcgccctgcgctactactacgacaagaacatcatgagcaaggtgcatggcaagcgctacgcctaccgcttcgacttccagggcctggcgcaggcctgccagccgccgcccgcgcacgctcatgccgccgccgcagctgctgccgccgccgcggccgcccaggacggcgcgctctacaagctgcccgccggcctcgccccgctgcccttccccggcctctccaaactcaacctcatggccgcctcggccggggtcgcgcccgccggcttctcctactggccgggcccgggccccgccgccaccgctgccgccgccaccgccgcgctctaccccagtcccagcttgcagcccccgcccgggcccttcggggccgtggccgcagcctcgcacttggggggccattaccactag

SEQ ID NO: 80 - DNA, 855 bp, Homo sapiens
atggactactcctacctcaattcgtacgactcgtgcgtggcggccatggaggcgtccgcctacggcgactttggcgcctgcagccagcccggcggcttccaatacagccccctgcggcccgctttccccgcggcagggccgccctgccccgcgctcggctcctccaactgcgcacttggcgccctacgcgaccaccagcccgcgccctactcggcagtgccctacaagttcttcccagagccatccggcctgcacgagaagcgcaagcagcggcgcatccgcaccacgttcaccagcgcgcagctcaaggagctggagcgcgttttcgctgagacccactaccccgacatttacacgcgtgaggagctggcgctcaagatcgacctcactgaggctcgcgtgcaggtctggttccagaaccgccgggccaagttccgcaaacaggagcgcgcggccagcgccaagggcgcggcgggcgcggcgggcgccaaaaagggcgaggcgcgctgctcctccgaggacgacgattccaaggagtccacgtgcagccccacgcccgatagcaccgcctcgctgccgccgccgcctgcgcccggcctggccagcccgcgcctgagccccagcccgctgcccgtcgcactgggctccgggccgggacctgggccggggccacagccgctcaagggcgcactgtgggccggtgtggcgggcggtgggggcggcgggcctggcgcgggagcggccgaactacttaaggcttggcagccggcggagtccggccccgggcccttctccggggttctgtcctcctttcaccggaagcccggccccgccctgaagaccaatctcttctag

SEQ ID NO: 81 - DNA, 945 bp, Homo sapiens
atgtataaaatggaatattcttacctcaattcctctgcctacgagtcctgtatggctgggatggacacctcgagcctggcttcagcctatgctgacttcagttcctgcagccaggccagtggcttccagtataacccgataaggaccacttttggggccacgtccggctgcccttccctcacgccgggatcctgcagcctgggcaccctcagggaccaccagagcagtccgtacgccgcagttccttacaaactcttcacggaccacggcggcctcaacgagaagcgcaagcagcggcgcatccgcaccactttcaccagtgcccagctcaaagagctggaaagggtcttcgcggagactcactaccccgacatctacactcgggaggagctggccctgaagatcgacctcacagaggcgcgagtccaggtgtggttccagaaccgccgcgccaagtttcgcaagcaggagcgcgcagcggcagccgcagcggccgcggccaagaacggctcctcgggcaaaaagtctgactcttccagggacgacgagagcaaagaggccaagagcactgacccggacagcactgggggcccaggtcccaatcccaaccccacccccagctgcggggcgaatggaggcggcggcggcgggcccagcccggctggagctccgggggcggcggggcccgggggcccgggaggcgaacccggcaagggcggcgcagcagcagcggcggcggccgcggcagcggcggcggcggcagcggcagcggcggcagctggaggcctggctgcggctgggggccctggacaaggctgggctcccggccccggccccatcacctccatcccggattcgcttgggggtcccttcgccagcgtcctatcttcgctccaaagacccaacggtgccaaagccgccttagtgaagagcagtatgttctga

SEQ ID NO: 82 - DNA, 876 bp, Homo sapiens
atgagttgccaagcttttacttcggctgatacctttatacctctgaattctgacgcctctgcaactctgcctctgataatgcatcacagtgctgccgagtgtctaccagtctccaaccatgccaccaatgtgatgtctacagcaacaggacttcattattctgttccttcctgtcattatggaaaccagccatcaacctatggagtgatggcaggtagtttaaccccttgtctttataaatttcctgaccacaccttgagtcatggatttcctcctatacaccagcctcttctggcagaggaccccacagctgctgatttcaagcaggaactcaggcggaaaagtaaattggtggaagagccaatagacatggattctccagaaatcagagaacttgaaaagtttgccaatgaatttaaagtgagacgaattaaattaggatacacccagacaaatgttggggaggccctggcagctgtgcatggctctgaattcagtcaaacaacaatctgccgatttgaaaatctgcagctcagctttaaaaatgcatgcaaactgaaagcaatattatccaaatggctggaggaagctgagcaagtaggagctttgtacaatgaaaaagtgggagcaaatgaaaggaaaagaaaacgaagaacaactataagcattgctgctaaagatgctctggagagacactttggagaacagaataaaccttcttctcaagagatcatgaggatggctgaagaactgaatctggagaaagaagtagtaagagtttggttttgcaaccggaggcagagagaaaaacgggtgaaaacaagtctgaatcagagtttattttctatttctaaggaacatcttgagtgcagataa

SEQ ID NO: 83 - DNA, 909 bp, Homo sapiens
atggagttcggcctgctcagcgaggcagaggcccggagccctgccctgtcgctgtcagacgctggcactccgcacccccagctcccagagcacggctgcaagggccaggagcacagcgactcagaaaaggcctcggcttcgctgcccggcggctccccagaggacggttcgctgaaaaagaagcagcggcggcagcgcacgcacttcaccagccagcagctacaggagctagaggcgaccttccagaggaaccgctaccccgacatgagcacgcgcgaggagatcgccgtgtggaccaacctcaccgaggcccgcgtgcgggtgtggttcaagaaccggcgcgccaaatggcggaagcgcgagcgcagccagcaggccgagctatgcaaaggcagcttcgcggcgccgctcggggggctggtgccgccctacgaggaggtgtaccccggctactcgtacggcaactggccgcccaaggctcttgccccgccgctcgccgccaagacctttccattcgccttcaactcggtcaacgtggggcctctggcttcgcagcccgtcttctcgccacccagctccatcgccgcctccatggtgccctccgccgcggctgccccgggcaccgtgccagggcctggggccctgcagggcctgggcgggggcccccccgggctggctccggccgccgtgtcctccggggccgtgtcctgcccttatgcctcggccgccgccgccgccgcggctgccgcctcttccccctacgtctatcgggacccgtgtaactcgagcctggccagcctgcggctcaaagccaaacagcacgcctccttcagctaccccgctgtgcacgggccgcccccggcagccaaccttagtccgtgccagtacgccgtggaaaggcccgtatga

SEQ ID NO: 84 - DNA, 1362 bp, Homo sapiens
atgcgtatccccgtagatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaacggcacccgacctgacagcgttcagcgacccgcgccagttccccgcgctgccctccatctccgacccccgcatgcactatccaggcgccttcacctactccccgacgccggtcacctcgggcatcggcatcggcatgtcggccatgggctcggccacgcgctaccacacctacctgccgccgccctaccccggctcgtcgcaagcgcagggaggcccgttccaagccagctcgccctcctaccacctgtactacggcgcctcggccggctcctaccagttctccatggtgggcggcgagcgctcgccgccgcgcatcctgccgccctgcaccaacgcctccaccggctccgcgctgctcaaccccagcctcccgaaccagagcgacgtggtggaggccgagggcagccacagcaactcccccaccaacatggcgccctccgcgcgcctggaggaggccgtgtggaggccctactga

SEQ ID NO: 85 - DNA, 1704 bp, Homo sapiens
atgcttcattcgcctcacaaacaaccacagaaccacaagtgcggtgcaaactttctccaggaggacagcaagaagtctctggtttttaaatggttaatctccgcaggtcactaccagccaccgagaccaacagagtcatttaaggctgcaagcagtatttacaacagagggtacaagttctatctgaaaaaaaaaggagggactatggcatcaaacagcctcttcagcacagtgacaccatgtcagcaaaacttcttttgggatccgagcaccagccggcgcttcagccccccctccagcagcctgcagcccggcaaaatgagcgacgtgagcccggtggtggctgcgcaacagcagcagcaacagcagcagcagcaacagcagcagcagcagcagcaacagcagcagcagcagcaggaggcggcggcggcggctgcggcggcggcggcggctgcggcggcggcagctgcagtgccccggttgcggccgccccacgacaaccgcaccatggtggagatcatcgccgaccacccggccgaactcgtccgcaccgacagccccaacttcctgtgctcggtgctgccctcgcactggcgctgcaacaagaccctgcccgtggccttcaaggtggtagccctcggagaggtaccagatgggactgtggttactgtcatggcgggtaacgatgaaaattattctgctgagctccggaatgcctctgctgttatgaaaaaccaagtagcaaggttcaacgatctgagatttgtgggccggagtggacgaggcaagagtttcaccttgaccataaccgtcttcacaaatcctccccaagtagctacctatcacagagcaattaaagttacagtagatggacctcgggaacccagaaggcacagacagaagcttgatgactctaaacctagtttgttctctgaccgcctcagtgatttagggcgcattcctcatcccagtatgagagtaggtgtcccgcctcagaacccacggccctccctgaactctgcaccaagtccttttaatccacaaggacagagtcagattacagaccccaggcaggcacagtcttccccgccgtggtcctatgaccagtcttacccctcctacctgagccagatgacgtccccgtccatccactctaccaccccgctgtcttccacacggggcactgggcttcctgccatcaccgatgtgcctaggcgcatttcaggtgcttcagaactgggccctttttcagaccccaggcagttcccaagcatttcatccctcactgagagccgcttctccaacccacgaatgcactatccagccacctttacttacaccccgccagtcacctcaggcatgtccctcggtatgtccgccaccactcactaccacacctacctgccaccaccctaccccggctcttcccaaagccagagtggacccttccagaccagcagcactccatatctctactatggcacttcgtcaggatcctatcagtttcccatggtgccggggggagaccggtctccttccagaatgcttccgccatgcaccaccacctcgaatggcagcacgctattaaatccaaatttgcctaaccagaatgatggtgttgacgctgatggaagccacagcagttccccaactgttttgaattctagtggcagaatggatgaatctgtttggcgaccatattga

SEQ ID NO: 86 - DNA, 1389 bp, Homo sapiens
atgctgctgctggcgagatgtctgctgctagtcctcgtctcctcgctgctggtatgctcgggactggcgtgcggaccgggcagggggttcgggaagaggaggcaccccaaaaagctgacccctttagcctacaagcagtttatccccaatgtggccgagaagaccctaggcgccagcggaaggtatgaagggaagatctccagaaactccgagcgatttaaggaactcacccccaattacaaccccgacatcatatttaaggatgaagaaaacaccggagcggacaggctgatgactcagaggtgtaaggacaagttgaacgctttggccatctcggtgatgaaccagtggccaggagtgaaactgcgggtgaccgagggctgggacgaagatggccaccactcagaggagtctctgcactacgagggccgcgcagtggacatcaccacgtctgaccgcgaccgcagcaagtacggcatgctggcccgcctggcggtggaggccggcttcgactgggtgtactacgagtccaaggcacatatccactgctcggtgaaagcagagaactcggtggcggccaaatcgggaggctgcttcccgggctcggccacggtgcacctggagcagggcggcaccaagctggtgaaggacctgagccccggggaccgcgtgctggcggcggacgaccagggccggctgctctacagcgacttcctcactttcctggaccgcgacgacggcgccaagaaggtcttctacgtgatcgagacgcgggagccgcgcgagcgcctgctgctcaccgccgcgcacctgctctttgtggcgccgcacaacgactcggccaccggggagcccgaggcgtcctcgggctcggggccgccttccgggggcgcactggggcctcgggcgctgttcgccagccgcgtgcgcccgggccagcgcgtgtacgtggtggccgagcgtgacggggaccgccggctcctgcccgccgctgtgcacagcgtgaccctaagcgaggaggccgcgggcgcctacgcgccgctcacggcccagggcaccattctcatcaaccgggtgctggcctcgtgctacgcggtcatcgaggagcacagctgggcgcaccgggccttcgcgcccttccgcctggcgcacgcgctcctggctgcactggcgcccgcgcgcacggaccgcggcggggacagcggcggcggggaccgcgggggcggcggcggcagagtagccctaaccgctccaggtgctgccgacgctccgggtgcgggggccaccgcgggcatccactggtactcgcagctgctctaccaaataggcacctggctcctggacagcgaggccctgcacccgctgggcatggcggtcaagtccagctga

SEQ ID NO: 87 - DNA, 1530 bp, Homo sapiens
atgaatctcctggaccccttcatgaagatgaccgacgagcaggagaagggcctgtccggcgcccccagccccaccatgtccgaggactccgcgggctcgccctgcccgtcgggctccggctcggacaccgagaacacgcggccccaggagaacacgttccccaagggcgagcccgatctgaagaaggagagcgaggaggacaagttccccgtgtgcatccgcgaggcggtcagccaggtgctcaaaggctacgactggacgctggtgcccatgccggtgcgcgtcaacggctccagcaagaacaagccgcacgtcaagcggcccatgaacgccttcatggtgtgggcgcaggcggcgcgcaggaagctcgcggaccagtacccgcacttgcacaacgccgagctcagcaagacgctgggcaagctctggagacttctgaacgagagcgagaagcggcccttcgtggaggaggcggagcggctgcgcgtgcagcacaagaaggaccacccggattacaagtaccagccgcggcggaggaagtcggtgaagaacgggcaggcggaggcagaggaggccacggagcagacgcacatctcccccaacgccatcttcaaggcgctgcaggccgactcgccacactcctcctccggcatgagcgaggtgcactcccccggcgagcactcggggcaatcccagggcccaccgaccccacccaccacccccaaaaccgacgtgcagccgggcaaggctgacctgaagcgagaggggcgccccttgccagaggggggcagacagccccctatcgacttccgcgacgtggacatcggcgagctgagcagcgacgtcatctccaacatcgagaccttcgatgtcaacgagtttgaccagtacctgccgcccaacggccacccgggggtgccggccacgcacggccaggtcacctacacgggcagctacggcatcagcagcaccgcggccaccccggcgagcgcgggccacgtgtggatgtccaagcagcaggcgccgccgccacccccgcagcagcccccacaggccccgccggccccgcaggcgcccccgcagccgcaggcggcgcccccacagcagccggcggcacccccgcagcagccacaggcgcacacgctgaccacgctgagcagcgagccgggccagtcccagcgaacgcacatcaagacggagcagctgagccccagccactacagcgagcagcagcagcactcgccccaacagatcgcctacagccccttcaacctcccacactacagcccctcctacccgcccatcacccgctcacagtacgactacaccgaccaccagaactccagctcctactacagccacgcggcaggccagggcaccggcctctactccaccttcacctacatgaaccccgctcagcgccccatgtacacccccatcgccgacacctctggggtcccttccatcccgcagacccacagcccccagcactgggaacaacccgtctacacacagctcactcgaccttga

SEQ ID NO: 88 - DNA, 1245 bp, Homo sapiens
atgagcagcccggatgcgggatacgccagtgacgaccagagccagacccagagcgcgctgcccgcggtgatggccgggctgggcccctgcccctgggccgagtcgctgagccccatcggggacatgaaggtgaagggcgaggcgccggcgaacagcggagcaccggccggggccgcgggccgagccaagggcgagtcccgtatccggcggccgatgaacgctttcatggtgtgggctaaggacgagcgcaagcggctggcgcagcagaatccagacctgcacaacgccgagttgagcaagatgctgggcaagtcgtggaaggcgctgacgctggcggagaagcggcccttcgtggaggaggcagagcggctgcgcgtgcagcacatgcaggaccaccccaactacaagtaccggccgcggcggcgcaagcaggtgaagcggctgaagcgggtggagggcggcttcctgcacggcctggctgagccgcaggcggccgcgctgggccccgagggcggccgcgtggccatggacggcctgggcctccagttccccgagcagggcttccccgccggcccgccgctgctgcctccgcacatgggcggccactaccgcgactgccagagtctgggcgcgcctccgctcgacggctacccgttgcccacgcccgacacgtccccgctggacggcgtggaccccgacccggctttcttcgccgccccgatgcccggggactgcccggcggccggcacctacagctacgcgcaggtctcggactacgctggccccccggagcctcccgccggtcccatgcacccccgactcggcccagagcccgcgggtccctcgattccgggcctcctggcgccacccagcgcccttcacgtgtactacggcgcgatgggctcgcccggggcgggcggcgggcgcggcttccagatgcagccgcaacaccagcaccagcaccagcaccagcaccaccccccgggccccggacagccgtcgccccctccggaggcactgccctgccgggacggcacggaccccagtcagcccgccgagctcctcggggaggtggaccgcacggaatttgaacagtatctgcacttcgtgtgcaagcctgagatgggcctcccctaccaggggcatgactccggtgtgaatctccccgacagccacggggccatttcctcggtggtgtccgacgccagctccgcggtatattactgcaactatcctgacgtgtga

SEQ ID NO: 89 - DNA, 987 bp, Homo sapiens
atgactggagtctttgacagtctagtggctgatatgcactcgacccagatcgccgcctccagcacgtaccaccagcaccagcagcccccgagcggcggcggcgccggcccgggtggcaacagcagcagcagcagcagcctccacaagccccaggagtcgcccacccttccggtgtccaccgccaccgacagcagctactacaccaaccagcagcacccggcgggcggcggcggcggcgggggctcgccctacgcgcacatgggttcctaccagtaccaagccagcggcctcaacaacgtcccttactccgccaagagcagctatgacctgggctacaccgccgcctacacctcctacgctccctatggaaccagttcgtccccagccaacaacgagcctgagaaggaggaccttgagcctgaaattcggatagtgaacgggaagccaaagaaagtccggaaaccccgcaccatctactccagtttccagctggcggctcttcagcggcgtttccaaaagactcaatacttggccttgccggagcgagccgagctggcggcctctctgggcctcacccagactcaggtcaaaatctggttccagaaccgccggtccaagttcaagaagatgtggaaaagtggtgagatcccctcggagcagcaccctggggccagcgcttctccaccttgtgcttcgccgccagtctcagcgccggcctcctgggactttggtgtgccgcagcggatggcgggcggcggtggtccgggcagtggcggcagcggcgccggcagctcgggctccagcccgagcagcgcggcctcggcttttctgggcaactacccctggtaccaccagacctcgggatccgcctcacacctgcaggccacggcgccgctgctgcaccccactcagaccccgcagccgcatcaccaccaccaccatcacggcggcgggggcgccccggtgagcgcggggacgattttctaa

SEQ ID NO: 90 - DNA, 870 bp, Homo sapiens
atgacaggagtgtttgacagaagggtccccagcatccgatccggcgacttccaagctccgttccagacgtccgcagctatgcaccatccgtctcaggaatcgccaactttgcccgagtcttcagctaccgattctgactactacagccctacggggggagccccgcacggctactgctctcctacctcggcttcctatggcaaagctctcaacccctaccagtatcagtatcacggcgtgaacggctccgccgggagctacccagccaaagcttatgccgactatagctacgctagctcctaccaccagtacggcggcgcctacaaccgcgtcccaagcgccaccaaccagccagagaaagaagtgaccgagcccgaggtgagaatggtgaatggcaaaccaaagaaagttcgtaaacccaggactatttattccagctttcagctggccgcattacagagaaggtttcagaagactcagtacctcgccttgccggaacgcgccgagctggccgcctcgctgggattgacacaaacacaggtgaaaatctggtttcagaacaaaagatccaagatcaagaagatcatgaaaaacggggagatgcccccggagcacagtcccagctccagcgacccaatggcgtgtaactcgccgcagtctccagcggtgtgggagccccagggctcgtcccgctcgctcagccaccaccctcatgcccaccctccgacctccaaccagtccccagcgtccagctacctggagaactctgcatcctggtacacaagtgcagccagctcaatcaattcccacctgccgccgccgggctccttacagcacccgctggcgctggcctccgggacactctattag

SEQ ID NO: 91 - DNA, 843 bp, Homo sapiens
atgccagctgatataatggagaaaaattcctcgtccccggtggctgctaccccagccagtgtcaacacgacaccggataaaccaaagacagcatctgagcacagaaagtcatcaaagcctattatggagaaaagacgaagagcaagaataaatgaaagtctgagccagctgaaaacactgattttggatgctctgaagaaagatagctcgcggcattccaagctggagaaggcggacattctggaaatgacagtgaagcacctccggaacctgcagcgggcgcagatgacggctgcgctgagcacagacccaagtgtgctggggaagtaccgagccggcttcagcgagtgcatgaacgaggtgacccgcttcctgtccacgtgcgagggcgttaataccgaggtgcgcactcggctgctcggccacctggccaactgcatgacccagatcaatgccatgacctaccccgggcagccgcaccccgccttgcaggcgccgccaccgcccccaccgggacccggcggcccccagcacgcgccgttcgcgccgccgccgccactcgtgcccatccccgggggcgcggcgccccctcccggcggcgccccctgcaagctgggcagccaggctggagaggcggctaaggtgtttggaggcttccaggtggtaccggctcccgatggccagtttgctttcctcattcccaacggggccttcgcgcacagcggccctgtcatccccgtctacaccagcaacagcggcacctccgtgggccccaacgcagtgtcaccttccagcggcccctcgcttacggcggactccatgtggaggccgtggcggaactga

SEQ ID NO: 92 - DNA, 648 bp, Homo sapiens
atgggcagcccccgctccgcgctgagctgcctgctgttgcacttgctggtcctctgcctccaagcccaggtaactgttcagtcctcacctaattttacacagcatgtgagggagcagagcctggtgacggatcagctcagccgccgcctcatccggacctaccaactctacagccgcaccagcgggaagcacgtgcaggtcctggccaacaagcgcatcaacgccatggcagaggacggcgaccccttcgcaaagctcatcgtggagacggacacctttggaagcagagttcgagtccgaggagccgagacgggcctctacatctgcatgaacaagaaggggaagctgatcgccaagagcaacggcaaaggcaaggactgcgtcttcacggagattgtgctggagaacaactacacagcgctgcagaatgccaagtacgagggctggtacatggccttcacccgcaagggccggccccgcaagggctccaagacgcggcagcaccagcgtgaggtccacttcatgaagcggctgccccggggccaccacaccaccgagcagagcctgcgcttcgagttcctcaactacccgcccttcacgcgcagcctgcgcggcagccagaggacttgggcccccgagccccgatag

SEQ ID NO: 93 - DNA, 975 bp, Homo sapiens
atgaactgcatgaaaggcccgcttcacttggagcaccgagcagcggggaccaagctgtcggccgtctcctcatcttcctgtcaccatccccagccgttagccatggcttcggttctggctcccggtcagccccggtcgctggactcctccaagcacaggctggaggtgcacaccatctccgacacctccagcccggaggccgcagagaaagataaaagccagcaggggaagaatgaggacgtgggcgccgaggacccgtctaagaagaagcggcaaaggcggcagcggactcactttaccagccagcagctccaggagctggaggccactttccagaggaaccgctacccggacatgtccacacgcgaagaaatcgctgtgtggaccaaccttacggaagcccgagtccgggtttggttcaagaatcgtcgggccaaatggagaaagagggagcgcaaccagcaggccgagctatgcaagaatggcttcgggccgcagttcaatgggctcatgcagccctacgacgacatgtacccaggctattcctacaacaactgggccgccaagggccttacatccgcctccctatccaccaagagcttccccttcttcaactctatgaacgtcaaccccctgtcatcacagagcatgttttccccacccaactctatctcgtccatgagcatgtcgtccagcatggtgccctcagcagtgacaggcgtcccgggctccagtctcaacagcctgaataacttgaacaacctgagtagcccgtcgctgaattccgcggtgccgacgcctgcctgtccttacgcgccgccgactcctccgtatgtttatagggacacgtgtaactcgagcctggccagcctgagactgaaagcaaagcagcactccagcttcggctacgccagcgtgcagaacccggcctccaacctgagtgcttgccagtatgcagtggaccggcccgtgtga

SEQ ID NO: 94 - DNA, 987 bp, Mus musculus
atggccacccaggtgatggggcagtcttctggaggaggcagtctcttcaacaacagtgccaacatgggcatggccttaaccaacgacatgtacgacctgcacgagctctcgaaagctgaactggcagcccctcagctcatcatgttagccaacgtggccctgacgggggaggcaagcggcagctgctgcgattacctggtcggtgaagagaggcagatggccgaattgatgcccgtgggagacaaccacttctcagaaagtgaaggagaaggcctggaagagtcggctgacctcaaagggctggaaaacatggaactgggaagtttggagctaagtgctgtagaaccccagcccgtatttgaagcctcagctgccccagaaatatacagcgccaataaagatcccgctccagaaacacccgtggcggaagacaaatgcaggagttctaaggccaagcccttccggtgtaagccttgccagtacgaagccgaatctgaagagcagtttgtgcatcacatccggattcacagcgctaagaagttctttgtggaggaaagtgcagagaaacaggccaaagcctgggagtcggggtcgtctccggccgaagagggcgagttctccaaaggccccatccgctgtgaccgctgtggctacaataccaaccggtatgaccactacatggcacacctgaagcaccacctgcgagctggcgagaacgagcgcatctacaagtgcatcatctgcacgtacacgacggtcagcgagtaccactggaggaaacacctgagaaaccatttccccaggaaagtctacacctgcagcaagtgcaactacttctcagacagaaaaaataactacgttcagcacgtgcgaactcacacaggagaacgcccgtataaatgtgaactttgtccttactcaagctctcagaagactcatctaacgcgacacatgcggactcattcagagtgtgatctagctgggtga

SEQ ID NO: 95 - DNA, 1025 bp, Homo sapiens
atgaccatggaatctggagccgagaaccagcagagtggagatgcagctgtaacagaagctgaaaaccaacaaatgacagttcaagcccagccacagattgccacattagcccaggtatctatgccagcagctcatgcaacatcatctgctcccaccgtaactctagtacagctgcccaatgggcagacagttcaagtccatggagtcattcaggcggcccagccatcagttattcagtctccacaagtccaaacagttcagtcttcctgtaaggacttaaaaagacttttctccggaacacagatttcaactattgcagaaagtgaagattcacaggagtcagtggatagtgtaactgattcccaaaagcgaagggaaattctttcaaggaggccttcctacaggaaaattttgaatgacttatcttctgatgcaccaggagtgccaaggattgaagaagagaagtctgaagaggagacttcagcacctgccatcaccactgtaacggtgccaactccaatttaccaaactagcagtggacagtatattgccattacccagggaggagcaatacagctggctaacaatggtaccgatggggtacagggcctgcaaacattaaccatgaccaatgcagcagccactcagccgggtactaccattctacagtatgcacagaccactgatggacagcagatcttagtgcccagcaaccaagttgttgttcaagctgcctctggagacgtacaaacataccagattcgcacagcacccactagcactattgcccctggagttgttatggcatcctccccagcacttcctacacagcctgctgaagaagcagcacgaaagagagaggtccgtctaatgaagaacagggaagcagctcgagagtgtcgtagaaagaagaaagaatatgtgaaatgtttagaaaacagagtggcagtgcttgaaaatcaaaacaagacattgattgaggagctaaaagcacttaaggacctttactgccacaaatcagatta

SEQ ID NO: 96 - DNA, 963 bp, Mus musculus
atggctgagggcaaaggggctcctctgaggccttcagttgagaagagatggaagctcatggaacccaagcagacccaggcagggatgttcaagaaaatgagccttgtggactctgacactgctgcaggaaagggtagccaagatgaggcctatactgaactgagcctgccaacagcaccgaacaagcctcgactggacaggcctcgggcctgcaaggcatacacagagcagaggcacaataccttcacagagctatcatgtctccaggagaggccaggggacatccaggcccagacgaggaagctggagaacccagaaggccagctcggccctcagcagctgccctcgagtttcctcagagcctcaggtgatggcacagtgtgttcagcatggccaggtgccccccggagtgagcagaaaagtgctttcagcaagccagccaaacgcccagcagagaaacctaagcgctctcccatgcttctggctggtggaagtgcagagggctcatgggagctctcaggactcatcaccactgtggacatcccatattgggctcatctgtcaactttcaagttcatgggtgatttctggaaattgcacacattgtcacagaacattctcctctgcaatgctttccagggggctcccacaccatggctggagcatacccaggtacaagcccccacatcctcagctccttcctccacagcctcccgggctctcttgccgcccacactctcctccttgggcttgtctactcagaactggtgtgcgaagtgcaacctagcctttcgcctgacagctgacctggtcttccacatgcggtcacatcacaaaagggaacacgtgggccctgacccacattctaagaaacgaagagaggaagttctcacttgccccgtttgccacgagtacttccgggagcgccaccatctgtccaggcatatggcttcacatagttag

SEQ ID NO: 97 - DNA, 1374 bp, Homo sapiens
atgctgggagcggtgaagatggaagggcacgagccgtccgactggagcagctactatgcagagcccgagggctactcctccgtgagcaacatgaacgccggcctggggatgaacggcatgaacacgtacatgagcatgtcggcggccgccatgggcagcggctcgggcaacatgagcgcgggctccatgaacatgtcgtcgtacgtgggcgctggcatgagcccgtccctggcggggatgtcccccggcgcgggcgccatggcgggcatgggcggctcggccggggcggccggcgtggcgggcatggggccgcacttgagtcccagcctgagcccgctcggggggcaggcggccggggccatgggcggcctggccccctacgccaacatgaactccatgagccccatgtacgggcaggcgggcctgagccgcgcccgcgaccccaagacctacaggcgcagctacacgcacgcaaagccgccctactcgtacatctcgctcatcaccatggccatccagcagagccccaacaagatgctgacgctgagcgagatctaccagtggatcatggacctcttccccttctaccggcagaaccagcagcgctggcagaactccatccgccactcgctctccttcaacgactgtttcctgaaggtgccccgctcgcccgacaagcccggcaagggctccttctggaccctgcaccctgactcgggcaacatgttcgagaacggctgctacctgcgccgccagaagcgcttcaagtgcgagaagcagctggcgctgaaggaggccgcaggcgccgccggcagcggcaagaaggcggccgccggagcccaggcctcacaggctcaactcggggaggccgccgggccggcctccgagactccggcgggcaccgagtcgcctcactcgagcgcctccccgtgccaggagcacaagcgagggggcctgggagagctgaaggggacgccggctgcggcgctgagccccccagagccggcgccctctcccgggcagcagcagcaggccgcggcccacctgctgggcccgccccaccacccgggcctgccgcctgaggcccacctgaagccggaacaccactacgccttcaaccacccgttctccatcaacaacctcatgtcctcggagcagcagcaccaccacagccaccaccaccaccaaccccacaaaatggacctcaaggcctacgaacaggtgatgcactaccccggctacggttcccccatgcctggcagcttggccatgggcccggtcacgaacaaaacgggcctggacgcctcgcccctggccgcagatacctcctactaccagggggtgtactcccggcccattatgaactcctcttaa

SEQ ID NO: 98 - DNA, 1221 bp, Homo sapiens
atggatggatggagaaggatgcctcgctggggactgctgctgctgctctggggctcctgtacctttggtctcccgacagacaccaccacctttaaacggatcttcctcaagagaatgccctcaatccgagaaagcctgaaggaacgaggtgtggacatggccaggcttggtcccgagtggagccaacccatgaagaggctgacacttggcaacaccacctcctccgtgatcctcaccaactacatggacacccagtactatggcgagattgggatcgggaccccaccccaaaccttcaaagtcgtctttgacactggttcgtccaatgtttgggtgccctcctccaagtgcagccgtctctacactgcctgtgtgtatcacaagctcttcgatgcttcggattcctccagctacaagcacaatggaacagaactcaccctccgctattcaacagggacagtcagtggctttctcagccaggacatcatcaccgtgggtggaatcacggtgacacagatgtttggagaggtcacggagatgcccgccttacccttcatgctggccgagtttgatggggttgtgggcatgggcttcattgaacaggccattggcagggtcacccctatcttcgacaacatcatctcccaaggggtgctaaaagaggacgtcttctctttctactacaacagagattccgagaattcccaatcgctgggaggacagattgtgctgggaggcagcgacccccagcattacgaagggaatttccactatatcaacctcatcaagactggtgtctggcagattcaaatgaagggggtgtctgtggggtcatccaccttgctctgtgaagacggctgcctggcattggtagacaccggtgcatcctacatctcaggttctaccagctccatagagaagctcatggaggccttgggagccaagaagaggctgtttgattatgtcgtgaagtgtaacgagggccctacactccccgacatctctttccacctgggaggcaaagaatacacgctcaccagcgcggactatgtatttcaggaatcctacagtagtaaaaagctgtgcacactggccatccacgccatggatatcccgccacccactggacccacctgggccctgggggccaccttcatccgaaagttctacacagagtttgatcggcgtaacaaccgcattggcttcgccttggcccgctga

SEQ ID NO: 99 - DNA, 654 bp, Homo sapiens
atgagtctggtaggtggttttccccaccacccggtggtgcaccacgagggctacccgtttgccgccgccgccgccgcagctgccgccgccgccgccagccgctgcagccatgaggagaacccctacttccatggctggctcatcggccaccccgagatgtcgccccccgactacagcatggccctgtcctacagccccgagtatgccagcggcgccgccggcctggaccactcccattacgggggggtgccgccgggcgccgggcccccgggcctgggggggccgcgcccggtgaagcgccgaggcaccgccaaccgcaaggagcggcgcaggactcagagcatcaacagcgccttcgccgaactgcgcgagtgcatccccaacgtacccgccgacaccaaactctccaaaatcaagaccctgcgcctggccaccagctacatcgcctacctcatggacctgctggccaaggacgaccagaatggcgaggcggaggccttcaaggcagagatcaagaagaccgacgtgaaagaggagaagaggaagaaggagctgaacgaaatcttgaaaagcacagtgagcagcaacgacaagaaaaccaaaggccggacgggctggccgcagcacgtctgggccctggagctcaagcagtga

SEQ ID NO: 100 - DNA, 942 bp, Homo sapiens
atgacttcttgtcacattgctgaagaacatatacaaaaggttgctatctttggaggaacccatgggaatgagctaaccggagtatttctggttaagcattggctagagaatggcgctgagattcagagaacagggctggaggtaaaaccatttattactaaccccagagcagtgaagaagtgtaccagatatattgactgtgacctgaatcgcatttttgaccttgaaaatcttggcaaaaaaatgtcagaagatttgccatatgaagtgagaagggctcaagaaataaatcatttatttggtccaaaagacagtgaagattcctatgacattatttttgaccttcacaacaccacctctaacatggggtgcactcttattcttgaggattccaggaataactttttaattcagatgtttcattacattaagacttctctggctccactaccctgctacgtttatctgattgagcatccttccctcaaatatgcgaccactcgttccatagccaagtatcctgtgggtatagaagttggtcctcagcctcaaggggttctgagagctgatatcttggatcaaatgagaaaaatgattaaacatgctcttgattttatacatcatttcaatgaaggaaaagaatttcctccctgcgccattgaggtctataaaattatagagaaagttgattacccccgggatgaaaatggagaaattgctgctatcatccatcctaatctgcaggatcaagactggaaaccactgcatcctggggatcccatgtttttaactcttgatgggaagacgatcccactgggcggagactgtaccgtgtaccccgtgtttgtgaatgaggccgcatattacgaaaagaaagaagcttttgcaaagacaactaaactaacgctcaatgcaaaaagtattcgctgctgtttacattag

SEQ ID NO: 101 - DNA, 1590 bp, Homo sapiens
atgacaagctccaggctttggttttcgctgctgctggcggcagcgttcgcaggacgggcgacggccctctggccctggcctcagaacttccaaacctccgaccagcgctacgtcctttacccgaacaactttcaattccagtacgatgtcagctcggccgcgcagcccggctgctcagtcctcgacgaggccttccagcgctatcgtgacctgcttttcggttccgggtcttggccccgtccttacctcacagggaaacggcatacactggagaagaatgtgttggttgtctctgtagtcacacctggatgtaaccagcttcctactttggagtcagtggagaattataccctgaccataaatgatgaccagtgtttactcctctctgagactgtctggggagctctccgaggtctggagacttttagccagcttgtttggaaatctgctgagggcacattctttatcaacaagactgagattgaggactttccccgctttcctcaccggggcttgctgttggatacatctcgccattacctgccactctctagcatcctggacactctggatgtcatggcgtacaataaattgaacgtgttccactggcatctggtagatgatccttccttcccatatgagagcttcacttttccagagctcatgagaaaggggtcctacaaccctgtcacccacatctacacagcacaggatgtgaaggaggtcattgaatacgcacggctccggggtatccgtgtgcttgcagagtttgacactcctggccacactttgtcctggggaccaggtatccctggattactgactccttgctactctgggtctgagccctctggcacctttggaccagtgaatcccagtctcaataatacctatgagttcatgagcacattcttcttagaagtcagctctgtcttcccagatttttatcttcatcttggaggagatgaggttgatttcacctgctggaagtccaacccagagatccaggactttatgaggaagaaaggcttcggtgaggacttcaagcagctggagtccttctacatccagacgctgctggacatcgtctcttcttatggcaagggctatgtggtgtggcaggaggtgtttgataataaagtaaagattcagccagacacaatcatacaggtgtggcgagaggatattccagtgaactatatgaaggagctggaactggtcaccaaggccggcttccgggcccttctctctgccccctggtacctgaaccgtatatcctatggccctgactggaaggatttctacgtagtggaacccctggcatttgaaggtacccctgagcagaaggctctggtgattggtggagaggcttgtatgtggggagaatatgtggacaacacaaacctggtccccaggctctggcccagagcaggggctgttgccgaaaggctgtggagcaacaagttgacatctgacctgacatttgcctatgaacgtttgtcacacttccgctgtgagttgctgaggcgaggtgtccaggcccaacccctcaatgtaggcttctgtgagcaggagtttgaacagacctga

SEQ ID NO: 102 - DNA, 657 bp, Homo sapiens
atggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaa

SEQ ID NO: 103 - DNA, 1956 bp, Homo sapiens
atggcccgggggtcggcggttgcctgggcggcgctcgggccgttgttgtggggctgcgcgctggggctgcagggcgggatgctgtacccccaggagagcccgtcgcgggagtgcaaggagctggacggcctctggagcttccgcgccgacttctctgacaaccgacgccggggcttcgaggagcagtggtaccggcggccgctgtgggagtcaggccccaccgtggacatgccagttccctccagcttcaatgacatcagccaggactggcgtctgcggcattttgtcggctgggtgtggtacgaacgggaggtgatcctgccggagcgatggacccaggacctgcgcacaagagtggtgctgaggattggcagtgcccattcctatgccatcgtgtgggtgaatggggtcgacacgctagagcatgaggggggctacctccccttcgaggccgacatcagcaacctggtccaggtggggcccctgccctcccggctccgaatcactatcgccatcaacaacacactcacccccaccaccctgccaccagggaccatccaatacctgactgacacctccaagtatcccaagggttactttgtccagaacacatattttgactttttcaactacgctggactgcagcggtctgtacttctgtacacgacacccaccacctacatcgatgacatcaccgtcaccaccagcgtggagcaagacagtgggctggtgaattaccagatctctgtcaagggcagtaacctgttcaagttggaagtgcgtcttttggatgcagaaaacaaagtcgtggcgaatgggactgggacccagggccaacttaaggtgccaggtgtcagcctctggtggccgtacctgatgcacgaacgccctgcctatctgtattcattggaggtgcagctgactgcacagacgtcactggggcctgtgtctgacttctacacactccctgtggggatccgcactgtggctgtcaccaagagccagttcctcatcaatgggaaacctttctatttccacggtgtcaacaagcatgaggatgcggacatccgagggaagggcttcgactggccgctgctggtgaaggacttcaacctgcttcgctggcttggtgccaacgctttccgtaccagccactacccctatgcagaggaagtgatgcagatgtgtgaccgctatgggattgtggtcatcgatgagtgtcccggcgtgggcctggcgctgccgcagttcttcaacaacgtttctctgcatcaccacatgcaggtgatggaagaagtggtgcgtagggacaagaaccaccccgcggtcgtgatgtggtctgtggccaacgagcctgcgtcccacctagaatctgctggctactacttgaagatggtgatcgctcacaccaaatccttggacccctcccggcctgtgacctttgtgagcaactctaactatgcagcagacaagggggctccgtatgtggatgtgatctgtttgaacagctactactcttggtatcacgactacgggcacctggagttgattcagctgcagctggccacccagtttgagaactggtataagaagtatcagaagcccattattcagagcgagtatggagcagaaacgattgcagggtttcaccaggatccacctctgatgttcactgaagagtaccagaaaagtctgctagagcagtaccatctgggtctggatcaaaaacgcagaaaatacgtggttggagagctcatttggaattttgccgatttcatgactgaacagtcaccgacgagagtgctggggaataaaaaggggatcttcactcggcagagacaaccaaaaagtgcagcgttccttttgcgagagagatactggaagattgccaatgaaaccaggtatccccactcagtagccaagtcacaatgtttggaaaacagcccgtttacttga

SEQ ID NO: 104 - DNA, 1671 bp, Homo sapiens
atggagctgtgcgggctggggctgccccggccgcccatgctgctggcgctgctgttggcgacactgctggcggcgatgttggcgctgctgactcaggtggcgctggtggtgcaggtggcggaggcggctcgggccccgagcgtctcggccaagccggggccggcgctgtggcccctgccgctctcggtgaagatgaccccgaacctgctgcatctcgccccggagaacttctacatcagccacagccccaattccacggcgggcccctcctgcaccctgctggaggaagcgtttcgacgatatcatggctatatttttggtttctacaagtggcatcatgaacctgctgaattccaggctaaaacccaggttcagcaacttcttgtctcaatcacccttcagtcagagtgtgatgctttccccaacatatcttcagatgagtcttatactttacttgtgaaagaaccagtggctgtccttaaggccaacagagtttggggagcattacgaggtttagagacctttagccagttagtttatcaagattcttatggaactttcaccatcaatgaatccaccattattgattctccaaggttttctcacagaggaattttgattgatacatccagacattatctgccagttaagattattcttaaaactctggatgccatggcttttaataagtttaatgttcttcactggcacatagttgatgaccagtctttcccatatcagagcatcacttttcctgagttaagcaataaaggaagctattctttgtctcatgtttatacaccaaatgatgtccgtatggtgattgaatatgccagattacgaggaattcgagtcctgccagaatttgatacccctgggcatacactatcttggggaaaaggtcagaaagacctcctgactccatgttacagtagacaaaacaagttggactcttttggacctataaaccctactctgaatacaacatacagcttccttactacatttttcaaagaaattagtgaggtgtttccagatcaattcattcatttgggaggagatgaagtggaatttaaatgttgggaatcaaatccaaaaattcaagatttcatgaggcaaaaaggctttggcacagattttaagaaactagaatctttctacattcaaaaggttttggatattattgcaaccataaacaagggatccattgtctggcaggaggtttttgatgataaagcaaagcttgcgccgggcacaatagttgaagtatggaaagacagcgcatatcctgaggaactcagtagagtcacagcatctggcttccctgtaatcctttctgctccttggtacttagatttgattagctatggacaagattggaggaaatactataaagtggaacctcttgattttggcggtactcagaaacagaaacaacttttcattggtggagaagcttgtctatggggagaatatgtggatgcaactaacctcactccaagattatggcctcgggcaagtgctgttggtgagagactctggagttccaaagatgtcagagatatggatgacgcctatgacagactgacaaggcaccgctgcaggatggtcgaacgtggaatagctgcacaacctctttatgctggatattgtaaccatgagaacatgtaa

SEQ ID NO: 105 - DNA, 1290 bp, Homo sapiens
atgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 106 - DNA, 1611 bp, Homo sapiens
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtga

SEQ ID NO: 107 - DNA, 642 bp, Homo sapiens
atgccccggagggcggagaactgggacgaggccgaggtaggcgcggaggaggcaggcgtcgaagagtacggccctgaagaagacggcggggaggagtcgggcgccgaggagtccggcccggaagagtccggcccggaggaactgggcgccgaggaggagatggaggccgggcggccgcggcccgtgctgcgctcggtgaactcgcgcgagccctcccaggtcatcttctgcaatcgcagtccgcgcgtcgtgctgcccgtatggctcaacttcgacggcgagccgcagccctacccaacgctgccgcctggcacgggccgccgcatccacagctaccgaggtcacctttggctcttcagagatgcagggacacacgatgggcttctggttaaccaaactgaattatttgtgccatctctcaatgttgacggacagcctatttttgccaatatcacactgccagtgtatactctgaaagagcgatgcctccaggttgtccggagcctagtcaagcctgagaattacaggagactggacatcgtcaggtcgctctacgaagatctggaagaccacccaaatgtgcagaaagacctggagcggctgacacaggagcgcattgcacatcaacggatgggagattga

SEQ ID NO: 108 - DNA, 444 bp, Homo sapiens
atggtgcatctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaa

SEQ ID NO: 109 - DNA, 951 bp, Homo sapiens
atgatagtgtttgtcaggttcaactccagccatggtttcccagtggaggtcgattctgacaccagcatcttccagctcaaggaggtggttgctaagcgacagggggttccggctgaccagttgcgtgtgattttcgcagggaaggagctgaggaatgactggactgtgcaggaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgttgggaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtag

(19) United States
(12) Patent Application Publication
(10) Pub. No.: US 2011/0217274 A1
(43) Pub. Date: Sep. 8, 2011

(54) METHODS FOR PRODUCTION AND USES OF MULTIPOTENT ,PLURIPOTENT, DIFFERENTIATED AND DISEASE-RESISTANT CELL POPULATIONS

(76) Inventor: Christopher B. Reld, Los Angeles, CA (US)

(21) Appl. No.: 12/601,819

(22) PCT Filed: May 28, 2008
(86) PCT No.: PCT/US08/65007

§ 371 Date: May 31, 2011

Related U.S. Application Data
(60) Provisional application No. 60/932,020, filed on May 29, 2007.
(60) Provisional application No. 60/933,133, filed on Jun. 5, 2007.
(60) Provisional application No. 60/933,670, filed on Jun. 8, 2007.
(60) Provisional application No. 61/006,449, filed on Jan. 14, 2008.
(60) Provisional application No. 61/064,761, filed on Mar. 25, 2008.

(51) Int. Cl. A61K 35/12; A61K 35/28; A61K 35/50; A61K 35/48; A61K 35/36; A61K 35/26; C12N 5/10; C12N 15/63

(52) U.S. Cl. 424/93.21; 435/325; 435/320.1

(57) ABSTRACT

The claimed invention is directed towards the generation of pluripotent, multipotent, and/or self-renewing cells which are capable of beginning to differentiate in culture into a variety of cell types and capable of further differentiation in vivo. The claimed invention is also directed towards the generation of desirable, differentiating cell populations transplantable to patients, genetic modification of endogenous cells, and the treatment of patients suffering from diseases that may be ameliorated by these methods. This invention also provides methods for preventing, treating, or retarding disease related to immunodeficiency virus (e.g. HIV-1, HIV-2, SIV, FIV, etc.) infection.

### CROSS-REFERENCE TO RELATED APPLICATIONS

This national phase application under 35 U.S.C. §371 is a continuation under 35 USC §120 of international application PCT/US2008/065007, filed May 28, 2008, and claims the benefit under 35 U.S.C. §119 to U.S. Provisional Application Ser. No. 60/932,020, filed May 29, 2007, U.S. Provisional Application Ser. No. 60/933,133, filed Jun. 5, 2007, U.S. Provisional Application Ser. No. 60/933,670, filed Jun. 8, 2007, U.S. Provisional Application Ser. No. 61/006,449, filed Jan. 14, 2008, and U.S. Provisional Application Ser. No. 61/064,761, filed Mar. 25, 2008, the entire contents of which applications are hereby incorporated by reference in their entireties.

### BACKGROUND OF THE INVENTION

The transcendent challenge for medicine in the 21st century will be replacing damaged, worn-out or genetically-compromised cells. Transcription factors binding specifically to DNA play a vital role in regulating gene expression. It is the particular complement of transcription factors within an individual cell, that determine which cellular programs are active and which are turned off. In this capacity transcription factors play a decisive role in determining and maintaining cellular identity, as well as determining cellular vulnerability.

### SUMMARY OF THE INVENTION

The ability to derive proliferating, self-renewing, multipotent and pluripotent cell population(s) from otherwise non-pluripotent, non-self renewing cells may have significant positive implications for all fields utilizing cellular therapies. These fields include bone marrow transplantation, transfusion medicine, and gene therapy and enable the production of patient-specific stem cells and other desired cell types. Likewise, the ability to initiate differentiation of cells into neural, muscle, and various other desirable cell populations is and will also be of significant value to medicine and commercial processes involving animals. Accordingly, the present invention provides methods for genetic production and uses of multipotent cell populations, pluripotent cell populations, neuronal cell populations, muscle cell populations, and other desired cell populations such as, for example, HIV resistant cell populations.

It is a proposition of the present invention that the efficient introduction or overexpression of specific transcription factors, alone or in combination with other cell fate determinants (such as notch, numb and numblike), enables the interconversion of what have been considered transitory (multipotent, pluripotent, and/or self-renewing) or fixed (differentiated or somatic) cellular phenotypes. The ability to reliably induce phenotypic conversion or cellular reprogramming allows the production of stem cells, replacement cells, tissues, and organs that match individual patients. In conjunction with gene therapy techniques and cell culture techniques, cell type interconversion also provides for the production of disease-resistant and genetically-repaired cells that are suitable for transplantation.

It is an object of this invention to provide various manners of generating proliferating, self-renewing, multipotent and/or pluripotent cell population(s), as well as other desirable cell populations, from either dividing or non-dividing cells without the use of oncogenes. Differentiating cell populations comprise cells expressing some, but not all markers associated with specific cell type categorization. It is disclosed herein that appropriate Numb isoform expression in combination with other transgenes (especially transcription factors) enables the production of dividing, pluripotent cell populations or differentiating cell populations. Moreover, the genetic vectors of the present invention may be used to produce genetic modification (e.g. expression of gene products deficient in the patient) and to transiently or permanently induce proliferation, self-renewal, or stem/progenitor cell behavior in endogenous cells in vivo, particularly those cells found in tissues which normally do not show or no longer show such behavior. Finally, other genetic vectors of the present invention may be used to produce genetic modification and/or to block proliferation, self-renewal, or stem/progenitor cell behavior in cells aberrantly displaying such behavior (e.g. cancer cells). It is also an object of the present invention to provide therapeutic vectors and cells capable of expressing synthetic oligonucleotide sequences predicted to attenuate disease processes. For example, the current invention discloses the use of synthetic oligonucleotides to reduce gene expression critical HIV and other immunodeficiency virus infection, propagation and spread.

The invention may be used with any suitable cells, including vertebrate cells, and including fish, mammalian, avian, amphibian, and reptilian cells.

### BRIEF DESCRIPTION OF THE DRAWING

FIG. 1. A schematized vector map corresponding to the vector sequence of Example 13.

### DETAILED DESCRIPTION

All patents, patent applications, and publications cited in this application are hereby incorporated by reference herein in their entireties.

As discussed herein, “DNA” refers to deoxyribonucleic acid and “RNA” refers to ribonucleic acid. As discussed herein, “cDNA” refers to complementary DNA; “mRNA” refers to messenger RNA; “siRNA” refers to small interfering RNA; “shRNA” refers to small hairpin RNA; “miRNA” refers to microRNA, such as single-stranded RNA molecules, typically about 20-30 nucleotides in length, which may regulate gene expression; “decoy” and “decoy RNA” and “RNA decoy” refer to an RNA molecule that mimics the natural binding domain for a ligand.

As used herein, the meaning of the term “ameliorating” includes lessening an effect, or reducing damage, or minimizing the effect or impact of an action, activity, or function, and includes, for example, lessening the deleterious effects of a disease or condition.

As used herein, the meaning of the term “retarding” includes slowing or lessening the progress of an effect or action, and includes, for example, slowing the progress of a disease, slowing the rate of infection, or otherwise acting to slow or reduce the advance or progress of a disease or condition.

As used herein, an “inducing agent” is an agent that aids or is alone effective to promote an action. For example, an exogenous agent that affects a promoter, e.g., by initiating or enhancing its activity, and so affects expression of a gene under control of the promoter, may be termed an inducing agent. For example, tetracycline may be used as an inducing agent; and doxycycline may be used as an inducing agent.

A nucleic acid sequence (e.g., a nucleic acid sequence encoding a polypeptide) is termed “operably linked” to another nucleic acid sequence (e.g., a promoter) when the first nucleic acid sequence is placed in a functional relationship with the second nucleic acid sequence. For example, a promoter is operably linked to a coding sequence if the promoter affects the transcription or expression of the coding sequence. As used herein, the term “driven by” refers to a gene or coding sequence that is operably linked to a promoter sequence, and that the promoter sequence affects the transcription or expression of the coding sequence.

As used herein, a “marker” is a molecule that is detectable, or codes for a detectable molecule, or acts on other molecules so that the presence of the marker is detectable. A “marker protein” or “marker polypeptide” is a protein or polypeptide that is detectable in a laboratory or clinical environment, and, in embodiments, may be detectable by eye. A “marker gene” encodes a marker protein or marker polypeptide.

As used herein, “HIV” refers to human immunodeficiency virus, and includes variants such as, e.g., HIV-1, HIV-2. Other immunodeficiency viruses include simian immunodeficiency virus (SIV) and feline immunodeficiency virus (FIV). Enzymes related to HIV may be termed “HIV enzymes” and include, for example,\integrase, protease, reverse transcriptase, and transactivating regulatory protein (TAT).

Infection by HIV is believed to involve receptors termed “HIV receptors.” There may be multiple such receptors, some of which may be termed “HIV co-receptors.” As discussed herein, HIV co-receptors include CXCR4 and CCR5.

A theoretical basis for the embodiments of the invention is described herein, however, this discussion is not in any way to be considered as binding or limiting on the present invention. Those of skill in the art will understand that the various embodiments of the invention may be practiced regardless of the model used to describe the theoretical underpinnings of the invention.

In a preferred embodiment, cells are “selected” from accessible, dividing or non-dividing cell populations for the purpose of generating the desired a) proliferating, multipotent or pluripotent cell population, differentiating b) populations of neuronal cells c) muscle cells, d) and/or any other desired cell population; moreover the desired cell population may be capable of further differentiation in vitro, in vivo, and/or tissue-appropriate and regionally-appropriate differentiation in vivo.

Sources of Cells Selected for Use in the Invention:

Selected cells may include any cell practicable in the present invention. Cells selected for use in the present invention (herein termed “selected cells”) may originate as endogenous cells of the patient—including cells derived from other organ systems; or from exogenous sources (including those derived from cell lines, cryopreserved sources, banked sources, and donors). Cells may also be selected from cells genetically-modified with synthetic or natural nucleic acid sequences. The term “selected cells” as used herein does not include human embryonic stem cells.

In embodiments of the present invention, in order that they may be isolated without the involvement of invasive procedures, selected cells will preferably be easily accessible cells (e.g. peripheral blood leukocytes, circulating hematopoietic stem cells, epithelial cells (e.g. buccal cheek cells (e.g. Michalczyk et al., 2004)), adipose tissue (e.g. Gimble et al., 2007; Ma et al., 2007), umbilical cord blood cells (e.g. Zhao, et al., 2006; Tian et al., 2007), etc.). However, bone marrow stem cells, spermatogonia (e.g. Guan et al., 2006; Takahashi et al., 2007), primordial germ cells (PGCs), stem cells isolated from amniotic membranes (e.g. Ilancheran et al., 2007), amniotic fluid (e.g. De Coppi et al., 2007), as well as cells isolated from the skin (e.g. Tumbar, 2006; Dunnwald et al., 2001; Szudal'tseva et al., 2007), etc., are also covered by the present invention. Such cells can be isolated from the tissues in which they reside by any means known to the art.

Spermatogonia cells can be isolated using a two-step enzymatic digestion followed by Percoll separation. Cells can then be resuspended in minimum essential medium (MEM) supplemented with bovine serum albumin to a final concentration of 10⁶/mL. In detail: Tubule fragments are accessed surgically and teased apart prior to treatment with 1 mg/ml trypsin, hyaluronidase, and collagenase, and then 1 mg/ml hyaluronidase and collagenase, in MEM containing 0.10% sodium bicarbonate, 4 mM L-glutamine, nonessential amino acids, 40 microgram/ml gentamycin, 100 IU to 100 microgram/ml penicillin-streptomycin, and 15 mM HEPES. Spermatogonia cells are further separated from tubule fragments by centrifugation at 30 times gravity. After filtration through nylon filters with 77- and/or 55-micron pore sizes, cells are collected and loaded onto a discontinuous Percoll density gradient. Fractions with a purity greater than 40% progenitor/stem/spermatogonia cells are washed and resuspended to a concentration of cells equivalent to 10⁶ progenitor/stem/spermatogonia cells per ml. Afterwards cells are cultured and/or stored by any cryopreservation technique known to the art.

The selected cells may be genetically-modified cells, especially cells that have been genetically modified by any means known to the art, to encode therapeutic or commercially useful deoxyribonucleic acid (DNA) or ribonucleic acid (RNA) sequences.

In accordance with an aspect of the present invention, there is provided a method of producing a desired cell population (e.g. pluripotent, neuronal, muscle, etc.) from the selected cells.

Achieving multipotent, pluripotent, and/or self renewing cell populations:

In order to achieve a) a population of proliferating, self renewing pluripotent cells, the selected cell(s) and/or their progeny are transfected with nucleotide sequence(s) including those encoding the “long” (PRR insert+) isoform(s) of the mammalian numb gene. At about the same time the selected cells may also be transfected with synthetic oligonucleotides targeting the short Numb isoforms and Numblike, then cultured under conditions which promote growth of the selected cells at an optimal growth rate. Selected cells are maintained under these conditions for the period of time sufficient to achieve the desired cell number.

The cells are grown at the (optimal) rate of growth achieved by incubation with LIF, steel factor, and/or equipotent concentrations of Il-6, hyper IL-6, IL-7, oncostatin-M and/or cardiotrophin-1; or that growth rate achieved in the presence of other growth enhancing cytokines (e.g. those conditions described for culturing pluripotent cells e.g. Guan et al., 2006). The growth rate is determined from the doubling times of the selected cells in said growth culture medium. Likewise, culture conditions such as those described in U.S. Pat. Nos. 6,432,711 and 5,453,357 may also be suitable for the propagation and expansion, at an optimal growth rate, of cells transfected with the long (PRR+) Numb isioform(s). Other appropriate protocols and reference cytokine concentrations have been taught by Koshimizu et al., 1996; Keller et al., 1996; Piquet-Pellorce, 1994; Rose et al., 1994; Park and Han, 2000; Guan et al., 2006; Dykstra et al., 2006; Zhang et al., 2007). However the practice of the present invention is not limited to the details of these teachings.

In a preferred embodiment, the selected cells are cultured in a standard growth medium (e.g. Minimal Essential Medium with or without supplements (e.g. glutamine, and beta.-mercaptoethanol). The medium may include basic fibroblast growth factor (bFGF), steel factor, leukemia inhibitory factor (LIF), and/or factors with LIF activity (e.g. LIF, LIF receptor (LIFR), ciliary Neurotrophic factor (CNTF), oncostatin M (OSM), OSM receptor (OSMR), cardiotrophin, interleukins (IL) such as IL-6, hyper IL-6, GP130, etc.) as well as horse serum. LIF, as well as other factors with LIF activity, prevents spontaneous differentiation of the cells. Under these conditions, selected cells transfected with the PRR+Numb isoform(s) and their progeny are expected to achieve multipotency, pluripotency and/or self-renewal.

In a preferred embodiment, the selected cell(s) and/or their progeny are transfected with nucleotide sequence(s) encoding the “long” (PRR insert+) Numb isoform(s) as well as sequences encoding other transgenes. Many of those transgenes are listed below along with their corresponding identification numbers (accession numbers) in the NCBI sequence database.

In another preferred embodiment, the selected cell(s) and/or their progeny are transfected with nucleotide sequence(s) encoding a portion of the “long” (PRR insert+) Numb isoform(s) as well as sequences encoding other transgenes. Many of those transgenes are listed below along with their corresponding identification (accession) numbers (codes) in the NCBI sequence database.

In a preferred embodiment, the selected cells and/or their progeny are transfected with long (PRR+) Numb isoform encoding sequences as well as sequences encoding other transgenes, including LIF.

In a preferred embodiment, the selected cells and/or their progeny are transfected with long (PRR+) Numb isoform encoding sequences as well as sequences encoding other transgenes, including ones with LIF activity.

In a preferred embodiment, the selected cells and/or their progeny are transfected with long (PRR+) Numb isoform(s) encoding sequences as well as sequences encoding other transgenes, including the LIFR.

In a preferred embodiment, the selected cells and/or their progeny are transfected with long (PRR+) Numb isoform(s) encoding sequences as well as sequences encoding other transgenes, including oncostatin M (OSM).

In a preferred embodiment, the selected cells and/or their progeny are transfected with long (PRR+) Numb isoform(s) encoding sequences as well as sequences encoding other transgenes, including oncostatin M receptor (OSMR).

In a preferred embodiment, the selected cells and/or their progeny are transfected with long (PRR+) Numb isoform(s) encoding sequences as well as sequences encoding other transgenes, including cardiotrophin-1.

In a preferred embodiment, the selected cells and/or their progeny are transfected with long (PRR+) Numb isoform(s) encoding sequences as well as sequences encoding other transgenes, including CNTF.

In a preferred embodiment, the selected cells and/or their progeny are transfected with long (PRR+) Numb isoform(s) encoding sequences as well as sequences encoding other transgenes, including OCT3/4 and SOX2.

In a preferred embodiment, the selected cells and/or their progeny are transfected with long (PRR+) Numb isoform(s) encoding sequences as well as sequences encoding other transgenes, including NANOG, OCT3/4 and SOX2.

In a preferred embodiment, the selected cells and/or their progeny are transfected with long (PRR+) Numb isoform(s) encoding sequences as well as sequences encoding other transgenes, including OCT3/4 and SOX2 and a transgene with LIF activity.

In a preferred embodiment, the selected cells and/or their progeny are transfected sequences encoding other transgenes, including OCT3/4 and SOX2 and a transgene with LIF activity.

In a preferred embodiment, the selected cells and/or their progeny are transfected with long (PRR+) Numb isoform(s) encoding sequences as well as sequences encoding other transgenes, including Notch (e.g. Gaiano et al., 2000).

In a preferred embodiment, the selected cells and/or their progeny are transfected with long (PRR+) Numb isoform(s) encoding sequences as well as sequences encoding other transgenes, including OCT3/4, SOX2 and Notch (e.g. notch 1 and/or notch 2).

In a preferred embodiment, the selected cells and/or their progeny are transfected with long (PRR+) Numb isoform(s) encoding sequences as well as sequences encoding other transgenes, including OCT3/4, SOX2, NANOG, and Notch.

In a preferred embodiment, the selected cells and/or their progeny are transfected with long (PRR+) Numb isoform(s) encoding sequences as well as sequences encoding other transgenes, including OCT3/4, SOX2, NANOG, and a transgene with LIF activity.

In a preferred embodiment, the selected cells and/or their progeny are transfected with long (PRR+) Numb isoform(s) encoding sequences as well as sequences encoding other transgenes, including OCT3/4, SOX2, NANOG, and multiple transgenes with LIF activity.

In a preferred embodiment, the selected cells and/or their progeny are transfected with long (PRR+) Numb isoform(s) encoding sequences as well as sequences encoding other transgenes, including OCT3/4, Notch, HOXB4 and SOX2.

Over time, other gene combinations differing from those described herein may be described or discovered capable of causing cells to become multipotent, pluripotent, capable of self-renewal, or to begin differentiating. However this patent application covers such “genetic reprogramming” of any nucleated cell utilizing nucleic acid or protein electroporation (see Gagne et al., 1991; Saito et al., 2001; Yuan, 2008; Huang et al., 2007; Xia and Zhang, 2007; Cemazar and Sersa 2007; Isaka and Imai, 2007; Luxembourg et al., 2007; Van Tendeloos, 2007; Takahashi, 2007; etc.), liposomes, nanocapsules, nanovaults, etc. (see Goldberg et al., 2007; Li et al., 2007), and/or another approach avoiding viral integration or other random alteration of the cell's genome, as such means increase safety and efficiency.

Excluded, of course, from the category of random alteration are approaches involving gene-targeting and site-directed methods designed to introduce or remove DNA at specific locations in the genome.

Likewise, this patent application covers the genetic reprogramming of any nucleated cell utilizing nucleic acid or protein electroporation, liposomes, nanocapsules, nanovaults, etc., and/or another approach avoiding retroviral/lentiviral integration or other random alteration of the cell's genome, as such means increase safety and efficiency. Such approaches and methods include all known to the art and practicable in the present invention.

In a preferred embodiment, nucleic acid(s) or protein(s) corresponding to a single gene, or portion thereof, (particularly those named herein, discovered according to methods described herein, discovered according to other published methods; or known to be multipotency, pluripotency, or self-renewal inducing) are the only nucleic acid(s) or protein(s) overexpressed and/or introduced to produce multipotent, pluripotent, and/or self-renewing cells from the selected cells.

In a preferred embodiment, nucleic acid(s) or protein(s) corresponding to a single gene, or portion thereof, (particularly those named herein, discovered according to methods described herein, discovered according to other published methods; or known to be multipotency, pluripotency, or self-renewal inducing) are the only nucleic acid(s) or protein(s) overexpressed and/or introduced to produce multipotent, pluripotent, and/or self-renewing cells from the selected cells and the method utilized is electroporation, liposomes, nanocapsules, nanovaults, and/or another approach avoiding retroviral/lentiviral integration or other random alteration of the cell's genome.

In a separate preferred embodiment, other nucleic acid(s) or protein(s) can be utilized in concert with the nucleic acid(s) or protein(s) corresponding to a single gene, or portion thereof, (particularly those named herein, discovered according to methods described herein, discovered according to other published methods; or known to be multipotency, pluripotency, or self-renewal inducing) so long as a population of multipotent, pluripotent, and/or self-renewing cells is produced from the selected cells.

In a separate preferred embodiment, other nucleic acid(s) or protein(s) can be utilized in concert with the nucleic acid(s) or protein(s) corresponding to a single gene, or portion thereof, (particularly those named herein, discovered according to methods described herein, discovered according to other published methods; or known to be multipotency, pluripotency, or self-renewal inducing) so long as a population of multipotent, pluripotent, and/or self-renewing cells is produced from the selected cells and the method utilized is electroporation, liposomes, nanocapsules, nanovaults, and/or another approach avoiding retroviral/lentiviral integration or other random alteration of the cell's genome.

In a preferred embodiment, nucleic acid(s) or protein(s) corresponding to Nanog are the only nucleic acid(s) or protein(s) overexpressed and/or introduced to produce multipotent, pluripotent, and/or self-renewing cells from the selected cells and the method utilized is electroporation, liposomes, nanocapsules, nanovaults, and/or another approach avoiding retroviral/lentiviral integration or other random alteration of the cell's genome.

In a separate preferred embodiment, other nucleic acid(s) or protein(s) can be utilized in concert with the nucleic acid(s) or protein(s) corresponding to Nanog so long as a population of multipotent, pluripotent, and/or self-renewing cells is produced from the selected cells and the method utilized is electroporation, liposomes, nanocapsules, nanovaults, and/or another approach avoiding viral integration or other random alteration of the cell's genome.

In a separate preferred embodiment, nucleic acid(s) or protein(s) corresponding to Oct4 and Sox2 are the only nucleic acid(s) or protein(s) overexpressed and/or introduced to produce multipotent, pluripotent, and/or self-renewing cells from the selected cells and the method utilized is electroporation, liposomes, nanocapsules, nanovaults, and/or another approach avoiding viral integration or other random alteration of the cell's genome.

In a separate preferred embodiment, other nucleic acid(s) or protein(s) are utilized in concert with the nucleic acid(s) or protein(s) corresponding to Oct4/Sox2 so long as a population of multipotent, pluripotent, and/or self-renewing cells is produced from the selected cells and the method utilized is electroporation, liposomes, nanocapsules, nanovaults, and/or another approach avoiding viral integration or other random alteration of the cell's genome.

In a separate preferred embodiment, nucleic acid(s) or protein(s) corresponding to Long (PRR+) Numb isoforms are the only nucleic acid(s) or protein(s) overexpressed and/or introduced to produce multipotent, pluripotent, and/or self-renewing cells from the selected cells and the method utilized is electroporation, liposomes, nanocapsules, nanovaults, and/or another approach avoiding viral integration or other random alteration of the cell's genome.

In a separate preferred embodiment, other nucleic acid(s) or protein(s) are utilized in concert with the nucleic acid(s) or protein(s) corresponding to Long (PRR+) Numb isoforms so long as a population of multipotent, pluripotent, and/or self-renewing cells is produced from the selected cells and the method is electroporation, liposomes, nanocapsules, nanovaults, and/or another approach avoiding viral integration or other random alteration of the cell's genome.

In a preferred embodiment, nucleic acid(s) or protein(s) corresponding to Nanog are the only nucleic acid(s) or protein(s) overexpressed and/or introduced to produce multipotent, pluripotent, and/or self-renewing cells from the selected cells.

In a preferred embodiment, nucleic acid(s) or protein(s) corresponding to Nanog are the only nucleic acid(s) or protein(s) overexpressed and/or introduced to produce multipotent, pluripotent, and/or self-renewing cells from the selected cells and the method utilized is electroporation, liposomes, nanocapsules, nanovaults, and/or another approach avoiding retroviral/lentiviral integration or other random alteration of the cell's genome.

In a separate preferred embodiment, other nucleic acid(s) or protein(s) can be utilized in concert with the nucleic acid(s) or protein(s) corresponding to Nanog so long as a population of multipotent, pluripotent, and/or self-renewing cells is produced from the selected cells.

In a separate preferred embodiment, other nucleic acid(s) or protein(s) can be utilized in concert with the nucleic acid(s) or protein(s) corresponding to Nanog so long as a population of multipotent, pluripotent, and/or self-renewing cells is produced from the selected cells and the method utilized is electroporation, liposomes, nanocapsules, nanovaults, and/or another approach avoiding retroviral/lentiviral integration or other random alteration of the cell's genome.

In a preferred embodiment, nucleic acid(s) or protein(s) corresponding to a gene with LIF activity are the only nucleic acid(s) or protein(s) overexpressed and/or introduced to produce multipotent, pluripotent, and/or self-renewing cells from the selected cells.

In a preferred embodiment, nucleic acid(s) or protein(s) corresponding to a gene with LIF activity are the only nucleic acid(s) or protein(s) overexpressed and/or introduced to produce multipotent, pluripotent, and/or self-renewing cells from the selected cells and the method utilized is electroporation, liposomes, nanocapsules, nanovaults, and/or another approach avoiding retroviral/lentiviral integration or other random alteration of the cell's genome.

In a separate preferred embodiment, other nucleic acid(s) or protein(s) can be utilized in concert with the nucleic acid(s) or protein(s) corresponding to to a gene with LIF activity so long as a population of multipotent, pluripotent, and/or self-renewing cells is produced from the selected cells.

In a separate preferred embodiment, other nucleic acid(s) or protein(s) can be utilized in concert with the nucleic acid(s) or protein(s) corresponding to a gene with LIF activity so long as a population of multipotent, pluripotent, and/or self-renewing cells is produced from the selected cells and the method utilized is electroporation, liposomes, nanocapsules, nanovaults, and/or another approach avoiding retroviral/lentiviral integration or other random alteration of the cell's genome.

In a preferred embodiment, nucleic acid(s) or protein(s) corresponding to Oct4 are the only nucleic acid(s) or protein(s) overexpressed and/or introduced to produce multipotent, pluripotent, and/or self-renewing cells from the selected cells.

In a preferred embodiment, nucleic acid(s) or protein(s) corresponding to Oct4 are the only nucleic acid(s) or protein(s) overexpressed and/or introduced to produce multipotent, pluripotent, and/or self-renewing cells from the selected cells and the method utilized is electroporation, liposomes, nanocapsules, nanovaults, and/or another approach avoiding retroviral/lentiviral integration or other random alteration of the cell's genome.

In a separate preferred embodiment, other nucleic acid(s) or protein(s) can be utilized in concert with the nucleic acid(s) or protein(s) corresponding to Oct4 so long as a population of multipotent, pluripotent, and/or self-renewing cells is produced from the selected cells.

In a separate preferred embodiment, other nucleic acid(s) or protein(s) can be utilized in concert with the nucleic acid(s) or protein(s) corresponding to Oct4 so long as a population of multipotent, pluripotent, and/or self-renewing cells is produced from the selected cells and the method utilized is electroporation, liposomes, nanocapsules, nanovaults, and/or another approach avoiding retroviral/lentiviral integration or other random alteration of the cell's genome.

In a preferred embodiment, nucleic acid(s) or protein(s) corresponding to Sox2 are the only nucleic acid(s) or protein(s) overexpressed and/or introduced to produce multipotent, pluripotent, and/or self-renewing cells from the selected cells.

In a preferred embodiment, nucleic acid(s) or protein(s) corresponding to Sox2 are the only nucleic acid(s) or protein(s) overexpressed and/or introduced to produce multipotent, pluripotent, and/or self-renewing cells from the selected cells and the method utilized is electroporation, liposomes, nanocapsules, nanovaults, and/or another approach avoiding retroviral/lentiviral integration or other random alteration of the cell's genome.

In a separate preferred embodiment, other nucleic acid(s) or protein(s) can be utilized in concert with the nucleic acid(s) or protein(s) corresponding to Sox2 so long as a population of multipotent, pluripotent, and/or self-renewing cells is produced from the selected cells.

In a separate preferred embodiment, other nucleic acid(s) or protein(s) can be utilized in concert with the nucleic acid(s) or protein(s) corresponding to Sox2 so long as a population of multipotent, pluripotent, and/or self-renewing cells is produced from the selected cells and the method utilized is electroporation, liposomes, nanocapsules, nanovaults, and/or another approach avoiding retroviral/lentiviral integration or other random alteration of the cell's genome.

In a preferred embodiment, nucleic acid(s) or protein(s) corresponding to lin28 are the only nucleic acid(s) or protein(s) overexpressed and/or introduced to produce multipotent, pluripotent, and/or self-renewing cells from the selected cells.

In a preferred embodiment, nucleic acid(s) or protein(s) corresponding to lin28 are the only nucleic acid(s) or protein(s) overexpressed and/or introduced to produce multipotent, pluripotent, and/or self-renewing cells from the selected cells and the method utilized is electroporation, liposomes, nanocapsules, nanovaults, and/or another approach avoiding retroviral/lentiviral integration or other random alteration of the cell's genome.

In a separate preferred embodiment, other nucleic acid(s) or protein(s) can be utilized in concert with the nucleic acid(s) or protein(s) corresponding to lin28 so long as a population of multipotent, pluripotent, and/or self-renewing cells is produced from the selected cells and the method utilized is electroporation, liposomes, nanocapsules, nanovaults, and/or another approach avoiding retroviral/lentiviral integration or other random alteration of the cell's genome.

In a preferred embodiment, nucleic acid(s) or protein(s) corresponding to c-myc are the only nucleic acid(s) or protein(s) overexpressed and/or introduced to produce multipotent, pluripotent, and/or self-renewing cells from the selected cells.

In a preferred embodiment, nucleic acid(s) or protein(s) corresponding to c-myc are the only nucleic acid(s) or protein(s) overexpressed and/or introduced to produce multipotent, pluripotent, and/or self-renewing cells from the selected cells and the method utilized is electroporation, liposomes, nanocapsules, nanovaults, and/or another approach avoiding retroviral/lentiviral integration or other random alteration of the cell's genome.

In a separate preferred embodiment, other nucleic acid(s) or protein(s) can be utilized in concert with the nucleic acid(s) or protein(s) corresponding to c-myc so long as a population of multipotent, pluripotent, and/or self-renewing cells is produced from the selected cells.

In a separate preferred embodiment, other nucleic acid(s) or protein(s) can be utilized in concert with the nucleic acid(s) or protein(s) corresponding to c-myc so long as a population of multipotent, pluripotent, and/or self-renewing cells is produced from the selected cells and the method utilized is electroporation, liposomes, nanocapsules, nanovaults, and/or another approach avoiding retroviral/lentiviral integration or other random alteration of the cell's genome.

In a separate preferred embodiment, nucleic acid(s) or protein(s) corresponding Oct4 and Sox2 are the only nucleic acid(s) or protein(s) overexpressed and/or introduced to produce multipotent, pluripotent, and/or self-renewing cells from the selected cells.

In a separate preferred embodiment, nucleic acid(s) or protein(s) corresponding to Oct4 and Sox2 are the only nucleic acid(s) or protein(s) overexpressed and/or introduced to produce multipotent, pluripotent, and/or self-renewing cells from the selected cells and the method utilized is electroporation, liposomes, nanocapsules, nanovaults, and/or another approach avoiding retroviral/lentiviral integration or other random alteration of the cell's genome.

In a separate preferred embodiment, other nucleic acid(s) or protein(s) are utilized in concert with the nucleic acid(s) or protein(s) corresponding to Oct4 and Sox2 so long as a population of multipotent, pluripotent, and/or self-renewing cells is produced from the selected cells.

In a separate preferred embodiment, other nucleic acid(s) or protein(s) are utilized in concert with the nucleic acid(s) or protein(s) corresponding to Oct4 and Sox2 so long as a population of multipotent, pluripotent, and/or self-renewing cells is produced from the selected cells and the method utilized is electroporation, liposomes, nanocapsules, nanovaults, and/or another approach avoiding retroviral/lentiviral integration or other random alteration of the cell's genome.

In a separate preferred embodiment, nucleic acid(s) or protein(s) corresponding to

Long (PRR+) Numb isoforms are the only nucleic acid(s) or protein(s) overexpressed and/or introduced to produce multipotent, pluripotent, and/or self-renewing cells from the selected cells.

In a separate preferred embodiment, nucleic acid(s) or protein(s) corresponding to

Long (PRR+) Numb isoforms are the only nucleic acid(s) or protein(s) overexpressed and/or introduced to produce multipotent, pluripotent, and/or self-renewing cells from the selected cells and the method utilized is electroporation, liposomes, nanocapsules, nanovaults, and/or another approach avoiding retroviral/lentiviral integration or other random alteration of the cell's genome.

In a separate preferred embodiment, other nucleic acid(s) or protein(s) are utilized in concert with the nucleic acid(s) or protein(s) corresponding to Long (PRR+) Numb Isoforms so long as a population of multipotent, pluripotent, and/or self-renewing cells is produced from the selected cells.

In a separate preferred embodiment, other nucleic acid(s) or protein(s) are utilized in concert with the nucleic acid(s) or protein(s) corresponding to Long (PRR+) Numb Isoforms so long as a population of multipotent, pluripotent, and/or self-renewing cells is produced from the selected cells and the method utilized is electroporation, liposomes, nanocapsules, nanovaults, and/or another approach avoiding retroviral/lentiviral integration or other random alteration of the cell's genome.

In a separate preferred embodiment, nucleic acid(s) or protein(s) corresponding to

Oct4, Sox2, and Nanog are the only nucleic acid(s) or protein(s) overexpressed and/or introduced to produce multipotent, pluripotent, and/or self-renewing cells from the selected cells.

In a separate preferred embodiment, nucleic acid(s) or protein(s) corresponding to

Oct4, Sox2, and Nanog are the only nucleic acid(s) or protein(s) overexpressed and/or introduced to produce multipotent, pluripotent, and/or self-renewing cells from the selected cells and the method utilized is electroporation, liposomes, nanocapsules, nanovaults, and/or another approach avoiding retroviral/lentiviral integration or other random alteration of the cell's genome.

In a separate preferred embodiment, other nucleic acid(s) or protein(s) are utilized in concert with the nucleic acid(s) or protein(s) corresponding to Oct4, Sox2, and Nanog so long as a population of multipotent, pluripotent, and/or self-renewing cells is produced from the selected cells.

In a separate preferred embodiment, other nucleic acid(s) or protein(s) are utilized in concert with the nucleic acid(s) or protein(s) corresponding to Oct4, Sox2, and Nanog so long as a population of multipotent, pluripotent, and/or self-renewing cells is produced from the selected cells and the method utilized is electroporation, liposomes, nanocapsules, nanovaults, and/or another approach avoiding retroviral/lentiviral integration or other random alteration of the cell's genome.

In a separate preferred embodiment, nucleic acid(s) or protein(s) corresponding to Long (PRR+) Numb isoforms are the only nucleic acid(s) or protein(s) overexpressed and/or introduced to produce multipotent, pluripotent, and/or self-renewing cells from the selected cells.

In a separate preferred embodiment, nucleic acid(s) or protein(s) corresponding to Long (PRR+) Numb isoforms are the only nucleic acid(s) or protein(s) overexpressed and/or introduced to produce multipotent, pluripotent, and/or self-renewing cells from the selected cells and the method is electroporation, liposomes, nanocapsules, nanovaults, and/or another approach avoiding retroviral/lentiviral integration or other random alteration of the cell's genome.

In a separate preferred embodiment, other nucleic acid(s) or protein(s) are utilized in concert with the nucleic acid(s) or protein(s) corresponding to Long (PRR+) Numb isoforms so long as a population of multipotent, pluripotent, and/or self-renewing cells is produced from the selected cells.

In a separate preferred embodiment, other nucleic acid(s) or protein(s) are utilized in concert with the nucleic acid(s) or protein(s) corresponding to Long (PRR+) Numb isoforms so long as a population of multipotent, pluripotent, and/or self-renewing cells is produced from the selected cells and the method is electroporation, liposomes, nanocapsules, nanovaults, and/or another approach avoiding retroviral/lentiviral integration or other random alteration of the cell's genome.

It is to be understood that any combination of nucleic acid or protein sequences described herein can be modified by excluding those corresponding to Numb and/or Numblike so long as the desired cell population or behavior is achieved.

Similarly, it should be understood that the methods described herein for initiating differentiation are applicable to any induced or non-induced multipotent, pluripotent, or self-renewing stem cells, other progenitor cells, or other selected cells, not only those obtained in the manner described herein.

It is to be understood that any combination of nucleic acid or protein sequences described herein can be modified by excluding nucleic acid sequences or proteins corresponding to Numb and/or Numblike so long as the desired cell population is achieved.

In another embodiment, the various nucleic acid or protein combinations described herein are employed with the exclusion of the nucleic acid or protein corresponding to the Numblike and/or Numb isoforms.

In a preferred embodiment, the selected cells and/or their progeny are cells that have been genetically-modified beforehand.

In a preferred embodiment, the transfection steps described herein represent transient transfection.

In a further preferred embodiment such transient transfection is accomplished using viral vectors that do not integrate into the host genome.

In another preferred embodiment, such transient transfection is accomplished using standard transfection techniques (electroporation, chemically mediated transfection, fusogenic or non-fusogenic liposomes, nanocapsules, nanovaults, etc.).

Over time, other gene combinations differing from those described herein may be described or discovered capable of causing cells to become multipotent, pluripotent, capable of self-renewal or to begin differentiating. However this patent application also covers the genetic reprogramming of any nucleated cell utilizing nucleic acid or protein electroporation (for example methods see Gagne et al., 1991; Saito et al., 2001; Yuan, 2008; Huang et al., 2007; Xia and Zhang, 2007; Cemazar and Sersa 2007; Isaka and Imai, 2007; Luxembourg et al., 2007; Van Tendeloos, 2007; Takahashi, 2007; etc.) electroporation, liposomes, nanocapsules, nanovaults, and/or another approach avoiding viral integration or other random alteration of the cell's genome as such means increase safety and efficiency.

In another preferred embodiment, transfection with long (PRR+) numb isoform encoding sequences (and/or synthetic oligonucleotides targeting numblike and short numb isoforms) is accompanied or replaced by transient or permanent transfection with other sequences including ones selected from those encoding human LIF (e.g. Du and Shi, 1996) oncostatin-M, cardiotrophin-1, IL-11, IL-6, IL6R, hyper IL-6, LIFR, gp130, OCT3 (OCT4), Nanog, SOX2, and/or FGF-4.

Simultaneous transfection with any subset of these distinct transgene sequences can be accomplished by any means known to the art including the use of a single genetic vector, multiple genetic vectors, serial transfection and selection based on distinct marker proteins and/or antibiotic resistances.

In another preferred embodiment, cells transfected with long (PRR+) numb isoform(s) are cultured in a cell culture promoting an optimal growth rate, such as described above, and that includes EGF, bFGF, oncostatin, LIF (e.g. Du and Shi, 1996), steel factor, IL-11, cardiotrophin-1, IL-6, hyper-IL-6, CNTF, and/or soluble gp130.

Assessment of Potency and Differentiation

Pluripotency and multipotency can be assessed by any means known to the art including 1) transplantation, 2) culture under conditions promoting embryoid body formation, 3) injection of cells into animal blastocyst stage embryos with subsequent development, and 4) RNA expression assays (e.g. RT-PCR and microarray based analyses) for gene expression associated with differentiation, multipotency, pluripotency, etc. (see Guan et al., 2006), 5) colony-formation, as well as by ES-like morphology. One approach disclosed herein for detecting pluripotency in selected cells and/or their progeny involves transfection with a reporter construct comprising the Nanog promoter operably linked to a fluorescent protein gene. This allows identification and enrichment of Nanog expressing cells using Fluorescence Activated Cell Sorting (FACS), etc.

In a preferred embodiment, endogenous cells (e.g. cells surrounding a burn or injury site) are transfected in vivo with genetic vectors encoding the long (PRR+) numb isoform(s) alone or in conjuction with other transgenes named herein to transiently promote renewed or increased cell proliferation. This approach can also be utilized clinically in the setting of hypoplastic tissues, disorders where stem/progenitor cells are abnormally depleted, and other disorders where the approach can be shown to be beneficial.

Achieving Differentiating Cell Populations

In order to achieve b) neural c) muscle d) and other cell populations capable of further environmentally-regulated differentiation in vivo, selected cell(s) and/or their progeny are optionally transfected with long (PRR+) Numb isoform sequence(s) and/or synthetic oligonucleotide sequences and expanded by growth for sufficient time to achieve the desirable number of cell progeny in vitro (as described above).

Following this optional step, the selected cells and/or their progeny are washed free of the cytokines and agents comprising the expansion/optimal growth media, and are optionally transfected with the nucleotide sequence(s) encoding the Numblike gene and/or “short” (PRR−) Numb isoform(s) and/or synthetic oligonucleotides targeting the long (PRR+) isoforms, etc. (e.g. Zaehres et al., 2005), then cultured under conditions which promote differentiation of the selected cells into the desired cell type(s).

In most instances, the cells are then cultured in the presence of 5-10% fetal bovine serum and agents(s) promoting differentiation of the selected cells and/or their progeny into a desired cell population. The presence of the fetal bovine and of the agents(s) provides for growth or proliferation at a rate that is less than the optimal (or expansion) growth rate, and favors differentiation of the cells into a desired cell population. The agents and precise culture conditions are selected according to the desired cell population as described below.

Achieving Neuronal or Neural Cell Populations

When the desired cell population is a neural cell population, the successfully transfected cells are cultured under conditions that promote growth at a rate which is less than the optimal rate and in the presence of agent(s) promoting differentiation of the cells into neural cells. Conditions promoting differentiation into neurons have been described in numerous publications including (Benninger et al., 2003; Chung et al. 2005; Harkany et al., 2004; Ikeda et al., 2004; Ikeda et al., 2005; Wernig et al., 2002; and Wernig et al., 2004). Furthermore, combining retinoic acid exposure with the presence of additional cytokines favors specific neuronal cell type differentiation in vitro (e.g. Soundararajan et al., 2006; Soundararajan et al., 2007; U.S. Pat. No. 6,432,711).

In a preferred embodiment, in vitro differentiation of neurons or neural cells occurs in the presence of 50 ng/mL nerve growth factor (NGF).

In a preferred embodiment, when a neuronal population is the desired cell population, transfection with sequences encoding short numb isoforms (and/or numblike) is accompanied or replaced by transient or permanent transfection with other sequences including ones selected from those encoding Nurr1, REN, Neurogenin1, Neurogenin2, Neurogenin3, Mash 1, Phox2b, Phox2a, dHand, Gata3, Shh, FGF8, Lmx1b, Nkx2.2, Pet1, Lbx1, and/or Rnx.

In another preferred embodiment, when dopaminergic neurons are the desired neuronal population, transfection with sequences encoding short numb isoforms (and/or numblike) is accompanied or replaced by transient or permanent transfection with other sequences including ones selected from those encoding Mash1, Ngn2, Nurr1, Lmx1b, and/or Ptx-3.

In another preferred embodiment, when serotonergic neurons are the desired neuronal population, transfection with sequences encoding short numb isoforms (and/or numblike) is accompanied or replaced by transient or permanent transfection with other sequences including ones selected from those encoding Mash1, Phox2b, Lmx1b, Nk×2.2, Gata2, Gata3 and/or Pet1.

In another preferred embodiment, when cholinergic neurons are the desired neuronal population, transfection with sequences encoding short numb isoforms (and/or numblike) is accompanied or replaced by transient or permanent transfection with other sequences including ones selected from those encoding MASH1, Phox2a and/or REST4.

In another preferred embodiment, when GABAergic neurons are the desired neuronal population, transfection with sequences encoding short numb isoforms (and/or numblike) is accompanied or replaced by transient or permanent transfection with other sequences including ones selected from those encoding MASH1, Phox2a and/or REST4, followed, optionally, by culture in media supplemented with LIF, Neurotrophin 3 (NT3), and/or nerve growth factor (NGF).

In another preferred embodiment, when noradrenergic neurons are the desired neuronal population, transfection with sequences encoding short numb isoforms (and/or numblike) is accompanied or replaced by transient or permanent transfection with other sequences including ones selected from those encoding Mash1, dHand, Phox2a, Phox2b, Gata2 and/or Gata3.

In another preferred embodiment, when GABAergic neurons are the desired neuronal population, transfection with sequences encoding short numb isoforms (and/or numblike) is accompanied or replaced by transient or permanent transfection with other sequences including ones selected from those encoding PITX2, D1x2, D1x5, antisense Hest RNA and/or other HES1 targeting synthetic oligonucleotides.

In another preferred embodiment, when a neuronal or neural cell population is the desired population, cells transfected with short (PRR−) numb isoforms (and/or numblike) are cultured in a cell culture medium promoting differentiation, such as described above and that includes one or more of the following agents: retinoic acid, NT3, NGF, glial cell-line derived growth factor (GDNF), and interferon gamma (IFN-gamma).

Achieving Muscle Cell Populations

When the desired cell population is a muscle population, the successfully transfected cells are cultured in the presence of an agent promoting differentiation of the cells into muscle cells and growth at a rate less than the optimal rate. Conditions promoting differentiation into muscle cells have also been described previously (Nakamura et al., 2003; Pal and Khanna, 2005; Pipes et al., 2005; Albilez et al., 2006; Pal and Khanna, 2007; Behfar et al., 2007; U.S. Pat. No. 6,432,711). Furthermore, exposure of selected cells and/or their progeny to hexamethylene bis-acrylamide or dimethylsulfoxide in the presence of additional cytokines favors the initiation of muscle type differentiation in vitro.

In a preferred embodiment, when a cardiac muscle cell population is the desired population, cells transfected with short (PRR−) numb isoforms (and/or numblike) are cultured in a cell culture medium promoting differentiation into cardiomycytes (He et al., 2003; Guan et al., 2007; etc.), or that includes specific agents at concentrations promoting cardiac cell differentiation (e.g. 0.75%-1% dimethyl sulfoxide (DMSO), 20% normal bovine serum (NBS), 10(−7) mM retinoic acid (RA) and 20% cardiomyocytes conditioned medium (Hua et al., 2006).

In another preferred embodiment, when a cardiac muscle cell population is the desired population, the cells are also transfected with nucleotide sequences including ones selected from those sequences encoding Gata 4, Gata 5, and Gata 6.

In a preferred embodiment, when a muscle cell population is the desired cell population, transfection with sequences encoding short numb isoforms (and/or numblike) is accompanied or replaced by transient or permanent transfection with other sequences including ones selected from those encoding muscle type specific bHLH-encoding sequences, MyoD, Myogenin, MyfS, Myf6, Mef2, Myocardin, Ifrd1 and/or other muscle transcription factors.

In a preferred embodiment, when a smooth muscle cell population is the desired cell population, transfection with sequences encoding short numb isoforms (and/or numblike) is accompanied or replaced by transient or permanent transfection with other sequences including ones selected from those encoding the muscle type specific Myocardin nucleotide sequence.

In a preferred embodiment, when a skeletal muscle cell population is the desired cell population, transfection with sequences encoding short numb isoforms (and/or numblike) is accompanied or replaced by transient or permanent transfection with other sequences including ones selected from those encoding the muscle type specific MyoD and myogenin nucleotide sequences.

In a preferred embodiment, when an oligodensrocyte cell population is the desired cell population, transfection with sequences encoding short numb isoforms (and/or numblike) is accompanied or replaced by transient or permanent transfection with other sequences including ones selected from those encoding the oligodendrocyte-specific OLIG1, OLIG2, and Zfp488 nucleotide sequences.

Simultaneous transfection with any subset of these distinct transgene sequences listed above can be accomplished by any means known to the art including the use of multiple genetic vectors, serial transfection as well as selection based on distinct marker proteins and/or antibiotic resistance.

When the desired cell population is a hematopoietic cell population, the differentiation medium includes specific agents at concentrations promoting differentiation into hematopoietic progenitor cells (e.g. vascular endothelial growth factor (VEGF), thrombopoietin, etc. (e.g. Ohmizono, 1997; Wang et al., 2005; Srivastava et al., 2007; Gupta et al., 2007) or differentiated hematopoietic cell types (according to methods known to the art for providing differentiated hematopoietic cell types from undifferentiated or pluripotent cells).

When the desired cell population is a germ cell population, the differentiation medium includes specific agents at concentrations promoting differentiation into germ cells (e.g. Nayernia et al. 2006a, 2006b).

When the desired cell population is an endoderm and pancreatic islet cell population, the differentiation media includes specific agents at concentrations promoting differentiation into endoderm and pancreatic islet cells (e.g. Xu et al., 2006; Denner et al., 2007; Shim et al., 2007; Jiang et al., 2007).

In a preferred embodiment, differentiation of selected cells and/or their progeny may occur in the differentiation medium in the absence of transfection with numblike, short Numb idsoforms or other transgenes, although the differentiation medium may be unchanged.

In embodiments, a single vector will be utilized which controls the expression of nucleotide sequence(s) encoding the “long” (PRR+) isoform(s) of the mammalian numb gene (and/or synthetic oligonucleotides targeting numblike or the short numb isoforms) under one regulable promoter (e.g. a tetracycline-regulated promoter), while the Numblike and short Numb isoforms (and/or synthetic oligonucleotides targeting the long (PRR+) isoforms) are expressed under the control of another, distinct, but also regulable promoter. Thus, the long (PRR+) numb isoform(s) can be expressed (and/or short isoforms repressed) when expansion of the selected cells is desired and an inducing agent (e.g. tetracycline) is added to the growth medium; later numblike and the short isoforms can be expressed (and/or long (PRR+) numb isoform(s) repressed) when differentiation is desired.

Alternatively, proteins and peptides corresponding to Numb isoforms, Notch, OCT3/4, SOX2, and other DNA sequences listed herein may be applied in analogous fashion to selected cells and/or their progeny via electroporation (e.g. Koken et al., 1994; Ritchie and Gilroy, 1998), using nano particles, cationic lipids, fusogenic liposomes (e.g. Yoshikawa et al., 2005; 2007), etc. in lieu of, or in combination with genetic transfection. Generally, electroporation allows for high transfection efficiency (and efficient production of the desired cells) without genomic integration of the transgene and is therefore associated with increased safety.

The DNA or RNA encoding protein(s) or polypeptide(s) promoting proliferation, multipotentiality, pluripotentiality or differentiation of the selected cells may be isolated in accordance with standard genetic engineering techniques (for example, by isolating such DNA from a cDNA library of the specific cell line) and placing it into an appropriate expression vector, which then is transfected into the selected cells.

In another preferred embodiment, endoderm and pancreatic islet cells are the desired population, and transfection with sequences encoding short numb isoforms (and/or numblike) is accompanied or replaced by transient or permanent transfection with other sequences including ones selected from those encoding Foxa2, Sox17, HLXB9 and/or Pdx1.

In another preferred embodiment, hepatocytes are the desired population, and transfection with sequences encoding short numb isoforms (and/or numblike) is accompanied or replaced by transient or permanent transfection with other sequences including ones selected from those encoding hepatic nuclear factor (HNF)-1, HNF-3, HNF-4, HNF-6 and creb-binding protein.

In another preferred embodiment, hematopoietic cells are the desired population, and transfection with sequences encoding short numb isoforms (and/or numblike) is accompanied or replaced by transient or permanent transfection with other sequences including ones selected from those encoding Runx1/AML1 and NOV(CCN3), and/or cell culture in the presence of colony stimulating factors specific for the desired cell populations. The Runx1/AML1a isoform is introduced when engraftment is desired and the b isoform when differentiation is desired (Creemers et al., 2006).

In another preferred embodiment, chondrocytes are the desired population, and transfection with sequences encoding short numb isoforms (and/or numblike) is accompanied or replaced by transient or permanent transfection of other sequences including ones encoding Sox9, CREB-binding protein, Gata6, and/or Runx2.

In another preferred embodiment, bone cells (especially osteoblasts) are the desired population, and transfection with sequences encoding short numb isoforms (and/or numblike) is accompanied or replaced by transient or permanent transfection of other sequences including Runx2.

In a preferred embodiment, the genetic vectors encoding the long Numb isoforms (such as those described herein) are introduced transiently or under the control of a regulable promoter, into endogenous cells in vivo in order to cause those cells proliferate transiently.

In a preferred embodiment, endogenous cells (e.g. ependymal zone cells of the central nervous system) are transfected in vivo with genetic vectors encoding either the shortest numb isoform or the numblike protein(s) alone or in conjuction with other transgenes named herein, in order to transiently or permanently promote renewed or increased differentiation (especially neuronal differentiation) and migration of progenitor/ependymal cells in the central nervous system). This renewal or increase is measured in terms of the number of cells showing new-onset expression of markers associated with differentiation. This may be accomplished by introduction of the genetic vectors into the organ system using methods suitable for that purpose (see examples).

In a preferred embodiment, endogenous cells (e.g. ependymal zone cells of the central nervous system) are transfected in vivo with genetic vectors encoding the long numb isoform(s) and/or other transgenes named herein, in order to transiently promote renewed or increased stem cell proliferation (with subsequent differentiation of progeny cells). This renewal or increase is measured in terms of the number of cells showing new-onset expression of marlers associated with dividing progenitors. This may be accomplished by introduction of the genetic vectors into the organ system using methods suitable for that purpose (see examples).

Likewise this approach is also be suitable for inducing renewed or increased differentiation from other stem cell populations in other tissues (such as the skin, etc). This approach can be utilized, for example, clinically in the setting of central nervous system injury, disorders of other tissues where normal differentiation or migration are inadequate, dysplastic disorders and other disorders where the approach is beneficial.

In a preferred embodiment, nucleic acid(s) or protein(s) corresponding to a single gene, or portion thereof, (particularly those named herein, discovered according to methods described herein, discovered according to other published methods; and/or known to be capable of initiating the desired manner of differentiation) are the only nucleic acid(s) or protein(s) overexpressed and/or introduced to initiate differentiation in the selected cells.

In a preferred embodiment, nucleic acid(s) or protein(s) corresponding to a single gene, or portion thereof, (particularly those named herein, discovered according to methods described herein, discovered according to other published methods; and/or known to be capable of initiating the desired manner of differentiation) are the only nucleic acid(s) or protein(s) overexpressed and/or introduced to initiate differentiation in the selected cells and the method utilized is electroporation, liposomes, nanocapsules, nanovaults, and/or another approach avoiding retroviral/lentiviral integration or other random alteration of the cell's genome.

In a separate preferred embodiment, other nucleic acid(s) or protein(s) can be utilized in concert with the nucleic acid(s) or protein(s) corresponding to a single gene, or portion thereof, (particularly those named herein, discovered according to methods described herein, discovered according to other published methods; and/or known to be capable of initiating the desirable manner of differentiation) so long as a population of differentiating cells is produced from the selected cells.

In a separate preferred embodiment, other nucleic acid(s) or protein(s) can be utilized in concert with the nucleic acid(s) or protein(s) corresponding to a single gene, or portion thereof, (particularly those named herein, discovered according to methods described herein, discovered according to other published methods; and/or known to be capable of initiating the desirable manner of differentiation) so long as a population of differentiating cells is produced from the selected cells and the method utilized is electroporation, liposomes, nanocapsules, nanovaults, and/or another approach avoiding retroviral/lentiviral integration or other random alteration of the cell's genome.

It is to be understood that any combination of nucleic acid or protein sequences described herein can be modified by excluding those corresponding to Numb and/or Numblike so long as the desired cell population or behavior is achieved.

Similarly, it should be understood that the methods described herein (or elsewhere) for initiating differentiation are applicable to any induced or non-induced multipotent, pluripotent, or self-renewing stem cells, or other selected cells, not only those obtained in the manner described herein.

Sources of Selected Cells

The population of selected cells may derive from various stem cells, progenitor cells and somatic cells. However somatic cells lacking nuclei (e.g. mature, human red blood cells) are specifically excluded. Selected stem cells may be derived from existing cell lines or isolated from stored, banked, or cryopreserved sources. Typical sources of stem cells include bone marrow, peripheral blood, placental blood, amniotic fluid (e.g. De Coppi et al., 2007), umbilical cord blood (e.g. Zhao, et al., 2006; Tian et al., 2007), adipose tissue (e.g. Gimble et al., 2007; Ma et al., 2007), non-human embryos, and others. Circulating leukocytes and other non-stem cells may likewise be selected and subjected to the same culture conditions as described above effective that they acquire multipotency, pluripotency and/or self-renewal as a result. Examples of other accessible somatic cells useful in this invention include lymphocytes and epithelial (e.g. buccal cheek) cells. Isolation and collection of cells selected for use within the present invention may be performed by any method known to the art.

In embodiments involving animals, stem cells isolated from prostate, testis, embryonic brain, and intestine are also disclosed as being preferred sources of selected cells.

In a preferred embodiment, the selected cells and/or their progeny are cultured in a three-dimensional format.

A further aim of the present invention is to provide cells for use in the production of patient-compatible and patient-specific tissues and organs for transplantation to patients deemed to be requiring such organs or tissues. It is disclosed herein that the pluripotent, multipotent, and/or differentiating cells provided by the methods described herein (or similar methods) be utilized in conjunction with techniques aimed at the production of such organs and/or tissues (e.g. Boland et al., 2006. Xu et al., 2006; Campbell and Weiss, 2007). Such utilization is specifically covered by the present invention.

For instance, pluripotent, multipotent, and/or differentiating cells produced or treated according to the methods described herein (or other published methods) may be grown in association with three-dimensional or two-dimensional scaffoldings engineered to replicate normal tissue structure and/or organ structures (e.g. Yarlagada et al., 2005; Kim et al, 1998; WO/2003/070084; EP1482871; WO03070084;U.S. Pat. Nos. 2,395,698; 7,297,540; 6,995,013; 6,800,753; Isenberg et al., 2006).

Similarly, scaffoldings to be occupied by the pluripotent, multipotent, and/or differentiating cells may be derived from cadaveric organ(s) or tissue(s) after the cadaveric organs or tissues (e.g. bone, heart, kidney, liver, lung, etc.) may be treated in such away that the host immune cells resident in that tissue, and other undesirable or ancillary host cells, are eliminated (e.g. by ionizing radiation, sterilization (e.g. Mroz et al., 2006), and/or various methods of decellularization (U.S. Pat. Nos. 6,734,018; 6,962,814; 6,479,064; 6,376,244; U.S. Pat. Nos. 5,032,508; 4,902,508; 4,956,178; 5,281,422, 5,554,389; 6,099,567; and 6,206,931; 4,361,552 and 6,576,618; 6,753,181; U.S. application Ser. No. 11/162,715; WO/2001/048153; WO/2002/024244; WO003002165; WO/2001/049210; WO/2007/025233; European Patents EP1482871; EP1246903; EP1244396; EP0987998; EP1244396; EP1333870; Rieder et al., 2004; Ott et al., 2008; Taylor et al., 1998)).

Likewise, it is anticipated that the pluripotent, multipotent, and/or differentiating cells of the present invention may be used in applications utilizing inkjet-style printing for tissue engineering (e.g. Boland et al., 2006. Xu et al., 2006; Campbell et al., 2007). Therefore such use of the cells produced or treated according to the methods described herein is covered.

In another preferred embodiment, the selected cells and/or their progeny are cultured in hanging drops.

In accordance with another aspect of the present invention, selected cells may be modified genetically beforehand.

In accordance with another aspect of the present invention, selected cells may be modified with DNA or RNA encoding protein(s) or polypeptide(s) promoting differentiation of the cell into a desired cell population.

### Screening Cell Populations

In one embodiment, the methods of this invention comprise screening cells from cell lines, donor sources, umbilical cord blood, and autologous or donor bone marrow, blood, spermatogonia, primordial germ cells, buccal cheek cells, or any other cell source effective in the current invention. Selected cells can be screened to confirm successful transfection with beneficial sequence(s) or therapeutic vector(s) as well as successful initiation of differentiation by any method known to the art (Guan et al., 2006; U.S. Pat. No. 6,432,711). In some embodiments, the cells are screened using standard PCR and nucleic acid hybridization-based methods or using rapid typing methods. In preferred embodiments, the cells are screened according to expression of reporter genes. In some embodiments, cells are screened by expression of a marker gene encoded by the transgene expressing vector(s) such as an antibiotic resistance gene or a fluorescent protein (e.g. GFP) gene.

Screening for Therapeutic Vectors and Beneficial Sequences

Cells can be screened for the presence of beneficial sequence(s) and therapeutic vector(s) using any method(s) known to the art for detection of specific sequences. Each cell sample can be screened for a variety of sequences simultaneously. Alternatively, multiple samples can be screened simultaneously.

Cell differentiation may be monitored by several means: including (i) morphological assessment, (ii) utilizing reverse transcriptase polymerase chain reaction (RT-PCR), Northern blot, or microarray techniques to monitor changes in gene expression, (iii) assaying cellular expression of specific markers such as beta tubulin III (for neurons) etc. (Ozawa, et al., 1985). In some embodiments, the cells are screened for successful initiation of differentiation using FACS sorting based on cell type specific markers or transgenic marker expression (e.g. antibiotic resistance or fluorescent protein expression) under the control of cell type specific promoters such as the myosin promoter in muscle cells; the human cardiac α-actin promoter in cardiomyocytes; the insulin promoter in insulin producing cells; the neuronal-specific enolase (NSE) promoter for neuronal differentiation, or neurotransmitter related promoters such as the tyrosine hydroxylase promoter in dopaminergic neurons; etc.).

In some embodiments, the cells are screened using standard PCR and nucleic acid hybridization-based methods. In a particularly preferred embodiment, the cells are screened using rapid typing methods.

Screening for Human Leukocyte Antigen (HLA) Type

In certain embodiments, the selected cells are selected with respect to compatible HLA typing. The HLA genotype can be determined by any means known to those of skill in the art.

The cells used for screening may consist of cells taken directly from a donor, or from cell lines established from donor cells, or other practicable cell sources. The cells can be screened for beneficial sequence(s), and/or therapeutic vector(s) and HLA type at once, or separately. Those cells successfully transfected with a beneficial sequence and showing an appropriate HLA genotype can be prepared for transplantation to a patient.

In certain embodiments, the transfected cells are transplanted without HLA typing. In other embodiments, the cells are HLA typed for compatibility.

Screening for Agents Promoting a Cellular Phenotype

The present invention also provides for a methods of screening proteins and agents for their ability to induce phenotypic changes or differentiation of the selected cells and/or their progeny into desired cell populations. Briefly, vectors encoding complementary DNAs (cDNAs) from appropriate cDNA libraries are transfected into the selected cells/and or their progeny. Once a specific cDNA that induces differentiation or other phenotypic change is identified, such cDNA then may be isolated and cloned into an appropriate expression vector for protein production in appropriate cells (e.g. COS cells) in vitro. Later the protein containing supernatant can be applied to the selected cell cultures to determine if any secreted proteins from such cells induce differentiation Alternatively, candidate agents can be applied to the selected cell cultures to determine if any secreted proteins from such cells induce differentiation (see U.S. Pat. No. 6,432,711).

The present invention also provides for methods of screening nucleic acids for their ability to induce multipotentiality, pluripotentiality, and/or self-renewal, or to initiate differentiation of selected cells and/or their progeny. In these methods, vectors encoding selected cDNAs (or cDNAs from appropriate cDNA libraries, or other sequences) are introduced into the selected cells/and or their progeny using electroporation, nanocapsules, nanovaults, liposomes, retroviruses, lentiviruses, and/or any other practicable means of transfection. Once a specific cDNA that induces a phenotypic change, multipotentiality, pluripotentiality, and/or self-renewal, is identified, such cDNA then may be isolated and cloned into an appropriate expression vector. Assays for determining such changes include those described elsewhere herein.

Likewise the protein corresponding to the identified cDNA may be produced in appropriate cells (e.g. COS cells) in vitro to determine whether the protein containing supernatant can be applied to the selected cell cultures and induce the desired changes.

Finally, proteins may be introduced into the selected cells/and or their progeny using electroporation, nanocapsules, nanovaults, liposomes, retroviruses, lentiviruses, and/or any other practicable means of transfection, and the resulting cells assessed as described herein for multipotentiality, pluripotentiality, self-renewal or the initiation of differentiation.

Transplantation of Cells into Patients

After screening, selected cells and/or their progeny may be cryopreserved, maintained as cell lines in culture, or may be administered to the patient. Selected cells can be cryopreserved or maintained in culture by any means known to the art and preserved for future transplantation procedures.

Preferably, the cells to be screened are obtained from accessible sources allowing easy collection.

With regard to producing HIV resistant cells: targeted somatic cells and stem cells of this invention can be of any type capable of differentiating into cells that can be infected by HIV, that can sustain the transcription and/or replication of HIV, that can alter the HIV immune response, or that can retard progression to AIDS. Such stem cells include, but are not limited to, pluripotent cells derived from spermatogonia, primordial germ cells, hematopoietic stem cells, peripheral blood cells, placental blood cells, amniotic fluid cells, umbilical cord blood cells, buccal cheek cells, adipose tissue cells (including stem cells derived from those tissues), reprogrammed cells, induced multipotent cells, induced pluripotent cells, etc., non-human embryos, and/or any other cell type that can form blood and immune cells, HIV target cells, and other cells.

Therapeutic vector(s) express “beneficial sequence(s)” intended to render transfected or infected cells less capable of sustaining HIV replication and transcription. The genetic vector expressing “beneficial sequence(s)” as well as any virus derived from such genetic vector, are herein termed “therapeutic vector”.

After screening, cells transfected with the desired therapeutic vector(s) and expressing beneficial sequence (with or without compatible HLA genotype) may be expanded ex vivo (in vitro) using standard methods to culture dividing cells and maintained as stable cell lines (U.S. Pat. Nos. 6,432,711 and 5,453,357 herein incorporated by reference). Alternatively, these cells can be administered to the patient and expanded in vivo.

Selected cells can be cryopreserved by any means known to the art and preserved for future transplantation procedures.

Transplantation of desirable cell populations into Patients

In certain embodiments, cell populations are enriched for stem cells prior to transplantation. Various methods to select for stem cells are well known in the art. For example, cell samples can be enriched by fluorescently labeled monoclonal antibodies recognizing cell-surface markers of undifferentiated hematopoietic stem cells (e.g., CD34, CD59, Thy1, CD38 low, C-kit low, lin-minus) for sorting via fluorescence-activated cell sorting (FACS).

In other embodiments, a sample of the selected cells is transplanted, without enrichment.

In some embodiments, the endogenous stem cells of the bone marrow are eliminated or reduced prior to transplantation of the therapeutic stem cells. Therapeutic stem cells are defined as those stem cells containing beneficial sequence(s) or therapeutic vector(s).

In some embodiments, the transplantation process may involve the following phases: (1) conditioning, (2) stem cell infusion, (3) neutropenic phase, (4) engraftment phase, and (5) postengraftment period.

In some embodiments, the endogenous stem cells that normally produce the desired cells (e.g. bone marrow stem cells) are eliminated or reduced prior to transplantation. Chemotherapy, radiation, etc. and/or methods analogous to those described in U.S. Pat. No. 6,217,867 may be used to condition the bone marrow for appropriate engraftment of the transplant. Finally, therapeutic stem cells may be transplanted into the patient using any method known to the art.

Design of Numb/Numblike and Other Transgene Encoding Vectors

In one embodiment transfection with nucleic acid sequence(s) encoding numblike/numb isoform(s) is accomplished via viral transfection. The term “Numb/Numblike encoding vector(s)” refers to the vectors incorporating the nucleic acid sequence(s) encoding numblike/numb isoform(s) and/or synthetic oligonucleotides targeting numblike or numb isoforms, as well as any additional transgene sequences, synthetic oligonucleoties, etc, and any associated viral supernatant incorporated in those vector sequences.

The Numb/Numblike encoding vector(s) may comprise an expression vector. Appropriate expression vectors are those that may be employed for transfecting DNA or RNA into eukaryotic cells. Such vectors include, but are not limited to, prokaryotic vectors such as, for example, bacterial vectors; eukaryotic vectors, such as, for example, yeast vectors and fungal vectors; and viral vectors, such as, but not limited to adenoviral vectors, adeno-associated viral vectors, and retroviral vectors. Examples of retroviral vectors which may be employed include, but are not limited to, those derived from Moloney Murine Leukemia Virus, Moloney Murine Sarcoma Virus, and Rous Sarcoma Virus, FIV, HIV, SIV and hybrid vectors.

It is disclosed that the Numb/Numblike encoding vector(s) may be used to transfect cells in vitro and/or in vivo. Transfection can be carried out by any means known to the art, especially through virus produced from viral packaging cells. Such virus may be encapsidated so as to be capable of infecting a variety of cell types. Nevertheless, any encapsidation technique allowing infection of selected cell types and/or their progeny is practicable within the context of the present invention.

Design of Human Immunodeficiency Virus (HIV) Gene Therapy Vector(s)

The “therapeutc vector(s)” may incorporate an expression vector. Appropriate expression vectors are those that may be employed for transfecting DNA or RNA into eukaryotic cells. Such vectors include, but are not limited to, prokaryotic vectors such as, for example, bacterial vectors; eukaryotic vectors, such as, for example, yeast vectors and fungal vectors; and viral vectors, such as, but not limited to adenoviral vectors, adeno-associated viral vectors, and retroviral vectors. Examples of retroviral vectors which may be employed include, but are not limited to, those derived from Moloney Murine Leukemia Virus, Moloney Murine Sarcoma Virus, and Rous Sarcoma Virus, feline immunodeficiency virus (FIV), HIV, simian immunodeficiency virus (SIV) and hybrid vectors.

It is disclosed herein that the therapeutic vector(s) may be used to transfect target cells in vitro and/or in vivo. Transfection can be carried out by any means known to the art, especially through virus produced from viral packaging cells. Such virus may be encapsidated so as to be capable of infecting CD34+ cells and/or CD4+ cells. However, in some instances, other cell types are transfected by means not involving the CD4 or CD34 proteins. Nevertheless, any encapsidation technique allowing infection of such cell types may therefore be included in the disclosure of the present invention.

Pseudotyping with different envelope proteins expands the range of host cells transduceable by viral vectors and therapeutic vectors, and allows the virus to be concentrated to high titers, especially when pseudotyped with the vesicular stomatitis virus envelope glycoprotein (VSV-G) (Li et al., 1998; Reiser et al., 2000).

Vector Construction

Viral vectors utilized in this invention may be of various types including hybrid vectors. Vectors may, for instance, be third-generation lentiviral vectors which include only a very small fraction of the native genome (Zufferey et al., 1998). Production of transgene encoding vector(s) may also involve self-inactivating transfer vectors (Zufferey et al., 1998; Miyoshi et al., 1998) eliminating the production of full-length vector RNA after infection of target cells.

Viral vectors may be utilized which are replication-incompetent due to failure to express certain viral proteins necessary for replication. However the possibility exists that helper virus may enable therapeutic virus replication. This likelihood can be reduced by the use of self-inactivating vectors.

In a preferred embodiment, transgene sequences are driven by a ubiquitin promoter, U6 promoter, EF1alpha promoter, CMV promoter, regulable promoters and/or desired cell type specific promoters.

Viral Tropism

In a preferred embodiment, virus derived from the Numb isoform/Numblike encoding vector(s), therapeutic vector(s) and/or other transgeneic vector(s) of this invention is pseudotyped with vesicular stomatitis virus envelope glycoprotein to enable concentration of the virus to high titers and to facilitate infection of CD34+ cells.

Sequence Selection

The use of any sequence with 70% or greater identity (or complementarity) to any sequence referred to as a NUMB or Numblike sequence (searchable using the Entrez-Pubmed database) is covered by the invention if utilized in the manner described in the present invention.

The current invention also relates in part to a genetic vector that includes sequences capable of markedly reducing the susceptibility of mammalian cells to infection by HIV 1 and HIV-2 viruses (both together referred to herein as HIV).

The current invention discloses the novel combination of synthetic oligonucleotides to reduce the expression of genes critical to the HIV/AIDS disease process.

The desirability of combining synthetic oligonucleotides to effect co-receptor “knock down” with expression of TAR and RRE decoy sequences arises from the proposition, expressed herein, that combining multiple gene therapy approaches simultaneously targeting 1) HIV infection, 2) HIV transcription, and 3) HIV replication in individual cells is likely to produce superior therapeutic benefits than any of these approaches in isolation.

Therapeutic vector(s) express “beneficial sequence(s)” intended to render transfected or infected cells less capable of sustaining HIV replication and transcription. The genetic vector expressing “beneficial sequence(s)” as well as any virus derived from such genetic vector, are herein termed “therapeutic vector”.

The present invention is directed in part to the genetic modification of cells susceptible to infection by HIV or capable of propagating HIV. Such cells are herein termed “target cells”.

The present invention provides a composition and method for using therapeutic viral vectors to reduce the susceptibility of mature or immature target cells, leukocytes, blood cells, any stem/progenitor cells, and/or their progeny to infection by HIV.

It follows that the present invention also provides a composition and method for using therapeutic viral vectors to reduce the susceptibility of reprogrammed cells, induced multipotent cells, induced pluripotent cells, and/or their progeny to infection by HIV.

It is a further objective of this invention to reduce the ability of mature or immature target cells, stem/progenitor cells, (including reprogrammed cells, induced multipotent cells, induced pluripotent cells) and/or their progeny to sustain immunodeficiency virus replication and transcription.

It is another objective of this invention to achieve efficient, long-term expression of the therapeutic sequences in mature or immature target cells, other quiescent cells, stem/progenitor cells, and/or their progeny.

In one aspect, this invention provides a method for preventing or treating HIV infection. The method involves transplanting stem cells transfected with therapeutic vector(s) or sequence(s), into patients with HIV infection.

Beneficial sequence(s) may be ones that reduce the ability of HIV to infect a cell, transcribe viral DNA, or replicate within an infected cell, or which enhances the ability of a cell to neutralize HIV infection.

In certain embodiments, the beneficial sequence(s) represent synthetic oligonucleotide(s) which interfere with HIV entry, including siRNA, shRNA, antisense RNA or miRNA directed against any of the HIV co-receptors (including, but not limited to, CXCR4, CCR5, CCR2b, CCR3, and CCR1).

In a preferred embodiment, the therapeutic vector(s) includes synthetic oligonucleotides targeting one or more HIV co-receptors including CXCR4, CCR5, CCR1, CCR2, CCR3, CXCR6 and/or BOB.

In another preferred embodiment the therapeutic vector(s) includes synthetic oligonucleotides targeting the major HIV co-receptors CXCR4 and CCR5

In a further preferred embodiment the therapeutic vector(s) includes synthetic oligonucleotides targeting one or more HIV enzymes such as HIV reverse transcriptase, integrase and protease.

Appropriate sequences for the synthetic oligonucleotides are those 1) predictable by computer algorithms to be effective in reducing targeted sequences, and 2) capable of successfully reduce the amount of targeted enzyme by >70% in standard quantitative RNA assays and in assays of enzymatic activity or to a lesser but therapeutic degree.

The phrase “targeted sequence” indicates that a particular sequence has a nucleotide base sequence that has at least 70% identity to a viral genomic nucleotide sequence or its complement (e.g., is the same as or complementary to such viral genomic sequence), or is a corresponding RNA sequence. In particular embodiments of the present invention, the term indicates that the sequence is at least 70% identical to a viral genomic sequence of the particular virus against which the oligonucleotide is directed, or to its complementary sequence.

Any of the various types of synthetic oligonucleotides may be expressed via therapeutic vector transfection, and the current invention is directed to all possible combinations of such oligonucleotides.

In a preferred embodiment, the synthetic oligonucleotide sequences are driven by target cell, specific promoter(s).

In another preferred embodiment, the synthetic oligonucleotide sequences are driven by U6 promoter(s).

Synthetic oligonucleotides, by the same token, may be included in the same therapeutic vector(s) with decoy RNA.

Decoy RNA

Decoy RNA are sequences of RNA that are effective at binding to certain proteins and inhibiting their function.

In a preferred embodiment, the therapeutic vector(s) comprise(s) multiple decoy RNA sequences.

In a further embodiment the decoy RNA sequences are flanked by sequences that provide for stability of the decoy sequence.

In another preferred embodiment the decoy RNA sequences are RRE and/or TAR decoy sequences.

In a preferred embodiment, the RRE and TAR decoy sequences are HIV-2 derived TAR and RRE sequences.

In another preferred embodiment the decoy sequences also include Psi element decoy sequences.

In a preferred embodiment, the decoy sequences are each driven by a U6 promoter.

In another preferred embodiment, the decoy sequences are driven by target-cell specific promoters.

In a preferred embodiment, the therapeutic vector targets multiple stages of the HIV life cycle by encoding synthetic nucleotide sequence(s) in combination with HIV-2 TAR and/or RRE decoy sequences.

In another preferred embodiment, the vector includes miRNA oligonucleotide sequences.

In another preferred embodiment, the vector includes shRNA oligonucleotide sequences.

In another preferred embodiment, the vector includes siRNA oligonucleotide sequences.

In another preferred embodiment, the vector includes RNAi oligonucleotide sequences.

In another preferred embodiment, the vector includes ribozyme sequences.

In another preferred embodiment, the vector includes a combination of synthetic oligonucleotide classes.

In a further embodiment, the synthetic nucleotide sequences target HIV co-receptors such as CCR5, CXCR4, etc.

In a further embodiment, the synthetic nucleotide sequences target HIV enzymes such as integrase, protease, reverse transcriptase, TAT, etc.

In a further embodiment, the ribozyme sequences target HIV co-receptors such as CCR5, CXCR4, etc, or HIV enzymes such as integrase, protease, reverse transcriptase, TAT, etc.

In a preferred embodiment, virus is generated using the therapeuic vector(s) and the virus is pseudotyped.

In a preferred embodiment, virus is generated using the therapeuic vector(s) and the virus is not pseudotyped and the virus shows native HIV tropism.

In a preferred embodiment, the therapeutic vector(s) is a viral vector.

In a preferred embodiment, the therapeutic vector(s) is a lentiviral vector.

In a preferred embodiment, the therapeutic vector(s) is a third generation lentiviral vector.

In a preferred embodiment, the therapeutic vector(s) includes a combination of synthetic oligonucleotide classes.

In a preferred embodiment, synthetic nucleotide sequence expression is driven by the EF-1 alpha promoter or other target-cell appropriate promoters.

In a preferred embodiment, synthetic nucleotide sequence expression is driven by the U6 promoter or other target-cell appropriate promoters.

In a preferred embodiment, synthetic nucleotide sequence expression is driven by a combination of EF-1 alpha and U6, and/or other target-cell appropriate promoters.

In a preferred embodiment, EF-1 alpha drives miRNA expression while the U6 promoter drives RNA decoy expression.

In a preferred embodiment, EF-1 alpha drives siRNA sequence expression while the U6 promoter drives RNA decoy expression.

In a preferred embodiment, EF-1 alpha drives shRNA sequence expression while the U6 promoter drives RNA decoy expression.

In a preferred embodiment, the therapeutic vector(s) includes multiple miRNA sequences directed against CXCR4, multiple miRNA sequences directed against CCR5, an HIV-2 RRE decoy sequence and an HIV-2 TAR decoy sequence, and the vector is a viral vector.

In a preferred embodiment, treatment involving the therapeutic vector(s) is combined with other modes of antiretroviral therapy including pharmacological therapies. Antiretroviral therapies appropriate for combination with the therapeutic vector(s) are those that have additive or synergistic effects in combination with the therapeutic vector.

Cells targeted for gene therapy in HIV may include, but are not necessarily be limited to mature peripheral blood T lymphocytes, monocytes, tissue macrophages, T cell progenitors, macrophage-monocyte progenitor cells, and/or multipotent hematopoietic stem cells, such as those found in umbilical cord blood, peripheral blood, and occupying bone marrow spaces.

The present invention also relates to transfection of CD4+ T cells, macrophages, T cell progenitors, macrophage-monocyte progenitors, CD 34+ stem/progenitor cells and/or any other quiescent cell, dividing cell, stem cell or progenitor cell capable of differentiation in vitro or in vivo into HIV target cells, CD4+ T cells, macrophages, T cell progenitors, macrophage-monocyte progenitors, and/or CD 34+ stem/progenitor cells. Transfected cells, therefore, can be endogenous cells in situ, or exogenous cells derived from other body regions or even other individual donors. Cells selected for this purpose are herein termed “selected cells”.

By the same token, self-renewing, multipotent and/or pluripotent stem cells (including reprogrammed and induced pluripotent cells) represent another logical target for HIV gene therapy, and their use is specifically covered by the present invention.

In one embodiment of this process, selected cells (e.g. hematopoietic stem cells, skin stem cells, umbilical cord cells, primordial germ cells (PGCs), spermatogonia, any accessible somatic cell, etc.) are 1) propagated in culture using one or more cytokines such as steel factor, leukemia inhibitory factor (LIF), cardiotropin-1, IL-11, IL-6, IL-6 R, GP-130, CNTF, IGF-I, bFGF, and/or oncostatin-M and 2) transfected with the therapeutic vector(s) or beneficial sequence(s) prior to differentiation using any methods known to the art, such as those described in U.S. Pat. No. 5,677,139 herein incorporated by reference, or by methods analogous to U.S. Pat. No. 5,677,139 with respect to other target cells.

In separate embodiments, it may be desirable to perform the various steps prior to transfection.

In separate embodiments, for the purpose of generating pluripotent stem cell populations, it may be desirable to perform only the incubation steps above.

Appropriate concentrations of LIF and steel factor for stem/progenitor cell propagation/proliferation as well as other cell culture conditions have been described previously (e.g. U.S. Pat. Nos. 6,432,711 and 5,453,357 herein incorporated by reference). Other appropriate protocols and reference cytokine concentrations have been taught by Koshimizu et al., 1996; Keller et al., 1996; Piquet-Pellorce, 1994; Rose et al., 1994; Park and Han, 2000; Guan et al., 2006; Dykstra et al., 2006).

The population of target cells may include somatic cells, stem cells and progenitor cells. Stem cells may be derived from existing cell lines or isolated from stored, banked, or cryopreserved sources. Typical sources of stem cells include marrow, peripheral blood, placental blood, amniotic fluid, umbilical cord blood, adipose tissue, non-human embryos, etc.

Somatic cells, especially circulating leukocytes and other non-progenitor/stem cells may likewise be subjected to the same culture conditions as described above for stem/progenitor cells effective that they acquire stem/progenitor cell properties as a result.

The invention also discloses the production (e.g. US Patent Application 20030099621) of target cells from stem/progenitor cells that may be made relatively resistant to HIV infection and/or HIV replication.

It is understood, however, that any method of differentiating previously propagated stem/progenitor/leukocyte cells into the desired target cells may be employed within the scope of the invention so long as functional target cells relatively resistant to HIV infection and/or HIV replication/and/or HIV transcription are produced.

In a preferred embodiment, the therapeutic viral vector is packaged with one or more envelope proteins from native HIV viruses conferring upon the therapeutic virus the capacity to infect any cell that native HIV strains are capable of infecting.

Cells selected for use in this invention will be in some instances accessible (e.g. umbilical cord stem cells, bone marrow stem cells, spermatogonia and primordial germ cells of the testis, stem cells isolated from amniotic fluid, stem cells isolated from the skin, etc.). Such cells can be isolated from the tissues in which they reside by any means known to the art.

Other selected cells may comprise reprogrammed cells, induced multipotent cells, induced pluripotent cells, etc.

In accordance with an aspect of the present invention, there is provided a method of producing a desired cell line, cell type, or cell class from the selected cells. Generally, the method comprises culturing the selected cells and/or their progeny under conditions which promote growth of the selected cells at an optimal growth rate. The resulting cell population is then cultured under conditions which promote cell growth at a rate which is typically less than the optimal rate, and in the presence of an agent promoting differentiation of the cells into the desired cell line, cell type, or cell class (e.g. CD4+ T cells).

The present invention also discloses the propagation of the selected cells and/or their progeny in culture, before or after transfection with the therapeutic vector, by any means known to the art (e.g. US Patent Application 20060099177). Such methods also include incubation with LIF, steel factor, Il-6, IL-7, oncostatin-M and/or cardiotropin-1 and other growth enhancing cytokines, etc.

The present invention further discloses the directed differentiation of cells transfected with the therapeutic vector(s) into desired cell types by further incubation in media containing the appropriate cytokines and growth factors such as colony stimulating factors such as M-CSF (CSF-1), GM-CSF, IL-7, any cytokine promoting CD4+ T cell differentiation, etc.

Transfection

Genetic modification of selected cells and target cells, whether they be exogenous cells or endogenous cells can be performed according to any published or unpublished method known to the art (e.g. U.S. Pat. No. 6,432,711, U.S. Pat. No. 05,593,875, U.S. Pat. No. 05,783,566, U.S. Pat. No. 5,928,944, U.S. Pat. No. 05,910,488, U.S. Pat. No. 05,824,547, etc.) or by other generally accepted means. Suitable methods for transforming host cells can be found in Sambrook et al. (Molecular Cloning: A Laboratory Manual, 2nd Edition, Cold Spring Harbor Laboratory press (1989)), and other laboratory textbooks.

Successfully transfected cells can be identified by selection protocols involving markers such as antibiotic resistance genes in addition to RNA expression assays and morphological analyses. Clones from successfully transfected cells, expressing the appropriate exogenous DNA at appropriate levels, can be preserved as cell lines by cryopreservation (utilizing any appropriate method of cryopreservation known to the art).

Selectable markers (e.g., antibiotics resistance genes) may include those which confer resistance to drugs, such as G418, hygromycin, ampicillin and blasticidin, etc. Cells containing the gene of interest can be identified by drug selection where cells that have incorporated the selectable marker gene survive, and others die.

A theoretical basis for the embodiments of the invention is described herein, however, this discussion is not in any way to be considered as binding or limiting on the present invention. Those of skill in the art will understand that the various embodiments of the invention may be practiced regardless of the model used to describe the theoretical underpinnings of the invention.

The invention will now be described and illustrated with respect to the following examples; however, the scope of the present invention is not intended to be limited thereby.

### Example 1

### Construction of the Transgenic Vectors Suitable for Use in the Present Invention

Suitable EGFP-Numb and EGFP-Numblike, and EGFP-X lentiviral vectors (where X is any transgene described in the present invention) can be produced by cloning into an appropriate viral vector (e.g. the two-gene HIV-EGFP-HSA vector (Reiser et al., 2000)). Adapter primers can be selected for PCR amplification of Numblike and Numb isoform cDNAs and cloning into a genetic vector. In preparation for cloning, the gene vector is digested with enzymes. Subsequently, the cDNA for each transgene is inserted into the nef coding region previously occupied by the HSA cDNA. EGFP (enhanced green fluorescent protein) and a cell population-appropriate promoter (e.g. CMV ie or EF1alpha) having been previously inserted into the viral coding region. Genetic constructs may include a vector backbone, and a transactivator which regulates a promoter operably linked to heterologous nucleic acid sequences.

Examples of retroviral vectors which may be employed include, but are not limited to, those derived from Moloney Murine Leukemia Virus, Moloney Murine Sarcoma Virus, and Rous Sarcoma Virus, FIV, and HIV. Appropriate expression vectors are those that may be employed for transfecting DNA or RNA into eukaryotic cells. Such vectors include, but are not limited to, prokaryotic vectors such as, for example, bacterial vectors; eukaryotic vectors, such as, for example, yeast vectors and fungal vectors; and viral vectors, such as, but not limited to, lentiviral vectors, adenoviral vectors, adeno-associated viral vectors, and retroviral vectors.

The replication incompetent pcDNA 6.2/EmGFP-Bsd/V5-DEST vector is an example of an appropriate expression vector (Invitrogen) and allows expression of synthetic oligonucleotides (e.g. miRNAs) transferred from the pcDNA 6.2 GW/miR vector that have the capacity to cleave targeted sequences. These vectors include flanking and loop sequences from endogenous miRNA to direct the excision of the engineered miRNA from a longer Pol II transcript (pre-miRNA).

Combining multiple miRNA sequences directed against specific endogenous RNA species increases the likelihood of success in reducing target sequence expression. miRNA sequences may be operably linked to regulable or tissue specific promoters.

By utilizing lentiviral vectors for gene expression, the resulting Numb/Numblike encoding vector(s) and/or other transgenic vector(s) of this invention, becomes capable of stably transducing both dividing and non-dividing cell types.

In a preferred embodiment, the resulting Numb/Numblike encoding vector(s), and/or other transgenic vector(s) of this invention contain multiple synthetic oligonucleotide sequences driven by one or more promoters so as to reduce expression of specific numb isoforms and/or numblike.

### Example 2

Another Example of a suitable vector is a retroviral vector. Retroviruses are RNA viruses that contain an RNA genome. The gag, pol, and env genes are flanked by long terminal repeat (LTR) sequences. The 5′ and 3′ LTR sequences promote transcription and polyadenylation of mRNA's.

The retroviral vector may provide a regulable transactivating element, an internal ribosome reentry site (IRES), a selection marker, and a target heterologous gene operated by a regulable promoter.

Alternatively, multiple sequences may be expressed under the control of multiple promoters. Finally, the retroviral vector may contain cis-acting sequences necessary for reverse transcription and integration. Upon infection, the RNA is reverse transcribed to DNA that integrates efficiently into the host genome. The recombinant retrovirus of this invention is genetically modified in such a way that some of the retroviral, infectious genes of the native virus have been removed and in certain instances replaced instead with a target nucleic acid sequence for genetic modification of the cell. The sequences may be exogenous DNA or RNA, in its natural or altered form.

### Example 3

### Example Methods for Generation of Numb/Numblike Encoding Vector(s), and/or Other Transgenic Vector(s) of this Invention

The methods for generation of the resulting Numb/Numblike encoding vector(s), and/or other transgenic vector(s) of this invention include those taught in Invitrogen's Viral Power Lentiviral Expression Systems Manual, 2007. Briefly, the EmGFP-bsd cassette is cloned as a Pm1I-B1pI fragment into the pLenti6/R4R2/V5-DEST vector, while the miR-long (PRR+) numb isoform or miR-short numb isoform/numblike cassettes are simultaneously transferred by BP reaction into pDONR221. Then the regulable promoter(s) and miR-isoform cassettes are Multisite LR crossed into the modified pLenti6/EmGFP-bsd/R4R2-DESTvector.

Multiple vectors can be generated in this manner comprising different combinations of synthetic oligonucleotides and transgene cassettes.

pLenti6/R4R2/V5-DEST vector sequence
(SEQ ID NO: 1):
aatgtagtcttatgcaatactcttgtagtcttgcaacatggtaacgatga
gttagcaacatgccttacaaggagagaaaaagcaccgtgcatgccgattg
gtggaagtaaggtggtacgatcgtgccttattaggaaggcaacagacggg
tctgacatggattggacgaaccactgaattgccgcattgcagagatattg
tatttaagtgcctagctcgatacataaacgggtctctctggttagaccag
atctgagcctgggagctctctggctaactagggaacccactgcttaagcc
tcaataaagcttgccttgagtgcttcaagtagtgtgtgcccgtctgttgt
gtgactctggtaactagagatccctcagacccttttagtcagtgtggaaa
atctctagcagtggcgcccgaacagggacttgaaagcgaaagggaaacca
gaggagctctctcgacgcaggactcggcttgctgaagcgcgcacggcaag
aggcgaggggcggcgactggtgagtacgccaaaaattttgactagcggag
gctagaaggagagagatgggtgcgagagcgtcagtattaagcgggggaga
attagatcgcgatgggaaaaaattcggttaaggccagggggaaagaaaaa
atataaattaaaacatatagtatgggcaagcagggagctagaacgattcg
cagttaatcctggcctgttagaaacatcagaaggctgtagacaaatactg
ggacagctacaaccatcccttcagacaggatcagaagaacttagatcatt
atataatacagtagcaaccctctattgtgtgcatcaaaggatagagataa
aagacaccaaggaagctttagacaagatagaggaagagcaaaacaaaagt
aagaccaccgcacagcaagcggccgctgatcttcagacctggaggaggag
atatgagggacaattggagaagtgaattatataaatataaagtagtaaaa
attgaaccattaggagtagcacccaccaaggcaaagagaagagtggtgca
gagagaaaaaagagcagtgggaataggagctttgttccttgggttcttgg
gagcagcaggaagcactatgggcgcagcgtcaatgacgctgacggtacag
gccagacaattattgtctggtatagtgcagcagcagaacaatttgctgag
ggctattgaggcgcaacagcatctgttgcaactcacagtctggggcatca
agcagctccaggcaagaatcctggctgtggaaagatacctaaaggatcaa
cagctcctggggatttggggttgctctggaaaactcatttgcaccactgc
tgtgccttggaatgctagttggagtaataaatctctggaacagatttgga
atcacacgacctggatggagtgggacagagaaattaacaattacacaagc
ttaatacactccttaattgaagaatcgcaaaaccagcaagaaaagaatga
acaagaattattggaattagataaatgggcaagtttgtggaattggttta
acataacaaattggctgtggtatataaaattattcataatgatagtagga
ggcttggtaggtttaagaatagtttttgctgtactttctatagtgaatag
agttaggcagggatattcaccattatcgtttcagacccacctcccaaccc
cgaggggacccgacaggcccgaaggaatagaagaagaaggtggagagaga
gacagagacagatccattcgattagtgaacggatctcgacggtatcgatg
tcgacgttaacgctagtgatatcaactttgtatagaaaagttgaacgaga
aacgtaaaatgatataaatatcaatatattaaattagattttgcataaaa
aacagactacataatactgtaaaacacaacatatccagtcactatggcgg
ccgcattaggcaccccaggctttacactttatgcttccggctcgtataat
gtgtggattttgagttaggatccgtcgagattttcaggagctaaggaagc
taaaatggagaaaaaaatcactggatataccaccgttgatatatcccaat
ggcatcgtaaagaacattttgaggcatttcagtcagttgctcaatgtacc
tataaccagaccgttcagctggatattacggcctttttaaagaccgtaaa
gaaaaataagcacaagttttatccggcctttattcacattcttgcccgcc
tgatgaatgctcatccggaattccgtatggcaatgaaagacggtgagctg
gtgatatgggatagtgttcacccttgttacaccgttttccatgagcaaac
tgaaacgttttcatcgctctggagtgaataccacgacgatttccggcagt
ttctacacatatattcgcaagatgtggcgtgttacggtgaaaacctggcc
tatttccctaaagggtttattgagaatatgtttttcgtctcagccaatcc
ctgggtgagtttcaccagttttgatttaaacgtggccaatatggacaact
tcttcgcccccgttttcaccatgggcaaatattatacgcaaggcgacaag
gtgctgatgccgctggcgattcaggttcatcatgccgtttgtgatggctt
ccatgtcggcagaatgcttaatgaattacaacagtactgcgatgagtggc
agggcggggcgtaaagatctggatccggcttactaaaagccagataacag
tatgcgtatttgcgcgctgatttttgcggtataagaatatatactgatat
gtatacccgaagtatgtcaaaaagaggtatgctatgaagcagcgtattac
agtgacagttgacagcgacagctatcagttgctcaaggcatatatgatgt
caatatctccggtctggtaagcacaaccatgcagaatgaagcccgtcgtc
tgcgtgccgaacgctggaaagcggaaaatcaggaagggatggctgaggtc
gcccggtttattgaaatgaacggctcttttgctgacgagaacagggactg
gtgaaatgcagtttaaggtttacacctataaaagagagagccgttatcgt
ctgtttgtggatgtacagagtgatattattgacacgcccgggcgacggat
ggtgatccccctggccagtgcacgtctgctgtcagataaagtctcccgtg
aactttacccggtggtgcatatcggggatgaaagctggcgcatgatgacc
accgatatggccagtgtgccggtctccgttatcggggaagaagtggctga
tctcagccaccgcgaaaatgacatcaaaaacgccattaacctgatgttct
ggggaatataaatgtcaggctccgttatacacagccagtctgcaggtcga
ccatagtgactggatatgttgtgttttacagtattatgtagtctgttttt
tatgcaaaatctaatttaatatattgatatttatatcatttttacgtttc
tcgttcagctttcttgtacaaagtggttgatatccagcacagtggcggcc
gctcgagtctagagggcccgcggttcgaaggtaagcctatccctaaccct
ctcctcggtctcgattctacgcgtaccggttagtaatgagtttggaatta
attctgtggaatgtgtgtcagttagggtgtggaaagtccccaggctcccc
agcaggcagaagtatgcaaagcatgcatctcaattagtcagcaaccaggt
gtggaaagtccccaggctccccagcaggcagaagtatgcaaagcatgcat
ctcaattagtcagcaaccatagtcccgcccctaactccgcccatcccgcc
cctaactccgcccagttccgcccattctccgccccatggctgactaattt
tttttatttatgcagaggccgaggccgcctctgcctctgagctattccag
aagtagtgaggaggcttttttggaggcctaggcttttgcaaaaagctccc
gggagcttgtatatccattttcggatctgatcagcacgtgttgacaatta
atcatcggcatagtatatcggcatagtataatacgacaaggtgaggaact
aaaccatggccaagcctttgtctcaagaagaatccaccctcattgaaaga
gcaacggctacaatcaacagcatccccatctctgaagactacagcgtcgc
cagcgcagctctctctagcgacggccgcatcttcactggtgtcaatgtat
atcattttactgggggaccttgtgcagaactcgtggtgctgggcactgct
gctgctgcggcagctggcaacctgacttgtatcgtcgcgatcggaaatga
gaacaggggcatcttgagcccctgcggacggtgccgacaggtgcttctcg
atctgcatcctgggatcaaagccatagtgaaggacagtgatggacagccg
acggcagttgggattcgtgaattgctgccctctggttatgtgtgggaggg
ctaagcacaattcgagctcggtacctttaagaccaatgacttacaaggca
gctgtagatcttagccactttttaaaagaaaaggggggactggaagggct
aattcactcccaacgaagacaagatctgctttttgcttgtactgggtctc
tctggttagaccagatctgagcctgggagctctctggctaactagggaac
ccactgcttaagcctcaataaagcttgccttgagtgcttcaagtagtgtg
tgcccgtctgttgtgtgactctggtaactagagatccctcagaccctttt
agtcagtgtggaaaatctctagcagtagtagttcatgtcatcttattatt
cagtatttataacttgcaaagaaatgaatatcagagagtgagaggaactt
gtttattgcagcttataatggttacaaataaagcaatagcatcacaaatt
tcacaaataaagcatttttttcactgcattctagttgtggtttgtccaaa
ctcatcaatgtatcttatcatgtctggctctagctatcccgcccctaact
ccgcccatcccgcccctaactccgcccagttccgcccattctccgcccca
tggctgactaattttttttatttatgcagaggccgaggccgcctcggcct
ctgagctattccagaagtagtgaggaggcttttttggaggcctagggacg
tacccaattcgccctatagtgagtcgtattacgcgcgctcactggccgtc
gttttacaacgtcgtgactgggaaaaccctggcgttacccaacttaatcg
ccttgcagcacatccccctttcgccagctggcgtaatagcgaagaggccc
gcaccgatcgcccttcccaacagttgcgcagcctgaatggcgaatgggac
gcgccctgtagcggcgcattaagcgcggcgggtgtggtggttacgcgcag
cgtgaccgctacacttgccagcgccctagcgcccgctcctttcgctttct
tcccttcctttctcgccacgttcgccggctttaccccgtcaagctctaaa
tcgggggctccctttagggttccgatttagtgctttacggcacctcgacc
ccaaaaaacttgattagggtgatggttcacgtagtgggccatcgccctga
tagacggtttttcgccctttgacgttggagtccacgttctttaatagtgg
actcttgttccaaactggaacaacactcaaccctatctcggtctattctt
ttgatttataagggattttgccgatttcggcctattggttaaaaaatgag
ctgatttaacaaaaatttaacgcgaattttaacaaaatattaacgcttac
aatttaggtggcacttttcggggaaatgtgcgcggaacccctatttgttt
atttttctaaatacattcaaatatgtatccgctcatgagacaataaccct
gataaatgcttcaataatattgaaaaaggaagagtatgagtattcaacat
ttccgtgtcgcccttattcccttttttgcggcattttgccttcctgtttt
tgctcacccagaaacgctggtgaaagtaaaagatgctgaagatcagttgg
gtgcacgagtgggttacatcgaactggatctcaacagcggtaagatcctt
gagagttttcgccccgaagaacgttttccaatgatgagcacttttaaagt
tctgctatgtggcgcggtattatcccgtattgacgccgggcaagagcaac
tcggtcgccgcatacactattctcagaatgacttggttgagtactcacca
gtcacagaaaagcatcttacggatggcatgacagtaagagaattatgcag
tgctgccataaccatgagtgataacactgcggccaacttacttctgacaa
cgatcggaggaccgaaggagctaaccgcttttttgcacaacatgggggat
catgtaactcgccttgatcgttgggaaccggagctgaatgaagccatacc
aaacgacgagcgtgacaccacgatgcctgtagcaatggcaacaacgttgc
gcaaactattaactggcgaactacttactctagcttcccggcaacaatta
atagactggatggaggcggataaagttgcaggaccacttctgcgctcggc
ccttccggctggctggtttattgctgataaatctggagccggtgagcgtg
ggtctcgcggtatcattgcagcactggggccagatggtaagccctcccgt
atcgtagttatctacacgacggggagtcaggcaactatggatgaacgaaa
tagacagatcgctgagataggtgcctcactgattaagcattggtaactgt
cagaccaagtttactcatatatactttagattgatttaaaacttcatttt
taatttaaaaggatctaggtgaagatcctttttgataatctcatgaccaa
aatcccttaacgtgagttttcgttccactgagcgtcagaccccgtagaaa
agatcaaaggatcttcttgagatcctttttttctgcgcgtaatctgctgc
ttgcaaacaaaaaaaccaccgctaccagcggtggtttgtttgccggatca
agagctaccaactctttttccgaaggtaactggcttcagcagagcgcaga
taccaaatactgttcttctagtgtagccgtagttaggccaccacttcaag
aactctgtagcaccgcctacatacctcgctctgctaatcctgttaccagt
ggctgctgccagtggcgataagtcgtgtcttaccgggttggactcaagac
gatagttaccggataaggcgcagcggtcgggctgaacggggggttcgtgc
acacagcccagcttggagcgaacgacctacaccgaactgagatacctaca
gcgtgagctatgagaaagcgccacgcttcccgaagggagaaaggcggaca
ggtatccggtaagcggcagggtcggaacaggagagcgcacgagggagctt
ccagggggaaacgcctggtatctttatagtcctgtcgggtttcgccacct
ctgacttgagcgtcgatttttgtgatgctcgtcaggggggcggagcctat
ggaaaaacgccagcaacgcggcctttttacggttcctggccttttgctgg
ccttttgctcacatgttctttcctgcgttatcccctgattctgtggataa
ccgtattaccgcctttgagtgagctgataccgctcgccgcagccgaacga
ccgagcgcagcgagtcagtgagcgaggaagcggaagagcgcccaatacgc
aaaccgcctctccccgcgcgttggccgattcattaatgcagctggcacga
caggtttcccgactggaaagcgggcagtgagcgcaacgcaattaatgtga
gttagctcactcattaggcaccccaggctttacactttatgcttccggct
cgtatgttgtgtggaattgtgagcggataacaatttcacacaggaaacag
ctatgaccatgattacgccaagcgcgcaattaaccctcactaaagggaac
aaaagctggagctgcaagctt

### Example 4

### Additional Methods for Generation of Therapeutic Vector(s)

“Packaging cell lines” derived from human and/or animal fibroblast cell lines result from transfecting or infecting normal cell lines with viral gag, pol, and env structural genes. On the other hand, packaging cell lines produce RNA devoid of the psi sequence, so that the viral particles produced from packaging cell do not contain the gag, pol, or env genes. Once the therapeutic vector's DNA containing the psi sequence (along with the therapeutic gene) is introduced into the packaging cell, by means of transfection or infection, the packaging cell may produce virions capable of transmitting the therapeutic RNA to the final target cell (e.g. a CD4+ cell).

The “infective range” of the therapeutic vector(s) is determined by the packaging cell line. A number of packaging cell lines are available for production of virus suitable for infecting a broad range of human cell types. These packaging cell lines are nevertheless generally capable of encapsidating viral vectors derived from viruses that in nature usually infect different animal species. For example, vectors derived from SIV or MMLV can be packaged by GP120 encapsidating cell lines.

An example protocol for producing a therapeutic viral supernatant is provided as follows:

1. Twenty micrograms of retrovirus vector are mixed with 2-3 micrograms of viral DNA containing the selectable marker gene (e.g. antibiotic resistance gene) by gentle tapping in 0.8-1 milliliter of Hepes buffered saline (pH=7.05) in a 1.5 ml plastic tube.

2. Seventy microliters of 2M CaCl² are added to the mixture by repeated gentle tapping.

3. When a blue precipitate first begins to appear within the tube, the product should be gently applied to a 30% confluent layer of packaging cells (from any number of commercial vendors). The DNA mixture should be applied only after first removing the medium from the packaging cells.

4. The packaging cells are set to incubate for 20-30 minutes at room temperature (25 degrees Celsius) before transferring them back to an incubator at 36-38 degrees Celsius for 3.5 hours.

5. Add 3.5-4 milliliters of Hepes buffered saline containing 15% glycerol for 3 minutes then wash cell with Dulbecco's Modified Eagle's Medium (DMEM)+10% FBS×2.

6. Add back DMEM+10% FBS, and incubate cells for 20 hours at 37 degrees Celsius.

7. Remove and filter medium containing therapeutic viral particles.

Excess viral supernatant is immediately stored or concentrated and stored at −80 degrees Celsius). Supernatant may stored with 5-8 micrograms of polybrene to increase the efficiency of target cell infection. Otherwise polybrene may be excluded or added just before infection.

8. Stable producer lines can be established by splitting packaging cell lines 1 to 20, or 1 to 40 and subsequently incubating these cells for up to 10 days (changing medium every three days) in medium containing selective drugs (e.g. certain antibiotics corresponding to transfected resistance genes).

9. After 10 days isolated colonies are picked, grown-up aliquoted and frozen for storage.

Assay of Retrovirus Infectivity/Titration is achieved by application of a defined volume of viral supernatant to a layer of confluent “test” cells such as NIH 3T3 cells plated at 20% confluence. After 2-3 cell division times (24-36 hours for NIH 3T3 cells) colonies of “test” cells incubated at 37 degrees in antibiotic-containing medium are counted. The supernatant's titer are estimated from these colony counts by the following formula:

Colony Forming Units/ml=colonies identified×0.5(split factor)/volume of virus (ml)

The accuracy of this estimate is increased by testing large volumes of supernatant over many plates of “test” cells.

Application of the therapeutic viral supernatant to target cells may be accomplished by various means appropriate to the clinical situation.

### Example 5

### Growth Medium for Selected Cells

Selected cells can be expanded/grown in Dulbecco's modified Minimal Essential Medium (DMEM) supplemented with glutamine, beta.-mercaptoethanol, 10% (by volume) horse serum, and human recombinant Leukemia Inhibitory Factor (LIF). LIF replaces the need for maintaining selected cells on feeder layers of cells, (which may also be employed) and is essential for maintaining selected cells in an undifferentiated, multipotent, or pluripotent state, such cells can be maintained in Dulbecco's modified Minimal Essential Medium (DMEM) supplemented with glutamine, beta.-mercaptoethanol, 10% (by volume) horse serum, and human recombinant Leukemia Inhibitory Factor (LIF). The LIF replaces the need for maintaining cells on feeder layers of cells, (which may also be employed) and is essential for maintaining cells in an undifferentiated state (per U.S. Pat. No. 6,432,711).

In order to initiate the differentiation of the selected cells into neuronal cells, the cells are trypsinized and washed free of LIF, and placed in DMEM supplemented with 10% fetal bovine serum (FBS). After resuspension in DMEM and 10% FBS, 1×10⁶ cells are plated in 5 ml DMEM, 10% FBS, 0.5 microM retinoic acid in a 60 mm Fisher bacteriological grade Petri dishes, where the cells are expected to form small aggregates. Aggregation aids in proper cell differentiation. High efficiency transfection with appropriate neuronal transcription factors can occur before or after plating in DMEM, FBS, and retinoic acid. (See U.S. Pat. Nos. 6,432,711 and 5,453,357 for additional details).

### Example 6

### HLA Matching

Selected cells (e.g. umbilical cord blood or cells from any other suitable source and/or their progeny), can be screened, genetically-modified (optional), expanded, and induced to begin differentiating into the desired cell type(s) (optional). The cells are then transplanted according to standard stem cell transplantation protocols. In certain instances, cells may be transplanted into patients without HLA matching.

### Example 7

In some rare instance, it may be appropriate to introduce transgene encoding vectors into patients in order to stimulate or inhibit cellular division or cellular differentiation, in vivo.

### Example 8

### Genetic Modification of Selected Cells

In vitro genetic modification of exogenous cells or patient's endogenous cells can be performed according to any published or unpublished method known to the art (e.g. U.S. Pat. Nos. 6,432,711, U.S. Pat. No. 05,593,875, U.S. Pat. No. 05,783,566, U.S. Pat. No. 5,928,944, U.S. Pat. No. 05,910,488, U.S. Pat. No. 05,824,547, etc.) or by other generally accepted means. Suitable methods for transforming host cells can be found in Sambrook et al. (Molecular Cloning: A Laboratory Manual, 2nd Edition, Cold Spring Harbor Laboratory press (1989)), and other laboratory textbooks.

Successfully transfected cells are identified by selection protocols involving markers such as antibiotic resistance genes in addition to RNA expression assays and morphological analyses. Clones from successfully transfected cells, expressing the appropriate exogenous DNA at appropriate levels, can be preserved as cell lines by cryopreservation (utilizing any appropriate method of cryopreservation known to the art).

Selectable markers (e.g., antibiotics resistance genes) may include those conferring resistance to drugs, such as G418, hygromycin and methotrexate. Cells containing the gene of interest can be identified by drug selection where cells that have incorporated the selectable marker gene survive, and others die.

The current invention discloses the selection of genetically-modified cells as “selected cells” of the invention. The term genetic modification refers to alteration of the cellular genotype by introducing natural or synthetic nucleic acids into selected cells and/or their progeny or immortalized cell lines and/or their progeny by any means known to the art. Alternatively culture conditions that induce permanent changes in gene expression patterns are considered herein to represent genetic modification. Modification of stem cells, whether they be derived from the host brain, endogenous donor sources, exogenous donor sources, or cell lines, represents a feasible approach to the treatment of certain human diseases, especially those of the human nervous system.

Genetic modifications covered by this disclosure include, but are not limited to: genetic modifications performed in vivo; modifications that alter the activity or amount of metabolic enzymes expressed by endogenous or exogenous selected cells and/or their progeny; modifications which alter the activity, amount, or antigenicity of cellular proteins; modifications which alter the activity or amount of proteins involved in signal transduction pathways; modifications which alter HLA type; modifications which alter cellular differentiation; modifications which alter neoplastic potential; modifications which alter cellular differentiation; modifications which alter the amount or activity of structural proteins; modifications which alter the amount or activity of membrane associated proteins (structural or enzymatic); modifications which alter the activity or amount of proteins involved in DNA repair and chromosome maintenance; modifications which alter the activity or amount of proteins involved in cellular transport; modifications which alter the activity or amount of enzymes; modifications which alter the activity or amount of proteins involved in synapse formation and maintenance; modifications which alter the activity or amount of proteins involved in neurite outgrowth or axon outgrowth and formation; modifications altering the amount or activity of antioxidant producing enzymes within the cell; modifications which lead to altered post-translational modification of cellular proteins; modifications which alter the activity or amount of proteins involved in other aspects of cellular repair, and alterations which increase the lifespan of the cell (such as production of telomerase). Such proteins as those mentioned above may be encoded for by DNA or RNA derived from the human genome or other animal, plant, viral, or bacterial genomes. This invention also covers sequences designed de novo.

In addition, this invention relates to the in situ, genetic modification of selected cells and/or their progeny cells for the treatment of disease. Endogenous stem cells may be modified in situ by direct injection or application of DNA or RNA vectors, including viruses, retroviruses, liposomes, etc, into the substance of the tissue or into the appropriate portion of the ventricular system of the brain. Since 1992, we have modified thousands of stem/progenitor cells and many thousand progeny cells in this manner. Our data shows that this manner of modifying progenitor cells results in a tremendous variety of modified cell types throughout the nervous system, and has never resulted in adverse effects.

### Example 9

### Introduction of Genetic Vectors into the Host

In a preferred embodiment, endogenous cells are transfected with vectors such as those described herein in vivo by introduction of the therapeutic vector(s) into the host blood, tissues, nervous system, bone marrow, etc. The greatest benefit may be achieved by modifying a large number of endogenous target cells. This may be accomplished by using an appropriately-sized, catheter-like device, or needle to inject the therapeutic vector(s) into the venous or arterial circulation, into a specific tissue, such as muscle tissue, or into the nervous system. In a preferred embodiment, the virus is pseudotyped with VSV-G envelope glycoprotein and native HIV-1 env proteins.

### Example 10

### Injection into the Nervous System

Transplantation of selected cells (from either the growth or differentiation media) into the fetal nervous system or genetic modification of endogenous fetal cells utilizing genetic vectors may be accomplished in the following manner: Under sterile conditions, the uterus and fetuses are visualized by ultrasound or other radiological guidance. Alternatively the uterus may be exposed surgically in order to facilitate direct identification of fetal skull landmarks. Selected cells can then be introduced by injection (using an appropriately-sized catheter or needle) into the ventricular system, germinal zone(s), or into the substance of the nervous system. Injections may be performed in certain instances, through the mother's abdominal wall, the uterine wall and fetal membranes into the fetus. The accuracy of the injection is monitored by direct observation, ultrasound, contrast, or radiological isotope based methods, or by any other means of radiological guidance known to the art.

Under appropriate sterile conditions, direct identification of fetal skull landmarks is accomplished visually as well as by physical inspection and palpation coupled with stereotaxic and radiologic guidance. Following cell culture, appropriate amounts of the selected or differentiating cells can then be introduced by injection or other means into the ventricular system, germinal zones, or into the substance of the nervous system. The accuracy of the injection may be monitored by direct observation, ultrasound, or other radiological guidance.
In certain, neurological diseases of the adult nervous system, such as Huntington's disease and Parkinson's disease, cells of a specific portion of the brain are selectively affected. In the case of Parkinson's disease, it is the dopaminergic cells of the substantia nigra. In such regionally-specific diseases affecting adults, localized transplantation of cells may be accomplished by radiologically-guided transplantation of differentiating cells under sterile conditions. Radiologic guidance may include the use of CT and/or MRI, and may take advantage contrast or isotope based techniques to monitor injected materials.
In certain neurologic diseases, such as some metabolic storage disorders, cells are affected across diverse regions of the nervous system, and the greatest benefit may be achieved by genetically-modifying endogenous cells or introducing selected cells of the present invention (either from the growth culture media or the differentiating medium) into the tissue in large numbers in a diffuse manner. In the nervous system, these diseases may be best approached by intraventricular injections (using an appropriately-sized, catheter-like device, or needle) (especially at early stages of development) which allows diffuse endogenous cell modification or diffuse engraftment of selected cells isolated from the growth and/or differentiation media. Nevertheless, injection of the cells into the circulatory system for the same purpose is also covered. However, with regard to any disorder affecting multiple organs or the body diffusely (e.g. lysosomal storage disorders, hemoglobinpathies, muscular dystrophy), the cells isolated from the growth and/or differentiation media may also be preferentially introduced directly into the circulation and/or visceral organs, such as the liver, kidney, gut, spleen, adrenal glands, pancreas, lungs, and thymus using endoscopic guidance and any appropriately-sized, catheter-like device, allowing diffuse engraftment of the cells throughout the body, as well as specific introduction and infiltration of the cells into the selected organs.

### Example 11

### Delivery of Cells by Injection in to the Circulatory Stream and Organs

Diseases of one organ system may be treatable with genetically modified cells from a separate organ system. Also, in some instances, it may become apparent that the selected cells may integrate and differentiate on their own, in vivo, in sufficient numbers if they are injected into blood stream either arterial, venous or hepatic, after culturing in the growth and/or differentiation media. This approach is covered by the present invention. The treatment of diffuse muscle (e.g. muscular dystrophies), organ, tissue, or blood disorders (e.g. Hereditary Spherocytosis, Sickle cell anemia, other hemoglobinopathies, etc.,) may, for instance, involve the injection of cells isolated from the growth media or differentiating media into the patient, especially the patient's circulation. This approach is also believed to ameliorate ischemic injuries such as myocardial infarction, stroke, etc., as well as traumatic injuries to brain and other tissues. Injection of such cells produced by the current invention, directly into the circulation, by needle or catheter, so that the cells are enabled to “home” to the bone marrow, muscle, kidneys, lungs, and/or any other other organ system, as well as injection directly into the bone marrow space is suitable for the practice of the present invention Likewise injection of the cells directly into a lesion site with or without radiologic, ultrasonic or fluoroscopic guidance is also suitable for the practice of the present invention.

Methods of isolating selected cells useful in the present invention include those described by Zhao et al., 2006.

In a preferred embodiment, genetic vectors encoding numblike and/or numb isoforms comprise regulable promoters operably linked to the Numb or numblike transgenes.

In another preferred embodiment, the mode of transfection may be selected from those modes of transfection that provide for transient rather than permanent expression of the numblike and numb isoforms.

### Example 12

### Example Genetic Modifications

It is believed that hundreds of diseases and clinical conditions are able to be treated and/or ameliorated by the methods of the present invention including, but in no way limited to Canavan's disease (ASP); Tay-Sach's disease (HEXA); Lesch-Nyhan syndrome (HRPT); Huntington's disease(HTT); Sly syndrome; type A and type B Niemann Pick disease; Sandhoffs disease (HEXB); Fabry's disease (GLA); type C Niemann-Pick disease(NPC1); Gaucher's disease (GBA); Parkinson's disease(PARK2, etc.); Von Hippel Lindau's disease, Sickle cell anemia (HBB) and other thalassemias as well as similar diseases. These transgenes may represent the coding region or portions of the coding region of the normal genes.

It is to be understood, however, that the scope of the present invention is not to be limited to the specific embodiments and examples described above. The invention may be practiced other than as particularly described and still be within the scope of the accompanying claims.

### Example 13

An Example sequence for a vector capable of rendering cells pluripotent and expressing a long Numb isoform, Oct-4, Sox-2, and EmGFP nucleic acid sequences under the control of tetracycline-sensitive promoters is (SEQ ID NO: 2):

aatgtagtcttatgcaatactcttgtagtcttgcaacatggtaacgatga
gttagcaacatgccttacaaggagagaaaaagcaccgtgcatgccgattg
gtggaagtaaggtggtacgatcgtgccttattaggaaggcaacagacggg
tctgacatggattggacgaaccactgaattgccgcattgcagagatattg
tatttaagtgcctagctcgatacataaacgggtctctctggttagaccag
atctgagcctgggagctctctggctaactaggaacccactgcttaagcct
caataaagcttgccttgagtgcttcaagtagtgtgtgcccgtctgttgtg
tgactctggtaactagagatccctcagaccttttagtcagtgtggaaaat
ctctagcagtggcgccgaacagggacttgaaagcgaaagggaaaccagag
gagctctctcgacgcaggactcggcttgctgaagcgcgcacggcaagagg
cgaggggcggcgactggtgagtacgccaaaaattttgactagcggaggct
agaaggagagagatgggtgcgagagcgtcagtattaagcgggggagaatt
agatcgcgatgggaaaaaattcggttaaggccagggggaaagaaaaaata
taaattaaaacatatagtatgggcaagcagggagctagaacgattcgcag
ttaatcctggcctgttagaaacatcagaaggctgtagacaaatactggga
cagctacaaccatcccttcagacaggatcagaagaacttagatcattata
taatacagtagcaaccctctattgtgtgcatcaaaggatagagataaaag
acaccaaggaagctttagacaagatagaggaagagcaaaacaaaagtaag
accaccgcacagcaagcggccgctgatcttcagacctggaggaggagata
tgagggacaattggagaagtgaattatataaatataaagtagtaaaaatt
gaaccattaggagtagcacccaccaaggcaaagagaagagtggtgcagag
agaaaaaagagcagtgggaataggagctttgttccttgggttcttgggag
cagcaggaagcactatgggcgcagcgtcaatgacgctgacggtacaggcc
agacaattattgtctggtatagtgcagcagcagaacaatttgctgagggc
tattgaggcgcaacagcatctgttgcaactcacagtctggggcatcaagc
agctccaggcaagaatcctggctgtggaaagatacctaaaggatcaacag
ctcctggggatttggggttgctctggaaaactcatttgcaccactgctgt
gccttggaatgctagttggagtaataaatctctggaacagatttggaatc
acacgacctggatggagtgggacagagaaattaacaattacacaagctta
atacactccttaattgaagaatcgcaaaaccagcaagaaaagaatgaaca
agaattattggaattagataaatgggcaagtttgtggaattggtttaaca
taacaaattggctgtggtatataaaattattcataatgatagtaggaggc
ttggtaggtttaagaatagtttttgctgtactttctatagtgaatagagt
taggcagggatattcaccattatcgtttcagacccacctcccaaccccga
ggggacccgacaggcccgaaggaatagaagaagaaggtggagagagagac
agagacagatccattcgattagtgaacggatctcgacggtatcgatgtcg
acgttaacgctagtgatatcaactttgtatagaaaagttgaacgagaaac
gtaaaatgatataaatatcaatatattaaattagattttgcataaaaaac
agactacataatactgtaaaacacaacatatccagtcactatgggacgga
tcgggagatctcccgatcccctatggtgcactctcagtacaatctgctct
gatgccgcatagttaagccagtatctgctccctgcttgtgtgttggaggt
cgctgagtagtgcgcgagcaaaatttaagctacaacaaggcaaggcttga
ccgacaattgcatgaagaatctgcttagggttaggcgttttgcgctgctt
cgcgatgtacgggccagatatacgcgttgacattgattattgactagtta
ttaatagtaatcaattacggggtcattagttcatagcccatatatggagt
tccgcgttacataacttacggtaaatggcccgcctggctgaccgcccaac
gacccccgcccattgacgtcaataatgacgtatgttcccatagtaacgcc
aatagggactttccattgacgtcaatgggtggagtatttacggtaaactg
cccacttggcagtacatcaagtgtatcatatgccaagtacgccccctatt
gacgtcaatgacggtaaatggcccgcctggcattatgcccagtacatgac
cttatgggactttcctacttggcagtacatctacgtattagtcatcgcta
ttaccatggtgatgcggttttggcagtacatcaatgggcgtggatagcgg
tttgactcacggggatttccaagtctccaccccattgacgtcaatgggag
tttgttttggaaccaaaatcaacgggactttccaaaatgtcgtaacaact
ccgccccattgacgcaaatgggcggtaggcgtgtacggtgggaggtctat
ataagcagagctctccctatcagtgatagagatctccctatcagtgatag
agatcgtcgacgagctcgtttagtgaaccgtcagatcgcctggagacgcc
atccacgctgttttgacctccatagaagacaccgggaccgatccagcctc
cggactctagcgtttaaacttaagcttaccatgccttcgcaagccctcat
ttcaccaggcccccggcttggggcgccttccttccccatggcgggacacc
tggcttcggatttcgccttctcgccccctccaggtggtggaggtgatggg
ccaggggggccggagccgggctgggttgatcctcggacctggctaagctt
ccaaggccctcctggagggccaggaatcgggccgggggttgggccaggct
ctgaggtgtgggggattcccccatgccccccgccgtatgagttctgtggg
gggatggcgtactgtgggccccaggttggagtggggctagtgccccaagg
cggcttggagacctctcagcctgagggcgaagcaggagtcggggtggaga
gcaactccgatggggcctccccggagccctgcaccgtcacccctggtgcc
gtgaagctggagaaggagaagctggagcaaaacccggaggagtcccagga
catcaaagctctgcagaaagaactcgagcaatttgccaagctcctgaagc
agaagaggatcaccctgggatatacacaggccgatgtggggctcaccctg
ggggttctatttgggaaggtattcagccaaacgaccatctgccgctttga
ggctctgcagcttagcttcaagaacatgtgtaagctgcggcccttgctgc
agaagtgggtggaggaagctgacaacaatgaaaatcttcaggagatatgc
aaagcagaaaccctcgtgcaggcccgaaagagaaagcgaaccagtatcga
gaaccgagtgagaggcaacctggagaatttgttcctgcagtgcccgaaac
ccacactgcagcagatcagccacatcgcccagcagcttgggctcgagaag
gatgtggtccgagtgtggttctgtaaccggcgccagaagggcaagcgatc
aagcagcgactatgcacaacgagaggattttgaggctgctgggtctcctt
tctcagggggaccagtgtcctttcctctggccccagggccccattttggt
accccaggctatgggagccctcacttcactgcactgtactcctcggtccc
tttccctgagggggaagcctttccccctgtctccgtcaccactctgggct
ctcccatgcattcaaactgaggtgcctgcccttctaggaatgggggacag
ggggaggggaggagctagggaaagaaaacctggagtttgtgccagggttt
ttgggattaagttcttcattcactaaggaaggaattgggaacacaaaggg
tgggggcaggggagtttggggcaactggttggagggaaggtgaagttcaa
tgatgctcttgattttaatccacatcatgtatcacttttttcttaaataa
agaagcctgggacacagtagatagacacacttaaaaaaaaaaacctcgac
tgtgccttctagttgccagccatctgttgtttgcccctcccccgtgcctt
ccttgaccctggaaggtgccactcccactgtcctttcctaataaaatgag
gaaattgcatcgcattgtctgagtaggtgtcattctattctggggggtgg
ggtggggcaggacagcaagggggaggattgggaagacaatagcaggcatg
ctggggatgcggtgggctctatgggacggatcgggagatctcccgatccc
ctatggtgcactctcagtacaatcttgctctgatgccgcatagttaagcc
agtatctgctccctgcttgtgtgttggaggtcgctgagtagtgcgcgagc
aaaatttaagctacaacaaggcaaggcttgaccgacaattgcatgaagaa
tctgcttagggttaggcgttttgcgctgcttcgcgatgtacgggccagat
atacgcgttgacattgattattgactagttattaatagtaatcaattacg
gggtcattagttcatagcccatatatggagttccgcgttacataacttac
ggtaaatggcccgcctggctgaccgcccaacgacccccgcccattgacgt
caataatgacgtatgttcccatagtaacgccaatagggactttccattga
cgtcaatgggtggagtatttacggtaaactgcccacttggcagtacatca
agtgtatcatatgccaagtacgccccctattgacgtcaatgacggtaaat
ggcccgcctggcattatgcccagtacatgaccttatgggactttcctact
tggcagtacatctacgtattagtcatcgctattaccatggtgatgcggtt
ttggcagtacatcaatgggcgtggatagcggtttgactcacggggatttc
caagtctccaccccattgacgtcaatgggagtttgttttggaaccaaaat
caacgggactttccaaaatgtcgtaacaactccgccccattgacgcaaat
gggcggtaggcgtgtacggtgggaggtctatataagcagagctctcccta
tcagtgatagagatctccctatcagtgatagagatcgtcgacgagctcgt
ttagtgaaccgtcagatcgcctggagacgccatccacgctgttttgacct
ccatagaagacaccgggaccgatccagcctccggactctagcgtttaaac
ttaagcttaccatgctattaacttgttcaaaaaagtatcaggagttgtca
aggcagagaagagagtgtttgcaaaagggggaaagtagtttgctgcctct
ttaagactaggactgagagaaagaagaggagagagaaagaaagggagaga
agtttgagccccaggcttaagcctttccaaaaaataataataacaatcat
cggcggcggcaggatcggccagaggaggagggaagcgctttttttgatcc
tgattccagtttgcctctctctttttttcccccaaattattcttcgcctg
attttcctcgcgagccctgcgctcccgacacccccgcccgcctcccctcc
tcctctccccccgcccgcgggccccccaaagtcccggccgggccgagggt
cggcggccgccggcgggccgggcccgcgcacagcgcccgcatgtacaaca
tgatggagacggagctgaagccgccgggcccgcagcaaacttcggggggc
ggcggcggcaactccaccgcggcggcggccggcggcaacagaaaaacagc
ccggaccgcgtcaagcggcccatgaatgccttcatggtgtggtcccgcgg
gcagcggcgcaagatggcccaggagaaccccaagatgcacaactcggaga
tcagcaagcgcctgggcgccgagtggaaacttttgtcggagacggagaag
cggccgttcatcgacgaggctaagcggctgcgagcgctgcacatgaagga
gcacccggattataaataccggccccggcggaaaaccaagacgctcatga
agaaggataagtacacgctgcccggcgggctgctggcccccggcggcaat
agcatggcgagcggggtcggggtgggcgccggcctgggcgcgggcgtgaa
ccagcgcatggacagttacgcgcacatgaacggctggagcaacggcagct
acagcatgatgcaggaccagctgggctacccgcagcacccgggcctcaat
gcgcacggcgcagcgcagatgcagcccatgcaccgctacgacgtgagcgc
cctgcagtacaactccatgaccagctcgcagacctacatgaacggctcgc
ccacctacagcatgtcctactcgcagcagggcacccctggcatggctctt
ggctccatgggttcggtggtcaagtccgaggccagctccagcccccctgt
ggttacctcttcctcccactccagggcgccctgccaggccggggacctcc
gggacatgatcagcatgtatctccccggcgccgaggtgccggaacccgcc
gcccccagcagacttcacatgtcccagcactaccagagcggcccggtgcc
cggcacggccattaacggcacactgcccctctcacacatgtgagggccgg
acagcgaactggaggggggagaaattttcaaagaaaaacgagggaaatgg
gaggggtgcaaaagaggagagtaagaaacagcatggagaaaacccggtac
gctcaaaaagaaaaaggaaaaaaaaaaatcccatcacccacagcaaatga
cagctgcaaaagagaacaccaatcccatccacactcacgcaaaaaccgcg
atgccgacaagaaaacttttatgagagagatcctggacttctttttgggg
gactatttttgtacagagaaaacctggggagggtggggagggcgggggaa
tggaccttgtatagatctggaggaaagaaagctacgaaaaactttttaaa
agttctagtggtacggtaggagctttgcaggaagtttgcaaaagtcttta
ccaataatatttagagctagtctccaagcgacgaaaaaaatgttttaata
tttgcaagcaacttttgtacagtatttatcgagataaacatggcaatcaa
aatgtccattgtttataagctgagaatttgccaatatttttcaaggagag
gcttcttgctgaattttgattctgcagctgaaatttaggacagttgcaaa
cgtgaaaagaagaaaattattcaaatttggacattttaattgtttaaaaa
ttgtacaaaaggaaaaaattagaataagtactggcgaaccatctctgtgg
tcttgtttaaaaagggcaaaagttttagactgtactaaattttataactt
actgttaaaagcaaaaatggccatgcaggttgacaccgttggtaatttat
aatagcttttgttcgatcccaactttccattttgttcagataaaaaaaac
catgaaattactgtgtttgaaatattttcttatggtttgtaatatttctg
taaatttattgtgatattttaaggttttcccccctttattttccgtagtt
gtattttaaaagattcggctctgtattatttgaatcagtctgccgagaat
ccatgtatatatttgaactaatatcatccttataacaggtacattttcaa
cttaagtttttactccattatgcacagtttgagataaataaatttttgaa
atatggacactgaaaaaaaaaaaaaaaaaacctcgactgtgccttctagt
tgccagccatctgttgtttgcccctcccccgtgccttccttgaccctgga
aggtgccactcccactgtcctttcctaataaaatgaggaaattgcatcgc
attgtctgagtaggtgtcattctattctggggggtggggtggggcaggac
agcaagggggaggattgggaagacaatagcaggcatgctggggatgcggt
gggctctatgggacggatcgggagatctcccgatcccctatggtgcactc
tcagtacaatctgctctgatgccgcatagttaagccagtatctgctccct
gcttgtgtgttggaggtcgctgagtagtgcgcgagcaaaatttaagctac
aacaaggcaaggcttgaccgacaattgcatgaagaatctgcttagggtta
ggcgttttgcgctgcttcgcgatgtacgggccagatatacgcgttgacat
tgattattgactagttattaatagtaatcaattacggggtcattagttca
tagcccatatatggagttccgcgttacataacttacggtaaatggcccgc
ctggctgaccgcccaacgacccccgcccattgacgtcaataatgacgtat
gttcccatagtaacgccaatagggactttccattgacgtcaatgggtgga
gtatttacggtaaactgcccacttggcagtacatcaagtgtatcatatgc
caagtacgccccctattgacgtcaatgacggtaaatggcccgcctggcat
tatgcccagtacatgaccttatgggactttcctacttggcagtacatcta
cgtattagtcatcgctattaccatggtgatgcggttttggcagtacatca
atgggcgtggatagcggtttgactcacggggatttccaagtctccacccc
attgacgtcaatgggagtttgttttggaaccaaaatcaacgggactttcc
aaaatgtcgtaacaactccgccccattgacgcaaatgggcggtaggcgtg
tacggtgggaggtctatataagcagagctctccctatcagtgatagagat
ctccctatcagtgatagagatcgtcgacgagctcgtttagtgaaccgtca
gatcgcctggagacgccatccacgctgttttgacctccatagaagacacc
gggaccgatccagcctccggactctagcgtttaaacttaagcttaccatg
gttgtcatgggggaggtggtggcgcttggtggccactggcggccgaggta
gaggcagtggcgcttgagttggtcgggggcagcggcagatttgaggctta
agcaacttcttccggggaagagtgccagtgcagccactgttacaattcaa
gatcttgatctatatccatagattggaatattggtgggccagcaatcctc
agacgcctcacttaggacaaatgaggaaactgaggcttggtgaagttacg
aaacttgtccaaaatcacacaacttgtaaagggcacagccaagattcaga
gccaggctgtaaaaattaaaatgaacaaattacggcaaagttttaggaga
aagaaggatgtttatgttccagaggccagtcgtccacatcagtggcagac
agatgaagaaggcgttcgcaccggaaaatgtagcttcccggttaagtacc
ttggccatgtagaagttgatgaatcaagaggaatgcacatctgtgaagat
gctgtaaaaagattgaaagctgaaaggaagttcttcaaaggcttctttgg
aaaaactggaaagaaagcagttaaagcagttctgtgggtctcagcagatg
gactcagagttgtggatgaaaaaactaaggacctcatagttgaccagacg
atagagaaagtttctttctgtgccccagacaggaactttgatagagcctt
ttcttacatatgccgtgatggcaccactcgtcgctggatctgtcactgct
tcatggctgtcaaggacacaggtgaaaggttgagccatgcagtaggctgt
gcttttgcagcctgtttagagcgcaagcagaagcgggagaaggaatgtgg
agtgactgctacttttgatgctagtcggaccacttttacaagagaaggat
cattccgtgtcacaacagccactgaacaagcagaaagagaggagatcatg
aaacaaatgcaagatgccaagaaagctgaaacagataagatagtcgttgg
ttcatcagttgcccctggcaacactgccccatccccatcctctcccacct
ctcctacttctgatgccacgacctctctggagatgaacaatcctcatgcc
atcccacgccggcatgctccaattgaacagcttgctcgccaaggctcttt
ccgaggttttcctgctcttagccagaagatgtcaccctttaaacgccaac
tatccctacgcatcaatgagttgccttccactatgcagaggaagactgat
ttccccattaaaaatgcagtgccagaagtagaaggggaggcagagagcat
cagctccctgtgctcacagatcaccaatgccttcagcacacctgaggacc
ccttctcatctgctccgatgaccaaaccagtgacagtggtggcaccacaa
tctcctaccttccaagctaatggcactgactcagccttccatgtgcttgc
taagccagcccatactgctctagcacccgtagcaatgcctgtgcgtgaaa
ccaacccttgggcccatgcccctgatgctgctaacaaggaaattgcagcc
acatgttcggggaccgagtggggtcaatcttctggtgctgcctctccagg
tctcttccaggccggtcatagacgtactccctctgaggccgaccgatggt
tagaagaggtgtctaagagcgtccgggctcagcagccccaggcctcagct
gctcctctgcagccagttctccagcctcctccacccactgccatctccca
gccagcatcacctttccaagggaatgcattcctcacctctcagcctgtgc
cagtgggtgtggtcccagccctgcaaccagcctttgtccctgcccagtcc
tatcctgtggccaatggaatgccctatccagcccctaatgtgcctgtggt
gggcatcactccctcccagatggtggccaacgtatttggcactgcaggcc
accctcaggctgcccatccccatcagtcacccagcctggtcaggcagcag
acattccctcactacgaggcaagcagtgctaccaccagtcccttctttaa
gcctcctgctcagcacctcaacggttctgcagctttcaatggtgtagatg
atggcaggttggcctcagcagacaggcatacagaggttcctacaggcacc
tgcccagtggatccttttgaagcccagtgggctgcattagaaaataagtc
caagcagcgtactaatccctcccctaccaaccctttctccagtgacttac
agaagacgtttgaaattgaactttaagcaatcattatggctatgtatctt
gtccataccagacagggagcagggggtagcggtcaaaggagcaaaacaga
ctttgtctcctgattagtactcttttcactaatcccaaaggtcccaagga
acaagtccaggcccagagtactgtgaggggtgattttgaaagacatggga
aaaagcattcctagagaaaagctgccttgcaattaggctaaagaagtcaa
ggaaatgttgctttctgtactccctcttcccttacccccttacaaatctc
tggcaacagagaggcaaagtatctgaacaagaatctatattccaagcaca
tttactgaaatgtaaaacacaacaggaagcaaagcaatctccctttgttt
ttcaggccattcacctgcctcctgtcagtagtggcctgtattagagatca
agaagagtggtttgtgctcaggctggggaacagagaggcacgctatgctg
ccagaattcccaggagggcatatcagcaactgcccagcagagctatattt
tgggggagaagttgagcttccattttgagtaacagaataaatattatata
tatcaaaagccaaaatctttatttttatgcatttagaatattttaaatag
ttctcagatattaagaagttgtatgagttgtaagtaatcttgccaaaggt
aaaggggctagttgtaagaaattgtacataagattgatttatcattgatg
cctactgaaataaaaagaggaaaggctggaagctgcagacaggatcccta
gcttgttttctgtcagtcattcattgtaagtagcacattgcaacaacaat
catgcttatgaccaatacagtcactaggttgtagttttttttaaataaag
gaaaagcagtattgtcctggttttaaacctatgatggaattctaatgtca
ttattttaatggaatcaatcgaaatatgctctatagagaatatatctttt
atatattgctgcagtttccttatgttaatcctttaacactaaggtaacat
gacataatcataccatagaagggaacacaggttaccatattggtttgtaa
tatgggtcttggtgggttttgttttatcctttaaattttgttcccatgag
ttttgtggggatggggattctggttttattagctttgtgtgtgtcctctt
cccccaaacccccttttggtgagaacatccccttgacagttgcagcctct
tgacctcggataacaataagagagctcatctcatttttacttttgaacgt
tggccttacaatcaaatgtaagttatatatatttgtactgatgaaaattt
ataatctgctttaacaaaaataaatgttcatggtagaagcttttaaaaaa
aaaaaaacctcgactgtgccttctagttgccagccatctgttgtttgccc
ctcccccgtgccttccttgaccctggaaggtgccactcccactgtccttt
cctaataaaatgaggaaattgcatcgcattgtctgagtaggtgtcattct
attctggggggtggggtggggcaggacagcaagggggaggattgggaaga
caatagcaggcatgctggggatgcggtgggctctatgggacggatcggga
gatctcccgatcccctatggtgcactctcagtacaatctgctctgatgcc
gcatagttaagccagtatctgctccctgcttgtgtgttggaggtcgctga
gtagtgcgcgagcaaaatttaagctacaacaaggcaaggcttgaccgaca
attgcatgaagaatctgcttagggttaggcgttttgcgctgcttcgcgat
gtacgggccagatatacgcgttgacattgattattgactagttattaata
gtaatcaattacggggtcattagttcatagcccatatatggagttccgcg
ttacataacttacggtaaatggcccgcctggctgaccgcccaacgacccc
cgcccattgacgtcaataatgacgtatgttcccatagtaacgccaatagg
gactttccattgacgtcaatgggtggagtatttacggtaaactgcccact
tggcagtacatcaagtgtatcatatgccaagtacgccccctattgacgtc
aatgacggtaaatggcccgcctggcattatgcccagtacatgaccttatg
ggactttcctacttggcagtacatctacgtattagtcatcgctattacca
tggtgatgcggttttggcagtacatcaatgggcgtggatagcggtttgac
tcacggggatttccaagtctccaccccattgacgtcaatgggagtttgtt
ttggaaccaaaatcaacgggactttccaaaatgtcgtaacaactccgccc
cattgacgcaaatgggcggtaggcgtgtacggtgggaggtctatataagc
agagctctccctatcagtgatagagatctccctatcagtgatagagatcg
tcgacgagctcgtttagtgaaccgtcagatcgcctggagacgccatccac
gctgttttgacctccatagaagacaccgggaccgatccagcctccggact
ctagcgtttaaacttaagcttaccatggtgagcaagggcgaggagctgtt
caccggggtggtgcccatcctggtcgagctggacggcgacgtaaacggcc
acaagttcagcgtgtccggcgagggcgagggcgatgccacctacggcaag
ctgaccctgaagttcatctgcaccaccggcaagctgcccgtgccctggcc
caccctcgtgaccaccttcacctacggcgtgcagtgcttcgcccgctacc
ccgaccacatgaagcagcacgacttcttcaagtccgccatgcccgaaggc
tacgtccaggagcgcaccatcttcttcaaggacgacggcaactacaagac
ccgcgccgaggtgaagttcgagggcgacaccctggtgaaccgcatcgagc
tgaagggcatcgacttcaaggacggcaacatcctggggcacaagctggag
tacaactacaacagccacaaggtctatatcaccgccgacaagcagaagaa
cggcatcaaggtgaacttcaagacccgccacaacatcgaggacggcagcg
tgcagctcgccgaccactaccagcagaacacccccatcggcgacggcccc
gtgctgctgcccgacaaccactacctgagcacccagtccgccctgagcaa
agaccccaacgagaagcgcgatcacatggtcctgctggagttcgtgaccg
ccgccgggatcactctcggcatggacgagctgtacaagtaacctcgactg
tgccttctagttgccagccatctgttgtttgcccctcccccgtgccttcc
ttgaccctggaaggtgccactcccactgtcctttcctaataaaatgagga
aattgcatcgcattgtctgagtaggtgtcattctattctggggggtgggg
tggggcaggacagcaagggggaggattgggaagacaatagcaggcatgct
ggggatgcggtgggctctatgggacggatcgggagatctcccgatcccct
atggtgcactctcagtacaatctgctctgatgccgcatagttaagccagt
atctgctccctgcttgtgtgttggaggtcgctgagtagtgcgcgagcaaa
atttaagctacaacaaggcaaggcttgaccgacaattgcatgaagaatct
gcttagggttaggcgttttgcgctgcttcgcgatgtacgggccagatata
cgcgttgacattgattattgactagttattaatagtaatcaattacgggg
tcattagttcatagcccatatatggagttccgcgttacataacttacggt
aaatggcccgcctggctgaccgcccaacgacccccgcccattgacgtcaa
taatgacgtatgttcccatagtaacgccaatagggactttccattgacgt
caatgggtggagtatttacggtaaactgcccacttggcagtacatcaagt
gtatcatatgccaagtacgccccctattgacgtcaatgacggtaaatggc
ccgcctggcattatgcccagtacatgaccttatgggactttcctacttgg
cagtacatctacgtattagtcatcgctattaccatggtgatgcggttttg
gcagtacatcaatgggcgtggatagcggtttgactcacggggatttccaa
gtctccaccccattgacgtcaatgggagtttgttttggaaccaaaatcaa
cgggactttccaaaatgtcgtaacaactccgccccattgacgcaaatggg
cggtaggcgtgtacggtgggaggtctatataagcagagctcgtgagtttg
gggacccttgattgttctttctttttcgctattgtaaaattcatgttata
tggagggggcaaagttttcagggtgttgtttagaatgggaagatgtccct
tgtatcaccatggaccctcatgataattttgtttctttcactttctactc
tgttgacaaccattgtctcctcttattttcttttcattttctgtaacttt
ttcgttaaactttagcttgcatttgtaacgaatttttaaattcacttttg
tttatttgtcagattgtaagtactttctctaatcacttttttttcaaggc
aatcagggtatattatattgtacttcagcacagttttagagaacaattgt
tataattaaatgataaggtagaatatttctgcatataaattctggctggc
gtggaaatattcttattggtagaaacaactacatcctggtcatcatcctg
cctttctctttatggttacaatgatatacactgtttgagatgaggataaa
atactctgagtccaaaccgggcccctctgctaaccatgttcatgccttct
tctttttcctacagctcctgggcaacgtgctggttattgtgctgtctcat
cattttggcaaagaattgtaatacgactcactatagggcgaattgatatg
tctagattagataaaagtaaagtgattaacagcgcattagagctgcatgt
ctagattagataaaagtaaagtgattaacagcgcattagagctgcttaat
gaggtcggaatcgaaggtttaacaacccgtaaactcgcccagaagctagg
tgtagagcagcctacattgtattggcatgtaaaaaataagcgggctttgc
tcgacgccttagccattgagatgttagataggcaccatactcacttttgc
cctttagaaggggaaagctggcaagattttttacgtaataacgctaaaag
ttttagatgtgctttactaagtcatcgcgatggagcaaaagtacatttag
gtacacggcctacagaaaaacagtatgaaactctcgaaaatcaattagcc
tttttatgccaacaaggtttttcactagagaatgcattatatgcactcag
cgctgtggggcattttactttaggttgcgtattggaagatcaagagcatc
aagtcgctaaagaagaaagggaaacacctactactgatagtatgccgcca
ttattacgacaagctatcgaattatttgatcaccaaggtgcagagccagc
cttcttattcggccttgaattgatcatatgcggattagaaaaacaactta
aatgtgaaagtgggtccgcgtacagcggatccgggaattcagatcttatt
aaagcagaacttgtttattgcagcttataatggttacaaataaagcaata
gcatcacaaatttcacaaataaagcatttttttcactgcattctagttgt
ggtttgtccaaactcatcaatgtatcttatcatgtctggtcaatgtgtgt
cagttagggtgtggaaagtccccaggctccccagcaggcagaagtatgca
aagcatgcatctcaattagtcagcaaccaggtgtggaaagtccccaggct
ccccagcaggcagaagtatgcaaagcatgcatctcaattagtcagcaacc
atagtcccgcccctaactccgcccatcccgcccctaactccgcccagttc
cgcccattctccgccccatggctgactaattttttttatttatgcagagg
ccgaggccgcctctgcctctgagctattccagaagtagtgaggaggcttt
tttggaggcctaggcttttgcaaaaagctccccatagtgactggatatgt
tgtgttttacagtattatgtagtctgttttttatgcaaaatctaatttaa
tatattgatatttatatcattttacgtttctcgttcagctttcttgtaca
aagtggttgatatccagcacagtggcggccgctcgagtctagagggcccg
cggttcgaaggtaagcctatccctaaccctctcctcggtctcgattctac
gcgtaccggttagtaatgagtttggaattaattctgtggaatgtgtgtca
gttagggtgtggaaagtccccaggctccccagcaggcagaagtatgcaaa
gcatgcatctcaattagtcagcaaccaggtgtggaaagtccccaggctcc
ccagcaggcagaagtatgcaaagcatgcatctcaattagtcagcaaccat
agtcccgcccctaactccgcccatcccgcccctaactccgcccagttccg
cccattctccgccccatggctgactaattttttttatttatgcagaggcc
gaggccgcctctgcctctgagctattccagaagtagtgaggaggcttttt
tggaggcctaggcttttgcaaaaagctcccgggagcttgtatatccattt
tcggatctgatcagcacgtgttgacaattaatcatcggcatagtatatcg
gcatagtataatacgacaaggtgaggaactaaaccatggccaagcctttg
tctcaagaagaatccaccctcattgaaagagcaacggctacaatcaacag
catccccatctctgaagactacagcgtcgccagcgcagctctctctagcg
acggccgcatcttcactggtgtcaatgtatatcattttactgggggacct
tgtgcagaactcgtggtgctgggcactgctgctgctgcggcagctggcaa
cctgacttgtatcgtcgcgatcggaaatgagaacaggggcatcttgagcc
cctgcggacggtgccgacaggtgcttctcgatctgcatcctgggatcaaa
gccatagtgaaggacagtgatggacagccgacggcagttgggattcgtga
attgctgccctctggttatgtgtgggagggctaagcacaattcgagctcg
gtacctttaagaccaatgacttacaaggcagctgtagatcttagccactt
tttaaaagaaaaggggggactggaagggctaattcactcccaacgaagac
aagatctgctttttgcttgtactgggtctctctggttagaccagatctga
gcctgggagctctctggctaactagggaacccactgcttaagcctcaata
aagcttgccttgagtgcttcaagtagtgtgtgcccgtctgttgtgtgact
ctggtaactagagatccctcagacccttttagtcagtgtggaaaatctct
agcagtagtagttcatgtcatcttattattcagtatttataacttgcaaa
gaaatgaatatcagagagtgagaggaacttgtttattgcagcttataatg
gttacaaataaagcaatagcatcacaaatttcacaaataaagcatttttt
tcactgcattctagttgtggtttgtccaaactcatcaatgtatcttatca
tgtctggctctagctatcccgcccctaactccgcccatcccgcccctaac
tccgcccagttccgcccattctccgccccatggctgactaatttttttta
tttatgcagaggccgaggccgcctcggcctctgagctattccagaagtag
tgaggaggcttttttggaggcctagggacgtacccaattcgccctatagt
gagtcgtattacgcgcgctcactggccgtcgttttacaacgtcgtgactg
ggaaaaccctggcgttacccaacttaatcgccttgcagcacatccccctt
tcgccagctggcgtaatagcgaagaggcccgcaccgatcgcccttcccaa
cagttgcgcagcctgaatggcgaatgggacgcgccctgtagcggcgcatt
aagcgcggcgggtgtggtggttacgcgcagcgtgaccgctacacttgcca
gcgccctagcgcccgctcctttcgctttcttcccttcctttctcgccacg
ttcgccggctttccccgtcaagctctaaatcgggggctccctttagggtt
ccgatttagtgctttacggcacctcgaccccaaaaaacttgattagggtg
atggttcacgtagtgggccatcgccctgatagacggtttttcgccctttg
acgttggagtccacgttctttaatagtggactcttgttccaaactggaac
aacactcaaccctatctcggtctattcttttgatttataagggattttgc
cgatttcggcctattggttaaaaaatgagctgatttaacaaaaatttaac
gcgaattttaacaaaatattaacgcttacaatttaggtggcacttttcgg
ggaaatgtgcgcggaacccctatttgtttatttttctaaatacattcaaa
tatgtatccgctcatgagacaataaccctgataaatgcttcaataatatt
gaaaaaggaagagtatgagtattcaacatttccgtgtcgcccttattccc
ttttttgcggcattttgccttcctgtttttgctcacccagaaacgctggt
gaaagtaaaagatgctgaagatcagttgggtgcacgagtgggttacatcg
aactggatctcaacagcggtaagatccttgagagttttcgccccgaagaa
cgttttccaatgatgagcacttttaaagttctgctatgtggcgcggtatt
atcccgtattgacgccgggcaagagcaactcggtcgccgcatacactatt
ctcagaatgacttggttgagtactcaccagtcacagaaaagcatcttacg
gatggcatgacagtaagagaattatgcagtgctgccataaccatgagtga
taacactgcggccaacttacttctgacaacgatcggaggaccgaaggagc
taaccgcttttttgcacaacatgggggatcatgtaactcgccttgatcgt
tgggaaccggagctgaatgaagccataccaaacgacgagcgtgacaccac
gatgcctgtagcaatggcaacaacgttgcgcaaactattaactggcgaac
tacttactctagcttcccggcaacaattaatagactggatggaggcggat
aaagttgcaggaccacttctgcgctcggcccttccggctggctggtttat
tgctgataaatctggagccggtgagcgtgggtctcgcggtatcattgcag
cactggggccagatggtaagccctcccgtatcgtagttatctacacgacg
gggagtcaggcaactatggatgaacgaaatagacagatcgctgagatagg
tgcctcactgattaagcattggtaactgtcagaccaagtttactcatata
tactttagattgatttaaaacttcatttttaatttaaaaggatctaggtg
aagatcctttttgataatctcatgaccaaaatcccttaacgtgagttttc
gttccactgagcgtcagaccccgtagaaaagatcaaaggatcttcttgag
atcctttttttctgcgcgtaatctgctgcttgcaaacaaaaaaaccaccg
ctaccagcggtggtttgtttgccggatcaagagctaccaactctttttcc
gaaggtaactggcttcagcagagcgcagataccaaatactgttcttctag
tgtagccgtagttaggccaccacttcaagaactctgtagcaccgcctaca
tacctccgctctgctaatcctgttaccagtggctgctgccagtggcgata
agtcgtgtcttaccgggttggactcaagacgatagttaccggataaggcg
cagcggtcgggctgaacggggggttcgtgcacacagcccagcttggagcg
aacgacctacaccgaactgagatacctacagcgtgagctatgagaaagcg
ccacgcttcccgaagggagaaaggcggacaggtatccggtaagcggcagg
gtcggaacaggagagcgcacgagggagcttccagggggaaacgcctggta
tctttatagtcctgtcgggtttcgccacctctgacttgagcgtcgatttt
tgtgatgctcgtcaggggggcggagcctatggaaaaacgccagcaacgcg
gcctttttacggttcctggccttttgctggccttttgctcacatgttctt
tcctgcgttatcccctgattctgtggataaccgtattaccgcctttgagt
gagctgataccgctcgccgcagccgaacgaccgagcgcagcgagtcagtg
agcgaggaagcggaagagcgcccaatacgcaaaccgcctctccccgcgcg
ttggccgattcattaatgcagctggcacgacaggtttcccgactggaaag
cgggcagtgagcgcaacgcaattaatgtgagttagctcactcattaggca
ccccaggctttacactttatgcttccggctcgtatgttgtgtggaattgt
gagcggataacaatttcacacaggaaacagctatgaccatgattacgcca
agcgcgcaattaaccctcactaaagggaacaaaagctggagctgcaagct
t

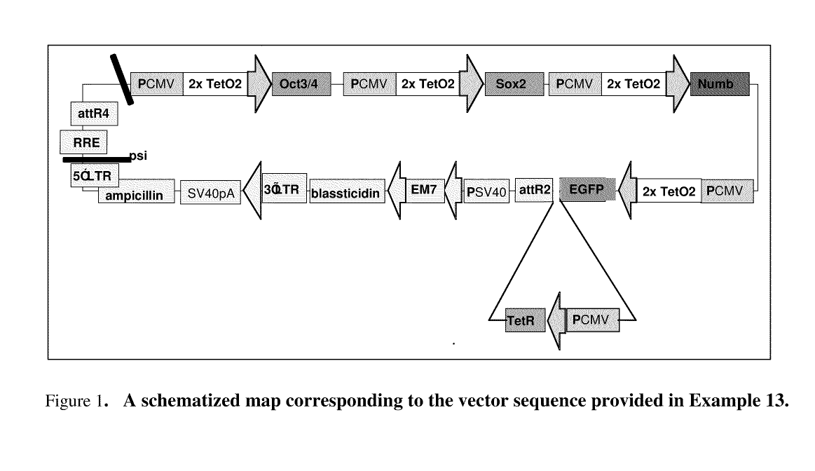

A schematized map corresponding to the vector sequence above is shown in FIG. 1.

The vector may be constructed fully through de novo gene synthesis, or in part through the cloning of the Numb, Sox and OCT3/4 cDNA sequences into the position occupied by LacZ in the Invitrogen pcDNA4tolacZ vector. Similarly, the tetR gene is found in the Invitrogen pcDNA6/TR vector. Coding sequences of genes referenced are also appropriate for cloning into the pcDNA4lacZ vector.

Alternatively, the tetR gene may be transfected into target cells separately utilizing the pcDNA6/TR vector in combination with a vector comprising the sequence here minus the tetR gene and its PCMV promoter.

Likewise, multiple vectors may be employed so long as elements similar to the elements included in the above sequence are present. This may reduce the likelihood of promoter competition. It is to be understood that other conditional promoter elements may be substituted for the tetracycline sensitive promoter elements.

### Example 14

It is expected that intravenous and other administration of pluripotent stem cells produced according to the methods described herein (or other published methods) one or more times can provide replacement cells to the body and that such administration may serve to extend the life or improve the health of the patient suffering age-related senescence.

### Example 15

### Production of Germ Cells

The current invention covers the derivation of germ cells from multipotent, pluripotent, and/or self-renewing stem cells produced according to the methods described herein (or according to other published methods). The production of such germ cells may be suitable for treating infertility and producing embryos in vitro (e.g. Hubner et al., 2003; Kehler et al., 2005; Nayernia et al., 2006a; Nayernia et al., 2006b; Drusenheimer et al., 2007; Moore et al., 2007; etc.)

### Example 16

### Generation of Transgenic Animals

The present invention covers the generation of transgenic animals. As with other pluripotent cells, the pluripotent cells produced by the methods described herein (or other published methods) may be utilized to produce transgenic animals by any method known to the art.

### Example 17

### Therapeutic Vector Construction

Examples of retroviral vectors which may be employed include, but are not limited to, those derived from Moloney Murine Leukemia Virus, Moloney Murine Sarcoma Virus, and Rous Sarcoma Virus, FIV, and HIV. Appropriate expression vectors are that may be employed for transfecting DNA or RNA into eukaryotic cells. Such vectors include, but are not limited to, prokaryotic vectors such as, for example, bacterial vectors; eukaryotic vectors, such as, for example, yeast vectors and fungal vectors; and viral vectors, such as, but not limited to, lentiviral vectors, adenoviral vectors, adeno-associated viral vectors, and retroviral vectors.

The replication incompetent pcDNA 6.2 GW/miR and pcDNA 6.2/EmGFP-Bsd/V5-DEST vectors are examples of an appropriate expression vectors (Invitrogen) and allow expression of synthetic oligonucleotides (e.g. miRNAs) that have the capacity to cleave targeted sequences. These vectors include flanking and loop sequences from endogenous miRNA to direct the excision of the engineered miRNA from a longer Pol II transcript (pre-miRNA).

Alternatively, inclusion of the HIV psi sequence allows the therapeutic vector to compete with native HIV genome for packaging into viral particles, also inhibiting HIV transmission.

Combining multiple miRNA sequences directed against a single target increases the likelihood of success in reducing target sequence expression. miRNA sequences may be operably linked to tissue specific promoters such as the EF-1 alpha promoter, any T cell specific promoter, or macrophage specific promoter to ensure expression in the desired cell types.

Utilizing Invitrogen's lentiviral destination (DEST) vectors for gene expression, the resulting therapeutic vector(s) becomes capable of stably transducing both dividing and non-dividing cell types.

In a preferred embodiment, the therapeutic vector(s) contains multiple synthetic oligonucleotide sequences driven by one or more promoters so as to reduce expression of CXCR4, CCR5, and/or any other cellular protein known to act as a co-receptor for HIV infection in target cells.

In one therapeutic vector (constructed in 2006), four miRNA sequences targeting CXCR4 and CCR5 co-receptors were cloned into the pcDNA 6.2 GW/miR vector along with decoy RNA sequences targeting HIV-2 TAR and RRE.

Genetic constructs may include a vector backbone, and a transactivator which regulates a promoter operably linked to heterologous nucleic acid sequences.

Another example of a suitable vector is a retroviral vector. Retroviruses are RNA viruses which contain an RNA genome. The gag, pol, and env genes are flanked by long terminal repeat (LTR) sequences. The 5′ and 3′ LTR sequences promote transcription and polyadenylation of mRNA's.

The retroviral vector may provide a regulable transactivating element, an internal ribosome reentry site (IRES), a selection marker, and a target heterologous gene operated by a regulable promoter.

Alternatively, multiple sequences may be expressed under the control of multiple promoters. Finally, the retroviral vector may contain cis-acting sequences necessary for reverse transcription and integration. Upon infection, the RNA is reverse transcribed to DNA which integrates efficiently into the host genome. The recombinant retrovirus of this invention is genetically modified in such a way that some of the retroviral, infectious genes of the native virus are removed and in embodiments replaced instead with a target nucleic acid sequence for genetic modification of the cell. The sequences may be exogenous DNA or RNA, in its natural or altered form.

### Example 18

### Example Methods for Generation of the Therapeutic Vector

The methods for generation of the therapeutic vector(s) include those taught in Invitrogen's Viral Power Lentiviral Expression Systems Manual (incorporated by reference herein). Briefly, the EmGFP-bsd cassette is cloned as a Pm1I-B1pI fragment into the pLenti6/R4R2/V5-DEST vector, while the miR-decoy cassette is simultaneously transferred by BP reaction into pDONR221. Then the EF1a promoter and miR-decoy are Multisite LR crossed into the modified pLenti6/EmGFP-bsd/R4R2-DESTvector.

pLenti6/R4R2/V5-DEST vector sequence
(SEQ ID NO: 1):
aatgtagtcttatgcaatactcttgtagtcttgcaacatggtaacgatga
gttagcaacatgccttacaaggagagaaaaagcaccgtgcatgccgattg
gtggaagtaaggtggtacgatcgtgccttattaggaaggcaacagacggg
tctgacatggattggacgaaccactgaattgccgcattgcagagatattg
tatttaagtgcctagctcgatacataaacgggtctctctggttagaccag
atctgagcctgggagctctctggctaactagggaacccactgcttaagcc
tcaataaagcttgccttgagtgcttcaagtagtgtgtgcccgtctgttgt
gtgactctggtaactagagatccctcagacccttttagtcagtgtggaaa
atctctagcagtggcgcccgaacagggacttgaaagcgaaagggaaacca
gaggagctctctcgacgcaggacctcggcttgctgaagcgcgcacggcaa
gaggcgaggggcggcgactggtgagtacgccaaaaattttgactagcgga
ggctagaaggagagagatgggtgcgagagcgtcagtattaagcgggggag
aattagatcgcgatgggaaaaaattcggttaaggccagggggaaagaaaa
aatataaattaaaacatatagtatgggcaagcagggagctagaacgattc
gcagttaatcctggcctgttagaaacatcagaaggctgtagacaaatact
gggacagctacaaccatcccttcagacaggatcagaagaacttagatcat
tatataatacagtagcaaccctctattgtgtgcatcaaaggatagagata
aaagacaccaaggaagctttagacaagatagaggaagagcaaaacaaaag
taagaccaccgcacagcaagcggccgctgatcttcagacctggaggagga
gatatgagggacaattggagaagtgaattatataaatataaagtagtaaa
aattgaaccattaggagtagcacccaccaaggcaaagagaagagtggtgc
agagagaaaaaagagcagtgggaataggagctttgttccttgggttcttg
ggagcagcaggaagcactatgggcgcagcgtcaatgacgctgacggtaca
ggccagacaattattgtctggtatagtgcagcagcagaacaatttgctga
gggctattgaggcgcaacagcatctgttgcaactcacagtctggggcatc
aagcagctccaggcaagaatcctggctgtggaaagatacctaaaggatca
acagctcctggggatttggggttgctctggaaaactcatttgcaccactg
ctgtgccttggaatgctagttggagtaataaatctctggaacagatttgg
aatcacacgacctggatggagtgggacagagaaattaacaattacacaag
cttaatacactccttaattgaagaatcgcaaaaccagcaagaaaagaatg
aacaagaattattggaattagataaatgggcaagtttgtggaattggttt
aacataacaaattggctgtggtatataaaattattcataatgatagtagg
aggcttggtaggtttaagaatagtttttgctgtactttctatagtgaata
gagttaggcagggatattcaccattatcgtttcagacccacctcccaacc
ccgaggggacccgacaggcccgaaggaatagaagaagaaggtggagagag
agacagagacagatccattcgattagtgaacggatctcgacggtatcgat
gtcgacgttaacgctagtgatatcaactttgtatagaaaagttgaacgag
aaacgtaaaatgatataaatatcaatatattaaattagattttgcataaa
aaacagactacataatactgtaaaacacaacatatccagtcactatggcg
gccgcattaggcaccccaggctttacactttatgcttccggctcgtataa
tgtgtggattttgagttaggatccgtcgagattttcaggagctaaggaag
ctaaaatggagaaaaaaatcactggatataccaccgttgatatatcccaa
tggcatcgtaaagaacattttgaggcatttcagtcagttgctcaatgtac
ctataaccagaccgttcagctggatattacggcctttttaaagaccgtaa
agaaaaataagcacaagttttatccggcctttattcacattcttgcccgc
ctgatgaatgctcatccggaattccgtatggcaatgaaagacggtgagct
ggtgatatgggatagtgttcacccttgttacaccgttttccatgagcaaa
ctgaaacgttttcatcgctctggagtgaataccacgacgatttccggcag
tttctacacatatattcgcaagatgtggcgtgttacggtgaaaacctggc
ctatttccctaaagggtttattgagaatatgtttttcgtctcagccaatc
cctgggtgagtttcaccagttttgatttaaacgtggccaatatggacaac
ttcttcgcccccgttttcaccatgggcaaatattatacgcaaggcgacaa
ggtgctgatgccgctggcgattcaggttcatcatgccgtttgtgatggct
tccatgtcggcagaatgcttaatgaattacaacagtactgcgatgagtgg
cagggcggggcgtaaagatctggatccggcttactaaaagccagataaca
gtatgcgtatttgcgcgctgatttttgcggtataagaatatatactgata
tgtatacccgaagtatgtcaaaaagaggtatgctatgaagcagcgtatta
cagtgacagttgacagcgacagctatcagttgctcaaggcatatatgatg
tcaatatctccggtctggtaagcacaaccatgcagaatgaagcccgtcgt
ctgcgtgccgaacgctggaaagcggaaaatcaggaagggatggctgaggt
cgcccggtttattgaaatgaacggctcttttgctgacgagaacagggact
ggtgaaatgcagtttaaggtttacacctataaaagagagagccgttatcg
tctgtttgtggatgtacagagtgatattattgacacgcccgggcgacgga
tggtgatccccctggccagtgcacgtctgctgtcagataaagtctcccgt
gaactttacccggtggtgcatatcggggatgaaagctggcgcatgatgac
caccgatatggccagtgtgccggtctccgttatcggggaagaagtggctg
atctcagccaccgcgaaaatgacatcaaaaacgccattaacctgatgttc
tggggaatataaatgtcaggctccgttatacacagccagtctgcaggtcg
accatagtgactggatatgttgtgttttacagtattatgtagtctgtttt
ttatgcaaaatctaatttaatatattgatatttatatcattttacgtttc
tcgttcagctttcttgtacaaagtggttgatatccagcacagtggcggcc
gctcgagtctagagggcccgcggttcgaaggtaagcctatccctaaccct
ctcctcggtctcgattctacgcgtaccggttagtaatgagtttggaatta
attctgtggaatgtgtgtcagttagggtgtggaaagtccccaggctcccc
agcaggcagaagtatgcaaagcatgcatctcaattagtcagcaaccaggt
gtggaaagtccccaggctccccagcaggcagaagtatgcaaagcatgcat
ctcaattagtcagcaaccatagtcccgcccctaactccgcccatcccgcc
cctaactccgcccagttccgcccattctccgccccatggctgactaattt
tttttatttatgcagaggccgaggccgcctctgcctctgagctattccag
aagtagtgaggaggcttttttggaggcctaggcttttgcaaaaagctccc
gggagcttgtatatccattttcggatctgatcagcacgtgttgacaatta
atcatcggcatagtatatcggcatagtataatacgacaaggtgaggaact
aaaccatggccaagcctttgtctcaagaagaatccaccctcattgaaaga
gcaacggctacaatcaacagcatccccatctctgaagactacagcgtcgc
cagcgcagctctctctagcgacggccgcatcttcactggtgtcaatgtat
atcattttactgggggaccttgtgcagaactcgtggtgctgggcactgct
gctgctgcggcagctggcaacctgacttgtatcgtcgcgatcggaaatga
gaacaggggcatcttgagcccctgcggacggtgccgacaggtgcttctcg
atctgcatcctgggatcaaagccatagtgaaggacagtgatggacagccg
acggcagttgggattcgtgaattgctgccctctggttatgtgtgggaggg
ctaagcacaattcgagctcggtacctttaagaccaatgacttacaaggca
gctgtagatcttagccactttttaaaagaaaaggggggactggaagggct
aattcactcccaacgaagacaagatctgctttttgcttgtactgggtctc
tctggttagaccagatctgagcctgggagctctctggctaactagggaac
ccactgcttaagcctcaataaagcttgccttgagtgcttcaagtagtgtg
tgcccgtctgttgtgtgactctggtaactagagatccctcagaccctttt
agtcagtgtggaaaatctctagcagtagtagttcatgtcatcttattatt
cagtatttataacttgcaaagaaatgaatatcagagagtgagaggaactt
gtttattgcagcttataatggttacaaataaagcaatagcatcacaaatt
tcacaaataaagcatttttttcactgcattctagttgtggtttgtccaaa
ctcatcaatgtatcttatcatgtctggctctagctatcccgcccctaact
ccgcccatcccgcccctaactccgcccagttccgcccattctccgcccca
tggctgactaattttttttatttatgcagaggccgaggccgcctcggcct
ctgagctattccagaagtagtgaggaggcttttttggaggcctagggacg
tacccaattcgccctatagtgagtcgtattacgcgcgctcactggccgtc
gttttacaacgtcgtgactgggaaaaccctggcgttacccaacttaatcg
ccttgcagcacatccccctttcgccagctggcgtaatagcgaagaggccc
gcaccgatcgcccttcccaacagttgcgcagcctgaatggcgaatgggac
gcgccctgtagcggcgcattaagcgcggcgggtgtggtggttacgcgcag
cgtgaccgctacacttgccagcgccctagcgcccgctcctttcgctttct
tcccttcctttctcgccacgttcgccggctttccccgtcaagctctaaat
cgggggctccctttagggttccgatttagtgctttacggcacctcgaccc
caaaaaacttgattagggtgatggttcacgtagtgggccatcgccctgat
agacggtttttcgccctttgacgttggagtccacgttctttaatagtgga
ctcttgttccaaactggaacaacactcaaccctatctcggtctattcttt
tgatttataagggattttgccgatttcggcctattggttaaaaaatgagc
tgatttaacaaaaatttaacgcgaattttaacaaaatattaacgcttaca
atttaggtggcacttttcggggaaatgtgcgcggaacccctatttgttta
tttttctaaatacattcaaatatgtatccgctcatgagacaataaccctg
ataaatgcttcaataatattgaaaaaggaagagtatgagtattcaacatt
tccgtgtcgcccttattcccttttttgcggcattttgccttcctgttttt
gctcacccagaaacgctggtgaaagtaaaagatgctgaagatcagttggg
tgcacgagtgggttacatcgaactggatctcaacagcggtaagatccttg
agagttttcgccccgaagaacgttttccaatgatgagcacttttaaagtt
ctgctatgtggcgcggtattatcccgtattgacgccgggcaagagcaact
cggtcgccgcatacactattctcagaatgacttggttgagtactcaccag
tcacagaaaagcatcttacggatggcatgacagtaagagaattatgcagt
gctgccataaccatgagtgataacactgcggccaacttacttctgacaac
gatcggaggaccgaaggagctaaccgcttttttgcacaacatgggggatc
atgtaactcgccttgatcgttgggaaccggagctgaatgaagccatacca
aacgacgagcgtgacaccacgatgcctgtagcaatggcaacaacgttgcg
caaactattaactggcgaactacttactctagcttcccggcaacaattaa
tagactggatggaggcggataaagttgcaggaccacttctgcgctcggcc
cttccggctggctggtttattgctgataaatctggagccggtgagcgtgg
gtctcgcggtatcattgcagcactggggccagatggtaagccctcccgta
tcgtagttatctacacgacggggagtcaggcaactatggatgaacgaaat
agacagatcgctgagataggtgcctcactgattaagcattggtaactgtc
agaccaagtttactcatatatactttagattgatttaaaacttcattttt
aatttaaaaggatctaggtgaagatcctttttgataatctcatgaccaaa
atcccttaacgtgagttttcgttccactgagcgtcagaccccgtagaaaa
gatcaaaggatcttcttgagatcctttttttctgcgcgtaatctgctgct
tgcaaacaaaaaaaccaccgctaccagcggtggtttgtttgccggatcaa
gagctaccaactctttttccgaaggtaactggcttcagcagagcgcagat
accaaatactgttcttctagtgtagccgtagttaggccaccacttcaaga
actctgtagcaccgcctacatacctcgctctgctaatcctgttaccagtg
gctgctgccagtggcgataagtcgtgtcttaccgggttggactcaagacg
atagttaccggataaggcgcagcggtcgggctgaacggggggttcgtgca
cacagcccagcttggagcgaacgacctacaccgaactgagatacctacag
cgtgagctatgagaaagcgccacgcttcccgaagggagaaaggcggacag
gtatccggtaagcggcagggtcggaacaggagagcgcacgagggagcttc
cagggggaaacgcctggtatctttatagtcctgtcgggtttcgccacctc
tgacttgagcgtcgatttttgtgatgctcgtcaggggggcggagcctatg
gaaaaacgccagcaacgcggcctttttacggttcctggccttttgctggc
cttttgctcacatgttctttcctgcgttatcccctgattctgtggataac
cgtattaccgcctttgagtgagctgataccgctcgccgcagccgaacgac
cgagcgcagcgagtcagtgagcgaggaagcggaagagcgcccaatacgca
aaccgcctctccccgcgcgttggccgattcattaatgcagctggcacgac
aggtttcccgactggaaagcgggcagtgagcgcaacgcaattaatgtgag
ttagctcactcattaggcaccccaggctttacactttatgcttccggctc
gtatgttgtgtggaattgtgagcggataacaatttcacacaggaaacagc
tatgaccatgattacgccaagcgcgcaattaaccctcactaaagggaaca
aaagctggagctgcaagctt
Example miR-decoy cassette sequence
(SEQ ID NO: 3):
gtcgaccagtggatcctggaggcttgctgaaggctgtatgctgatcgggt
gtaaactgagcttggttttggccactgactgaccaagctcattacacccg
atcaggacacaaggcctgttactagcactcacatggaacaaatggcccag
atcctggaggcttgctgaaggctgtatgctgataccaggcaggataaggc
cagttttggccactgactgactggccttactgcctggtatcaggacacaa
ggcctgttactagcactcacatggaacaaatggcccagatcctggaggct
tgctgaaggctgtatgctgtgaccaggatgaccaatccatgttttggcca
ctgactgacatggattgcatcctggtcacaggacacaaggcctgttacta
gcactcacatggaacaaatggcccagatcctggaggcttgctgaaggctg
tatgctgatagcttggtccaacctgttagttttggccactgactgactaa
caggtgaccaagctatcaggacacaaggcctgttactagcactcacatgg
aacaaatggcccagatctccccagtggaaagacgcgcaggcaaaacgcac
cacgtgacggagcgtgaccgcgcgccgagcgcgcgccaaggtcgggcagg
aagagggcctatttcccatgattccttcatatttgcatatacgatacaag
gctgttagagagataattagaattaatttgactgtaaacacaaagatatt
agtacaaaatacgtgacgtagaaagtaataatttcttgggtagtttgcag
ttttaaaattatgttttaaaatggactatcatatgcttaccgtaacttga
aagtatttcgatttcttgggtttatatatcttgtggaaaggacggtgctc
gcttcggcagcacgtcgtgctagggttcttgggttttctcgcaacagcag
gttctgcaatgggcgcggcgtccctgaccgtgtcggctcagtcccggact
ttactggccgggatagtgcagcaacagcaacagctgttggacgtggtcaa
gagacaacaagaactgttgcgactgaccgtctggggaacgaaaaacctcc
aggcaagagtcactgctatagagaagtacctacaggaccaggcgcggcta
aattcatggggatgtctagacctagagcggacttcggtccgctttttccc
cagtggaaagacgcgcaggcaaaacgcaccacgtgacggagcgtgaccgc
gcgccgagcgcgcgccaaggtcgggcaggaagagggcctatttcccatga
ttccttcatatttgcatatacgatacaaggctgttagagagataattaga
attaatttgactgtaaacacaaagatattagtacaaaatacgtgacgtag
aaagtaataatttcttgggtagtttgcagttttaaaattatgttttaaaa
tggactatcatatgcttaccgtaacttgaaagtatttcgatttcttgggt
ttatatatcttgtggaaaggacggtgctcgcttcggcagcacgtcggtcg
ctctgcggagaggctggcagattgagccctgggaggttctctccagcact
agcaggtagagcctgggtgttccctgctagactctcaccagtgcttggcc
ggcactgggcagacggctccacgcttgcttgcttaaagacctcttaataa
agctgctctagacctagagcggacttcggtccgcttttttacgtactcga
g

### Example 19

### Methods for Propagating/Proliferating Stem/Progenitor Cells In Vivo

In order to obtain large numbers of target cells that are relatively resistant to 1) HIV infection and/or 2) HIV replication and/or 3) HIV transcription, progenitor/stem cells can be grown in Dulbecco's modified Minimal Essential Medium (DMEM) supplemented with glutamine, beta.-mercaptoethanol, 10% (by volume) horse serum, and human recombinant Leukemia Inhibitory Factor (LIF). The LIF replaces the need for maintaining progenitor/stem cells on feeder layers of cells, (which may also be employed) and is essential for maintaining progenitor/stem cells in an undifferentiated state.

### Example 20

Stem cells are collected from individuals, the cells are transfected with the therapeutic vectors, then prepared for transplantation by standard methods, with or without HLA typing and matching.

### Example 21

Umbilical cord blood samples are obtained from umbilical blood cord bank. The cells are then transfected with the therapeutic vector of beneficial sequences, then prepared for transplantation by standard methods, with or without HLA typing and matching.

### Example 22

### Examples of Synthetic Oligonucleotide Sequences Suitable for Inclusion In the Therapeutic Vector

Any synthetic oligonucleotide sequences that successfully reduce the protein expression of targeted sequences >70% is covered by the present invention.

Any synthetic oligonucleotide sequences that successfully reduce the ability of target cells to sustain HIV replication by >70% or to a lesser but therapeutic degree or HIV viral activity by >70% or to a lesser but therapeutic degree are also covered by this invention.

Examples of miRNA sequences include miRNA sequences derived by IVGN algorithm(Invitrogen). miRNA sequences targeting the CXCR4 gene include top strand: 5′-TGCTGATACCAGGCAGGATAAGGCCAGTTTTGGCCACTGACTGACTGGCCTTACTGCCT GGTAT-3′ (SEQ ID NO: 4) and bottom strand: 5′-CCTGATACCAGGCAGTAAGGCCAGTCAGTCAGTGGCCAAAACTGGCCTTATCCTGCCTG GTATC-3′ (SEQ ID NO: 5); as well as top strand: 5′-TGCTGTGACCAGGATGACCAATCCATGTTTTGGCCACTGACTGACATGGATTGCATCCTG GTCA-3′ (SEQ ID NO: 6) and bottom strand: 5′-CCTGTGACCAGGATGCAATCCATGTCAGTCAGTGGCCAAAACATGGATTGGTCATCCTG GTCAC-3′ (SEQ ID NO: Similarly, miRNA sequences targeting the CCR5 gene include top strand: 5′-TGCTGATCGGGTGTAAACTGAGCTTGGTTTTGGCCACTGACTGACCAAGCTCATT ACACCCGAT-3′ (SEQ ID NO: 8) and bottom strand: 5′-CCTGATCGGGTGTAATGAGCTTGGTCAGTCAGTGGCCAAAACCAAGCTCAGTTT ACACCCGATC-3′ SE ID NO: 9; as well as top strand5′-TGCTGATAGCTTGGTCCAACCTGTTAGTTTTGGCCACTGACTGACTAACAGGTGA CCAAGCTAT-3′ (SEQ ID NO: 10) and bottom strand: 5′-CCTGATAGCTTGGTCACCTGTTAGTCAGTCAGTGGCCAAAACTAACAGGTTGGA CCAAGCTATC-3′ (SEQ ID NO: II).

### Example 23

Examples of Decoy RNA suitable for inclusion in the therapeutic vector. Any decoy sequences that successfully reduce the ability of target cells to sustain HIV replication by >70% or to a lesser but therapeutic degree or HIV viral activity by >70% or to a lesser but therapeutic degree are covered by this invention.

An example TAR decoy sequence is (SEQ ID NO: 12)

gtcgctctgcggagaggctggcagattgagccctgggaggttctctccag
cactagcaggtagagcctgggtgttccctgctagactctcaccagtgctt
ggccggcactgggcagacggctccacgcttgcttgcttaaagacctctta
ataaagctgc (Browning et al., 1999)

An example RRE decoy sequence is (SEQ ID NO: 13)

tgctagggttcttgggttttctcgcaacagcaggttctgcaatgggcgcg
gcgtccctgaccgtgtcggctcagtcccggactttactggccgggatagt
gcagcaacagcaacagctgttggacgtggtcaagagacaacaagaactgt
tgcgactgaccgtctggggaacgaaaaacctccaggcaagagtcactgct
atagagaagtacctacaggaccaggcgcggctaaattcatggggatg
(Dillon et al., 1990).

### Example 24

### Flanking Sequences Providing Stability for RNA Decoys

Examples of appropriate flanking sequences for RNA decoys are as follows:

(SEQ ID NO: 14)
GUGCUCGCUUCGGCAGCACGTCGAC ---TAR DECOY SEQ---
(SEQ ID NO: 15)
UCUAGAGCGGACUUCGGUCCGCUUUU
(SEQ ID NO: 16)
GUGCUCGCUUCGGCAGCACGTCGAC ---RRE DECOY SEQ---
(SEQ ID NO: 17)
UCUAGAGCGGACUUCGGUCCGCUUUU

Previously, it was demonstrated that decoy sequences flanked by hairpins on either side, 19 nucleotides (ntds) of the U6 RNA on the 5′ side as well as a 3′ stem immediately preceding a poly U terminator for POLIII, showed greater stability. This arrangement is expected to protect against 3′-5′ exonuclease attack, and to reduce the chances of the 3′ trailer interfering with the insert RNA folding. Since only the first ¾ of the tRNA sequence is present, the 5′ end of the insert should be protected and export from the nucleus should be prevented (Good et al., 1997).

### Example 25

### Introduction of Therapeutic Vector to the Host

In a preferred embodiment, blood stem/progenitor cells, and target cells are transfected with the therapeutic vector(s) (or associated therapeutic virus) in vivo by introduction of the therapeutic vector(s) into the host blood, tissues, or bone marrow, etc. The greatest benefit may be achieved by modifying a large number of endogenous target and stem/progenitor cells. This may be accomplished by using an appropriately-sized, catheter-like device, or needle to inject the therapeutic vector(s) into the venous or arterial circulation. In a preferred embodiment, the virus is pseudotyped with VSV-G envelope glycoprotein and native HIV-1 env proteins.

### Example 26

### Introduction of Genetically-Modified Cells into the Host

Blood cells, such as mature peripheral blood T lymphocytes, monocytes, macrophages, T cell progenitors, macrophage-monocyte progenitor cells, and/or pluripotent hematopoietic stem cells (such as those found in umbilical cord blood and occupying bone marrow spaces) as well as other stem/progenitor cells can be transfected using the therapeutic vector(s) in vitro. Appropriate concentrations of the therapeutic vector(s) may be those consistent with Browning et al., 1999. Subsequently, cells are expanded (propagated) in vitro, and are then transferred to the host via introduction of the cells to the venous or arterial circulation using a intravenous needle or catheter. Subsequently, cells transfected with the therapeutic vectors are able to “home” to the bone marrow and other tissues.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Example 27

### Examples of Expressed or Targeted Transgenes Utilized in the Present Invention

Any transgene sequences effective in fulfilling the present invention is suitable for use in the present invention. Suitable nucleotide sequences may be drawn from any species so long as the desired cells or behavior is achieved. Likewise the method of naming such sequences, either in lower case or upper case letters herein, does not imply a particular species. The following sequences stored in the NCBI database (listed by accession number) represent examples of sequences referenced above in the present application. They are also examples of specific transgene encoding sequences (cds) suitable for use in the present invention, but do not in any way limit the practice of the invention:

cardiotrophin1: U43030 (SEQ ID NO: 18):
atgagccggagggagggaagtctggaagacccccagactgattcctcagt
ctcacttcttccccacttggaggccaagatccgtcagacacacagccttg
cgcacctcctcaccaaatacgctgagcagctgctccaggaatatgtgcag
ctccagggagaccccttcgggctgcccagcttctcgccgccgcggctgcc
ggtggccggcctgagcgccccggctccgagccacgcggggctgccagtgc
acgagcggctgcggctggacgcggcggcgctggccgcgctgcccccgctg
ctggacgcagtgtgtcgccgccaggccgagctgaacccgcgcgcgccgcg
cctgctgcgccgcctggaggacgcggcgcgccaggcccgggccctgggcg
ccgccgtggaggccttgctggccgcgctgggcgccgccaaccgcgggccc
cgggccgagccccccgccgccaccgcctcagccgcctccgccaccggggt
cttccccgccaaggtgctggggctccgcgtttgcggcctctaccgcgagt
ggctgagccgcaccgagggcgacctgggccagctgctgcccgggggctcg
gcctga; NM_001330; NM_013246; BC064416; BC036787;
BC049822.
CNTF: BC074964 (SEQ ID NO: 19):
atggctttcacagagcattcaccgctgacccctcaccgtcgggacctctg
tagccgctctatctggctagcaaggaagattcgttcagacctgactgctc
ttacggaatcctatgtgaagcatcagggcctgaacaagaacatcaacctg
gactctgcggatgggatgccagtggcaagcactgatcagtggagtgagct
gaccgaggcagagcgactccaagagaaccttcaagcttatcgtaccttcc
atgttttgttggccaggctcttagaagaccagcaggtgcattttacccca
accgaaggtgacttccatcaagctatacatacccttcttctccaagtcgc
tgcctttgcataccagatagaggagttaatgatactcctggaatacaaga
tcccccgcaatgaggctgatgggatgcctattaatgttggagatggtggt
ctctttgagaagaagctgtggggcctaaaggtgctgcaggagctttcaca
gtggacagtaaggtccatccatgaccttcgtttcatttcttctcatcaga
ctgggatcccagcacgtgggagccattatattgctaacaacaagaaaatg
tag; NM_000614; NM_147164; NM_001842.
GP130: NM_175767 (SEQ ID NO: 20):
atgttgacgttgcagacttggctagtgcaagccttgtttattttcctcac
cactgaatctacaggtgaacttctagatccatgtggttatatcagtcctg
aatctccagttgtacaacttcattctaatttcactgcagtttgtgtgcta
aaggaaaaatgtatggattattttcatgtaaatgctaattacattgtctg
gaaaacaaaccattttactattcctaaggagcaatatactatcataaaca
gaacagcatccagtgtcacctttacagatatagcttcattaaatattcag
ctcacttgcaacattcttacattcggacagcttgaacagaatgtttatgg
aatcacaataatttcaggcttgcctccagaaaaacctaaaaatttgagtt
gcattgtgaacgaggggaagaaaatgaggtgtgagtgggatggtggaagg
gaaacacacttggagacaaacttcactttaaaatctgaatgggcaacaca
caagtttgctgattgcaaagcaaaacgtgacacccccacctcatgcactg
ttgattattctactgtgtattttgtcaacattgaagtctgggtagaagca
gagaatgcccttgggaaggttacatcagatcatatcaattttgatcctgt
atataaagtgaagcccaatccgccacataatttatcagtgatcaactcag
aggaactgtctagtatcttaaaattgacatggaccaacccaagtattaag
agtgttataatactaaaatataacattcaatataggaccaaagatgcctc
aacttggagccagattcctcctgaagacacagcatccacccgatcttcat
tcactgtccaagaccttaaaccttttacagaatatgtgtttaggattcgc
tgtatgaaggaagatggtaagggatactggagtgactggagtgaagaagc
aagtgggatcacctatgaagataacattgcctccttttga;
NM_002184; EF442778.
IL6: BC015511 (SEQ ID NO: 21):
atgaactccttctccacaagcgccttcggtccagttgccttctccctggg
gctgctcctggtgttgcctgctgccttccctgccccagtacccccaggag
aagattccaaagatgtagccgccccacacagacagccactcacctcttca
gaacgaattgacaaacaaattcggtacatcctcgacggcatctcagccct
gagaaaggagacatgtaacaagagtaacatgtgtgaaagcagcaaagagg
cactggcagaaaacaacctgaaccttccaaagatggctgaaaaagatgga
tgcttccaatctggattcaatgaggagacttgcctggtgaaaatcatcac
tggtcttttggagtttgaggtatacctagagtacctccagaacagatttg
agagtagtgaggaacaagccagagctgtgcagatgagtacaaaagtcctg
atccagttcctgcagaaaaaggcaaagaatctagatgcaataaccacccc
tgacccaaccacaaatgccagcctgctgacgaagctgcaggcacagaacc
agtggctgcaggacatgacaactcatctcattctgcgcagctttaaggag
ttcctgcagtccagcctgagggctcttcggcaaatgtag; AB107656.
HOXB4: NM_024015 (SEQ ID NO: 22):
atggctatgagttcttttttgatcaactcaaactatgtcgaccccaagtt
ccctccatgcgaggaatattcacagagcgattacctacccagcgaccact
cgcccgggtactacgccggcggccagaggcgagagagcagcttccagccg
gaggcgggcttcgggcggcgcgcggcgtgcaccgtgcagcgctacgcggc
ctgccgggaccctgggcccccgccgcctccgccaccacccccgccgcccc
cgccaccgcccggtctgtcccctcgggctcctgcgccgccacccgccggg
gccctcctcccggagcccggccagcgctgcgaggcggtcagcagcagccc
cccgccgcctccctgcgcccagaaccccctgcaccccagcccgtcccact
ccgcgtgcaaagagcccgtcgtctacccctggatgcgcaaagttcacgtg
agcacggtaaaccccaattacgccggcggggagcccaagcgctctcggac
cgcctacacgcgccagcaggtcttggagctggagaaggaatttcactaca
accgctacctgacacggcgccggagggtggagatcgcccacgcgctctgc
ctctccgagcgccagatcaagatctggttccagaaccggcgcatgaagtg
gaaaaaagaccacaagttgcccaacaccaagatccgctcgggtggtgcgg
caggctcagccggagggccccctggccggcccaatggaggcccccgcgcg
ctctag; NM_010459.
IL6R: NM_000565 (SEQ ID NO: 23):
atgctggccgtcggctgcgcgctgctggctgccctgctggccgcgccggg
agcggcgctggccccaaggcgctgccctgcgcaggaggtggcgagaggcg
tgctgaccagtctgccaggagacagcgtgactctgacctgcccgggggta
gagccggaagacaatgccactgttcactgggtgctcaggaagccggctgc
aggctcccaccccagcagatgggctggcatgggaaggaggctgctgctga
ggtcggtgcagctccacgactctggaaactattcatgctaccgggccggc
cgcccagctgggactgtgcacttgctggtggatgttccccccgaggagcc
ccagctctcctgcttccggaagagccccctcagcaatgttgtttgtgagt
ggggtcctcggagcaccccatccctgacgacaaaggctgtgctcttggtg
aggaagtttcagaacagtccggccgaagacttccaggagccgtgccagta
ttcccaggagtcccagaagttctcctgccagttagcagtcccggagggag
acagctctttctacatagtgtccatgtgcgtcgccagtagtgtcgggagc
aagttcagcaaaactcaaacctttcagggttgtggaatcttgcagcctga
tccgcctgccaacatcacagtcactgccgtggccagaaacccccgctggc
tcagtgtcacctggcaagacccccactcctggaactcatctttctacaga
ctacggtttgagctcagatatcgggctgaacggtcaaagacattcacaac
atggatggtcaaggacctccagcatcactgtgtcatccacgacgcctgga
gcggcctgaggcacgtggtgcagcttcgtgcccaggaggagttcgggcaa
ggcgagtggagcgagtggagcccggaggccatgggcacgccttggacaga
atccaggagtcctccagctgagaacgaggtgtccacccccatgcaggcac
ttactactaataaagacgatgataatattctcttcagagattctgcaaat
gcgacaagcctcccagtgcaagattcttcttcagtaccactgcccacatt
cctggttgctggagggagcctggccttcggaacgctcctctgcattgcca
ttgttctgaggttcaagaagacgtggaagctgcgggctctgaaggaaggc
aagacaagcatgcatccgccgtactctttggggcagctggtcccggagag
gcctcgacccaccccagtgcttgttcctctcatctccccaccggtgtccc
ccagcagcctggggtctgacaatacctcgagccacaaccgaccagatgcc
agggacccacggagcccttatgacatcagcaatacagactacttcttccc
cagatag; NM_181359.
IL11: NM_133519 (SEQ ID NO: 24):
atgaactgtgtttgtcgcctggtcctggtggtgctgagcctctggccaga
tagagtcgttgcccctgggccaccagctggctcccctcgagtgtcttcag
accctcgtgcagatctggatagcgctgtcctcttgaccaggtccctcctg
gcagacacacggcaactagctgcacagatgagagacaaattcccagctga
tggagaccacaatctggactccctacctaccttggccatgagcgctggga
cactgggatctttgcagcttcctggagtgctgacaaggcttcgagtagac
ttaatgtcctacttccgacatgtacagtggttgcgccgggcagctggtcc
ttccctaaagactctggagccagagctgggtgccctgcaagcccgactgg
aacggctacttcgtcgcttacagctcttgatgtctcgcctagccttgccc
caggcagccccggaccaacctgcggtccctctgggccctcctgcctcggc
ctggggaagcatccgggcagctcatgccatcctaggagggctgcacctga
ccttggactgggccgtgcggggcctgctgttgttaaagactcggctgta
a; NM_008350.
LIF: NM_002309 (SEQ ID NO: 25):
atgaaggtcttggcggcaggagttgtgcccctgctgttggttctgcactg
gaaacatggggcggggagccccctccccatcacccctgtcaacgccacct
gtgccatacgccacccatgtcacaacaacctcatgaaccagatcaggagc
caactggcacagctcaatggcagtgccaatgccctctttattctctatta
cacagcccagggggagccgttccccaacaacctggacaagctatgtggcc
ccaacgtgacggacttcccgcccttccacgccaacggcacggagaaggcc
aagctggtggagctgtaccgcatagtcgtgtaccttggcacctccctggg
caacatcacccgggaccagaagatcctcaaccccagtgccctcagcctcc
acagcaagctcaacgccaccgccgacatcctgcgaggcctccttagcaac
gtgctgtgccgcctgtgcagcaagtaccacgtgggccatgtggacgtgac
ctacggccctgacacctcgggtaaggatgtcttccagaagaagaagctgg
gctgtcaactcctggggaagtataagcagatcatcgccgtgttggcccag
gccttctag ; NM_008501; BB235045.
LIFR: NM_002310 (SEQ ID NO: 26):
atgatggatatttacgtatgtttgaaacgaccatcctggatggtggacaa
taaaagaatgaggactgcttcaaatttccagtggctgttatcaacattta
ttcttctatatctaatgaatcaagtaaatagccagaaaaagggggctcct
catgatttgaagtgtgtaactaacaatttgcaagtgtggaactgttcttg
gaaagcaccctctggaacaggccgtggtactgattatgaagtttgcattg
aaaacaggtcccgttcttgttatcagttggagaaaaccagtattaaaatt
ccagctctttcacatggtgattatgaaataacaataaattctctacatga
ttttggaagttctacaagtaaattcacactaaatgaacaaaacgtttcct
taattccagatactccagagatcttgaatttgtctgctgatttctcaacc
tctacattatacctaaagtggaacgacaggggttcagtttttccacaccg
ctcaaatgttatctgggaaattaaagttctacgtaaagagagtatggagc
tcgtaaaattagtgacccacaacacaactctgaatggcaaagatacactt
catcactggagttgggcctcagatatgcccttggaatgtgccattcattt
tgtggaaattagatgctacattgacaatcttcatttttctggtctcgaag
agtggagtgactggagccctgtgaagaacatttcttggatacctgattct
cagactaaggtttttcctcaagataaagtgatacttgtaggctcagacat
aacattttgttgtgtgagtcaagaaaaagtgttatcagcactgattggcc
atacaaactgccccttgatccatcttgatggggaaaatgttgcaatcaag
attcgtaatatttctgtttctgcaagtagtggaacaaatgtagtttttac
aaccgaagataacatatttggaaccgttatttttgctggatatccaccag
atactcctcaacaactgaattgtgagacacatgatttaaaagaaattata
tgtagttggaatccaggaagggtgacagcgttggtgggcccacgtgctac
aagctacactttagttgaaagtttttcaggaaaatatgttagacttaaaa
gagctgaagcacctacaaacgaaagctatcaattattatttcaaatgctt
ccaaatcaagaaatatataattttactttgaatgctcacaatccgctggg
tcgatcacaatcaacaattttagttaatataactgaaaaagtttatcccc
atactcctacttcattcaaagtgaaggatattaattcaacagctgttaaa
ctttcttggcatttaccaggcaactttgcaaagattaattttttatgtga
aattgaaattaagaaatctaattcagtacaagagcagcggaatgtcacaa
tcaaaggagtagaaaattcaagttatcttgttgctctggacaagttaaat
ccatacactctatatacttttcggattcgttgttctactgaaactttctg
gaaatggagcaaatggagcaataaaaaacaacatttaacaacagaagcca
gtccttcaaaggggcctgatacttggagagagtggagttctgatggaaaa
aatttaataatctattggaagcctttacccattaatgaagctaatggaaa
aatactttcctacaatgtatcgtgttcatcagatgaggaaacacagtccc
tttctgaaatccctgatcctcagcacaaagcagagatacgacttgataag
aatgactacatcatcagcgtagtggctaaaaattctgtgggctcatcacc
accttccaaaatagcgagtatggaaattccaaatgatgatacaaaataga
acaagttgagggatgggaaaggggattctcctcacctggcattacgaccc
caacatgacttgcgactacgtcattaagtggtgtaactcgtctcggtcgg
aaccatgccttatggactggagaaaagttccctcaaacagcactgaaact
gtaatagaatctgatgagtttcgaccaggtataagatataattttttcct
gtatggatgcagaaatcaaggatatcaattattacgctccatgattggat
atatagaagaattggctcccattgttgcaccaaattttactgttgaggat
acttctgcagattcgatattagtaaaatgggaagacattcctgtggaaga
acttagaggctttttaagaggatatttgttttactttggaaaaggagaaa
gagacacatctaagatgagggttttagaatcaggtcgttctgacataaaa
gttaagaatattactgacatatcccagaagacactgagaattgctgatct
tcaaggtaaaacaagttaccacctggtcttgcgagcctatacagatggtg
gagtgggcccggagaagagtatgtatgtggtgacaaaggaaaattctgtg
ggattaattattgccattctcatcccagtggcagtggctgtcattgttgg
agtggtgacaagtatcctttgctatcggaaacgagaatggattaaagaaa
ccttctaccctgatattccaaatccagaaaactgtaaagcattacagttt
caaaagagtgtctgtgagggaagcagtgctcttaaaacattggaaatgaa
tccttgtaccccaaataatgttgaggttctggaaactcgatcagcatttc
ctaaaatagaagatacagaaataatttccccagtagctgagcgtcctgaa
gatcgctctgatgcagagcctgaaaaccatgtggttgtgtcctattgtcc
acccatcattgaggaagaaataccaaacccagccgcagatgaagctggag
ggactgcacaggttatttacattgatgttcagtcgatgtatcagcctcaa
gcaaaaccagaagaagaacaagaaaatgaccctgtaggaggggcaggcta
taagccacagatgcacctccccattaattctactgtggaagatatagctg
cagaagaggacttagataaaactgcgggttacagacctcaggccaatgta
aatacatggaatttagtgtctccagactctcctagatccatagacagcaa
cagtgagattgtctcatttggaagtccatgctccattaattcccgacaat
ttttgattcctcctaaagatgaagactctcctaaatctaatggaggaggg
tggtcctttacaaacttttttcagaacaaaccaaacgattaa;
NM_013584; NM_031048.
STAT3: NM_003150 (SEQ ID NO: 27):
atggcccaatggaatcagctacagcagcttgacacacggtacctggagca
gctccatcagctctacagtgacagcttcccaatggagctgcggcagtttc
tggccccttggattgagagtcaagattgggcatatgcggccagcaaagaa
tcacatgccactttggtgtttcataatctcctgggagagattgaccagca
gtatagccgcttcctgcaagagtcgaatgttctctatcagcacaatctac
gaagaatcaagcagtttcttcagagcaggtatcttgagaagccaatggag
attgcccggattgtggcccggtgcctgtgggaagaatcacgccttctaca
gactgcagccactgcggcccagcaagggggccaggccaaccaccccacag
cagccgtggtgacggagaagcagcagatgctggagcagcaccttcaggat
gtccggaagagagtgcaggatctagaacagaaaatgaaagtggtagagaa
tctccaggatgactttgatttcaactataaaaccctcaagagtcaaggag
acatgcaagatctgaatggaaacaaccagtcagtgaccaggcagaagatg
cagcagctggaacagatgctcactgcgctggaccagatgcggagaagcat
cgtgagtgagctggcggggcttttgtcagcgatggagtacgtgcagaaaa
ctctcacggacgaggagctggctgactggaagaggcggcaacagattgcc
tgcattggaggcccgcccaacatctgcctagatcggctagaaaactggat
aacgtcattagcagaatctcaacttcagacccgtcaacaaattaagaaac
tggaggagttgcagcaaaaagtttcctacaaaggggaccccattgtacag
caccggccgatgctggaggagagaatcgtggagctgtttagaaacttaat
gaaaagtgcctttgtggtggagcggcagccctgcatgcccatgcatcctg
accggcccctcgtcatcaagaccggcgtccagttcactactaaagtcagg
ttgctggtcaaattccctgagttgaattatcagcttaaaattaaagtgtg
cattgacaaagactctggggacgttgcagctctcagaggatcccggaaat
ttaacattctgggcacaaacacaaaagtgatgaacatggaagaatccaac
aacggcagcctctctgcagaattcaaacacttgaccctgagggagcagag
atgtgggaatgggggccgagccaattgtgatgcttccctgattgtgactg
aggagctgcacctgatcacctttgagaccgaggtgtatcaccaaggcctc
aagattgacctagagacccactccttgccagttgtggtgatctccaacat
ctgtcagatgccaaatgcctgggcgtccatcctgtggtacaacatgctga
ccaacaatcccaagaatgtaaacttttttaccaagcccccaattggaacc
tgggatcaagtggccgaggtcctgagctggcagttctcctccaccaccaa
gcgaggactgagcatcgagcagctgactacactggcagagaaactcttgg
gacctggtgtgaattattcagggtgtcagatcacatgggctaaattttgc
aaagaaaacatggctggcaagggcttctccttctgggtctggctggacaa
tatcattgaccttgtgaaaaagtacatcctggccctttggaacgaagggt
acatcatgggctttatcagtaaggagcgggagcgggccatcttgagcact
aagcctccaggcaccttcctgctaagattcagtgaaagcagcaaagaagg
aggcgtcactttcacttgggtggagaaggacatcagcggtaagacccaga
tccagtccgtggaaccatacacaaagcagcagctgaacaacatgtcattt
gctgaaatcatcatgggctataagatcatggatgctaccaatatcctggt
gtctccactggtctatctctatcctgacattcccaaggaggaggcattcg
gaaagtattgtcggccagagagccaggagcatcctgaagctgacccaggc
gctgccccatacctgaagaccaagtttatctgtgtgacaccaacgacctg
cagcaataccattgacctgccgatgtccccccgcactttagattcattga
tgcagtttggaaataatggtgaaggtgctgaaccctcagcaggagggcag
tttgagtccctcacctttgacatggagttgacctcggagtgcgctacctc
ccccatgtga; NM_213662; NM_139276.
NUMB: AF171938 (SEQ ID NO: 28):
atgaacaaattacggcaaagttttaggagaaagaaggatgtttatgttcc
agaggccagtcgtccacatcagtggcagacagatgaagaaggcgttcgca
ccggaaaatgtagcttcccggttaagtaccttggccatgtagaagttgat
gaatcaagaggaatgcacatctgtgaagatgctgtaaaaagattgaaagc
tgaaaggaagttcttcaaaggcttctttggaaaaactggaaagaaagcag
ttaaagcagttctgtgggtctcagcagatggactcagagttgtggatgaa
aaaactaaggacctcatagttgaccagacgatagagaaagtttctttctg
tgccccagacaggaactttgatagagccttttcttacatatgccgtgatg
gcaccactcgtcgctggatctgtcactgcttcatggctgtcaaggacaca
ggtgaaaggttgagccatgcagtaggctgtgcttttgcagcctgtttaga
gcgcaagcagaagcgggagaaggaatgtggagtgactgctacttttgatg
ctagtcggaccacttttacaagagaaggatcattccgtgtcacaacagcc
actgaacaagcagaaagagaggagatcatgaaacaaatgcaagatgccaa
gaaagctgaaacagataagatagtcgttggttcatcagttgcccctggca
acactgccccatccccatcctctcccacctctcctacttctgatgccacg
acctctctggagatgaacaatcctcatgccatcccacgccggcatgctcc
aattgaacagcttgctcgccaaggctctttccgaggttttcctgctctta
gccagaagatgtcaccctttaaacgccaactatccctacgcatcaatgag
ttgccttccactatgcagaggaagactgatttccccattaaaaatgcagt
gccagaagtagaaggggaggcagagagcatcagctccctgtgctcacaga
tcaccaatgccttcagcacacctgaggaccccttctcatctgctccgatg
accaaaccagtgacagtggtggcaccacaatctcctaccttccaagctaa
tggcactgactcagccttccatgtgcttgctaagccagcccatactgctc
tagcacccgtagcaatgcctgtgcgtgaaaccaacccttgggcccatgcc
cctgatgctgctaacaaggaaattgcagccacatgttcggggaccgagtg
gggtcaatcttctggtgctgcctctccaggtctcttccaggccggtcata
gacgtactccctctgaggccgaccgatggttagaagaggtgtctaagagc
gtccgggctcagcagccccaggcctcagctgctcctctgcagccagttct
ccagcctcctccacccactgccatctcccagccagcatcacctttccaag
ggaatgcattcctcacctctcagcctgtgccagtgggtgtggtcccagcc
ctgcaaccagcctttgtccctgcccagtcctatcctgtggccaatggaat
gccctatccagcccctaatgtgcctgtggtgggcatcactccctcccaga
tggtggccaacgtatttggcactgcaggccaccctcaggctgcccatccc
catcagtcacccagcctggtcaggcagcagacattccctcactacgaggc
aagcagtgctaccaccagtcccttctttaagcctcctgctcagcacctca
acggttctgcagctttcaatggtgtagatgatggcaggttggcctcagca
gacaggcatacagaggttcctacaggcacctgcccagtggatccttttga
agcccagtgggctgcattagaaaataagtccaagcagcgtactaatccct
cccctaccaaccctttctccagtgacttacagaagacgtttgaaattgaa
ctttaa;
AF171939 (SEQ ID NO: 29):
atgaacaaattacggcaaagttttaggagaaagaaggatgtttatgttcc
agaggccagtcgtccacatcagtggcagacagatgaagaaggcgttcgca
ccggaaaatgtagcttcccggttaagtaccttggccatgtagaagttgat
gaatcaagaggaatgcacatctgtgaagatgctgtaaaaagattgaaagc
tgaaaggaagttcttcaaaggcttctttggaaaaactggaaagaaagcag
ttaaagcagttctgtgggtctcagcagatggactcagagttgtggatgaa
aaaactaaggacctcatagttgaccagacgatagagaaagtttctttctg
tgccccagacaggaactttgatagagccttttcttacatatgccgtgatg
gcaccactcgtcgctggatctgtcactgcttcatggctgtcaaggacaca
ggtgaaaggttgagccatgcagtaggctgtgcttttgcagcctgtttaga
gcgcaagcagaagcgggagaaggaatgtggagtgactgctacttttgatg
ctagtcggaccacttttacaagagaaggatcattccgtgtcacaacagcc
actgaacaagcagaaagagaggagatcatgaaacaaatgcaagatgccaa
gaaagctgaaacagataagatagtcgttggttcatcagttgcccctggca
acactgccccatccccatcctacccacctctcctacttctgatgccacga
cctctctggagatgaacaatcctcatgccatcccacgccggcatgctcca
attgaacagcttgctcgccaaggctctttccgaggttttcctgctcttag
ccagaagatgtcaccctttaaacgccaactatccctacgcatcaatgagt
tgccttccactatgcagaggaagactgatttccccattaaaaatgcagtg
ccagaagtagaaggggaggcagagagcatcagctccctgtgctcacagat
caccaatgccttcagcacacctgaggaccccttctcatctgctccgatga
ccaaaccagtgacagtggtggcaccacaatctcctaccttccaagggacc
gagtggggtcaatcttctggtgctgcctctccaggtctcttccaggccgg
tcatagacgtactccctctgaggccgaccgatggttagaagaggtgtcta
agagcgtccgggctcagcagccccaggcctcagctgctcctctgcagcca
gttctccagcctcctccacccactgccatctcccagccagcatcaccttt
ccaagggaatgcattcctcacctctcagcctgtgccagtgggtgtggtcc
cagccctgcaaccagcctttgtccctgcccagtcctatcctgtggccaat
ggaatgccctatccagcccctaatgtgcctgtggtgggcatcactccctc
ccagatggtggccaacgtatttggcactgcaggccaccctcaggctgccc
atccccatcagtcacccagcctggtcaggcagcagacattccctcactac
gaggcaagcagtgctaccaccagtcccttctttaagcctcctgctcagca
cctcaacggttctgcagctttcaatggtgtagatgatggcaggttggcct
cagcagacaggcatacagaggttcctacaggcacctgcccagtggatcct
tttgaagcccagtgggctgcattagaaaataagtccaagcagcgtactaa
tccctcccctaccaaccctttctccagtgacttacagaagacgtttgaaa
ttgaactttaa
AF171940 (SEQ ID NO: 30):
atgaacaaattacggcaaagttttaggagaaagaaggatgtttatgttcc
agaggccagtcgtccacatcagtggcagacagatgaagaaggcgttcgca
ccggaaaatgtagcttcccggttaagtaccttggccatgtagaagttgat
gaatcaagaggaatgcacatctgtgaagatgctgtaaaaagattgaaagc
tactggaaagaaagcagttaaagcagttctgtgggtctcagcagatggac
tcagagttgtggatgaaaaaactaaggacctcatagttgaccagacgata
gagaaagtttctttctgtgccccagacaggaactttgatagagccttttc
ttacatatgccgtgatggcaccactcgtcgctggatctgtcactgcttca
tggctgtcaaggacacaggtgaaaggttgagccatgcagtaggctgtgct
tttgcagcctgtttagagcgcaagcagaagcgggagaaggaatgtggagt
gactgctacttttgatgctagtcggaccacttttacaagagaaggatcat
tccgtgtcacaacagccactgaacaagcagaaagagaggagatcatgaaa
caaatgcaagatgccaagaaagctgaaacagataagatagtcgttggttc
atcagttgcccctggcaacactgccccatccccatcctctcccacctctc
ctacttctgatgccacgacctctctggagatgaacaatcctcatgccatc
ccacgccggcatgctccaattgaacagcttgctcgccaaggctctttccg
aggttttcctgctcttagccagaagatgtcaccctttaaacgccaactat
ccctacgcatcaatgagttgccttccactatgcagaggaagactgatttc
cccattaaaaatgcagtgccagaagtagaaggggaggcagagagcatcag
ctccctgtgctcacagatcaccaatgccttcagcacacctgaggacccct
tctcatctgctccgatgaccaaaccagtgacagtggtggcaccacaatct
cctaccttccaagctaatggcactgactcagccttccatgtgcttgctaa
gccagcccatactgctctagcacccgtagcaatgcctgtgcgtgaaacca
acccttgggcccatgcccctgatgctgctaacaaggaaattgcagccaca
tgttcggggaccgagtggggtcaatcttctggtgctgcctctccaggtct
cttccaggccggtcatagacgtactccctctgaggccgaccgatggttag
aagaggtgtctaagagcgtccgggctcagcagccccaggcctcagctgct
cctctgcagccagttctccagcctcctccacccactgccatctcccagcc
agcatcacctttccaagggaatgcattcctcacctctcagcctgtgccag
tgggtgtggtcccagccctgcaaccagcctttgtccctgcccagtcctat
cctgtggccaatggaatgccctatccagcccctaatgtgcctgtggtggg
catcactccctcccagatggtggccaacgtatttggcactgcaggccacc
ctcaggctgcccatccccatcagtcacccagcctggtcaggcagcagaca
ttccctcactacgaggcaagcagtgctaccaccagtcccttctttaagcc
tcctgctcagcacctcaacggttctgcagctttcaatggtgtagatgatg
gcaggttggcctcagcagacaggcatacagaggttcctacaggcacctgc
ccagtggatccttttgaagcccagtgggctgcattagaaaataagtccaa
gcagcgtactaatccctcccctaccaaccctttctccagtgacttacaga
agacgtttgaaattgaactttaa;
AF171941 (SEQ ID NO: 31):
atgaacaaattacggcaaagttttaggagaaagaaggatgtttatgttcc
agaggccagtcgtccacatcagtggcagacagatgaagaaggcgttcgca
ccggaaaatgtagcttcccggttaagtaccttggccatgtagaagttgat
gaatcaagaggaatgcacatctgtgaagatgctgtaaaaagattgaaagc
tactggaaagaaagcagttaaagcagttctgtgggtctcagcagatggac
tcagagttgtggatgaaaaaactaaggacctcatagttgaccagacgata
gagaaagtttctttctgtgccccagacaggaactttgatagagccttttc
ttacatatgccgtgatggcaccactcgtcgctggatctgtcactgcttca
tggctgtcaaggacacaggtgaaaggttgagccatgcagtaggctgtgct
tttgcagcctgtttagagcgcaagcagaagcgggagaaggaatgtggagt
gactgctactttgatgctagtcggaccacttttacaagagaaggatcatt
ccgtgtcacaacagccactgaacaagcagaaagagaggagatcatgaaac
aaatgcaagatgccaagaaagctgaaacagataagatagtcgttggttca
tcagttgcccctggcaacactgccccatccccatcctctcccacctctcc
tacttctgatgccacgacctctctggagatgaacaatcctcatgccatcc
cacgccggcatgctccaattgaacagcttgctcgccaaggctctttccga
ggttttcctgctcttagccagaagatgtcaccctttaaacgccaactatc
cctacgcatcaatgagttgccttccactatgcagaggaagactgatttcc
ccattaaaaatgcagtgccagaagtagaaggggaggcagagagcatcagc
tccctgtgctcacagatcaccaatgccttcagcacacctgaggacccctt
ctcatctgctccgatgaccaaaccagtgacagtggtggcaccacaatctc
ctaccttccaagggaccgagtggggtcaatcttctggtgctgcctctcca
ggtctcttccaggcggtcatagacgtactccctctgaggccgaccgatgg
ttagaagaggtgtctaagagcgtccgggctcagcagccccaggcctcagc
tgctcctctgcagccagttctccagcctcctccacccactgccatctccc
agccagcatcacctttccaagggaatgcattcctcacctctcagcctgtg
ccagtgggtgtggtcccagccctgcaaccagcctttgtccctgcccagtc
ctatcctgtggccaatggaatgccctatccagcccctaatgtgcctgtgg
tgggcatcactccctcccagatggtggccaacgtatttggcactgcaggc
caccctcaggctgcccatccccatcagtcacccagcctggtcaggcagca
gacattccctcactacgaggcaagcagtgctaccaccagtcccttcttta
agcctcctgctcagcacctcaacggttctgcagctttcaatggtgtagat
gatggcaggttggcctcagcagacaggcatacagaggttcctacaggcac
ctgcccagtggatccttttgaagcccagtgggctgcattagaaaataagt
ccaagcagcgtactaatccctcccctaccaaccctttctccagtgactta
cagaagacgtttgaaattgaactttaa; NM_010949; NM_133287;
BB483123; NM_010950; NM_010949; NM_004756;
DQ022744.
Numblike: NM_00475 (SEQ ID NO: 32):
atgtcccgcagcgcggcggccagcggcggaccccggaggcctgagcggca
cctgcccccagccccctgtggggccccggggcccccagaaacctgcagga
cggagccagacggggcgggcaccatgaacaagttacggcagagcctgcgg
cggaggaagccagcctacgtgcccgaggcgtcgcgcccgcaccagtggca
ggcagacgaggacgcggtgcggaagggcacgtgcagcttcccggtcaggt
acctgggtcacgtggaggtagaggagtcccggggaatgcacgtgtgtgaa
gatgcggtgaagaagctgaaggcgatgggccgaaagtccgtgaagtctgt
cctgtgggtgtcagccgatgggctccgagtggtggacgacaaaaccaagg
atcttctggtcgaccagaccatcgaaaaggtctccttttgtgctcctgac
cgcaacctggacaaggctttctcctatatctgtcgtgacgggactacccg
ccgctggatctgccactgttttctggcactgaaggactccggcgagaggc
tgagccacgctgtgggctgtgcttttgccgcctgcctggagcgaaaacag
cgacgggagaaggaatgtggggtcacggccgccttcgatgccagccgcac
cagcttcgcccgcgagggctccttccgcctgtctgggggtgggcggcctg
ctgagcgagaggccccggacaagaagaaagcagaggcagcagctgccccc
actgtggctcctggccctgcccagcctgggcacgtgtccccgacaccagc
caccacatcccctggtgagaagggtgaggcaggcacccctgtggctgcag
gcaccactgcggccgccatcccccggcgccatgcacccctggagcagctg
gttcgccagggctccttccgtgggttcccagcactcagccagaagaactc
gcctttcaaacggcagctgagcctacggctgaatgagctgccatccacgc
tgcagcgccgcactgacttccaggtgaagggcacagtgcctgagatggag
cctcctggtgccggcgacagtgacagcatcaacgctctgtgcacacagat
cagttcatcttttgccagtgctggagcgccagcaccagggccaccacctg
ccacaacagggacttctgcctggggtgagccctccgtgccccctgcagct
gccttccagcctgggcacaagcggacaccttcagaggctgagcgatggct
ggaggaggtgtcacaggtggccaaggcccagcagcagcagcagcagcaac
agcaacagcagcagcagcagcagcagcaacagcagcaagcagcctcagtg
gccccagtgcccaccatgcctcctgccctgcagcctttccccgcccccgt
ggggccctttgacgctgcacctgcccaagtggccgtgttcctgccacccc
cacacatgcagcccccttttgtgcccgcctacccgggcttgggctaccca
ccgatgccccgggtgcccgtggtgggcatcacaccctcacagatggtggc
aaacgccttctgctcagccgcccagctccagcctcagcctgccactctgc
ttgggaaagctggggccttcccgccccctgccatacccagtgcccctggg
agccaggcccgccctcgccccaatggggccccctggccccctgagccagc
gcctgccccagctccagagttggacccctttgaggcccagtgggcggcat
tagaaggcaaagccactgtagagaaaccctccaaccccttttctggcgac
ctgcaaaagacattcgagattgaactgtag; U964411;
NM_010950; DQ022744.
NANOG: NM_024865 (SEQ ID NO: 33):
atgagtgtggatccagcttgtccccaaagcttgccttgctttgaagcatc
cgactgtaaagaatcttcacctatgcctgtgatttgtgggcctgaagaaa
actatccatccttgcaaatgtcttctgctgagatgcctcacacggagact
gtctctcctcttccttcctccatggatctgcttattcaggacagccctga
ttcttccaccagtcccaaaggcaaacaacccacttctgcagagaagagtg
tcgcaaaaaaggaagacaaggtcccggtcaagaaacagaagaccagaact
gtgttctcttccacccagctgtgtgtactcaatgatagatttcagagaca
gaaatacctcagcctccagcagatgcaagaactctccaacatcctgaacc
tcagctacaaacaggtgaagacctggttccagaaccagagaatgaaatct
aagaggtggcagaaaaacaactggccgaagaatagcaatggtgtgacgca
gaaggcctcagcacctacctaccccagcctttactcttcctaccaccagg
gatgcctggtgaacccgactgggaaccttccaatgtggagcaaccagacc
tggaacaattcaacctggagcaaccagacccagaacatccagtcctggag
caaccactcctggaacactcagacctggtgcacccaatcctggaacaatc
aggcctggaacagtcccttctataactgtggagaggaatctctgcagtcc
tgcatgcagttccagccaaattctcctgccagtgacttggaggctgcctt
ggaagctgctggggaaggccttaatgtaatacagcagaccactaggtatt
ttagtactccacaaaccatggatttattcctaaactactccatgaacatg
caacctgaagacgtgtga; BC137873; NM_028016;
NM_013633; BC160187
OncostatinM(OSM): NM_020530 (SEQ ID NO: 34):
atgggggtactgctcacacagaggacgctgctcagtctggtccttgcact
cctgtttccaagcatggcgagcatggcggctataggcagctgctcgaaag
agtaccgcgtgctccttggccagctccagaagcagacagatctcatgcag
gacaccagcagactcctggacccctatatacgtatccaaggcctggatgt
tcctaaactgagagagcactgcagggagcgccccggggccttccccagtg
aggagaccctgagggggctgggcaggcggggcttcctgcagaccctcaat
gccacactgggctgcgtcctgcacagactggccgacttagagcagcgcct
ccccaaggcccaggatttggagaggtctgggctgaacatcgaggacttgg
agaagctgcagatggcgaggccgaacatcctcgggctcaggaacaacatc
tactgcatggcccagctgctggacaactcagacacggctgagcccacgaa
ggctggccggggggcctctcagccgcccacccccacccctgcctcggatg
cttttcagcgcaagctggagggctgcaggttcctgcatggctaccatcgc
ttcatgcactcagtggggcgggtcttcagcaagtggggggagagcccgaa
ccggagccggagacacagcccccaccaggccctgaggaagggggtgcgca
ggaccagaccctccaggaaaggcaagagactcatgaccaggggacagctg
ccccggtag; NM_001013365; NP_065391; NP_001013383.
OSMR: NM_003999 (SEQ ID NO: 35):
atggctctatttgcagtctttcagacaacattcttcttaacattgctgtc
cttgaggacttaccagagtgaagtcttggctgaacgtttaccattgactc
ctgtatcacttaaagtttccaccaattctacgcgtcagagtttgcactta
caatggactgtccacaaccttccttatcatcaggaattgaaaatggtatt
tcagatccagatcagtaggattgaaacatccaatgtcatctgggtgggga
attacagcaccactgtgaagtggaaccaggttctgcattggagctgggaa
tctgagctccctttggaatgtgccacacactttgtaagaataaagagttt
ggtggacgatgccaagttccctgagccaaatttctggagcaactggagtt
cctgggaggaagtcagtgtacaagattctactggacaggatatattgttc
gttttccctaaagataagctggtggaagaaggcaccaatgttaccatttg
ttacgtttctaggaacattcaaaataatgtatcctgttatttggaaggga
aacagattcatggagaacaacttgatccacatgtaactgcattcaacttg
aatagtgtgcctttcattaggaataaagggacaaatatctattgtgaggc
aagtcaaggaaatgtcagtgaaggcatgaaaggcatcgttctttttgtct
caaaagtacttgaggagcccaaggacttttcttgtgaaaccgaggacttc
aagactttgcactgtacttgggatcctgggacggacactgccttggggtg
gtctaaacaaccttcccaaagctacactttatttgaatcattttctgggg
aaaagaaactttgtacacacaaaaactggtgtaattggcaaataactcaa
gactcacaagaaacctataacttcacactcatagctgaaaattacttaag
gaagagaagtgtcaatatcctttttaacctgactcatcgagtttatttaa
tgaatccttttagtgtcaactttgaaaatgtaaatgccacaaatgccatc
atgacctggaaggtgcactccataaggaataatttcacatatttgtgtca
gattgaactccatggtgaaggaaaaatgatgcaatacaatgtttccatca
aggtgaacggtgagtacttcttaagtgaactggaacctgccacagagtac
atggcgcgagtacggtgtgctgatgccagccacttctggaaatggagtga
atggagtggtcagaacttcaccacacttgaagctgctccctcagaggccc
ctgatgtctggagaattgtgagcttggagccaggaaatcatactgtgacc
ttattctggaagccattatcaaaactgcatgccaatggaaagatcctgtt
ctataatgtagttgtagaaaacctagacaaaccatccagttcagagctcc
attccattccagcaccagccaacagcacaaaactaatccttgacaggtgt
tcctaccaaatctgcgtcatagccaacaacagtgtgggtgcttctcctgc
ttctgtaatagtcatctctgcagaccccgaaaacaaagaggttgaggaag
aaagaattgcaggcacagagggtggattctctctgtcttggaaaccccaa
cctggagatgttataggctatgttgtggactggtgtgaccatacccagga
tgtgctcggtgatttccagtggaagaatgtaggtcccaataccacaagca
cagtcattagcacagatgcttttaggccaggagttcgatatgacttcaga
atttatgggttatctacaaaaaggattgcttgtttattagagaaaaaaac
aggatactctcaggaacttgctccttcagacaaccctcacgtgctggtgg
atacattgacatcccactccttcactctgagttggaaagattactctact
gaatctcaacctggttttatacaagggtaccatgtctatctgaaatccaa
ggcgaggcagtgccacccacgatttgaaaaggcagttctttcagatggtt
cagaatgttgcaaatacaaaattgacaacccggaagaaaaggcattgatt
gtggacaacctaaagccagaatccttctatgagtttttcatcactccatt
cactagtgctggtgaaggccccagtgctacgttcacgaaggtcacgactc
cggatgaacactcctcgatgctgattcatatcctactgcccatggttttc
tgcgtcttgctcatcatggtcatgtgctacttgaaaagtcagtggatcaa
ggagacctgttatcctgacatccctgacccttacaagagcagcatcctgt
cattaataaaattcaaggagaaccctcacctaataataatgaatgtcagt
gactgtatcccagatgctattgaagttgtaagcaagccagaagggacaaa
gatacagttcctaggcactaggaagtcactcacagaaaccgagttgacta
agcctaactacctttatctccttccaacagaaaagaatcactctggccct
ggcccctgcatctgttttgagaacttgacctataaccaggcagcttctga
ctctggctcttgtggccatgttccagtatccccaaaagccccaagtatgc
tgggactaatgacctcacctgaaaatgtactaaaggcactagaaaaaaac
tacatgaactccctgggagaaatcccagctggagaaacaagtttgaatta
tgtgtcccagttggcttcacccatgtttggagacaaggacagtctcccaa
caaacccagtagaggcaccacactgttcagagtataaaatgcaaatggca
gtctccctgcgtcttgccttgcctcccccgaccgagaatagcagcctctc
ctcaattacccttttagatccaggtgaacactactgctaa;
NP_003990.1
OCT3/4(POU5F1): NM_203289 (SEQ ID NO: 36):
atgcacttctacagactattccttggggccacacgtaggttcttgaatcc
cgaatggaaaggggagattgataactggtgtgtttatgttcttacaagtc
ttctgccttttaaaatccagtcccaggacatcaaagctctgcagaaagaa
ctcgagcaatttgccaagctcctgaagcagaagaggatcaccctgggata
tacacaggccgatgtggggctcaccctgggggttctatttgggaaggtat
tcagccaaacgaccatctgccgctttgaggctctgcagcttagcttcaag
aacatgtgtaagctgcggcccttgctgcagaagtgggtggaggaagctga
caacaatgaaaatcttcaggagatatgcaaagcagaaaccctcgtgcagg
cccgaaagagaaagcgaaccagtatcgagaaccgagtgagaggcaacctg
gagaatttgttcctgcagtgcccgaaacccacactgcagcagatcagcca
catcgcccagcagcttgggctcgagaaggatgtggtccgagtgtggttct
gtaaccggcgccagaagggcaagcgatcaagcagcgactatgcacaacga
gaggattttgaggctgctgggtctcctttctcagggggaccagtgtcctt
tcctctggccccagggccccattttggtaccccaggctatgggagccctc
acttcactgcactgtactcctcggtccctttccctgagggggaagccttt
ccccctgtctccgtcaccactctgggctctcccatgcattcaaactga;
NM_002701 (SEQ ID NO: 37):
atggcgggacacctggcttcggatttcgccttctcgccccctccaggtgg
tggaggtgatgggccaggggggccggagccgggctgggttgatcctcgga
cctggctaagcttccaaggccctcctggagggccaggaatcgggccgggg
gttgggccaggctctgaggtgtgggggattcccccatgccccccgccgta
tgagttctgtggggggatggcgtactgtgggccccaggttggagtggggc
tagtgccccaaggcggcttggagacctctcagcctgagggcgaagcagga
gtcggggtggagagcaactccgatggggcctccccggagccctgcaccgt
cacccctggtgccgtgaagctggagaaggagaagctggagcaaaacccgg
aggagtcccaggacatcaaagctctgcagaaagaactcgagcaatttgcc
aagctcctgaagcagaagaggatcaccctgggatatacacaggccgatgt
ggggctcaccctgggggttctatttgggaaggtattcagccaaacgacca
tctgccgctttgaggctctgcagcttagcttcaagaacatgtgtaagctg
cggcccttgctgcagaagtgggtggaggaagctgacaacaatgaaaatct
tcaggagatatgcaaagcagaaaccctcgtgcaggcccgaaagagaaagc
gaaccagtatcgagaaccgagtgagaggcaacctggagaatttgttcctg
cagtgcccgaaacccacactgcagcagatcagccacatcgcccagcagct
tgggctcgagaaggatgtggtccgagtgtggttctgtaaccggcgccaga
agggcaagcgatcaagcagcgactatgcacaacgagaggattttgaggct
gctgggtctcctttctcagggggaccagtgtcctttcctctggccccagg
gccccattttggtaccccaggctatgggagccctcacttcactgcactgt
actcctcggtccctttccctgagggggaagcctttccccctgtctccgtc
accactctgggctctcccatgcattcaaactga; NM_013633;
EF032593; NM_131112; NM_001114955.
SOX2: NM_003106 (SEQ ID NO: 38):
atgtacaacatgatggagacggagctgaagccgccgggcccgcagcaaac
ttcggggggcggcggcggcaactccaccgcggcggcggccggcggcaacc
agaaaaacagcccggaccgcgtcaagcggcccatgaatgccttcatggtg
tggtcccgcgggcagcggcgcaagatggcccaggagaaccccaagatgca
caactcggagatcagcaagcgcctgggcgccgagtggaaacttttgtcgg
agacggagaagcggccgttcatcgacgaggctaagcggctgcgagcgctg
cacatgaaggagcacccggattataaataccggccccggcggaaaaccaa
gacgctcatgaagaaggataagtacacgctgcccggcgggctgctggccc
ccggcggcaatagcatggcgagcggggtcggggtgggcgccggcctgggc
gcgggcgtgaaccagcgcatggacagttacgcgcacatgaacggctggag
caacggcagctacagcatgatgcaggaccagctgggctacccgcagcacc
cgggcctcaatgcgcacggcgcagcgcagatgcagcccatgcaccgctac
gacgtgagcgccctgcagtacaactccatgaccagctcgcagacctacat
gaacggctcgcccacctacagcatgtcctactcgcagcagggcacccctg
gcatggctcttggctccatgggttcggtggtcaagtccgaggccagctcc
agcccccctgtggttacctcttcctcccactccagggcgccctgccaggc
cggggacctccgggacatgatcagcatgtatctccccggcgccgaggtgc
cggaacccgccgcccccagcagacttcacatgtcccagcactaccagagc
ggcccggtgcccggcacggccattaacggcacactgcccctctcacacat
gtga; NM_011443; NM_00110918.
FGF4: NM_002007 (SEQ ID NO: 39):
atgtcggggcccgggacggccgcggtagcgctgctcccggcggtcctgct
ggccttgctggcgccctgggcgggccgagggggcgccgccgcacccactg
cacccaacggcacgctggaggccgagctggagcgccgctgggagagcctg
gtggcgctctcgttggcgccgcctgccggtggcagcgcagcccaaggagg
cggccgtccagagcggcgccggcgactacctgctgggcatcaagcggctg
cggcggctctactgcaacgtgggcatcggcttccacctccaggcgctccc
cgacggccgcatcggcggcgcgcacgcggacacccgcgacagcctgctgg
agctctcgcccgtggagcggggcgtggtgagcatcttcggcgtggccagc
cggttcttcgtggccatgagcagcaagggcaagctctatggctcgccctt
cttcaccgatgagtgcacgttcaaggagattctccttcccaacaactaca
acgcctacgagtcctacaagtaccccggcatgttcatcgccctgagcaag
aatgggaagaccaagaaggggaaccgagtgtcgcccaccatgaaggtcac
ccacttcctccccaggctgtg; NM_010202; NM_004380;
NM_001025432; NM_004379.2; NP_004370; NM_134442;
NP_604391
Gata2: NM_032638 (SEQ ID NO: 40):
atggaggtggcgccggagcagccgcgctggatggcgcacccggccgtgct
gaatgcgcagcaccccgactcacaccacccgggcctggcgcacaactaca
tggaacccgcgcagctgctgcctccagacgaggtggacgtcttcttcaat
cacctcgactcgcagggcaacccctactatgccaaccccgctcacgcgcg
ggcgcgcgtctcctacagccccgcgcacgcccgcctgaccggaggccaga
tgtgccgcccacacttgttgcacagcccgggtttgccctggctggacggg
ggcaaagcagccctctctgccgctgcggcccaccaccacaacccctggac
cgtgagccccttctccaagacgccactgcacccctcagctgctggaggcc
ctggaggcccactctctcgtgtacccaggggctgggggtgggagcggggg
aggcagcgggagctcagtggcctccctcacccctacagcagcccactctg
gctcccaccttttcggcttcccacccacgccacccaaagaagtgtctcct
gaccctagcaccacgggggctgcgtctccagcctcatcttccgcgggggg
tagtgcagcccgaggagaggacaaggacggcgtcaagtaccaggtgtcac
tgacggagagcatgaagatggaaagtggcagtcccctgcgcccaggccta
gctactatgggcacccagcctgctacacaccaccccatccccacctaccc
ctcctatgtgccggcggctgcccacgactacagcagcggactcttccacc
ccggaggcttcctggggggaccggcctccagcttcacccctaagcagcgc
agcaaggctcgttcctgttcagaaggccgggagtgtgtcaactgtggggc
cacagccacccctctctggcggcgggacggcaccggccactacctgtgca
atgcctgtggcctctaccacaagatgaatgggcagaaccgaccactcatc
aagcccaagcgaagactgtcggccgccagaagagccggcacctgttgtgc
aaattgtcagacgacaaccaccaccttatggcgccgaaacgccaacgggg
accctgtctgcaacgcctgtggcctctactacaagctgcacaatgttaac
aggccactgaccatgaagaaggaagggatccagactcggaaccggaagat
gtccaacaagtccaagaagagcaagaaaggggcggagtgcttcgaggagc
tgtcaaagtgcatgcaggagaagtcatcccccttcagtgcagctgccctg
gctggacacatggcacctgtgggccacctcccgcccttcagccactccgg
acacatcctgcccactccgacgcccatccacccctcctccagcctctcct
tcggccacccccacccgtccagcatggtgaccgccatgggctag;
AB102789; AB102790; NM_008090.
Gata3: NM_001002295 (SEQ ID NO: 41):
atggaggtgacggcggaccagccgcgctgggtgagccaccaccaccccgc
cgtgctcaacgggcagcacccggacacgcaccacccgggcctcagccact
cctacatggacgcggcgcagtacccgctgccggaggaggtggatgtgctt
tttaacatcgacggtcaaggcaaccacgtcccgccctactacggaaactc
ggtcagggccacggtgcagaggtaccctccgacccaccacgggagccagg
tgtgccgcccgcctctgcttcatggatccctaccctggctggacggcggc
aaagccctgggcagccaccacaccgcctccccctggaatctcagcccctt
ctccaagacgtccatccaccacggctccccggggcccctctccgtctacc
ccccggcctcgtcctcctccttgtcggggggccacgccagcccgcacctc
ttcaccttcccgcccaccccgccgaaggacgtctccccggacccatcgct
gtccaccccaggctcggccggctcggcccggcaggacgagaaagagtgcc
tcaagtaccaggtgcccctgcccgacagcatgaagctggagtcgtcccac
tcccgtggcagcatgaccgccctgggtggagcctcctcgtcgacccacca
ccccatcaccacctacccgccctacgtgcccgagtacagctccggactct
tcccccccagcagcctgctgggcggctcccccaccggcttcggatgcaag
tccaggcccaaggcccggtccagcacagaaggcagggagtgtgtgaactg
tggggcaacctcgaccccactgtggcggcgagatggcacgggacactacc
tgtgcaacgcctgcgggctctatcacaaaatgaacggacagaaccggccc
ctcattaagcccaagcgaaggctgtctgcagccaggagagcagggacgtc
ctgtgcgaactgtcagaccaccacaaccacactctggaggaggaatgcca
atggggaccctgtctgcaatgcctgtgggctctactacaagcttcacaat
attaacagacccctgactatgaagaaggaaggcatccagaccagaaaccg
aaaaatgtctagcaaatccaaaaagtgcaaaaaagtgcatgactcactgg
aggacttccccaagaacagctcgtttaacccggccgccctctccagacac
atgtcctccctgagccacatctcgcccttcagccactccagccacatgct
gaccacgcccacgccgatgcacccgccatccagcctgtcctttggaccac
accacccctccagcatggtcaccgccatgggttag; NM_08091;
AM392688; AM392571; NM_001002295; NM_002051
Gata4: BC101580 (SEQ ID NO: 42):
atgtatcagagcttggccatggccgccaaccacgggccgccccccggtgc
ctacgaggcgggcggccccggcgccttcatgcacggcgcgggcgccgcgt
cctcgccagtctacgtgcccacaccgcgggtgccctcctccgtgctgggc
ctgtcctacctccagggcggaggcgcgggctctgcgtccggaggcgcctc
gggcggcagctccggtggggccgcgtctggtgcggggcccgggacccagc
agggcagcccgggatggagccaggcgggagccgacggagccgcttacacc
ccgccgccggtgtcgccgcgcttctccttcccggggaccaccgggtccct
ggcggccgccgccgccgctgccgcggcccgggaagctgcggcctacagca
gtggcggcggagcggcgggtgcgggcctggcgggccgcgagcagtacggg
cgcgccggcttcgcgggctcctactccagcccctacccggcttacatggc
cgacgtgggcgcgtcctgggccgcagccgccgccgcctccgccggcccct
tcgacagcccggtcctgcacagcctgcccggccgggccaacccggccgcc
cgacaccccaatctcgatatgtttgacgacttctcagaaggcagagagtg
tgtcaactgtggggctatgtccaccccgctctggaggcgagatgggacgg
gtcactatctgtgcaacgcctgcggcctctaccacaagatgaacggcatc
aaccggccgctcatcaagcctcagcgccggctgtccgcctcccgccgagt
gggcctctcctgtgccaactgccagaccaccaccaccacgctgtggcgcc
gcaatgcggagggcgagcctgtgtgcaatgcctgcggcctctacatgaag
ctccacggggtccccaggcctcttgcaatgcggaaagaggggatccaaac
cagaaaaacggaagcccaagaacctgaataaatctaagacaccagcagct
ccttcaggcagtgagagccttcctcccgccagcggtgcttccagcaactc
cagcaacgccaccaccagcagcagcgaggagatgcgtcccatcaagacgg
agcctggcctgtcatctcactacgggcacagcagctccgtgtcccagacg
ttctcagtcagtgcgatgtctggccatgggccctccatccaccctgtcct
ctcggccctgaagctctccccacaaggctatgcgtctcccgtcagccagt
ctccacagaccagctccaagcaggactcttggaacagcctggtcttggcc
gacagtcacggggacataatcactgcgtaa; AF179424;
DQ666280.
Gata5: BC117356 (SEQ ID NO: 43):
atgtaccagagcctggcgctggccgcgagcccccgccaggccgcctacgc
cgactcgggctccttcctgcacgctccgggcgccggctctccgatgtttg
tgccgccggcgcgcgtcccctcgatgctgtcctacctgtccgggtgtgag
ccgagcccgcagccccccgagctcgctgcgcgccccggctgggcgcagac
agccaccgcggattcgtcggccttcggcccgggcagtccgcaccccccag
ccgcgcacccgcccggggccaccgccttccctttcgcgcacagcccctcg
gggcccggcagcggcggcagcgcggggggccgagacggcagtgcctacca
gggcgcgctgttgcctcgagaacagttcgcggccccgcttgggcggccgg
tggggacctcgtactccgccacctacccggcctacgtgagccccgacgtg
gcccagtcctggactgccgggcccttcgatggcagcgtcctgcacggcct
cccaggccgcaggcccaccttcgtgtccgacttcttggaggagttcccgg
gtgagggtcgtgagtgtgtcaactgcggggccctgtccacaccgctgtgg
cgccgagatggcaccggccactacctgtgcaatgcctgcggcctctacca
caagatgaatggcgtcaaccggccgctcgttcggcctcagaagcgcctgt
cctcgtcccgccgcgccggcctctgctgcaccaactgccacacgaccaac
accacgctgtggcggcggaactcggagggggagcccgtgtgcaatgcctg
cggcctctacatgaagctgcacggggtgccgcggcctctggctatgaaga
aagaaagcatccagacacggaagcggaagccaaagaccatcgccaaggcc
aggggctcctcaggatccacaaggaatgcctcggcctccccatctgctgt
cgccagcactgacagctcagcagccacttccaaagccaagcccagcctgg
cgtccccagtgtgccctgggcccagcatggccccccaggcctctggccag
gaggatgactctcttgcccccggccacttggagttcaagttcgagcctga
ggactttgccttcccctccacggccccgagcccccaggctggcctcaggg
gggctctgcgccaagaggcctggtgtgcgctggccttggcctag;
BC105654.
Gata6: NM_005257 (SEQ ID NO: 44):
atggccttgactgacggcggctggtgcttgccgaagcgcttcggggccgc
gggtgcggacgccagcgactccagagcctttccagcgcgggagccctcca
cgccgccttcccccatctcttcctcgtcctcctcctgctcccggggcgga
gagcggggccccggcggcgccagcaactgcgggacgcctcagctcgacac
ggaggcggcggccggacccccggcccgctcgctgctgctcagttcctacg
cttcgcatcccttcggggctccccacggaccttcggcgcctggggtcgcg
ggccccgggggcaacctgtcgagctgggaggacttgctgctgttcactga
cctcgaccaagccgcgaccgccagcaagctgctgtggtccagccgcggcg
ccaagctgagccccttcgcacccgagcagccggaggagatgtaccagacc
ctcgccgctctctccagccagggtccggccgcctacgacggcgcgcccgg
cggcttcgtgcactctgcggccgcggcggcagcagccgcggcggcggcca
gctccccggtctacgtgcccaccacccgcgtgggttccatgctgcccggc
ctaccgtaccacctgcaggggtcgggcagtgggccagccaaccacgcggg
cggcgcgggcgcgcaccccggctggcctcaggcctcggccgacagccctc
catacggcagcggaggcggcgcggctggcggcggggccgcggggcctggc
ggcgctggctcagccgcggcgcacgtctcggcgcgcttcccctactctcc
cagcccgcccatggccaacggcgccgcgcgggagccgggaggctacgcgg
cggcgggcagtgggggcgcgggaggcgtgagcggcggcggcagtagcctg
gcggccatgggcggccgcgagccccagtacagctcgctgtcggccgcgcg
gccgctgaacgggacgtaccaccaccaccaccaccaccaccaccaccatc
cgagcccctactcgccctacgtgggggcgccactgacgcctgcctggccc
gccggacccttcgagaccccggtgctgcacagcctgcagagccgcgccgg
agccccgctcccggtgccccggggtcccagtgcagacctgctggaggacc
tgtccgagagccgcgagtgcgtgaactgcggctccatccagacgccgctg
tggcggcgggacggcaccggccactacctgtgcaacgcctgcgggctcta
cagcaagatgaacggcctcagccggcccctcatcaagccgcagaagcgcg
tgccttcatcacggcggcttggattgtcctgtgccaactgtcacaccaca
actaccaccttatggcgcagaaacgccgagggtgaacccgtgtgcaatgc
ttgtggactctacatgaaactccatggggtgcccagaccacttgctatga
aaaaagagggaattcaaaccaggaaacgaaaacctaagaacataaataaa
tcaaagacttgctctggtaatagcaataattccattcccatgactccaac
ttccacctcttctaactcagatgattgcagcaaaaatacttcccccacaa
cacaacctacagcctcaggggcgggtgccccggtgatgactggtgcggga
gagagcaccaatcccgagaacagcgagctcaagtattcgggtcaagatgg
gctctacataggcgtcagtctcgcctcgccggccgaagtcacgtcctccg
tgcgaccggattcctggtgcgccctggccctggcctga; AF179425;
EF444980; NM_005257; NP_005248.
HNF1: NM_000458 (SEQ ID NO: 45):
atggtgtccaagctcacgtcgctccagcaagaactcctgagcgccctgct
gagctccggggtcaccaaggaggtgctggttcaggccttggaggagttgc
tgccatccccgaacttcggggtgaagctggagacgctgcccctgtcccct
ggcagcggggccgagcccgacaccaagccggtcttccatactctcaccaa
cggccacgccaagggccgcttgtccggcgacgagggctccgaggacggcg
acgactatgacacacctcccatcctcaaggagctgcaggcgctcaacacc
gaggaggcggcggagcagcgggcggaggtggaccggatgctcagtgagga
cccttggagggctgctaaaatgatcaagggttacatgcagcaacacaaca
tcccccagagggaggtggtcgatgtcaccggcctgaaccagtcgcacctc
tcccagcatctcaacaagggcacccctatgaagacccagaagcgtgccgc
tctgtacacctggtacgtcagaaagcaacgagagatcctccgacaattca
accagacagtccagagttctggaaatatgacagacaaaagcagtcaggat
cagctgctgtttctctttccagagttcagtcaacagagccatgggcctgg
gcagtccgatgatgcctgctctgagcccaccaacaagaagatgcgccgca
accggttcaaatgggggcccgcgtcccagcaaatcttgtaccaggcctac
gatcggcaaaagaaccccagcaaggaagagagagaggccttagtggagga
atgcaacagggcagaatgtttgcagcgaggggtgtccccctccaaagccc
acggcctgggctccaacttggtcactgaggtccgtgtctacaactggttt
gcaaaccgcaggaaggaggaggcattccggcaaaagctggccatggacgc
ctatagctccaaccagactcacagcctgaaccctctgctctcccacggct
ccccccaccaccagcccagctcctctcctccaaacaagctgtcaggagtg
cgctacagccagcagggaaacaatgagatcacttcctcctcaacaatcag
tcaccatggcaacagcgccatggtgaccagccagtcggttttacagcaag
tctccccagccagcctggacccaggccacaatctcctctcacctgatggt
aaaatgatctcagtctcaggaggaggtttgcccccagtcagcaccttgac
gaatatccacagcctctcccaccataatccccagcaatctcaaaacctca
tcatgacacccctctctggagtcatggcaattgcacaaagcctcaacacc
tcccaagcacagagtgtccctgtcatcaacagtgtggccggcagcctggc
agccctgcagcccgtccagttctcccagcagctgcacagccctcaccagc
agcccctcatgcagcagagcccaggcagccacatggcccagcagcccttc
atggcagctgtgactcagctgcagaactcacacatgtacgcacacaagca
ggaacccccccagtattcccacacctcccggtttccatctgcaatggtgg
tcacagataccagcagcatcagtacactcaccaacatgtcttcaagtaaa
cagtgtcctctacaagcctggtga; NM_013103; NM_000454;
NM_009327;
NM_012669 (SEQ ID NO: 46):
atggtttctaagttgagccagctgcagacggagctcctggctgctctgct
cgagtcgggcctgagcaaagaggctctgatccaggctctgggggagcccg
ggccctacctgatggttggagatggtcccctggacaagggggagtcctgc
ggtgggactcgaggggacctgaccgagctgcccaatggcctgggggagac
gcgtggctcggaagatgacacggatgacgatggggaagacttcgcgccac
ccattctgaaagagctggagaacctcagcccagaggaggcagcccaccag
aaagccgtggtggagtcacttcttcaggaggacccatggcgcgtggcaaa
gatggtcaagtcgtacctgcagcaacacaacatcccccagcgggaggtgg
tggacactacgggtctcaaccagtcccacctgtcccagcacctcaacaag
ggcacccccatgaagacgcagaagcgggccgcgctgtacacctggtacgt
ccgcaagcagcgagaggtggctcagcaattcacccacgcggggcagggcg
gactgattgaagagcccacaggtgatgagctgccaaccaaaaaggggcgg
aggaaccggttcaagtggggccccgcatcccagcagatcctgttccaggc
ttacgagaggcagaagaaccccagcaaggaagagcgagagaccttggtgg
aggagtgcaatagggcggagtgcatccagagaggggtgtcaccatcgcag
gcccaggggctaggctccaaccttgtcaccgaggtgcgtgtctacaactg
gtttgccaaccggcgcaaggaagaagcctttcggcataagctggccatgg
acacgtataacgggcctccacccgggccaggccccggccctgcgctacct
gcccacagttccccgggcctgcccacaaccaccctctctcccagtaaggt
ccacggtgtgcggtatggacagtctgcaaccagcgaggcagctgaggtgc
cctccagcagcggaggtcccttagtcacagtgtctgcggccttacaccaa
gtgtcccccacaggcttggagcccagcagcctgctgagcaccgaggccaa
gctggtctcagccacggggggtcccctgcctcccgtcagcaccctgacag
cactgcacagcttggagcagacgtctccaggtctcaaccagcagccgcag
aaccttatcatggcctcgctgcctggggtcatgaccatcggcccagggga
gcccgcctccctgggtcccacgttcactaacacgggtgcctctaccctgg
tcattggtctggcctccacacaggcacagagctgtccagtcatcaacagc
atggggagcagcctgaccaccctgcagccggtccagttttcccagccact
gcacccttcctatcagcagcctctcatgccccctgtacagagccacgtgg
cccagagtcccttcatggcaaccatggcccagctgcagagcccccacgcc
ctgtacagccacaagcctgaggtggcccagtacacgcatacaagcctgct
tccgcagaccatgctgatcacagacaccaacctcagcacccttgccagcc
tcacgcccaccaagcaggtcttcacctcagacacagaggcctccagtgag
cctgggcttcatgagccgtcgtctccagccacaaccattcacatccccag
ccaggacccgtcaaacatccagcacctgcagcctgctcaccggctcagca
ccagtcccacagtgtcctccagcagcctggtgttgtaccagagttctgac
tccaacgggcacagccacctgctgccatccaaccacggtgtcatcgagac
ttttatctccacccagatggcctcctcctcccagtaa; NM_009330.
HNF3: X74936 (SEQ ID NO: 47):
atgttagggactgtgaagatggaagggcatgagagcaacgactggaacag
ctactacgcggacacgcaggaggcctactcctctgtccctgtcagcaaca
tgaactccggcctgggctctatgaactccatgaacacctacatgaccatg
aacaccatgaccacgagcggcaacatgaccccggcttccttcaacatgtc
ctacgccaacacgggcttaggggccggcctgagtcccggtgctgtggctg
gcatgccaggggcctctgcaggcgccatgaacagcatgactgcggcgggc
gtcacggccatgggtacggcgctgagcccgggaggcatgggctccatggg
cgcgcagcccgtcacctccatgaacggcctgggtccctacgccgccgcca
tgaacccgtgcatgagtcccatggcgtacgcgccgtccaacctgggccgc
agccgcgcggggggcggcggcgacgccaagacattcaagcgcagctaccc
tcacgccaagccgccttactcctacatctcgctcatcacgatggccatcc
agcaggcgcccagcaagatgctcacgctgagcgagatctaccagtggatc
atggacctcttcccctattaccgccagaaccagcagcgctggcagaactc
catccgccactcgctgtccttcaacgattgtttcgtcaaggtggcacgat
ccccagacaagccaggcaagggctcctactggacgctgcacccggactcc
ggcaacatgttcgagaacggctgctacttgcgccgccaaaagcgcttcaa
gtgtgagaagcagccgggggccggaggtgggagtgggggcggcggctcca
aagggggcccagaaagtcgcaaggacccctcaggcccggggaaccccagc
gccgagtcaccccttcattggggtgtgcacggaaaggctagccagctaga
gggcgcgccggcccccgggcccgccgccagcccccagactctggaccaca
gcggggccacggcgacagggggcgcttcggagttgaagtctccagcgtct
tcatctgcgccccccataagctccgggccaggggcgctggcatctgtacc
cccctctcacccggctcacggcctggcaccccacgaatctcagctgcatc
tgaaaggggatccccactactcctttaatcaccccttctccatcaacaac
ctcatgtcctcctccgagcaacagcacaagctggacttcaaggcatacga
gcaggcgctgcagtactctccttatggcgctaccttgcccgccagtctgc
cccttggcagcgcctcagtggccacgaggagccccatcgagccctcagcc
ctggagccagcctactaccaaggtgtgtattccagacccgtgctaaatac
ttcctag
HNF3gammaX74938M (SEQ ID NO: 48):
atgctgggctcagtgaagatggaggctcatgacctggccgagtggagcta
ctacccggaggcgggcgaggtgtattctccagtgaatcctgtgcccacca
tggcccctctcaactcctacatgaccttgaacccactcagctctccctac
cctcccggagggcttcaggcctccccactgcctacaggacccctggcacc
cccagcccccactgcgcccttggggcccaccttcccaagcttgggcactg
gtggcagcaccggaggcagtgcttccgggtatgtagccccagggcccggg
cttgtacatggaaaagagatggcaaaggggtaccggcggccactggccca
cgccaaaccaccatattcctacatctctctcataaccatggctattcagc
aggctccaggcaagatgctgaccctgagtgaaatctaccaatggatcatg
gacctcttcccgtactaccgggagaaccagcaacgttggcagaactccat
ccggcattcgctgtccttcaatgactgcttcgtcaaggtggcacgctccc
cagacaagccaggcaaaggctcctactgggccttgcatcccagctctggg
aacatgtttgagaacggatgctatctccgccggcagaagcgcttcaagct
ggaggagaaggcaaagaaaggaaacagcgccacatcggccagcaggaatg
gtactgcggggtcagccacctctgccaccactacagctgccactgcagtc
acctccccggctcagccccagcctacgccatctgagcccgaggcccagag
tggggatgatgtggggggtctggactgcgcctcacctccttcgtccacac
cttatttcagcggcctggagctcccgggggaactaaagttggatgcgccc
tataacttcaaccaccctttctctatcaacaacctgatgtcagaacagac
atcgacaccttccaaactggatgtggggtttgggggctacggggctgaga
gtggggagcctggagtctactaccagagcctctattcccgctctctgctt
aatgcatcctag
HNF3betaX74937 (SEQ ID NO: 49):
atgctgggagccgtgaagatggaagggctcgagccatccgactggagcag
ctactacgcggagcccgagggctactcttccgtgagcaacatgaacgccg
gcctggggatgaatggcatgaacacatacatgagcatgtccgcggctgcc
atgggcggcggttccggcaacatgagcgcgggctccatgaacatgtcatc
ctatgtgggcgctggaatgagcccgtcgctagctggcatgtccccgggcg
ccggcgccatggcgggcatgagcggctcagccggggcggccggcgtggcg
ggcatgggacctcacctgagtccgagtctgagcccgctcgggggacaggc
ggccggggccatgggtggccttgccccctacgccaacatgaactcgatga
gccccatgtacgggcaggccggcctgagccgcgctcgggaccccaagaca
taccgacgcagctacacacacgccaaacctccctactcgtacatctcgct
catcaccatggccatccagcagagccccaacaagatgctgacgctgagcg
agatctatcagtggatcatggacctcttccctttctaccggcagaaccag
cagcgctggcagaactccatccgccactctctctccttcaacgactgctt
tctcaaggtgccccgctcgccagacaagcctggcaagggctccttctgga
ccctgcacccagactcgggcaacatgttcgagaacggctgctacctgcgc
cgccagaagcgcttcaagtgtgagaagcaactggcactgaaggaagccgc
gggtgcggccagtagcggaggcaagaagaccgctcctgggtcccaggcct
ctcaggctcagctcggggaggccgcgggctcggcctccgagactccggcg
ggcaccgagtccccccattccagcgcttctccgtgtcaggagcacaagcg
aggtggcctaagcgagctaaagggagcacctgcctctgcgctgagtcctc
ccgagccggcgccctcgcctgggcagcagcagcaggctgcagcccacctg
ctgggcccacctcaccacccaggcctgccaccagaggcccacctgaagcc
cgagcaccattacgccttcaaccaccccttctctatcaacaacctcatgt
cgtccgagcagcaacatcaccacagccaccaccaccatcagccccacaaa
atggacctcaaggcctacgaacaggtcatgcactacccagggggctatgg
ttcccccatgccaggcagcttggccatgggcccagtcacgaacaaagcgg
gcctggatgcctcgcccctggctgcagacacttcctactaccaaggagtg
tactccaggcctattatgaactcatcctaa
HNF3G: AH008133 (SEQ ID NO: 50):
atgctgggctcagtgaagatggaggcccatgacctggccgagtggagcta
ctacccggaggcgggcgaggtctactcgccggtgaccccagtgcccacca
tggcccccctcaactcctacatgaccctgaatcctctaagctctccctat
ccccctggggggctccctgcctccccactgccctcaggacccctggcacc
cccagcacctgcagcccccctggggcccactttcccaggcctgggtgtca
gcggtggcagcagcagctccgggtacggggccccgggtcctgggctggtg
cacgggaaggagatgccgaaggggtatcggcggcccctggcacacgccaa
gccaccgtattcctatatctcactcatcaccatggccatccagcaggcgc
cgggcaagatgctgaccttgagtgaaatctaccagtggatcatggaccta
tcccttactaccgggagaatcagcagcgctggcagaactccattcgccac
tcgctgtctttcaacgactgcttcgtcaaggtggcgcgttccccagacaa
gcctggcaagggctcctactgggccctacaccccagctcagggaacatgt
ttgagaatggctgctacctgcgccgccagaaacgcttcaagctggaggag
aaggtgaaaaaagggggcagcggggctgccaccaccaccaggaacgggac
agggtctgctgcctcgaccaccacccccgcggccacagtcacctccccgc
cccagcccccgcctccagcccctgagcctgaggcccagggcggggaagat
gtgggggctctggactgtggctcacccgcttcctccacaccctatttcac
tggcctggagctcccaggggagctgaagctggacgcgccctacaacttca
accaccctttctccatcaacaacctaatgtcagaacagacaccagcacct
cccaaactggacgtggggtttgggggctacggggctgaaggtggggagcc
tggagtctactaccagggcctctattcccgctctttgcttaatgcatcct
ag
HNF3A: AH008132 (SEQ ID NO: 51):
atgttaggaactgtgaagatggaagggcatgaaaccagcgactggaacag
ctactacgcagacacgcaggaggcctactcctcggtcccggtcagcaaca
tgaactcaggcctgggctccatgaactccatgaacacctacatgaccatg
aacaccatgactacgagcggcaacatgaccccggcgtccttcaacatgtc
ctatgccaacccggccttaggggccggcctgagtcccggcgcagtagccg
gcatgccggggggctcggcgggcgccatgaacagcatgactgcggccggc
gtgacggccatgggtacggcgctgagcccgagcggcatgggcgccatggg
tgcgcagcaggcggcctccatgatgaatggcctgggcccctacgcggccg
ccatgaacccgtgcatgagccccatggcgtacgcgccgtccaacctgggc
cgcagccgcgcgggcggcggcggcgacgccaagacgttcaagcgcagtta
cccgcacgccaagccgccctactcgtacatctcgctcatcaccatggcca
tccagcgggcgcccagcaagatgctcacgctgagcgagatctaccagtgg
atcatggacctcttcccctattaccggcagaaccagcagcgctggcagaa
ctccatccgccactcgctgtccttcaatgactgcttcgtcaaggtggcac
gctccccggacaagccgggcaagggctcctactggacgctgcacccggac
tccggcaacatgttcgagaacggctgctacttgcgccgccagaagcgctt
caagtgcgagaagcagccgggggccggcggcgggggcgggagcggaagcg
ggggcagcggcgccaagggcggccctgagagccgcaaggacccctctggc
gcctctaaccccagcgccgactcgcccctccatcggggtgtgcacgggaa
gaccggccagctagagggcgcgccggccccgggcccggccgccagccccc
agactctggaccacagtggggcgacggcgacagggggcgcctcggagttg
aagactccagcctcctcaactgcgccccccataagctccgggcccggggc
gctggcctctgtgcccgcctctcacccggcacacggcttggcaccccacg
agtcccagctgcacctgaaaggggacccccactactcatcaaccacccgt
tctccatcaacaacctcatgtcctcctcggagcagcagcataagctggac
ttcaaggcatacgaacaggcactgcaatactcgccttacggctctacgtt
gcccgccagcctgcctctaggcagcgcctcggtgaccaccaggagcccca
tcgagccctcagccctggagccggcgtactaccaaggtgtgtattccaga
cccgtcctaaacacttcctag
HNF4alpha: NM_008261 (SEQ ID NO: 52):
atgcgactctctaaaacccttgccggcatggatatggccgactacagcgc
tgccctggacccagcctacaccaccctggagtttgaaaatgtgcaggtgt
tgaccatgggcaatgacacgtccccatctgaaggtgccaacctcaattca
tccaacagcctgggcgtcagtgccctgtgcgccatctgtggcgaccgggc
caccggcaaacactacggagcctcgagctgtgacggctgcaaggggttct
tcaggaggagcgtgaggaagaaccacatgtactcctgcaggtttagccga
caatgtgtggtagacaaagataagaggaaccagtgtcgttactgcaggct
taagaagtgcttccgggctggcatgaagaaggaagctgtccaaaatgagc
gggaccggatcagcacgcggaggtcaagctacgaggacagcagcctgccc
tccatcaacgcgctcctgcaggcagaggttctgtcccagcagatcacctc
tcccatctctgggatcaatggcgacattcgggcaaagaagattgccaaca
tcacagacgtgtgtgagtctatgaaggagcagctgctggtcctggtcgag
tgggccaagtacatcccggccttctgcgaactccttctggatgaccaggt
ggcgctgctcagggcccacgccggtgagcatctgctgcttggagccacca
agaggtccatggtgtttaaggacgtgctgctcctaggcaatgactacatc
gtccctcggcactgtccagagctagcggagatgagccgtgtgtccatccg
catcctcgatgagctggtcctgcccttccaagagctgcagattgatgaca
atgaatatgcctgcctcaaagccatcatcttctttgatccagatgccaag
gggctgagtgacccgggcaagatcaagcggctgcggtcacaggtgcaagt
gagcctggaggattacatcaacgaccggcagtacgactctcggggccgct
ttggagagctgctgctgctgttgcccacgctgcagagcatcacctggcag
atgatcgaacagatccagttcatcaagctcttcggcatggccaagattga
caacctgctgcaggagatgcttctcggagggtctgccagtgatgcacccc
acacccaccaccccctgcaccctcacctgatgcaagaacacatgggcacc
aatgtcattgttgctaacacgatgccctctcacctcagcaatggacagat
gtgtgagtggccccgacccagggggcaggcagccactcccgagactccac
agccatcaccaccaagtggctcgggatctgaatcctacaagctcctgcca
ggagccatcaccaccatcgtcaagcctccctctgccattccccagccaac
gatcaccaagcaagaagccatctag
HNF4a: NM_022180 (SEQ ID NO: 53):
atggacatggctgactacagtgctgccttggacccagcctacaccaccct
ggagtttgaaaatgtgcaggtgttgaccatgggcaatgacacatccccat
ctgaaggtgccaacctcaactcatccaacagcctgggtgtcagtgccctg
tgtgccatctgtggcgatcgggccactggcaaacactacggagcctcaag
ctgtgacggctgcaagggattcttcaggaggagcgtgaggaagaaccaca
tgtactcctgcaggtttagcaggcagtgcgtggtagacaaagataaagag
gaaccagtgtcgttactgcaggctcaagaagtgcttccgggctggcatga
agaaagaagccgtccaaaatgagcgggatgccatcagcacgcggaggtca
agctacgaggacagcagcctaccctccattaatgcgctcctgcaggcaga
ggtcctgtctcagcagatcacctcccccatctctgggatcaatggcgaca
ttcgggccaagaagattgccaacatcacggatgtgtgtgagtctatgaag
gagcagctgctggttctggtcgaatgggccaagtacatcccggccttctg
tgaacttcttctggatgaccaggtggcgctgctcagagcccacgctggtg
agcacctgctgcttggagccaccaagaggtccatggtgttcaaggatgtg
ctgctcctaggcaatgactacatcgtccctcggcactgtccagagctagc
agagatgagccgtgtgtccattcgcatcctcgatgagctggtcttgccct
tccaagagctgcagatcgatgataatgaatacgcctgcctcaaagccatc
atcttctttgacccagatgccaaggggctgagtgacccaggcaagatcaa
gcggctgcggtcacaggtgcaggtgagcctggaggattacatcaacgacc
ggcagtatgactctcggggtcgttttggagagctgctgctgctcctgccc
actctgcagagcattacctggcagatgatcgagcagatccagttcatcaa
gctctttggcatggccaagattgacaacctgctgcaggagatgctgcttg
gagggtctgccagtgacgcgccccacgcccaccaccccctgcaccctcac
ctgatgcaagaacacatgggcaccaatgtcatagttgccaacacgatgcc
ctctcacctcagcaatggacagatgtgtgagtggccccggcccagggggc
aggcagccacccctgagactccacagccatcaccaccaagtggctctgga
tctgaatcctacaagctcctgccaggagccatcaccaccatcgtcaagcc
tccctctgccatcccccagccaacgatcaccaagcaggaagccatctag
HNF6: U95945 (SEQ ID NO: 54):
atgaacgcacagctgaccatggaggcgatcggcgagctgcacggggtgag
ccatgagccggtgcccgcccctgctgacctgctgggcggcagccctcacg
cgcgcagctccgtgggacaccgcggcagccacctgcctcccgcgcacccg
cgttccatgggcatggcgtccctgctggacggcggcagcggaggcagcga
ttaccaccaccaccaccgcgcccctgagcacagcttggctggccccctgc
accccaccatgaccatggcctgtgaaactcccccaggtatgagcatgccc
accacctacactaccttaacccctctgcagccgctgccgcccatctccac
cgtgtccgacaagttccctcaccatcatcaccaccaccatcaccaccacc
acccacaccaccaccagcgcctggcgggcaacgtgagcggtagtttcaca
cttatgcgggatgagcgcgggctggcctctatgaataacctctatacccc
ctaccacaaggacgtggctggcatgggccagagcctctcgcccctctctg
gctccggtctgggcagcattcacaactcccagcaaggacttccccactat
gctcatcccggcgcggctatgcccaccgacaagatgctcaccccaaatgg
ctttgaagcccaccaccctgccatgctcggtcgccacggggagcagcacc
tcacgcccacctcggccggcatggtacccatcaacggccttcctccgcac
catcctcatgcccacctgaatgcccagggccacggacagctcctgggcac
agcccgagagcccaacccttcggtgaccggcgcgcaggtcagcaatggaa
gtaattcagggcagatggaagagatcaataccaaagaggtggcgcagcgt
atcaccaccgagctcaaacgttacagcatcccacaggccatcttcgcgca
gagggtgctctgccgttcccaggggaccctttcggacctgctgcgaaacc
ccaagccctggagcaaactcaagtcgggtcgggagaccttccggaggatg
tggaagtggctgcaggagccggagttccagcgcatgtcggcgctccgctt
agcagcctgcaaacggaaagagcaagaacatgggaaggacagaggcaaca
cccccaaaaagcccaggctggtcttcacagacgtccaacgtcgaactcta
catgcaatattcaaggaaaataagcgtccgtccaaagaattacaaatcac
catctcccagcagctggggttggagctgagcactgtcagcaacttcttca
tgaatgccagaaggaggagtctggacaagtggcaggacgagggcggctcc
aactcaggcagttcatcgtcctcatcgagcacttgtaccaaagcatga
HLXB9: NM_001096823 (SEQ ID NO: 55):
atggagaagtccaagaatttcaggattgacgctctcctggcgatagatcc
ccccaaggctcagacctccccattggctctggtcacctcgctgtcctcct
cgtctctctccgggagccccccgtccgagcacactgacagcctcaggact
gactccccctcccctccaaggacttgtggactggtccctaaaccaggttt
cctgagcagccaccagcaccccccaaacatgatgtcattgcacccccagg
ctgctccagggatcccccctcaggccctgtatggacacccgatgtacagc
tacttggcagcggggcagcacccagctctgtcctacccctactcccagat
gcagagcagccaccacccccaccccatggaccccatcaagatcagcgctg
gcaccttccaactggaccagtggctcagagcctccactgccggcatgatg
ctgcccaaaatggcagactttaactcccaggcccaatccaacctgctggg
aaagtgcagaagaccaaggacagcgtttaccagtcagcagctgttggaac
tggagcaccaattcaagctgaacaagtacctctccaggccgaaacgcttt
gaagtggccacttccctgatgctcactgagacgcaggtgaagatctggtt
ccagaacaggcgcatgaaatggaagaggagtaagaaagccaaggagcagg
cggcgcaggactcagcagagaaacagcagagggcaggcagggcagcagcg
aggagaagtgctcggatgagctgcaggaagagaagaaatcctaccatctc
catcccaggggggagcccatcaaagggaacggccgcctgcagcccagaga
ctatacagacagcgaagaggacgaggaggaggacagggaagaggaggaag
aggaagatcacagaggggaggggaagcggttttaccatcattcttctgac
tgcacatccgaggaagaggagaacagccacaataagcagagcggccactg
a; NM_019944.
(SEQ ID NO: 56)
atggaaaaatccaaaaatttccgcatcgacgccctgctggccgtggatcc
cccgcgagccgcctccacgcagagcgcgcctctggccttggtcacttccc
tcgcgactacagtatctggtcccggccgcggcggcagcggcggcgggggg
accagtagcggggcgagccgtagctgcagtcccgcatcctcggaggccac
tgcagcgcccggtgaccggctgagagctgagagcccgtcgcccccacgct
tgctggctgcacactgcgcgctgctgcccaagcccggattcctgggcgcc
ggaggaggcggcggcgcggcgggtgggccgggcactccccaccaccacgc
gcaccctggtgcagcagccgccgcggctgccgctgccgctgccgcggctg
ccggtggcctggcactggggctgcacccggggggcgcacagggcggcgcg
ggcctccctgcacaggcggctctctatggacacccggtctacagttattc
ggcagcagctgcagcggccgcgctagctggccagcacccggcgctttcct
actcataccctcaggtgcagggcgcgcaccctgcgcaccctgccgacccc
atcaagctgggtgccagcaccttccaactggaccagtggctgcgcgcgtc
tactgcgggcatgatcctgcccaagatgccggacttcagctgtcaggcgc
agtcgaacctcttggggaagtgccgaaggcctcgcacggccttcaccagc
cagcagctgttggagctggaacaccagttcaagctcaacaagtacctgtc
tcgacccaagcgttttgaggtggctacctcgctcatgctcaccgagactc
aggtgaagatttggttccagaaccgccgaatgaaatggaaacgcagcaaa
aaggccaaagagcaggctgcgcaggaggcggagaagcagaagggcggcgg
cgggggcaccggcaaaggcggcagtgaggagaagacggaagaggagctga
tggggcctccggtttcgggggacaaggcaagcggccgtcgcctgcgggac
ttgcgggacagtgaccctgatgaggacgaggatgatgaagaagaggacaa
cttcccgtacagcaatggtgccggtgcccatgctgcctcatccgactgct
catctgaggacgactcgcctcctccaagactaggcgggcctggacaccaa
cctctgccccagtag
NM_005515 (SEQ ID NO: 57):
atggaaaaatccaaaaatttccgcatcgacgccctgctggccgtggatcc
cccgcgagccgcctccacgcagagcgcgcctctggccttggtcacttccc
tcgcgactacagtatctggtcccggccgcggcggcagcggcggcgggggg
accagtagcggggcgagccgtagctgcagtcccgcatcctcggaggccac
tgcagcgcccggtgaccggctgagagctgagagcccgtcgcccccacgct
tgctggctgcacactgcgcgctgctcccaagcccggattcctgggcgccg
gaggaggcggcggcgcggcgggtgggccgggcactccccaccaccacgcg
caccctggtgcagcagccgccgcggctgccgctgccgctgccgcggctgc
cggtggcctggcactggggctgcacccggggggcgcacagggcggcgcgg
gcctccctgcacaggcggctctctatggacacccggtctacagttattcg
gcagcagctgcagcggccgcgctagctggccagcacccggcgctttccta
ctcataccctcaggtgcagggcgcgcaccctgcgcaccctgccgacccca
tcaagctgggtgccagcaccttccaactggaccagtggctgcgcgcgtct
actgcgggcatgatcctgcccaagatgccggacttcagctgtcaggcgca
gtcgaacctcttggggaagtgccgaaggcctcgcacggccttcaccagcc
agcagctgttggagctggaacaccagttcaagctcaacaagtacctgtct
cgacccaagcgttttgaggtggctacctcgctcatgctcaccgagactca
ggtgaagatttggttccagaaccgccgaatgaaatggaaacgcagcaaaa
aggccaaagagcaggctgcgcaggaggcggagaagcagaagggcggcggc
gggggcaccggcaaaggcggcagtgaggagaagacggaagaggagctgat
ggggcctccggtttcgggggacaaggcaagcggccgtcgcctgcgggact
tgcgggacagtgaccctgatgaggacgaggatgatgaagaagaggacaac
ttcccgtacagcaatggtgccggtgcccatgctgcctcatccgactgctc
atctgaggacgactcgcctcctccaagactaggcgggcctggacaccaac
ctctgccccagtag
Lbx1: NM_006562 (SEQ ID NO: 58):
atgacttccaaggaggacggcaaggcggcgccgggggaggagcggcggcg
cagcccgctggaccacctgcctccgcctgccaactccaacaagccactga
cgccgttcagcatcgaggacatcctcaacaagccgtctgtgcggagaagt
tactcgctgtgcggggcggcgcacctgctggccgccgcggacaagcacgc
gcagggcggcttgcccctggcgggccgcgcgctgctctcgcagacctcgc
cgctgtgcgcgctggaggagctcgccagcaagacgtttaaggggctggag
gtcagcgttctgcaggcagccgaaggccgcgacggtatgaccatctttgg
gcagcggcagacccctaagaagcggcgaaagtcgcgcacggccttcacca
accaccagatctatgaattggaaaagcgctttctataccagaagtacctg
tcccccgccgatcgcgaccaaatcgcgcagcagctgggcctcaccaacgc
gcaagtcatcacctggttccagaatcggcgcgctaagctcaagcgggacc
tggaggagatgaaggccgacgtagagtccgccaagaaactgggccccagc
gggcagatggacatcgtggcgctggccgaactcgagcagaactcggaggc
cacagccggcggtggcggcggctgcggcagggccaagtcgaggcccggct
ctccggtcctccccccaggcgccccgaaggccccgggcgctggcgccctg
cagctctcgcctgcctctccgctcacggaccagccggccagcagccagga
ctgctcggaggacgaggaagacgaagagatcgacgtggacgattga;
NM_010691.
Lmx1b (SEQ ID NO: 59):
atgttggacggcatcaagatggaggagcacgccctgcgccccgggcccgc
cactctgggggtgctgctgggctccgactgcccgcatcccgccgtctgcg
agggctgccagcggcccatctccgaccgcttcctgatgcgagtcaacgag
tcgtcctggcacgaggagtgtttgcagtgcgcggcgtgtcagcaagccct
caccaccagctgctacttccgggatcggaaactgtactgcaaacaagact
accaacagctcttcgcggccaagtgcagcggctgcatggagaagatcgcc
cccaccgagttcgtgatgcgggcgctggagtgcgtgtaccacctgggctg
cttctgctgctgcgtgtgtgaacggcagctacgcaagggcgacgaattcg
tgctcaaggagggccagctgctgtgcaagggtgactacgagaaggagaag
gacctgctcagctccgtgagccccgacgagtccgactccgtgaagagcga
ggatgaagatggggacatgaagccggccaaggggcagggcagtcagagca
agggcagcggggatgacgggaaggacccgcggaggcccaagcgaccccgg
accatcctcaccacgcagcagcgaagagccttcaaggcctccttcgaggt
ctcgtcgaagccttgccgaaaggtccgagagacactggcagctgagacgg
gcctcagtgtgcgcgtggtccaggtctggtttcagaaccaaagagcaaag
atgaagaagctggcgcggcggcaccagcagcagcaggagcagcagaactc
ccagcggctgggccaggaggtcctgtccagccgcatggagggcatgatgg
cttcctacacgccgctggccccaccacagcagcagatcgtggccatggaa
cagagcccctacggcagcagcgaccccttccagcagggcctcacgccgcc
ccaaatgccagggaacgactccatcttccatgacatcgacagcgatacct
ccttaaccagcctcagcgactgcttcctcggctcctcagacgtgggctcc
ctgcaggcccgcgtggggaaccccatcgaccggctctactccatgcagag
ttcctacttcgcctcctga; NM_010725
Neurogenin(NEUROG1): NM_006161 (SEQ ID NO: 60):
atgccagcccgccttgagacctgcatctccgacctcgactgcgccagcag
cagcggcagtgacctatccggcttcctcaccgacgaggaagactgtgcca
gactccaacaggcagcctccgcttcggggccgcccgcgccggcccgcagg
ggcgcgcccaatatctcccgggcgtctgaggttccaggggcacaggacga
cgagcaggagaggcggcggcgccgcggccggacgcgggtccgctccgagg
cgctgctgcactcgcttcgcaggagccggcgcgtcaaggccaacgatcgc
gagcgcaaccgcatgcacaacttgaacgcggccctggacgcactgcgcag
cgtgctgccctcgttccccgacgacaccaagctcaccaaaatcgagacgc
tgcgcttcgcctacaactacatctgggctctggccgagacactgcgcctg
gcggatcaagggctgcccggaggcggtgcccgggagcgcctcctgccgcc
gcagtgcgtcccctgcctgcccggtcccccaagccccgccagcgacgcgg
agtcctggggctcaggtgccgccgccgcctccccgctctctgaccccagt
agcccagccgcctccgaagacttcacctaccgccccggcgaccctgtttt
ctccttcccaagcctgcccaaagacttgctccacacaacgccctgtttca
ttccttaccactag; BQ169355.
Neurogenin2(NEUROG2): NM_024019 (SEQ ID NO: 61):
atgttcgtcaaatccgagccttggagttgaaggaggaagaggacgtggtt
agtgctgctcggatcggcctcccccgccttggcggccctgaccccgctgt
catccagcgccgacgaagaagaggaggaggagccgggcgcgtcaggcggg
gcgcgtcggcagcgcggggctgaggccgggcagggggcgcggggcggcgt
ggctgcgggtgcggagggctgccggcccgcacggctgctgggtctggtac
acgattgcaaacggcgcccttcccgggcgcgggccgtctcccgaggcgcc
aagacggccgagacggtgcagcgcatcaagaagacccgtagactgaaggc
caacaaccgcgagcgaaaccgcatgcacaacctcaacgcggcactggacg
cgctgcgcgaggtgctccccacgttccccgaggacgccaagctcaccaag
atcgagaccctgcgcttcgcccacaactacatctgggcactcaccgagac
cctgcgcctggcggatcactgcgggggcggcggcgggggcctgccggggg
cgctcttctccgaggcagtgttgctgagcccgggaggcgccagcgccgcc
ctgagcagcagcggagacagcccctcgcccgcctccacgtggagttgcac
caacagccccgcgccgtcctcctccgtgtcctccaattccacctccccct
acagctgcactttatcgcccgccagcccggccgggtcagacatggactat
tggcagcccccacctcccgacaagcaccgctatgcacctcacctccccat
agccagggattgtatctag; DR001447.
Neurogenin3(NEUROG3) (SEQ ID NO: 62):
atgacgcctcaaccctcgggtgcgcccactgtccaagtgacccgtgagac
ggagcggtccttccccagagcctcggaagacgaagtgacctgccccacgt
ccgccccgcccagccccactcgcacacgggggaactgcgcagaggcggaa
gagggaggctgccgaggggccccgaggaagctccgggcacggcgcggggg
acgcagccggcctaagagcgagttggcactgagcaagcagcgacggagtc
ggcgaaagaaggccaacgaccgcgagcgcaatcgaatgcacaacctcaac
tcggcactggacgccctgcgcggtgtcctgcccaccttcccagacgacgc
gaagctcaccaagatcgagacgctgcgcttcgcccacaactacatctggg
cgctgactcaaacgctgcgcatagcggaccacagcttgtacgcgctggag
ccgccggcgccgcactgcggggagctgggcagcccaggcggttcccccgg
ggactgggggtccctctactccccagtctcccaggctggcagcctgagtc
ccgccgcgtcgctggaggagcgacccgggctgctgggggccaccttttcc
gcctgcttgagcccaggcagtctggctttctcagattttctgtga;
NM_009719.
MASH1: NM_0004316 (SEQ ID NO: 63):
atggaaagctctgccaagatggagagcggcggcgccggccagcagcccca
gccgcagccccagcagcccttcctgccgcccgcagcctgtttctttgcca
cggccgcagccgcggcggccgcagccgccgcagcggcagcgcagagcgcg
cagcagcagcagcagcagcagcagcagcagcagcaggcgccgcagctgag
accggcggccgacggccagccctcagggggcggtcacaagtcagcgccca
agcaagtcaagcgacagcgctcgtcttcgcccgaactgatgcgctgcaaa
cgccggctcaacttcagcggctttggctacagcctgccgcagcagcagcc
ggccgccgtggcgcgccgcaacgagcgcgagcgcaaccgcgtcaagttgg
tcaacctgggctttgccacccttcgggagcacgtccccaacggcgcggcc
aacaagaagatgagtaaggtggagacactgcgctcggcggtcgagtacat
ccgcgcgctgcagcagctgctggacgagcatgacgcggtgagcgccgcct
tccaggcaggcgtcctgtcgcccaccatctcccccaactactccaacgac
ttgaactccatggccggctcgccggtctcatcctactcgtcggacgaggg
ctcttacgacccgctcagccccgaggagcaggagcttctcgacttcacca
actggttctga; NM_008553.
MyoD: NM_010866 (SEQ ID NO: 64):
atggagcttctatcgccgccactccgggacatagacttgacaggccccga
cggctctctctgctcctttgagacagcagacgacttctatgatgacccgt
gtttcgactcaccagacctgcgcttttttgaggacctggacccgcgcctg
gtgcacatgggagccctcctgaaaccggaggagcacgcacacttccctac
tgcggtgcacccaggcccaggcgctcgtgaggatgagcatgtgcgcgcgc
ccagcgggcaccaccaggcgggtcgctgcttgctgtgggcctgcaaggcg
tgcaagcgcaagaccaccaacgctgatcgccgcaaggccgccaccatgcg
cgagcgccgccgcctgagcaaagtgaatgaggccttcgagacgctcaagc
gctgcacgtccagcaacccgaaccagcggctacccaaggtggagatcctg
cgcaacgccatccgctacatcgaaggtctgcaggctctgctgcgcgacca
ggacgccgcgccccctggcgccgctgccttctacgcacctggaccgctgc
ccccaggccgtggcagcgagcactacagtggcgactcagatgcatccagc
ccgcgctccaactgctctgatggcatgatggattacagcggccccccaag
cggcccccggcggcagaatggctacgacaccgcctactacagtgaggcgg
cgcgcgagtccaggccagggaagagtgcggctgtgtcgagcctcgactgc
ctgtccagcatagtggagcgcatctccacagacagccccgctgcgcctgc
gctgcttttggcagatgcaccaccagagtcgcctccgggtccgccagagg
gggcatccctaagcgacacagaacagggaacccagaccccgtctcccgac
gccgcccctcagtgtcctgcaggctcaaaccccaatgcgatttatcaggt
gctttga;
NM_002478 (SEQ ID NO: 65):
atggagctactgtcgccaccgctccgcgacgtagacctgacggcccccga
cggctctctctgctcctttgccacaacggacgacttctatgacgacccgt
gtttcgactccccggacctgcgcttcttcgaagacctggacccgcgcctg
atgcacgtgggcgcgctcctgaaacccgaagagcactcgcacttccccgc
ggcggtgcacccggccccgggcgcacgtgaggacgagcatgtgcgcgcgc
ccagcgggcaccaccaggcgggccgctgcctactgtgggcctgcaaggcg
tgcaagcgcaagaccaccaacgccgaccgccgcaaggccgccaccatgcg
cgagcggcgccgcctgagcaaagtaaatgaggcctttgagacactcaagc
gctgcacgtcgagcaatccaaaccagcggttgcccaaggtggagatcctg
cgcaacgccatccgctatatcgagggcctgcaggctctgctgcgcgacca
ggacgccgcgccccctggcgccgcagccgccttctatgcgccgggcccgc
tgcccccgggccgcggcggcgagcactacagcggcgactccgacgcgtcc
agcccgcgctccaactgctccgacggcatgatggactacagcggcccccc
gagcggcgcccggcggcggaactgctacgaaggcgcctactacaacgagg
cgcccagcgaacccaggcccgggaagagtgcggcggtgtcgagcctagac
tgcctgtccagcatcgtggagcgcatctccaccgagagccctgcggcgcc
cgccctcctgctggcggacgtgccttctgagtcgcctccgcgcaggcaag
aggctgccgcccccagcgagggagagagcagcggcgaccccacccagtca
ccggacgccgccccgcagtgccctgcgggtgcgaaccccaacccgatata
ccaggtgctctga.
Myf5: NM_005993 (SEQ ID NO: 66):
atggacgtgatggatggctgccagttctcaccttctgagtacttctacga
cggctcctgcataccgtcccccgagggtgaatttggggacgagtttgtgc
cgcgagtggctgccttcggagcgcacaaagcagagctgcagggctcagat
gaggacgagcacgtgcgagcgcctaccggccaccaccaggctggtcactg
cctcatgtgggcctgcaaagcctgcaagaggaagtccaccaccatggatc
ggcggaaggcagccactatgcgcgagcggaggcgcctgaagaaggtcaac
caggctttcgaaaccctcaagaggtgtaccacgaccaaccccaaccagag
gctgcccaaggtggagatcctcaggaatgccatccgctacatcgagagcc
tgcaggagttgctgagagagcaggtggagaactactatagcctgccggga
cagagctgctcggagcccaccagccccacctccaactgctctgatggcat
gcccgaatgtaacagtcctgtctggtccagaaagagcagtacttttgaca
gcatctactgtcctgatgtatcaaatgtatatgccacagataaaaactcc
ttatccagcttggattgcttatccaacatagtggaccggatcacctcctc
agagcaacctgggttgcctctccaggatctggcttctctctctccagttg
ccagcaccgattcacagcctgcaactccaggggcttctagttccaggctt
atctatcatgtgctatga; NM_131576; NM_008656.
Myf6: NM_002469 (SEQ ID NO: 67):
atgatgatggacctttttgaaactggctcctatttcttctacttggatgg
ggaaaatgttactctgcagccattagaagtggcagaaggctctcctttgt
atccagggagtgatggtaccttgtccccctgccaggaccaaatgcccccg
gaagcggggagcgacagcagcggagaggaacatgtcctggcgcccccggg
cctgcagcctccacactgccccggccagtgtctgatctgggcttgcaaga
cctgcaagagaaaatctgcccccactgaccggcgaaaagccgccaccctg
cgcgaaaggaggaggctaaagaaaatcaacgaggccttcgaggcactgaa
gcggcgaactgtggccaaccccaaccagaggctgcccaaggtggagattc
tgcggagcgccatcagctatattgagcggctgcaggacctgctgcaccgg
ctggatcagcaggagaagatgcaggagctgggggtggaccccttcagcta
cagacccaaacaagaaaatcttgagggtgcggatttcctgcgcacctgca
gctcccagtggccaagtgtttccgatcattccagggggctcgtgataaac
ggctaaggaaggaggagcaagtattgattcgtcagcctcgagtagccttc
gatgcctttcttccatcgtggacagtatttcctcggaggaacgcaaactc
ccctgcgtggaggaagtggtggagaagtaa
NM_008657; NM_008657; NM_013172.
Ifrd1: NM_001007245 (SEQ ID NO: 68):
atgccgaagaacaagaagcggaacactccccqccgcggtagcagtgctgg
cggcggcgggtcaggagcagccgcagcgacggcggcgacagcaggtggcc
agcatcgaaatgttcagccttttagtgatgaagatgcatccaattgaaac
aatgagccattgcagtggttatagcgatccttccagttttgctgaagatg
gaccagaagtccttgatgaggaaggaactcaagaagacctagagtacaag
ttgaagggattaattgacctaaccctggataagagtgcgaagacaaggca
agcagctcttgaaggtattaaaaatgcactggcttcaaaaatgctgtatg
aatttattctggaaaggagaatgactttaactgatagcattgaacgctgc
ctgaaaaaaggtaagagtgatgagcaacgtgcagctgcagcgttagcatc
tgttctttgtattcagctgggccctggaattgaaagtgaagagattttga
aaactcttggaccaatcctaaagaaaatcatttgtgatgggtcagctagt
atgcaggctaggcaaacttgtgcaacttgctttggtgtttgctgttttat
tgccacagatgacattactgaactatactcaactctggaatgtttggaaa
atatcttcactaaatcctatctcaaagagaaagacactactgttatttgc
agcactcctaatacagtgcttcatatcagctctcttcttgcatggacact
actgctgaccatatgcccaatcaatgaagtgaagaaaaagcttgagatgc
atttccataagcttccaagcctcctctcttgtgatgatgtaaacatgaga
atagctgctggtgaatctttggcacttctctttgaattggccagaggaat
agagagtgactttttttatgaagacatggagtccttgacgcagatgctta
gggccttggcaacagatggaaataaacaccgggccaaagtggacaagaga
aagcagcggtcagttttcagagatgtcctgagggcagtggaggaacggga
ttttccaacagaaaccattaaatttggtcctgaacgcatgtatattgatt
gctgggtaaaaaaacacacctatgacacctttaaggaggttcttggatca
gggatgcagtaccacttgcagtcaaatgaattccttcgaaatgtatttga
acttggacccccagtgatgcttgatgctgcaacgcttaaaacgatgaaga
tttctcgtttcgaaaggcatttatataactctgcagccttcaaagctcga
accaaagctagaagcaaatgtcgagataagagagcagatgttggagaatt
cttctag.
Mef2A: NM_013172 (SEQ ID NO: 69):
atggggcggaagaaaatacaaatcacacgcataatggatgaaaggaaccg
acaggtcacttttacaaagagaaagtttggattaatgaagaaagcctatg
aacttagtgtgctctgtgactgtgaaatagcactcatcattttcaacagc
tctaacaaactgtttcaatatgctagcactgatatggacaaagttcttct
caagtatacagaatataatgaacctcatgaaagcagaaccaactcggata
ttgttgaggctctgaacaagaaggaacacagagggtgcgacagcccagac
cctgatacttcatatgtgctaactccacatacagaagaaaaatataaaaa
aattaatgaggaatttgataatatgatgcggaatcataaaatcgcacctg
gtctgccacctcagaacttttcaatgtctgtcacagttccagtgaccagc
cccaatgctttgtcctacactaacccagggagttcactggtgtccccatc
tttggcagccagctcaacgttaacagattcaagcatgctctctccacctc
aaaccacattacatagaaatgtgtctcctggagctcctcagagaccacca
agtactggcaatgcaggtgggatgttgagcactacagacctcacagtgcc
aaatggagctggaagcagtccagtggggaatggatttgtaaactcaagag
cttctccaaatttgattggagctactggtgcaaatagcttaggcaaagtc
atgcctacaaagtctccccctccaccaggtggtggtaatcttggaatgaa
cagtaggaaaccagatcttcgagttgtcatccccccttcaagcaagggca
tgatgcctccactatcggaggaagaggaattggagttgaacacccaaagg
atcagtagttctcaagccactcaacctcttgctaccccagtcgtgtctgt
gacaaccccaagcttgcctccgcaaggacttgtgtactcagcaatgccga
ctgcctacaacactgattattcactgaccagcgctgacctgtcagccctt
caaggcttcaactcgccaggaatgctgtcgctgggacaggtgtcggcctg
gcagcagcaccacctaggacaagcagccctcagctctcttgttgctggag
ggcagttatctcagggttccaatttatccattaataccaaccaaaacatc
agcatcaagtccgaaccgatttcacctcctcgggatcgtatgaccccatc
gggcttccagcagcagcagcagcagcagcagcagcagcagccgccgccac
caccgcagccccagccacaacccccgcagccccagccccgacaggaaatg
gggcgctcccctgtggacagtctgagcagctctagtagctcctatgatgg
cagtgatcgggaggatccacggggcgacttccattctccaattgtgcttg
gccgacccccaaacactgaggacagagaaagcccttctgtaaagcgaatg
aggatggacgcgtgggtgacctaa.
Myogenin: NM_002479 (SEQ ID NO: 70):
Atggagctgtatgagacatccccctacttctaccaggaaccccgcttcta
tgatggggaaaactacctgcctgtccacctccagggcttcgaaccaccag
gctacgagcggacggagctcaccctgagccccgaggccccagggcccctt
gaggacaaggggctggggacccccgagcactgtccaggccagtgcctgcc
gtgggcgtgtaaggtgtgtaagaggaagtcggtgtccgtggaccggcggc
gggcggccacactgagggagaagcgcaggctcaagaaggtgaatgaggcc
ttcgaggccctgaagagaagcaccctgctcaaccccaaccagcggctgcc
caaggtggagatcctgcgcagtgccatccagtacatcgagcgcctccagg
ccctgctcagctccctcaaccaggaggagcgtgacctccgctaccggggc
gggggcgggccccagccaggggtgcccagcgaatgcagctctcacagcgc
ctcctgcagtccagagtggggcagtgcactggagttcagcgccaacccag
gggatcatctgctcacggctgaccctacagatgcccacaacctgcactcc
ctcacctccatctgttacagcatcacagtggaagatgtgtctgtggcctt
cccagatgaaaccatgcccaactga; BC05389; BC068019;
AB257560.
Nkx2.2: NM_002509 (SEQ ID NO: 71):
atgtcgctgaccaacacaaagacggggttttcggtcaaggacatcttaga
cctgccggacaccaacgatgaggagggctctgtggccgaaggtccggagg
aagagaacgaggggcccgagccagccaagagggccgggccgctggggcag
ggcgccctggacgcggtgcagagcctgcccctgaagaaccccttctacga
cagcagcgacaacccgtacacgcgctggctggccagcaccgagggccttc
agtactccctgcacggtctggctgccggggcgccccctcaggactcaagc
tccaagtccccggagccctcggccgacgagtcaccggacaatgacaagga
gaccccgggcggcgggggggacgccggcaagaagcgaaagcggcgagtgc
ttttctccaaggcgcagacctacgagctggagcggcgctttcggcagcag
cggtacctgtcggcgcccgagcgcgaacacctggccagcctcatccgcct
cacgcccacgcaggtcaagatctggttccagaaccaccgctacaagatga
agcgcgcccgggccgagaaaggtatggaggtgacgcccctgccctcgccg
cgccgggtggccgtgcccgtcttggtcagggacggcaaaccatgtcacgc
gctcaaagcccaggacctggcagccgccaccttccaggcgggcattccct
tttctgcctacagcgcgcagtcgctgcagcacatgcagtacaacgcccag
tacagctcggccagcaccccccagtacccgacagcacaccccctggtcca
ggcccagcagtggacttggtga; NM_001077632; NM_010919.
Notch
Notch1: NM_017617 (SEQ ID NO: 72):
atgccgccgctcctggcgcccctgctctgcctggcgctgctgcccgcgct
cgccgcacgaggcccgcgatgctcccagcccggtgagacctgcctgaatg
gcgggaagtgtgaagcggccaatggcacggaggcctgcgtctgtggcggg
gccttcgtgggcccgcgatgccaggaccccaacccgtgcctcagcacccc
ctgcaagaacgccgggacatgccacgtggtggaccgcagaggcgtggcag
actatgcctgcagctgtccctgggcttctctgggcccctctgcctgacac
ccctggacaatgcctgcctcaccaacccctgccgcaacgggggcacctgc
gacctgctcacgctgacggagtacaagtgccgctgcccgcccggctggtc
agggaaatcgtgccagcaggctgacccgtgcgcctccaacccctgcgcca
acggtggccagtgcctgcccttcgaggcctcctacatctgccactgccca
cccagcttccatggccccacctgccggcaggatgtcaacgagtgtggcca
gaagcccgggctttgccgccacggaggcacctgccacaacgaggtcggct
cctaccgctgcgtctgccgcgccacccacactggccccaactgcgagcgg
ccctacgtgccctgcagcccctcgccctgccagaacgggggcacctgccg
ccccacgggcgacgtcacccacgagtgtgcctgcctgccaggcttcaccg
gccagaactgtgaggaaaatatcgacgattgtccaggaaacaactgcaag
aacgggggtgcctgtgtggacggcgtgaacacctacaactgccgctgccc
gccagagtggacaggtcagtactgtaccgaggatgtggacgagtgccagc
tgatgccaaatgcctgccagaacggcgggacctgccacaacacccacggt
ggctacaactgcgtgtgtgtcaacggctggactggtgaggactgcagcga
gaacattgatgactgtgccagcgccgcctgcttccacggcgccacctgcc
atgaccgtgtggcctccttctactgcgagtgtccccatggccgcacaggt
ctgctgtgccacctcaacgacgcatgcatcagcaacccctgtaacgaggg
ctccaactgcgacaccaaccctgtcaatggcaaggccatctgcacctgcc
cctcggggtacacgggcccggcctgcagccaggacgtggatgagtgctcg
ctgggtgccaacccctgcgagcatgcgggcaagtgcatcaacacgctggg
ctccttcgagtgccagtgtctgcagggctacacgggcccccgatgcgaga
tcgacgtcaacgagtgcgtctcgaacccgtgccagaacgacgccacctgc
ctggaccagattggggagttccagtgcatctgcatgcccggctacgaggg
tgtgcactgcgaggtcaacacagacgagtgtgccagcagcccctgcctgc
acaatggccgctgcctggacaagatcaatgagttccagtgcgagtgcccc
acgggcttcactgggcatctgtgccagtacgatgtggacgagtgtgccag
caccccctgcaagaatggtgccaagtgcctggacggacccaacacttaca
cctgtgtgtgcacggaagggtacacggggacgcactgcgaggtggacatc
gatgagtgcgaccccgacccctgccactacggctcctgcaaggacggcgt
cgccaccttcacctgcctctgccgcccaggctacacgggccaccactgcg
agaccaacatcaacgagtgctccagccagccctgccgccacgggggcacc
tgccaggaccgcgacaacgcctacctctgcttctgcctgaaggggaccac
aggacccaactgcgagatcaacctggatgactgtgccagcagcccctgcg
actcgggcacctgtctggacaagatcgatggctacgagtgtgcctgtgag
ccgggctacacagggagcatgtgtaacatcaacatcgatgagtgtgcggg
caacccctgccacaacgggggcacctgcgaggacggcatcaatggcttca
cctgccgctgccccgagggctaccacgaccccacctgcctgtctgaggtc
aatgagtgcaacagcaacccctgcgtccacggggcctgccgggacagcct
caacgggtacaagtgcgactgtgaccctgggtggagtgggaccaactgtg
acatcaacaacaatgagtgtgaatccaacccttgtgtcaacggcggcacc
tgcaaagacatgaccagtggctacgtgtgcacctgccgggagggcttcag
cggtcccaactgccagaccaacatcaacgagtgtgcgtccaacccatgtc
tgaaccagggcacgtgtattgacgacgttgccgggtacaagtgcaactgc
ctgctgccctacacaggtgccacgtgtgaggtggtgctggccccgtgtgc
ccccagcccctgcagaaacggcggggagtgcaggcaatccgaggactatg
agagcttctcctgtgtctgccccacgggctggcaagggcagacctgtgag
gtcgacatcaacgagtgcgttctgagcccgtgccggcacggcgcatcctg
ccagaacacccacggcggctaccgctgccactgccaggccggctacagtg
ggcgcaactgcgagaccgacatcgacgactgccggcccaacccgtgtcac
aacgggggctcctgcacagacggcatcaacacggccttctgcgactgcct
gcccggcttccggggcactttctgtgaggaggacatcaacgagtgtgcca
gtgacccctgccgcaacggggccaactgcacggactgcgtggacagctac
acgtgcacctgccccgcaggcttcagcgggatccactgtgagaacaacac
gcctgactgcacagagagctcctgcttcaacggtggcacctgcgtggacg
gcatcaactcgttcacctgcctgtgtccacccggcttcacgggcagctac
tgccagcacgatgtcaatgagtgcgactcacagccctgcctgcatggcgg
cacctgtcaggacggctgcggctcctacaggtgcacctgcccccagggct
acactggccccaactgccagaaccttgtgcactggtgtgactcctcgccc
tgcaagaacggcggcaaatgctggcagacccacacccagtaccgctgcga
gtgccccagcggctggaccggcctttactgcgacgtgcccagcgtgtcct
gtgaggtggctgcgcagcgacaaggtgttgacgttgcccgcctgtgccag
catggagggctctgtgtggacgcgggcaacacgcaccactgccgctgcca
ggcgggctacacaggcagctactgtgaggacctggtggacgagtgctcac
ccagcccctgccagaacggggccacctgcacggactacctgggcggctac
tcctgcaagtgcgtggccggctaccacggggtgaactgctctgaggagat
cgacgagtgcctctcccacccctgccagaacgggggcacctgcctcgacc
tccccaacacctacaagtgctcctgcccacggggcactcagggtgtgcac
tgtgagatcaacgtggacgactgcaatccccccgttgaccccgtgtcccg
gagccccaagtgctttaacaacggcacctgcgtggaccaggtgggcggct
acagctgcacctgcccgccgggcttcgtgggtgagcgctgtgagggggat
gtcaacgagtgcctgtccaatccctgcgacgcccgtggcacccagaactg
cgtgcagcgcgtcaatgacttccactgcgagtgccgtgctggtcacaccg
ggcgccgctgcgagtccgtcatcaatggctgcaaaggcaagccctgcaag
aatgggggcacctgcgccgtggcctccaacaccgcccgcgggttcatctg
caagtgccctgcgggcttcgagggcgccacgtgtgagaatgacgctcgta
cctgcggcagcctgcgctgcctcaacggcggcacatgcatctccggcccg
cgcagccccacctgcctgtgcctgggccccttcacgggccccgaatgcca
gttcccggccagcagcccctgcctgggcggcaacccctgctacaaccagg
ggacctgtgagcccacatccgagagccccttctaccgttgcctgtgcccc
gccaaattcaacgggctcttgtgccacatcctggactacagcttcggggg
tggggccgggcgcgacatccccccgccgctgatcgaggaggcgtgcgagc
tgcccgagtgccaggaggacgcgggcaacaaggtctgcagcctgcagtgc
aacaaccacgcgtgcggctgggacggcggtgactgctccctcaacttcaa
tgacccctggaagaactgcacgcagtctctgcagtgctggaagtacttca
gtgacggccactgtgacagccagtgcaactcagccggctgcctcttcgac
ggctttgactgccagcgtgcggaaggccagtgcaaccccctgtacgacca
gtactgcaaggaccacttcagcgacgggcactgcgaccagggctgcaaca
gcgcggagtgcgagtgggacgggctggactgtgcggagcatgtacccgag
aggctggcggccggcacgctggtggtggtggtgctgatgccgccggagca
gctgcgcaacagctccttccacttcctgcgggagctcagccgcgtgctgc
acaccaacgtggtcttcaagcgtgacgcacacggccagcagatgatcttc
ccctactacggccgcgaggaggagctgcgcaagcaccccatcaagcgtgc
cgccgagggctgggccgcacctgacgccctgctgggccaggtgaaggcct
cgctgctccctggtggcagcgagggtgggcggcggcggagggagctggac
cccatggacgtccgcggctccatcgtctacctggagattgacaaccggca
gtgtgtgcaggcctcctcgcagtgcttccagagtgccaccgacgtggccg
cattcctgggagcgctcgcctcgctgggcagcctcaacatcccctacaag
atcgaggccgtgcagagtgagaccgtggagccgcccccgccggcgcagct
gcacttcatgtacgtggcggcggccgcctttgtgcttctgttcttcgtgg
gctgcggggtgctgctgtcccgcaagcgccggcggcagcatggccagctc
tggttccctgagggcttcaaagtgtctgaggccagcaagaagaagcggcg
ggagcccctcggcgaggactccgtgggcctcaagcccctgaagaacgctt
cagacggtgccctcatggacgacaaccagaatgagtggggggacgaggac
ctggagaccaagaagttccggttcgaggagcccgtggttctgcctgacct
ggacgaccagacagaccaccggcagtggactcagcagcacctggatgccg
ctgacctgcgcatgtctgccatggcccccacaccgccccagggtgaggtt
gacgccgactgcatggacgtcaatgtccgcgggcctgatggcttcacccc
gctcatgatcgcctcctgcagcgggggcggcctggagacgggcaacagcg
aggaagaggaggacgcgccggccgtcatctccgacttcatctaccagggc
gccagcctgcacaaccagacagaccgcacgggcgagaccgccttgcacct
ggccgcccgctactcacgctctgatgccgccaagcgcctgctggaggcca
gcgcagatgccaacatccaggacaacatgggccgcaccccgctgcatgcg
gctgtgtctgccgacgcacaaggtgtcttccagatcctgatccggaaccg
agccacagacctggatgcccgcatgcatgatggcacgacgccactgatcc
tggctgcccgcctggccgtggagggcatgctggaggacctcatcaactca
cacgccgacgtcaacgccgtagatgacctgggcaagtccgccctgcactg
ggccgccgccgtgaacaatgtggatgccgcagttgtgctcctgaagaacg
gggctaacaaagatatgcagaacaacagggaggagacacccctgtttctg
gccgcccgggagggcagctacgagaccgccaaggtgctgctggaccactt
tgccaaccgggacatcacggatcatatggaccgcctgccgcgcgacatcg
cacaggagcgcatgcatcacgacatcgtgaggctgctggacgagtacaac
ctggtgcgcagcccgcagctgcacggagccccgctggggggcacgcccac
cctgtcgcccccgctctgctcgcccaacggctacctgggcagcctcaagc
ccggcgtgcagggcaagaaggtccgcaagcccagcagcaaaggcctggcc
tgtggaagcaaggaggccaaggacctcaaggcacggaggaagaagtccca
ggacggcaagggctgcctgctggacagctccggcatgctctcgcccgtgg
actccctggagtcaccccatggctacctgtcagacgtggcctcgccgcca
ctgctgccctccccgttccagcagtctccgtccgtgcccctcaaccacct
gcctgggatgcccgacacccacctgggcatcgggcacctgaacgtggcgg
ccaagcccgagatggcggcgctgggtgggggcggccggctggcctttgag
actggcccacctcgtctctcccacctgcctgtggcctctggcaccagcac
cgtcctgggctccagcagcggaggggccctgaatttcactgtgggcgggt
ccaccagtttgaatggtcaatgcgagtggctgtcccggctgcagagcggc
atggtgccgaaccaatacaaccctctgcgggggagtgtggcaccaggccc
cctgagcacacaggccccctccctgcagcatggcatggtaggcccgctgc
acagtagccttgctgccagcgccctgtcccagatgatgagctaccagggc
ctgcccagcacccggctggccacccagcctcacctggtgcagacccagca
ggtgcagccacaaaacttacagatgcagcagcagaacctgcagccagcaa
acatccagcagcagcaaagcctgcagccgccaccaccaccaccacagccg
caccttggcgtgagctcagcagccagcggccacctgggccggagcttcct
gagtggagagccgagccaggcagacgtgcagccactgggccccagcagcc
tggcggtgcacactattctgccccaggagagccccgccctgcccacgtcg
ctgccatcctcgctggtcccacccgtgaccgcagcccagttcctgacgcc
cccctcgcagcacagctactcctcgcctgtggacaacacccccagccacc
agctacaggtgcctgagcaccccttcctcaccccgtcccctgagtcccct
gaccagtggtccagctcgtccccgcattccaacgtctccgactggtccga
gggcgtctccagccctcccaccagcatgcagtcccagatcgcccgcattc
cggaggccttcaagtaa; AF159231.
NOTCH2: NM_024408; NM_010928.
NOTCH3: NM_000435 (SEQ ID NO: 73):
atggggccgggggcccgtggccgccgccgccgccgtcgcccgatgtcgcc
gccaccgccaccgccacccgtgcgggcgctgcccctgctgctgctgctag
cggggccgggggctgcagcccccccttgcctggacggaagcccgtgtgca
aatggaggtcgttgcacccagctgccctcccgggaggctgcctgcctgtg
cccgcctggctgggtgggtgagcggtgtcagctggaggacccctgtcact
caggcccctgtgctggccgtggtgtctgccagagttcagtggtggctggc
accgcccgattctcatgccggtgcccccgtggcttccgaggccctgactg
ctccctgccagatccctgcctcagcagcccttgtgcccacggtgcccgct
gctcagtggggcccgatggacgcttcctctgctcctgcccacctggctac
cagggccgcagctgccgaagcgacgtggatgagtgccgggtgggtgagcc
ctgccgccatggtggcacctgcctcaacacacctggctccttccgctgcc
agtgtccagctggctacacagggccactatgtgagaaccccgcggtgccc
tgtgcaccctcaccatgccgtaacgggggcacctgcaggcagagtggcga
cctcacttacgactgtgcctgtcttcctgggtttgagggtcagaattgtg
aagtgaacgtggacgactgtccaggacaccgatgtctcaatggggggaca
tgcgtggatggcgtcaacacctataactgccagtgccctcctgagtggac
aggccagttctgcacggaggacgtggatgagtgtcagctgcagcccaacg
cctgccacaatgggggtacctgcttcaacacgctgggtggccacagctgc
gtgtgtgtcaatggctggacaggcgagagctgcagtcagaatatcgatga
ctgtgccacagccgtgtgcttccatggggccacctgccatgaccgcgtgg
cttctttctactgtgcctgccccatgggcaagactggcctcctgtgtcac
ctggatgacgcctgtgtcagcaacccctgccacgaggatgctatctgtga
cacaaatccggtgaacggccgggccatttgcacctgtcctcccggcttca
cgggtggggcatgtgaccaggatgtggacgagtgctctatcggcgccaac
ccctgcgagcacttgggcaggtgcgtgaacacgcagggctccttcctgtg
ccagtgcggtcgtggctacactggacctcgctgtgagaccgatgtcaacg
agtgtctgtcggggccctgccgaaaccaggccacgtgcctcgaccgcata
ggccagttcacctgtatctgtatggcaggcttcacaggaacctattgcga
ggtggacattgacgagtgtcagagtagcccctgtgtcaacggtggggtct
gcaaggaccgagtcaatggcttcagctgcacctgcccctcgggcttcagc
ggctccacgtgtcagctggacgtggacgaatgcgccagcacgccctgcag
gaatggcgccaaatgcgtggaccagcccgatggctacgagtgccgctgtg
ccgagggctttgagggcacgctgtgtgatcgcaacgtggacgactgctcc
cctgacccatgccaccatggtcgctgcgtggatggcatcgccagcttctc
atgtgcctgtgctcctggctacacgggcacacgctgcgagagccaggtgg
acgaatgccgcagccagccctgccgccatggcggcaaatgcctagacctg
gtggacaagtacctctgccgctgcccttctgggaccacaggtgtgaactg
cgaagtgaacattgacgactgtgccagcaacccctgcacctttggagtct
gccgtgatggcatcaaccgctacgactgtgtctgccaacctggcttcaca
gggcccctttgtaacgtggagatcaatgagtgtgcttccagcccatgcgg
cgagggaggttcctgtgtggatggggaaaatggcttccgctgcctctgcc
cgcctggctccttgcccccactctgcctccccccgagccatccctgtgcc
catgagccctgcagtcacggcatctgctatgatgcacctggcgggttccg
ctgtgtgtgtgagcctggctggagtggcccccgctgcagccagagcctgg
cccgagacgcctgtgagtcccagccgtgcagggccggtgggacatgcagc
agcgatggaatgggtttccactgcacctgcccgcctggtgtccagggacg
tcagtgtgaactcctctccccctgcaccccgaacccctgtgagcatgggg
gccgctgcgagtctgcccctggccagctgcctgtctgctcctgcccccag
ggctggcaaggcccacgatgccagcaggatgtggacgagtgtgctggccc
cgcaccctgtggccctcatggtatctgcaccaacctggcagggagtttca
gctgcacctgccatggagggtacactggcccttcctgcgatcaggacatc
aatgactgtgaccccaacccatgcctgaacggtggctcgtgccaagacgg
cgtgggctccttttcctgctcctgcctccctggtttcgccggcccacgat
gagcccgcgatgtggatgagtgcctgagcaacccctgcggcccgggcacc
tgtaccgaccacgtggcctccttcacctgcacctgcccgccaggctacgg
aggcttccactgcgaacaggacctgcccgactgcagccccagctcctgct
tcaatggcgggacctgtgtggacggcgtgaactcgttcagctgcctgtgc
cgtcccggctacacaggagcccactgccaacatgaggcagacccctgcct
ctcgcggccctgcctacacgggggcgtctgcagcgccgcccaccctggct
tccgctgcacctgcctcgagagcttcacgggcccgcagtgccagacgctg
gtggattggtgcagccgccagccttgtcaaaacgggggtcgctgcgtcca
gactggggcctattgcctttgtccccctggatggagcggacgcctctgtg
acatccgaagcttgccctgcagggaggccgcagcccagatcggggtgcgg
ctggagcagctgtgtcaggcgggtgggcagtgtgtggatgaagacagctc
ccactactgcgtgtgcccagagggccgtactggtagccactgtgagcagg
aggtggacccctgcttggcccagccctgccagcatggggggacctgccgt
ggctatatggggggctacatgtgtgagtgtcttcctggctacaatggtga
taactgtgaggacgacgtggacgagtgtgcctcccagccctgccagcacg
ggggttcatgcattgacctcgtggcccgctatctctgctcctgtccccca
ggaacgctgggggtgctctgcgagattaatgaggatgactgcggcccagg
cccaccgctggactcagggccccggtgcctacacaatggcacctgcgtgg
acctggtgggtggtttccgctgcacctgtcccccaggatacactggtttg
cgctgcgaggcagacatcaatgagtgtcgctcaggtgcctgccacgcggc
acacacccgggactgcctgcaggacccaggcggaggtttccgttgccttt
gtcatgctggcttctcaggtcctcgctgtcagactgtcctgtctccctgc
gagtcccagccatgccagcatggaggccagtgccgtcctagcccgggtcc
tgggggtgggctgaccttcacctgtcactgtgcccagccgttctggggtc
cgcgttgcgagcgggtggcgcgctcctgccgggagctgcagtgcccggtg
ggcgtcccatgccagcagacgccccgcgggccgcgctgcgcctgcccccc
agggttgtcgggaccctcctgccgcagcttcccggggtcgccgccggggg
ccagcaacgccagctgcgcggccgccccctgtctccacgggggctcctgc
cgccccgcgccgctcgcgcccttcttccgctgcgcttgcgcgcagggctg
gaccgggccgcgctgcgaggcgcccgccgcggcacccgaggtctcggagg
agccgcggtgcccgcgcgccgcctgccaggccaagcgcggggaccagcgc
tgcgaccgcgagtgcaacagcccaggctgcggctgggacggcggcgactg
ctcgctgagcgtgggcgacccctggcggcaatgcgaggcgctgcagtgct
ggcgcctcttcaacaacagccgctgcgaccccgcctgcagctcgcccgcc
tgcctctacgacaacttcgactgccacgccggtggccgcgagcgcacttg
caacccggtgtacgagaagtactgcgccgaccactttgccgacggccgct
gcgaccagggctgcaacacggaggagtgcggctgggatgggctggattgt
gccagcgaggtgccggccctgctggcccgcggcgtgctggtgctcacagt
gctgctgccgccagaggagctactgcgttccagcgccgactttctgcagc
ggctcagcgccatcctgcgcacctcgctgcgcttccgcctggacgcgcac
ggccaggccatggtcttcccttaccaccggcctagtcctggctccgaacc
ccgggcccgtcgggagctggcccccgaggtgatcggctcggtagtaatgc
tggagattgacaaccggctctgcctgcagtcgcctgagaatgatcactgc
ttccccgatgcccagagcgccgctgactacctgggagcgttgtcagcggt
ggagcgcctggacttcccgtacccactgcgggacgtgcggggggagccgc
tggagcctccagaacccagcgtcccgctgctgccactgctagtggcgggc
gctgtcttgctgctggtcattctcgtcctgggtgtcatggtggcccggcg
caagcgcgagcacagcacctctggttccctgagggcttctcactgcacaa
ggacgtggcctctggtcacaagggccggcgggaacccgtgggccaggacg
cgctgggcatgaagaacatggccaagggtgagagcctgatgggggaggtg
gccacagactggatggacacagagtgcccagaggccaagcggctaaaggt
agaggagccaggcatgggggctgaggaggctgtggattgccgtcagtgga
ctcaacaccatctggttgctgctgacatccgcgtggcaccagccatggca
ctgacaccaccacagggcgacgcagatgctgatggcatggatgtcaatgt
gcgtggcccagatggcttcaccccgctaatgctggcttccttctgtgggg
gggctctggagccaatgccaactgaagaggatgaggcagatgacacatca
gctagcatcatctccgacctgatctgccagggggctcagcttggggcacg
gactgaccgtactggcgagactgctttgcacctggctgcccgttatgccc
gtgctgatgcagccaagcggctgctggatgctggggcagacaccaatgcc
caggaccactcaggccgcactcccctgcacacagctgtcacagccgatgc
ccagggtgtcttccagattctcatccgaaaccgctctacagacttggatg
cccgcatggcagatggctcaacggcactgatcctggcggcccgcctggca
gtagagggcatggtggaagagctcatcgccagccatgctgatgtcaatgc
tgtggatgagcttgggaaatcagccttacactgggctgcggctgtgaaca
acgtggaagccactttggccctgctcaaaaatggagccaataaggacatg
caggatagcaaggaggagacccccctattcctggccgcccgcgagggcag
ctatgaggctgccaagctgctgttggaccactttgccaaccgtgagatca
ccgaccacctggacaggctgccgcgggacgtagcccaggagagactgcac
caggacatcgtgcgcttgctggatcaacccagtgggccccgcagcccccc
cggtccccacggcctggggcctctgctctgtcctccaggggccttcctcc
ctggcctcaaagcggcacagtcggggtccaagaagagcaggaggcccccc
gggaaggcggggctggggccgcaggggccccgggggcggggcaagaagct
gacgctggcctgcccgggccccctggctgacagctcggtcacgctgtcgc
ccgtggactcgctggactccccgcggcctttcggtgggccccctgcttcc
cctggtggcttcccccttgaggggccctatgcagctgccactgccactgc
agtgtctctggcacagcttggtggcccaggccgggcgggtctagggcgcc
agccccctggaggatgtgtactcagcctgggcctgctgaaccctgtggct
gtgcccctcgattgggcccggctgcccccacctgcccctccaggcccctc
gttcctgctgccactggcgccgggaccccagctgctcaacccagggaccc
ccgtctccccgcaggagcggcccccgccttacctggcagtcccaggacat
ggcgaggagtacccggcggctggggcacacagcagccccccaaaggcccg
cttcctgcgggttcccagtgagcacccttacctgaccccatcccccgaat
cccctgagcactgggccagcccctcacctccctccctctcagactggtcc
gaatccacgcctagcccagccactgccactggggccatggccaccaccac
tggggcactgcctgcccagccacttcccttgtctgttcccagctcccttg
ctcaggcccagacccagctggggccccagccggaagttacccccaagagg
caagtgttggcctga.
Nurr1: NM_006186 (SEQ ID NO: 74):
atgccttgtgttcaggcgcagtatgggtcctcgcctcaaggagccagccc
cgcttctcagagctacagttaccactcttcgggagaatacagctccgatt
tcttaactccagagtttgtcaagtttagcatggacctcaccaacactgaa
atcactgccaccacttctctccccagcttcagtacctttatggacaacta
cagcacaggctacgacgtcaagccaccttgcttgtaccaaatgcccctgt
ccggacagcagtcctccattaaggtagaagacattcagatgcacaactac
cagcaacacagccacctgcccccccagtctgaggagatgatgccgcactc
cgggtcggtttactacaagccctcctcgcccccgacgcccaccaccccgg
gcttccaggtgcagcacagccccatgtgggacgacccgggatctctccac
aacttccaccagaactacgtggccactacgcacatgatcgagcagaggaa
aacgccagtctcccgcctctccctcttctcctttaagcaatcgccccctg
gcaccccggtgtctagttgccagatgcgcttcgacgggcccctgcacgtc
cccatgaacccggagcccgccggcagccaccacgtggtggacgggcagac
cttcgctgtgcccaaccccattcgcaagcccgcgtccatgggcttcccgg
gcctgcagatcggccacgcgtctcagctgctcgacacgcaggtgccctca
ccgccgtcgcggggctccccctccaacgaggggctgtgcgctgtgtgtgg
ggacaacgcggcctgccaacactacggcgtgcgcacctgtgagggctgca
aaggcttctttaagcgcacagtgcaaaaaaatgcaaaatacgtgtgttta
gcaaataaaaactgcccagtggacaagcgtcgccggaatcgctgtcagta
ctgccgatttcagaagtgcctggctgttgggatggtcaaagaagtggttc
gcacagacagtttaaaaggccggagaggtcgtttgccctcgaaaccgaag
agcccacaggagccctctcccccttcgcccccggtgagtctgatcagtgc
cctcgtcagggcccatgtcgactccaacccggctatgaccagcctggact
attccaggttccaggcgaaccctgactatcaaatgagtggagatgacacc
cagcatatccagcaattctatgatctcctgactggctccatggagatcat
ccggggctgggcagagaagatccctggcttcgcagacctgcccaaagccg
accaagacctgctttttgaatcagctttcttagaactgtttgtccttcga
ttagcatacaggtccaacccagtggagggtaaactcatcttttgcaatgg
ggtggtcttgcacaggttgcatgcgttcgtggctttggggaatggattga
ttccattgttgaattctcctccaacttgcagaatatgaacatcgacattt
ctgccttctcctgcattgctgccctggctatggtcacagagagacacggg
ctcaaggaacccaagagagtggaagaactgcaaaacaagattgtaaattg
tctcaaagaccacgtgactttcaacaatggggggttgaaccgccccaatt
atttgtccaaactgttggggaagctcccagaacttcgtaccctttgcaca
caggggctacagcgcattttctacctgaaattggaagacttggtgccacc
gccagcaataattgacaaacttttcctggacactttacctttctaa;
NM_019328.
NOV(CCN3): NM_002514 (SEQ ID NO: 75):
atgcagagtgtgcagagcacgagcttttgtctccgaaagcagtgcctttg
cctgaccttcctgcttctccatctcctgggacaggtcgctgcgactcagc
gctgccctccccagtgcccgggccggtgccctgcgacgccgccgacctgc
gcccccggggtgcgcgcggtgctggacggctgctcatgctgtctggtgtg
tgcccgccagcgtggcgagagctgctcagatctggagccatgcgacgaga
gcagtggcctctactgtgatcgcagcgcggaccccagcaaccagactggc
atctgcacggcggtagagggagataactgtgtgttcgatggggtcatcta
ccgcagtggagagaaatttcagccaagctgcaaattccagtgcacctgca
gagatgggcagattggctgtgtgccccgctgtcagctggatgtgctactg
cctgagcctaactgcccagctccaagaaaagttgaggtgcctggagagtg
ctgtgaaaagtggatctgtggcccagatgaggaggattcactgggaggcc
ttacccttgcagcttacaggccagaagccaccctaggagtagaagtctct
gactcaagtgtcaactgcattgaacagaccacagagtggacagcatgctc
caagagctgtggtatggggttctccacccgggtcaccaataggaaccgtc
aatgtgagatgctgaaacagactcggctctgcatggtgcggccctgtgaa
caagagccagagcagccaacagataagaaaggaaaaaagtgtctccgcac
caagaagtcactcaaagccatccacctgcagttcaagaactgcaccagcc
tgcacacctacaagcccaggttctgtggggtctgcagtgatggccgctgc
tgcactccccacaataccaaaaccatccaggcagagtttcagtgctcccc
agggcaaatagtcaagaagccagtgatggtcattgggacctgcacctgtc
acaccaactgtcctaagaacaatgaggccttcctccaggagctggagctg
aagactaccagagggaaaatgtaa; NM_010930; NM_030868;
BC015028.
OLIG1: NM_138983 (SEQ ID NO: 76):
atgctgcggccacagcggcccggagacttgcagctcggggcctccctcta
cgagctggtgggctacaggcagccgccctcctcctcctcctcctccacct
cctccacctcctccacttcctcctcctccacgacggcccccctcctcccc
aaggctgcgcgcgagaagccggaggcgccggccgagcctccaggccccgg
gcccgggtcaggcgcgcacccgggcggcagcgcccggccggacgccaagg
aggagcagcagcagcagctgcggcgcaagatcaacagccgcgagcggaag
cgcatgcaggacctgaacctggccatggacgccctgcgcgaggtcatcct
gccctactcagcggcgcactgccagggcgcgcccggccgcaagctctcca
agatagccacgctgctgctcgcccgcaactacatcctactgctgggcagc
tcgctgcaggagctgcgccgcgcgctgggcgagggcgccgggcccgccgc
gccgcgcctgctgctggccgggctgcccctgctcgccgccgcgcccggct
ccgtgttgctggcgcccggcgccgtaggaccccccgacgcgctgcgcccc
gccaagtacctgtcgctggcgctggacgagccgccgtgcggccagttcgc
tctccccggcggcgcgcaggcggccccggcctctgcacctgcgccgtgtg
caagttcccgcacctggtcccggccagcctgggcctggccgccgtgcagg
cgcaattctccaagtga; NM_016968; NM_001020796.
OLIG2: NM_005806 (SEQ ID NO: 77):
atggactcggacgccagcctggtgtccagccgcccgtcgtcgccagagcc
cgatgacctttttctgccggcccggagtaagggcagcagcggcagcgcct
tcactgggggcaccgtgtcctcgtccaccccgagtgactgcccgccggag
ctgagcgccgagctgcgcggcgctatgggctctgcgggcgcgcatcctgg
ggacaagctaggaggcagtggcttcaagtcatcctcgtccagcacctcgt
cgtctacgtcgtcggcggctgcgtcgtccaccaagaaggacaagaagcaa
atgacagagccggagctgcagcagctgcgtctcaagatcaacagccgcga
gcgcaagcgcatgcacgacctcaacatcgccatggatggcctccgcgagg
tcatgccgtacgcacacggcccttcggtgcgcaagctttccaagatcgcc
acgctgctgctggcgcgcaactacatcctcatgctcaccaactcgctgga
ggagatgaagcgactggtgagcgagatctacgggggccaccacgctggct
tccacccgtcggcctgcggcggcctggcgcactccgcgcccctgcccgcc
gccaccgcgcacccggcagcagcagcgcacgccgcacatcaccccgcggt
gcaccaccccatcctgccgcccgccgccgcagcggctgctgccgccgctg
cagccgcggctgtgtccagcgcctctctgcccggatccgggctgccgtcg
gtcggctccatccgtccaccgcacggcctactcaagtctccgtctgctgc
cgcggccgccccgctggggggcgggggcggcggcagtggggcgagcgggg
gcttccagcactggggcggcatgccctgcccctgcagcatgtgccaggtg
ccgccgccgcaccaccacgtgtcggctatgggcgccggcagcctgccgcg
cctcacctccgacgccaagtga.
Pdx1: NM_000209 (SEQ ID NO: 78):
atgaacggcgaggagcagtactacgcggccacgcagctttacaaggaccc
atgcgcgttccagcgaggcccggcgccggagttcagcgccagcccccctg
cgtgcctgtacatgggccgccagcccccgccgccgccgccgcacccgttc
cctggcgccctgggcgcgctggagcagggcagccccccggacatctcccc
gtacgaggtgccccccctcgccgacgaccccgcggtggcgcaccttcacc
accacctcccggctcagctcgcgctcccccacccgcccgccgggcccttc
ccggagggagccgagccgggcgtcctggaggagcccaaccgcgtccagct
gcctttcccatggatgaagtctaccaaagctcacgcgtggaaaggccagt
gggcaggcggcgcctacgctgcggagccggaggagaacaagcggacgcgc
acggcctacacgcgcgcacagctgctagagctggagaaggagttcctatt
caacaagtacatctcacggccgcgccgggtggagctggctgtcatgttga
acttgaccgagagacacatcaagatctggttccaaaaccgccgcatgaag
tggaaaaaggaggaggacaagaagcgcggcggcgggacagctgtcggggg
tggcggggtcgcggagcctgagcaggactgcgccgtgacctccggcgagg
agcttctggcgctgccgccgccgccgccccccggaggtgctgtgccgccc
gctgcccccgttgccgcccgagagggccgcctgccgcctggccttagcgc
gtcgccacagccctccagcgtcgcgcctcggcggccgcaggaaccacgat
ga; NM_008814; NM_022852.
Pet1(FEV): BC138435; NM_017521 (SEQ ID NO: 79):
atgagacagagcggcgcctcccagcccctgctgatcaacatgtacctgcc
agatcccgtcggagacggtctcttcaaggacgggaagaacccgagctggg
ggccgctgagccccgcggttcagaaaggcagcggacagatccagctgtgg
cagtttctgctggagctgctggctgaccgcgcgaacgccggctgcatcgc
gtgggagggcggtcacggcgagttcaagctcacggacccggacgaggtgg
cgcggcggtggggcgagcgcaagagcaagcccaacatgaactacgacaag
ctgagccgcgccctgcgctactactacgacaagaacatcatgagcaaggt
gcatggcaagcgctacgcctaccgcttcgacttccagggcctggcgcagg
cctgccagccgccgcccgcgcacgctcatgccgccgccgcagctgctgcc
gccgccgcggccgcccaggacggcgcgctctacaagctgcccgccggcct
cgccccgctgcccttccccggcctctccaaactcaacctcatggccgcct
cggccggggtcgcgcccgccggcttctcctactggccgggcccgggcccc
gccgccaccgctgccgccgccaccgccgcgctctaccccagtcccagctt
gcagcccccgcccgggcccttcggggccgtggccgcagcctcgcacttgg
ggggccattaccactag; NG_002690; NP_059991.
Phox2a: NM_005169 (SEQ ID NO: 80):
atggactactcctacctcaattcgtacgactcgtgcgtggcggccatgga
ggcgtccgcctacggcgactttggcgcctgcagccagcccggcggcttcc
aatacagccccctgcggcccgctttccccgcggcagggccgccctgcccc
gcgctcggctcctccaactgcgcacttggcgccctacgcgaccaccagcc
cgcgccctactcggcagtgccctacaagttcttcccagagccatccggcc
tgcacgagaagcgcaagcagcggcgcatccgcaccacgttcaccagcgcg
cagctcaaggagctggagcgcgttttcgctgagacccactaccccgacat
ttacacgcgtgaggagctggcgctcaagatcgacctcactgaggctcgcg
tgcaggtctggttccagaaccgccgggccaagttccgcaaacaggagcgc
gcggccagcgccaagggcgcggcgggcgcggcgggcgccaaaaagggcga
ggcgcgctgctcctccgaggacgacgattccaaggagtccacgtgcagcc
ccacgcccgatagcaccgcctcgctgccgccgccgcctgcgcccggcctg
gccagcccgcgcctgagccccagcccgctgcccgtcgcactgggctccgg
gccgggacctgggccggggccacagccgctcaagggcgcactgtgggccg
gtgtggcgggcggtgggggcggcgggcctggcgcgggagcggccgaacta
cttaaggcttggcagccggcggagtccggccccgggcccttctccggggt
tctgtcctcctttcaccggaagcccggccccgccctgaagaccaatctct
tctag; AJ320270; AY371497; AY371496.
Phox2b: NM_003924 (SEQ ID NO: 81):
atgtataaaatggaatattcttacctcaattcctctgcctacgagtcctg
tatggctgggatggacacctcgagcctggcttcagcctatgctgacttca
gttcctgcagccaggccagtggcttccagtataacccgataaggaccact
tttggggccacgtccggctgcccttccctcacgccgggatcctgcagcct
gggcaccctcagggaccaccagagcagtccgtacgccgcagttccttaca
aactcttcacggaccacggcggcctcaacgagaagcgcaagcagcggcgc
atccgcaccactttcaccagtgcccagctcaaagagctggaaagggtctt
cgcggagactcactaccccgacatctacactcgggaggagctggccctga
agatcgacctcacagaggcgcgagtccaggtgtggttccagaaccgccgc
gccaagtttcgcaagcaggagcgcgcagcggcagccgcagcggccgcggc
caagaacggctcctcgggcaaaaagtctgactcttccagggacgacgaga
gcaaagaggccaagagcactgacccggacagcactgggggcccaggtccc
aatcccaaccccacccccagctgcggggcgaatggaggcggcggcggcgg
gcccagcccggctggagctccgggggcggcggggcccgggggcccgggag
gcgaacccggcaagggcggcgcagcagcagcggcggcggccgcggcagcg
gcggcggcggcagcggcagcggcggcagctggaggcctggctgcggctgg
gggccctggacaaggctgggctcccggccccggccccatcacctccatcc
cggattcgcttgggggtcccttcgccagcgtcctatcttcgctccaaaga
cccaacggtgccaaagccgccttagtgaagagcagtatgttctga;
NM_008888; AY371498; Y14493.
Pit1: NM_000306 (SEQ ID NO: 82):
atgagttgccaagcttttacttcggctgatacctttatacctctgaattc
tgacgcctctgcaactctgcctctgataatgcatcacagtgctgccgagt
gtctaccagtctccaaccatgccaccaatgtgatgtctacagcaacagga
cttcattattctgttccttcctgtcattatggaaaccagccatcaaccta
tggagtgatggcaggtagtttaaccccttgtctttataaatttcctgacc
acaccttgagtcatggatttcctcctatacaccagcctcttctggcagag
gaccccacagctgctgatttcaagcaggaactcaggcggaaaagtaaatt
ggtggaagagccaatagacatggattctccagaaatcagagaacttgaaa
agtttgccaatgaattaaagtgagacgaattaaattaggatacacccaga
caaatgttggggaggccctggcagctgtgcatggctctgaattcagtcaa
acaacaatctgccgatttgaaaatctgcagctcagctttaaaaatgcatg
caaactgaaagcaatattatccaaatggctggaggaagctgagcaagtag
gagctttgtacaatgaaaaagtgggagcaaatgaaaggaaaagaaaacga
agaacaactataagcattgctgctaaagatgctctggagagacactttgg
agaacagaataaaccttcttctcaagagatcatgaggatggctgaagaac
tgaatctggagaaagaagtagtaagagtttggttttgcaaccggaggcag
agagaaaaacgggtgaaaacaagtctgaatcagagtttattttctatttc
taaggaacatcttgagtgcagataa; M23253.
PITX3: NM_005029 (SEQ ID NO: 83):
atggagttcggcctgctcagcgaggcagaggcccggagccctgccctgtc
gctgtcagacgctggcactccgcacccccagctcccagagcacggctgca
agggccaggagcacagcgactcagccccggcctcggcttcgctgcccggc
gctccccagaggacggttcgctgaaaaagaagcagcggcggcagcgcacg
cacttcaccagccagcagctacaggagctagaggcgaccttccagaggaa
ccgctaccccgacatgagcacgcgcgaggagatcgccgtgtggaccaacc
tcaccgaggcccgcgtgcgggtgtggttcaagaaccggcgcgccaaatgg
cggaagcgcgagcgcagccagcaggccgagctatgcaaaggcagcttcgc
ggcgccgctcggggggctggtgccgccctacgaggaggtgtaccccggct
actcgtacggcaactggccgcccaaggctcttgccccgccgctcgccgcc
aagacctttccattcgccttcaactcggtcaacgtggggcctctggcttc
gcagcccgtcttctcgccacccagctccatcgccgcctccatggtgccct
ccgccgcggctgccccgggcaccgtgccagggcctggggccctgcagggc
ctgggcgggggcccccccgggctggctccggccgccgtgtcctccggggc
cgtgtcctgcccttatgcctcggccgccgccgccgccgcggctgccgcct
cttccccctacgtctatcgggacccgtgtaactcgagcctggccagcctg
cggctcaaagccaaacagcacgcctccttcagctaccccgctgtgcacgg
gccgcccccggcagccaaccttagtccgtgccagtacgccgtggaaaggc
ccgtatga; NM_008852; NM_008987;
RUNX1: NM_001001890 (SEQ ID NO: 84):
atgcgtatccccgtagatgccagcacgagccgccgcttcacgccgccttc
caccgcgctgagcccaggcaagatgagcgaggcgttgccgctgggcgccc
cggacgccggcgctgccctggccggcaagctgaggagcggcgaccgcagc
atggtggaggtgctggccgaccacccgggcgagctggtgcgcaccgacag
ccccaacttcctctgctccgtgctgcctacgcactggcgctgcaacaaga
ccctgcccatcgctttcaaggtggtggccctaggggatgttccagatggc
actctggtcactgtgatggctggcaatgatgaaaactactcggctgagct
gagaaatgctaccgcagccatgaagaaccaggttgcaagatttaatgacc
tcaggtttgtcggtcgaagtggaagagggaaaagcttcactctgaccatc
actgtcttcacaaacccaccgcaagtcgccacctaccacagagccatcaa
aatcacagtggatgggccccgagaacctcgaagacatcggcagaaactag
atgatcagaccaagcccgggagcttgtccttttccgagcggctcagtgaa
ctggagcagctgcggcgcacagccatgagggtcagcccacaccacccagc
ccccacgcccaaccctcgtgcctccctgaaccactccactgcctttaacc
ctcagcctcagagtcagatgcaggatacaaggcagatccaaccatcccca
ccgtggtcctacgatcagtcctaccaatacctgggatccattgcctctcc
ttctgtgcacccagcaacgcccatttcacctggacgtgccagcggcatga
caaccctctctgcagaactttccagtcgactctcaacggcacccgacctg
acagcgttcagcgacccgcgccagttccccgcgctgccctccatctccga
cccccgcatgcactatccaggcgccttcacctactccccgacgccggtca
cctcgggcatcggcatcggcatgtcggccatgggctcggccacgcgctac
cacacctacctgccgccgccctaccccggctcgtcgcaagcgcagggagg
cccgttccaagccagctcgccctcctaccacctgtactacggcgcctcgg
ccggctcctaccagttctccatggtgggcggcgagcgctcgccgccgcgc
atcctgccgccctgcaccaacgcctccaccggctccgcgctgctcaaccc
cagcctcccgaaccagagcgacgtggtggaggccgagggcagccacagca
actcccccaccaacatggcgccctccgcgcgcctggaggaggccgtgtgg
aggccctactga; AY509916; AY509915; NM_001001890.2;
NP_001001890.1; NM_001122607.1; NP_001116079.1;
NM_001754.4; NP_001745.2
Runx2: NM_001015051 (SEQ ID NO: 85):
atgcttcattcgcctcacaaacaaccacagaaccacaagtgcggtgcaaa
ctttctccaggaggacagcaagaagtctctggtttttaaatggttaatct
ccgcaggtcactaccagccaccgagaccaacagagtcatttaaggctgca
agcagtatttacaacagagggtacaagttctatctgaaaaaaaaaggagg
gactatggcatcaaacagcctcttcagcacagtgacaccatgtcagcaaa
acttcttttgggatccgagcaccagccggcgcttcagccccccctccagc
agcctgcagcccggcaaaatgagcgacgtgagcccggtggtggctgcgca
acagcagcagcaacagcagcagcagcaacagcagcagcagcagcagcaac
agcagcagcagcagcaggaggcggcggcggcggctgcggcggcggcggcg
gctgcggcggcggcagctgcagtgccccggttgcggccgccccacgacaa
ccgcaccatggtggagatcatcgccgaccacccggccgaactcgtccgca
ccgacagccccaacttcctgtgctcggtgctgccctcgcactggcgctgc
aacaagaccctgcccgtggccttcaaggtggtagccctcggagaggtacc
agatgggactgtggttactgtcatggcgggtaacgatgaaaattattctg
ctgagctccggaatgcctctgctgttatgaaaaaccaagtagcaaggttc
aacgatctgagatttgtgggccggagtggacgaggcaagagtttcacctt
gaccataaccgtcttcacaaatcctccccaagtagctacctatcacagag
caattaaagttacagtagatggacctcgggaacccagaaggcacagacag
aagcttgatgactctaaacctagtttgttctctgaccgcctcagtgattt
agggcgcattcctcatcccagtatgagagtaggtgtcccgcctcagaacc
cacggccctccctgaactctgcaccaagtccttttaatccacaaggacag
agtcagattacagaccccaggcaggcacagtcttccccgccgtggtccta
tgaccagtcttacccctcctacctgagccagatgacgtccccgtccatcc
actctaccaccccgctgtcttccacacggggcactgggatcctgccatca
ccgatgtgcctaggcgcatttcaggtgcttcagaactgggccctttttac
agaccccaggcagttcccaagcatttcatccctcactgagagccgcttct
ccaacccacgaatgcactatccagccacctttacttacaccccgccagtc
acctcaggcatgtccctcggtatgtccgccaccactcactaccacaccta
cctgccaccaccctaccccggctcttcccaaagccagagtggacccttcc
agaccagcagcactccatatctctactatggcacttcgtcaggatcctat
cagtttcccatggtgccggggggagaccggtctccttccagaatgcttcc
gccatgcaccaccacctcgaatggcagcacgctattaaatccaaatttgc
ctaaccagaatgatggtgttgacgctgatggaagccacagcagttcccca
actgttttgaattctagtggcagaatggatgaatctgtttggcgaccata
ttga; NM_001015051.2; NP_001015051.2;
NM_001015051; NM_001024630.2; NP_001019801.2;
NM_004348.3; NP_004339.3
Shh: NM_000193 (SEQ ID NO: 86):
atgctgctgctggcgagatgtctgctgctagtcctcgtctcctcgctgct
ggtatgctcgggactggcgtgcggaccgggcagggggttcgggaagagga
ggcaccccaaaaagctgctccctttagcctacaagcagtttatccccaat
gtggccgagaagaccctaggcgccagcggaaggtatgaagggaagatctc
cagaaactccgagcgatttaaggaactcacccccaattacaaccccgaca
tcatatttaaggatgaagaaaacaccggagcggacaggctgatgactcag
aggtgtaaggacaagttgaacgctttggccatctcggtgatgaaccagtg
gccaggagtgaaactgcgggtgaccgagggctgggacgaagatggccacc
actcagaggagtctctgcactacgagggccgcgcagtggacatcaccacg
tctgaccgcgaccgcagcaagtacggcatgctggcccgcctggcggtgga
ggccggcttcgactgggtgtactacgagtccaaggcacatatccactgct
cggtgaaagcagagaactcggtggcggccaaatcgggaggctgcttcccg
ggctcggccacggtgcacctggagcagggcggcaccaagctggtgaagga
cctgagccccggggaccgcgtgctggcggcggacgaccagggccggctgc
tctacagcgacttcctcactttcctggaccgcgacgacggcgccaagaag
gtcttctacgtgatcgagacgcgggagccgcgcgagcgcctgctgctcac
cgccgcgcacctgctctttgtggcgccgcacaacgactcggccaccgggg
agcccgaggcgtcctcgggctcggggccgccttccgggggcgcactgggg
cctcgggcgctgttcgccagccgcgtgcgcccgggccagcgcgtgtacgt
ggtggccgagcgtgacggggaccgccggctcctgcccgccgctgtgcaca
gcgtgaccctaagcgaggaggccgcgggcgcctacgcgccgctcacggcc
cagggcaccattctcatcaaccgggtgctggcctcgtgctacgcggtcat
cgaggagcacagctgggcgcaccgggccttcgcgcccttccgcctggcgc
acgcgctcctggctgcactggcgcccgcgcgcacggaccgcggcggggac
agcggcggcggggaccgcgggggcggcggcggcagagtagccctaaccgc
tccaggtgctgccgacgctccgggtgcgggggccaccgcgggcatccact
ggtactcgcagctgctctaccaaataggcacctggctcctggacagcgag
gccctgcacccgctgggcatggcggtcaagtccagctga;
NP_000184; NM_009170; NP_033196; NM_204821;
NP_990152.
Sox9: NM_000346 (SEQ ID NO: 87):
atgaatctcctggaccccttcatgaagatgaccgacgagcaggagaaggg
cctgtccggcgcccccagccccaccatgtccgaggactccgcgggctcgc
cctgcccgtcgggctccggctcggacaccgagaacacgcggccccaggag
aacacgttccccaagggcgagcccgatctgaagaaggagagcgaggagga
caagttccccgtgtgcatccgcgaggcggtcagccaggtgctcaaaggct
acgactggacgctggtgcccatgccggtgcgcgtcaacggctccagcaag
aacaagccgcacgtcaagcggcccatgaacgccttcatggtgtgggcgca
ggcggcgcgcaggaagctcgcggaccagtacccgcacttgcacaacgccg
agctcagcaagacgctgggcaagctctggagacttctgaacgagagcgag
aagcggcccttcgtggaggaggcggagcggctgcgcgtgcagcacaagaa
ggaccacccggattacaagtaccagccgcggcggaggaagtcggtgaaga
acgggcaggcggaggcagaggaggccacggagcagacgcacatctccccc
aacgccatcttcaaggcgctgcaggccgactcgccacactcctcctccgg
catgagcgaggtgcactcccccggcgagcactcggggcaatcccagggcc
caccgaccccacccaccacccccaaaaccgacgtgcagccgggcaaggct
gacctgaagcgagaggggcgccccttgccagaggggggcagacagccccc
tatcgacttccgcgacgtggacatcggcgagctgagcagcgacgtcatct
ccaacatcgagaccttcgatgtcaacgagtttgaccagtacctgccgccc
aacggccacccgggggtgccggccacgcacggccaggtcacctacacggg
cagctacggcatcagcagcaccgcggccaccccggcgagcgcgggccacg
tgtggatgtccaagcagcaggcgccgccgccacccccgcagcagccccca
caggccccgccggccccgcaggcgcccccgcagccgcaggcggcgccccc
acagcagccggcggcacccccgcagcagccacaggcgcacacgctgacca
cgctgagcagcgagccgggccagtcccagcgaacgcacatcaagacggag
cagctgagccccagccactacagcgagcagcagcagcactcgccccaaca
gatcgcctacagccccttcaacctcccacactacagcccctcctacccgc
ccatcacccgctcacagtacgactacaccgaccaccagaactccagctcc
tactacagccacgcggcaggccagggcaccggcctctactccaccttcac
ctacatgaaccccgctcagcgccccatgtacacccccatcgccgacacct
ctggggtcccttccatcccgcagacccacagcccccagcactgggaacaa
cccgtctacacacagctcactcgaccttga; NM_000346;
NP_000337; NM_011448; NP_035578; XM_343981;
NP_343982.
Sox17: NM_022454 (SEQ ID NO: 88):
atgagcagcccggatgcgggatacgccagtgacgaccagagccagaccca
gagcgcgctgcccgcggtgatggccgggctgggcccctgcccctgggccg
agtcgctgagccccatcggggacatgaaggtgaagggcgaggcgccggcg
aacagcggagcaccggccggggccgcgggccgagccaagggcgagtcccg
tatccggcggccgatgaacgctttcatggtgtgggctaaggacgagcgca
agcggctggcgcagcagaatccagacctgcacaacgccgagttgagcaag
atgctgggcaagtcgtggaaggcgctgacgctggcggagaagcggccctt
cgtggaggaggcagagcggctgcgcgtgcagcacatgcaggaccacccca
actacaagtaccggccgcggcggcgcaagcaggtgaagcggctgaagcgg
gtggagggcggcttcctgcacggcctggctgagccgcaggcggccgcgct
gggccccgagggcggccgcgtggccatggacggcctgggcctccagttcc
ccgagcagggcttccccgccggcccgccgctgctgcctccgcacatgggc
ggccactaccgcgactgccagagtctgggcgcgcctccgctcgacggcta
cccgttgcccacgcccgacacgtccccgctggacggcgtggaccccgacc
cggctttcttcgccgccccgatgcccggggactgcccggcggccggcacc
tacagctacgcgcaggtctcggactacgctggccccccggagcctcccgc
cggtcccatgcacccccgactcggcccagagcccgcgggtccctcgattc
cgggcctcctggcgccacccagcgcccttcacgtgtactacggcgcgatg
ggctcgcccggggcgggcggcgggcgcggcttccagatgcagccgcaaca
ccagcaccagcaccagcaccagcaccaccccccgggccccggacagccgt
cgccccctccggaggcactgccctgccgggacggcacggaccccagtcag
cccgccgagctcctcggggaggtggaccgcacggaatttgaacagtatct
gcacttcgtgtgcaagcctgagatgggcctcccctaccaggggcatgact
ccggtgtgaatctccccgacagccacggggccatttcctcggtggtgtcc
gacgccagctccgcggtatattactgcaactatcctgacgtgtga;
BC140307; NM_011441.
DLX2: NM_004405 (SEQ ID NO: 89):
atgactggagtctttgacagtctagtggctgatatgcactcgacccagat
cgccgcctccagcacgtaccaccagcaccagcagcccccgagcggcggcg
gcgccggcccgggtggcaacagcagcagcagcagcagcctccacaagccc
caggagtcgcccacccttccggtgtccaccgccaccgacagcagctacta
caccaaccagcagcacccggcgggcggcggcggcggcgggggctcgccct
acgcgcacatgggttcctaccagtaccaagccagcggcctcaacaacgtc
ccttactccgccaagagcagctatgacctgggctacaccgccgcctacac
ctcctacgctccctatggaaccagttcgtccccagccaacaacgagcctg
agaaggaggaccttgagcctgaaattcggatagtgaacgggaagccaaag
aaagtccggaaaccccgcaccatctactccagtttccagctggcggctct
tcagcggcgtttccaaaagactcaatacttggccttgccggagcgagccg
agctggcggcctctctgggcctcacccagactcaggtcaaaatctggttc
cagaaccgccggtccaagttcaagaagatgtggaaaagtggtgagatccc
ctcggagcagcaccctggggccagcgcttctccaccttgtgcttcgccgc
cagtctcagcgccggcctcctgggactttggtgtgccgcagcggatggcg
ggcggcggtggtccgggcagtggcggcagcggcgccggcagctcgggctc
cagcccgagcagcgcggcctcggcttttctgggcaactacccctggtacc
accagacctcgggatccgcctcacacctgcaggccacggcgccgctgctg
caccccactcagaccccgcagccgcatcaccaccaccaccatcacggcgg
cgggggcgccccggtgagcgcggggacgattttctaa;
NP_004396.1; NM_010054.
DLX5: NM_005221 (SEQ ID NO: 90):
atgacaggagtgtttgacagaagggtccccagcatccgatccggcgactt
ccaagctccgttccagacgtccgcagctatgcaccatccgtctcaggaat
cgccaactttgcccgagtcttcagctaccgattctgactactacagccct
acggggggagccccgcacggctactgctctcctacctcggcttcctatgg
caaagctctcaacccctaccagtatcagtatcacggcgtgaacggctccg
ccgggagctacccagccaaagcttatgccgactatagctacgctagctcc
taccaccagtacggcggcgcctacaaccgcgtcccaagcgccaccaacca
gccagagaaagaagtgaccgagcccgaggtgagaatggtgaatggcaaac
caaagaaagttcgtaaacccaggactatttattccagctttcagctggcc
gcattacagagaaggtttcagaagactcagtacctcgccttgccggaacg
cgccgagctggccgcctcgctgggattgacacaaacacaggtgaaaatct
ggtttcagaacaaaagatccaagatcaagaagatcatgaaaaacggggag
atgcccccggagcacagtcccagctccagcgacccaatggcgtgtaactc
gccgcagtctccagcggtgtgggagccccagggctcgtcccgctcgctca
gccaccaccctcatgcccaccctccgacctccaaccagtccccagcgtcc
agctacctggagaactctgcatcctggtacacaagtgcagccagctcaat
caattcccacctgccgccgccgggctccttacagcacccgctggcgctgg
cctccgggacactctattag; NM_005221; NP_005212.
HES1: NM_005524 (SEQ ID NO: 91):
atgccagctgatataatggagaaaaattcctcgtccccggtggctgctac
cccagccagtgtcaacacgacaccggataaaccaaagacagcatctgagc
acagaaagtcatcaaagcctattatggagaaaagacgaagagcaagaata
aatgaaagtctgagccagctgaaaacactgattttggatgctctgaagaa
agatagctcgcggcattccaagctggagaaggcggacattctggaaatga
cagtgaagcacctccggaacctgcagcgggcgcagatgacggctgcgctg
agcacagacccaagtgtgctggggaagtaccgagccggcttcagcgagtg
catgaacgaggtgacccgcttcctgtccacgtgcgagggcgttaataccg
aggtgcgcactcggctgctcggccacctggccaactgcatgacccagatc
aatgccatgacctaccccgggcagccgcaccccgccttgcaggcgccgcc
accgcccccaccgggacccggcggcccccagcacgcgccgttcgcgccgc
cgccgccactcgtgcccatccccgggggcgcggcgccccctcccggcggc
gccccctgcaagctgggcagccaggctggagaggcggctaaggtgtttgg
aggcttccaggtggtaccggctcccgatggccagtttgctttcctcattc
ccaacggggccttcgcgcacagcggccctgtcatccccgtctacaccagc
aacagcggcacctccgtgggccccaacgcagtgtcaccttccagcggccc
ctcgcttacggcggactccatgtggaggccgtggcggaactga;
NP_0055151.1; NM_008235; NP_032261.
FGF8: NM_006119 (SEQ ID NO: 92):
atgggcagcccccgctccgcgctgagctgcctgctgttgcacttgctggt
cctctgcctccaagcccaggtaactgttcagtcctcacctaattttacac
agcatgtgagggagcagagcctggtgacggatcagctcagccgccgcctc
atccggacctaccaactctacagccgcaccagcgggaagcacgtgcaggt
cctggccaacaagcgcatcaacgccatggcagaggacggcgaccccttcg
caaagctcatcgtggagacggacacctttggaagcagagttcgagtccga
ggagccgagacgggcctctacatctgcatgaacaagaaggggaagctgat
cgccaagagcaacggcaaaggcaaggactgcgtcttcacggagattgtgc
tggagaacaactacacagcgctgcagaatgccaagtacgagggctggtac
atggccttcacccgcaagggccggccccgcaagggctccaagacgcggca
gcaccagcgtgaggtccacttcatgaagcggctgccccggggccaccaca
ccaccgagcagagcctgcgcttcgagttcctcaactacccgcccttcacg
cgcagcctgcgcggcagccagaggacttgggcccccgagccccgatag;
NM_010205; NP_034335; NM_010205; NP_034335;
NP_006110 NM_033163; NP_149353; NM_033164;
NP_149354; NM_033165; NP_149355.
PITX2: NM_000325 (SEQ ID NO: 93):
atgaactgcatgaaaggcccgcttcacttggagcaccgagcagcggggac
caagctgtcggccgtctcctcatcttcctgtcaccatccccagccgttag
ccatggcttcggttctggctcccggtcagccccggtcgctggactcctcc
aagcacaggctggaggtgcacaccatctccgacacctccagcccggaggc
cgcagagaaagataaaagccagcaggggaagaatgaggacgtgggcgccg
aggacccgtctaagaagaagcggcaaaggcggcagcggactcactttacc
agccagcagctccaggagctggaggccactttccagaggaaccgctaccc
ggacatgtccacacgcgaagaaatcgctgtgtggaccaaccttacggaag
cccgagtccgggtttggttcaagaatcgtcgggccaaatggagaaagagg
gagcgcaaccagcaggccgagctatgcaagaatggcttcgggccgcagtt
caatgggctcatgcagccctacgacgacatgtacccaggctattcctaca
acaactgggccgccaagggccttacatccgcctccctatccaccaagagc
ttccccttcttcaactctatgaacgtcaaccccctgtcatcacagagcat
gttttccccacccaactctatctcgtccatgagcatgtcgtccagcatgg
tgccctcagcagtgacaggcgtcccgggctccagtctcaacagcctgaat
aacttgaacaacctgagtagcccgtcgctgaattccgcggtgccgacgcc
tgcctgtccttacgcgccgccgactcctccgtatgtttatagggacacgt
gtaactcgagcctggccagcctgagactgaaagcaaagcagcactccagc
ttcggctacgccagcgtgcagaacccggcctccaacctgagtgcttgcca
gtatgcagtggaccggcccgtgtga; NM_000325; NP_000316;
NM_153426; NP_700475; NM_153427; NP_700476;
NM_001042502; NP_001035967; NM_001042504;
NP_001035969.
REST4: DQ644039 (SEQ ID NO: 94):
atggccacccaggtgatggggcagtcttctggaggaggcagtctcttcaa
caacagtgccaacatgggcatggccttaaccaacgacatgtacgacctgc
acgagctctcgaaagctgaactggcagcccctcagctcatcatgttagcc
aacgtggccctgacgggggaggcaagcggcagctgctgcgattacctggt
cggtgaagagaggcagatggccgaattgatgcccgtgggagacaaccact
tctcagaaagtgaaggagaaggcctggaagagtcggctgacctcaaaggg
ctggaaaacatggaactgggaagtttggagctaagtgctgtagaacccca
gcccgtatttgaagcctcagctgccccagaaatatacagcgccaataaag
atcccgctccagaaacacccgtggcggaagacaaatgcaggagttctaag
gccaagcccttccggtgtaagccttgccagtacgaagccgaatctgaaga
gcagtttgtgcatcacatccggattcacagcgctaagaagttctttgtgg
aggaaagtgcagagaaacaggccaaagcctgggagtcggggtcgtctccg
gccgaagagggcgagttctccaaaggccccatccgctgtgaccgctgtgg
ctacaataccaaccggtatgaccactacatggcacacctgaagcaccacc
tgcgagctggcgagaacgagcgcatctacaagtgcatcatctgcacgtac
acgacggtcagcgagtaccactggaggaaacacctgagaaaccatttccc
caggaaagtctacacctgcagcaagtgcaactacttctcagacagaaaaa
ataactacgttcagcacgtgcgaactcacacaggagaacgcccgtataaa
tgtgaactttgtccttactcaagctctcagaagactcatctaacgcgaca
catgcggactcattcagagtgtgatctagctgggtga.
CREB_binding_protein: NM_134442 (SEQ ID NO: 95):
atgaccatggaatctggagccgagaaccagcagagtggagatgcagctgt
aacagaagctgaaaaccaacaaatgacagttcaagcccagccacagattg
ccacattagcccaggtatctatgccagcagctcatgcaacatcatctgct
cccaccgtaactctagtacagctgcccaatgggcagacagttcaagtcca
tggagtcattcaggcggcccagccatcagttattcagtctccacaagtcc
aaacagttcagtcttcctgtaaggacttaaaaagacttttctccggaaca
cagatttcaactattgcagaaagtgaagattcacaggagtcagtggatag
tgtaactgattcccaaaagcgaagggaaattctttcaaggaggccttcct
acaggaaaattttgaatgacttatcttctgatgcaccaggagtgccaagg
attgaagaagagaagtctgaagaggagacttcagcacctgccatcaccac
tgtaacggtgccaactccaatttaccaaactagcagtggacagtatattg
ccattacccagggaggagcaatacagctggctaacaatggtaccgatggg
gtacagggcctgcaaacattaaccatgaccaatgcagcagccactcagcc
gggtactaccattctacagtatgcacagaccactgatggacagcagatct
tagtgcccagcaaccaagttgttgttcaagctgcctctggagacgtacaa
acataccagattcgcacagcacccactagcactattgcccctggagttgt
tatggcatcctccccagcacttcctacacagcctgctgaagaagcagcac
gaaagagagaggtccgtctaatgaagaacagggaagcagctcgagagtgt
cgtagaaagaagaaagaatatgtgaaatgtttagaaaacagagtggcagt
gcttgaaaatcaaaacaagacattgattgaggagctaaaagcacttaagg
acctttactgccacaaatcagatta; NM_004379; NP_004370;
NP_604391.
Zfp488: NM_001013777 (SEQ ID NO: 96):
atggctgagggcaaaggggctcctctgaggccttcagttgagaagagatg
gaagctcatggaacccaagcagacccaggcagggatgttcaagaaaatga
gccttgtggactctgacactgctgcaggaaagggtagccaagatgaggcc
tatactgaactgagcctgccaacagcaccgaacaagcctcgactggacag
gcctcgggcctgcaaggcatacacagagcagaggcacaataccttcacag
agctatcatgtctccaggagaggccaggggacatccaggcccagacgagg
aagctggagaacccagaaggccagctcggccctcagcagctgccctcgag
tttcctcagagcctcaggtgatggcacagtgtgttcagcatggccaggtg
ccccccggagtgagcagaaaaagtgctttcagcaagccagccaaacgccc
agcagagaaacctaagcgctctcccatgcttctggctggtggaagtgcag
agggctcatgggagctctcaggactcatcaccactgtggacatcccatat
tgggctcatctgtcaactttcaagttcatgggtgatttctggaaattgca
cacattgtcacagaacattctcctctgcaatgctttccagggggctccca
caccatggctggagcatacccaggtacaagcccccacatcctcagctcct
tcctccacagcctcccgggctctcttgccgcccacactctcctccttggg
cttgtctactcagaactggtgtgcgaagtgcaacctagcctttcgcctga
cagctgacctggtcttccacatgcggtcacatcacaaaagggaacacgtg
ggccctgacccacattctaagaaacgaagagaggaagttctcacttgccc
cgtttgccacgagtacttccgggagcgccaccatctgtccaggcatatgg
cttcacatagttag; BC089025; XM_224697; XP_224697.
Foxa2: NM_021784 (SEQ ID NO: 97):
atgctgggagcggtgaagatggaagggcacgagccgtccgactggagcag
ctactatgcagagcccgagggctactcctccgtgagcaacatgaacgccg
gcctggggatgaacggcatgaacacgtacatgagcatgtcggcggccgcc
atgggcagcggctcgggcaacatgagcgcgggctccatgaacatgtcgtc
gtacgtgggcgctggcatgagcccgtccctggcggggatgtcccccggcg
cgggcgccatggcgggcatgggcggctcggccggggcggccggcgtggcg
ggcatggggccgcacttgagtcccagcctgagcccgctcggggggcaggc
ggccggggccatgggcggcctggccccctacgccaacatgaactccatga
gccccatgtacgggcaggcgggcctgagccgcgcccgcgaccccaagacc
tacaggcgcagctacacgcacgcaaagccgccctactcgtacatctcgct
catcaccatggccatccagcagagccccaacaagatgctgacgctgagcg
agatctaccagtggatcatggacctcttccccttctaccggcagaaccag
cagcgctggcagaactccatccgccactcgctctccttcaacgactgttt
cctgaaggtgccccgctcgcccgacaagcccggcaagggctccttctgga
cctgcaccctgactcgggcaacatgttcgagaacggctgctacctgcgcc
gccagaagcgcttcaagtgcgagaagcagctggcgctgaaggaggccgca
ggcgccgccggcagcggcaagaaggcggccgccggagcccaggcctcaca
ggctcaactcggggaggccgccgggccggcctccgagactccggcgggca
ccgagtcgcctcactcgagcgcctccccgtgccaggagcacaagcgaggg
ggcctgggagagctgaaggggacgccggctgcggcgctgagccccccaga
gccggcgccctctcccgggcagcagcagcaggccgcggcccacctgctgg
gcccgccccaccacccgggcctgccgcctgaggcccacctgaagccggaa
caccactacgccttcaaccacccgttctccatcaacaacctcatgtcctc
ggagcagcagcaccaccacagccaccaccaccaccaaccccacaaaatgg
acctcaaggcctacgaacaggtgatgcactaccccggctacggttccccc
atgcctggcagcttggccatgggcccggtcacgaacaaaacgggcctgga
cgcctcgcccctggccgcagatacctcctactaccagggggtgtactccc
ggcccattatgaactcctcttaa; NP_068556; NM_012743;
NP_036875; NM_010446; NP_034576.
Rnx
REN: NM_000537 (SEQ ID NO: 98):
atggatggatggagaaggatgcctcgctggggactgctgctgctgctctg
gggctcctgtacctttggtctcccgacagacaccaccacctttaaacgga
tcttcctcaagagaatgccctcaatccgagaaagcctgaaggaacgaggt
gtggacatggccaggcttggtcccgagtggagccaacccatgaagaggct
gacacttggcaacaccacctcctccgtgatcctcaccaactacatggaca
cccagtactatggcgagattgggatcgggaccccaccccaaaccttcaaa
gtcgtctttgacactggttcgtccaatgtttgggtgccctcctccaagtg
cagccgtctctacactgcctgtgtgtatcacaagctcttcgatgcttcgg
attcctccagctacaagcacaatggaacagaactcaccctccgctattca
acagggacagtcagtggctttctcagccaggacatcatcaccgtgggtgg
aatcacggtgacacagatgtttggagaggtcacggagatgcccgccttac
ccttcatgctggccgagtttgatggggttgtgggcatgggcttcattgaa
caggccattggcagggtcacccctatcttcgacaacatcatctcccaagg
ggtgctaaaagaggacgtcttctctttctactacaacagagattccgaga
attcccaatcgctgggaggacagattgtgctgggaggcagcgacccccag
cattacgaagggaatttccactatatcaacctcatcaagactggtgtctg
gcagattcaaatgaagggggtgtctgtggggtcatccaccttgctctgtg
aagacggctgcctggcattggtagacaccggtgcatcctacatctcaggt
tctaccagctccatagagaagctcatggaggccttgggagccaagaagag
gctgtttgattatgtcgtgaagtgtaacgagggccctacactccccgaca
tctctttccacctgggaggcaaagaatacacgctcaccagcgcggactat
gtatttcaggaatcctacagtagtaaaaagctgtgcacactggccatcca
cgccatggatatcccgccacccactggacccacctgggccctgggggcca
ccttcatccgaaagttctacacagagtttgatcggcgtaacaaccgcatt
ggcttcgccttggcccgctga;
dHAND(HAND2): NM_021973 (SEQ ID NO: 99):
atgagtaggtaggtggttttccccaccacccggtggtgcaccacgagggc
tacccgtttgccgccgccgccgccgcagctgccgccgccgccgccagccg
ctgcagccatgaggagaacccctacttccatggctggctcatcggccacc
ccgagatgtcgccccccgactacagcatggccctgtcctacagccccgag
tatgccagcggcgccgccggcctggaccactcccattacgggggggtgcc
gccgggcgccgggcccccgggcctgggggggccgcgcccggtgaagcgcc
gaggcaccgccaaccgcaaggagcggcgcaggactcagagcatcaacagc
gccttcgccgaactgcgcgagtgcatccccaacgtacccgccgacaccaa
actctccaaaatcaagaccctgcgcctggccaccagctacatcgcctacc
tcatggacctgctggccaaggacgaccagaatggcgaggcggaggccttc
aaggcagagatcaagaagaccgacgtgaaagaggagaagaggaagaagga
gctgaacgaaatcttgaaaagcacagtgagcagcaacgacaagaaaacca
aaggccggacgggctggccgcagcacgtctgggccctggagctcaagcag
tga; NM_010402;
aspartoacylase (Canavan disease) (ASPA):
NM_000049 (SEQ ID NO: 100):
atgacttcttgtcacattgctgaagaacatatacaaaaggttgctatctt
tggaggaacccatgggaatgagctaaccggagtatttctggttaagcatt
ggctagagaatggcgctgagattcagagaacagggctggaggtaaaacca
tttattactaaccccagagcagtgaagaagtgtaccagatatattgactg
tgacctgaatcgcatttttgaccttgaaaatcttggcaaaaaaatgtcag
aagatttgccatatgaagtgagaagggctcaagaaataaatcatttattt
ggtccaaaagacagtgaagattcctatgacattatttttgaccttcacaa
caccacctctaacatggggtgcactcttattcttgaggattccaggaata
actttttaattcagatgtttcattacattaagacttctctggctccacta
ccctgctacgtttatctgattgagcatccttccctcaaatatgcgaccac
tcgttccatagccaagtatcctgtgggtatagaagttggtcctcagcctc
aaggggttctgagagctgatatcttggatcaaatgagaaaaatgattaaa
catgctcttgattttatacatcatttcaatgaaggaaaagaatttcctcc
ctgcgccattgaggtctataaaattatagagaaagttgattacccccggg
atgaaaatggagaaattgctgctatcatccatcctaatctgcaggatcaa
gactggaaaccactgcatcctggggatcccatgtttttaactcttgatgg
gaagacgatcccactgggcggagactgtaccgtgtaccccgtgtttgtga
atgaggccgcatattacgaaaagaaagaagcttttgcaaagacaactaaa
ctaacgctcaatgcaaaaagtattcgctgctgtttacattag;
NM_023113.
hexosaminidaseA(HEXA):
NM_000520 (SEQ ID NO: 101):
atgacaagctccaggctttggttttcgctgctgctggcggcagcgttcgc
aggacgggcgacggccctctggccaggcctcagaacttccaaacctccga
ccagcgctacgtcctttacccgaacaactttcaattccagtacgatgtca
gctcggccgcgcagcccggctgctcagtcctcgacgaggccttccagcgc
tatcgtgacctgcttttcggttccgggtcttggccccgtccttacctcac
agggaaacggcatacactggagaagaatgtgttggttgtctctgtagtca
cacctggatgtaaccagcttcctactttggagtcagtggagaattatacc
ctgaccataaatgatgaccagtgtttactcctctctgagactgtctgggg
agctctccgaggtctggagacttttagccagcttgtttggaaatctgctg
agggcacattctttatcaacaagactgagattgaggactttccccgcttt
cctcaccggggcttgctgttggatacatctcgccattacctgccactctc
tagcatcctggacactctggatgtcatggcgtacaataaattgaacgtgt
tccactggcatctggtagatgatccttccttcccatatgagagcttcact
tttccagagacatgagaaaggggtcctacaaccctgtcacccacatctac
acagcacaggatgtgaaggaggtcattgaatacgcacggctccggggtat
ccgtgtgcttgcagagtttgacactcctggccacactttgtcctggggac
caggtatccctggattactgactccttgctactctgggtctgagccctct
ggcacctttggaccagtgaatcccagtctcaataatacctatgagttcat
gagcacattcttcttagaagtcagctctgtcttcccagatttttatcttc
atcttggaggagatgaggttgatttcacctgctggaagtccaacccagag
atccaggactttatgaggaagaaaggcttcggtgaggacttcaagcagct
ggagtccttctacatccagacgctgctggacatcgtctcttcttatggca
agggctatgtggtgtggcaggaggtgtttgataataaagtaaagattcag
ccagacacaatcatacaggtgtggcgagaggatattccagtgaactatat
gaaggagctggaactggtcaccaaggccggcttccgggcccttctctctg
ccccctggtacctgaaccgtatatcctatggccctgactggaaggatttc
tacgtagtggaacccctggcatttgaaggtacccctgagcagaaggctct
ggtgattggtggagaggcttgtatgtggggagaatatgtggacaacacaa
acctggtccccaggctctggcccagagcaggggctgttgccgaaaggctg
tggagcaacaagttgacatctgacctgacatttgcctatgaacgtttgtc
acacttccgctgtgagttgctgaggcgaggtgtccaggcccaacccctca
atgtaggcttctgtgagcaggagtttgaacagacctga.
Lesch_Nyhan_syndrome(HRPT):
NM_000194 (SEQ ID NO: 102):
atggcgacccgcagccctggcgtcgtgattagtgatgatgaaccaggtta
tgaccttgatttattttgcatacctaatcattatgctgaggatttggaaa
gggtgtttattcctcatggactaattatggacaggactgaacgtcttgct
cgagatgtgatgaaggagatgggaggccatcacattgtagccctctgtgt
gctcaaggggggctataaattctttgctgacctgctggattacatcaaag
cactgaatagaaatagtgatagatccattcctatgactgtagattttatc
agactgaagagctattgtaatgaccagtcaacaggggacataaaagtaat
tggtggagatgatctctcaactttaactggaaagaatgtcttgattgtgg
aagatataattgacactggcaaaacaatgcagactttgctttccttggtc
aggcagtataatccaaagatggtcaaggtcgcaagcttgctggtgaaaag
gaccccacgaagtgttggatataagccagactttgttggatttgaaattc
cagacaagtttgttgtaggatatgcccttgactataatgaatacttcagg
gatttgaatcatgtttgtgtcattagtgaaactggaaaagcaaaatacaa
agcctaa; NM_204848.
Huntingtin; NM_010414;
GUSB; NM_000181 (SEQ ID NO: 103):
atggcccgggggtcggcggttgcctgggcggcgctcgggccgttgttgtg
gggctgcgcgctggggctgcagggcgggatgctgtacccccaggagagcc
cgtcgcgggagtgcaaggagctggacggcctctggagcttccgcgccgac
ttctctgacaaccgacgccggggcttcgaggagcagtggtaccggcggcc
gctgtgggagtcaggccccaccgtggacatgccagttccctccagcttca
atgacatcagccaggactggcgtctgcggcattttgtcggctgggtgtgg
tacgaacgggaggtgatcctgccggagcgatggacccaggacctgcgcac
aagagtggtgctgaggattggcagtgcccattcctatgccatcgtgtggg
tgaatggggtcgacacgctagagcatgaggggggctacctccccttcgag
gccgacatcagcaacctggtccaggtggggcccctgccctcccggctccg
aatcactatcgccatcaacaacacactcacccccaccaccctgccaccag
ggaccatccaatacctgactgacacctccaagtatcccaagggttacttt
gtccagaacacatattttgactttttcaactacgctggactgcagcggtc
tgtacttctgtacacgacacccaccacctacatcgatgacatcaccgtca
ccaccagcgtggagcaagacagtgggctggtgaattaccagatctctgtc
aagggcagtaacctgttcaagttggaagtgcgtcttttggatgcagaaaa
caaagtcgtggcgaatgggactgggacccagggccaacttaaggtgccag
gtgtcagcctctggtggccgtacctgatgcacgaacgccctgcctatctg
tattcattggaggtgcagctgactgcacagacgtcactggggcctgtgtc
tgacttctacacactccctgtggggatccgcactgtggctgtcaccaaga
gccagttcctcatcaatgggaaacctttctatttccacggtgtcaacaag
catgaggatgcggacatccgagggaagggcttcgactggccgctgctggt
gaaggacttcaacctgcttcgctggcttggtgccaacgctttccgtacca
gccactacccctatgcagaggaagtgatgcagatgtgtgaccgctatggg
attgtggtcatcgatgagtgtcccggcgtgggcctggcgctgccgcagtt
cttcaacaacgtttctagcatcaccacatgcaggtgatggaagaagtggt
gcgtagggacaagaaccaccccgcggtcgtgatgtggtctgtggccaacg
agcctgcgtcccacctagaatctgctggctactacttgaagatggtgatc
gctcacaccaaatccttggacccctcccggcctgtgacctttgtgagcaa
ctctaactatgcagcagacaagggggctccgtatgtggatgtgatctgtt
tgaacagctactactcttggtatcacgactacgggcacctggagttgatt
cagctgcagctggccacccagtttgagaactggtataagaagtatcagaa
gcccattattcagagcgagtatggagcagaaacgattgcagggtttcacc
aggatccacctctgatgttcactgaagagtaccagaaaagtctgctagag
cagtaccatctgggtctggatcaaaaacgcagaaaatacgtggttggaga
gctcatttggaattttgccgatttcatgactgaacagtcaccgacgagag
tgctggggaataaaaaggggatcttcactcggcagagacaaccaaaaagt
gcagcgttccttttgcgagagagatactggaagattgccaatgaaaccag
gtatccccactcagtagccaagtcacaatgtttggaaaacagcccgttta
cttga; NM_010368.
NPC1: NM_000271; NM_006432.
hexosaminidaseB: NM_000521 (SEQ ID NO: 104):
atggagctgtgcgggctggggctgccccggccgcccatgctgctggcgct
gctgcttggcgacactgctggcggcgatgttggcgctgctgactcaggtg
gcgctggtggtgcaggtggcggaggcggctcgggccccgagcgtctcggc
caagccggggccggcgctgtggcccctgccgctctcggtgaagatgaccc
cgaacctgctgcatctcgccccggagaacttctacatcagccacagcccc
aattccacggcgggcccctcctgcaccctgctggaggaagcgtttcgacg
atatcatggctatatttttggtttctacaagtggcatcatgaacctgctg
aattccaggctaaaacccaggttcagcaacttcttgtctcaatcaccctt
cagtcagagtgtgatgctttccccaacatatcttcagatgagtcttatac
tttacttgtgaaagaaccagtggctgtccttaaggccaacagagtttggg
gagcattacgaggtttagagacctttagccagttagtttatcaagattct
tatggaactttcaccatcaatgaatccaccattattgattctccaaggtt
ttctcacagaggaattttgattgatacatccagacattatctgccagtta
agattattcttaaaactctggatgccatggcttttaataagtttaatgtt
cttcactggcacatagttgatgaccagtctttcccatatcagagcatcac
ttttcctgagttaagcaataaaggaagctattctttgtctcatgtttata
caccaaatgatgtccgtatggtgattgaatatgccagattacgaggaatt
cgagtcctgccagaatttgatacccctgggcatacactatcttggggaaa
aggtcagaaagacctcctgactccatgttacagtagacaaaacaagttgg
actcttttggacctataaaccctactctgaatacaacatacagcttcctt
actacatttttcaaagaaattagtgaggtgtttccagatcaattcattca
tttgggaggagatgaagtggaatttaaatgttgggaatcaaatccaaaaa
ttcaagatttcatgaggcaaaaaggctttggcacagattttaagaaacta
gaatctttctacattcaaaaggttttggatattattgcaaccataaacaa
gggatccattgtctggcaggaggtttttgatgataaagcaaagcttgcgc
cgggcacaatagttgaagtatggaaagacagcgcatatcctgaggaactc
agtagagtcacagcatctggcttccctgtaatcctttctgctccctggta
cttagatttgattagctatggacaagattggaggaaatactataaagtgg
aacctcttgattttggcggtactcagaaacagaaacaacttttcattggt
ggagaagcttgtctatggggagaatatgtggatgcaactaacctcactcc
aagattatggcctcgggcaagtgctgttggtgagagactctggagttcca
aagatgtcagagatatggatgacgcctatgacagactgacaaggcaccgc
tgcaggatggtcgaacgtggaatagctgcacaacctctttatgctggata
ttgtaaccatgagaacatgtaa.
galactosidase, alpha(GLA):
NM_000169 (SEQ ID NO: 105):
atgcagctgaggaacccagaactacatctgggctgcgcgcttgcgcttcg
cttcctggccctcgtttcctgggacatccctggggctagagcactggaca
atggattggcaaggacgcctaccatgggctggctgcactgggagcgcttc
atgtgcaaccttgactgccaggaagagccagattcctgcatcagtgagaa
gctcttcatggagatggcagagctcatggtctcagaaggctggaaggatg
caggttatgagtacctctgcattgatgactgttggatggctccccaaaga
gattcagaaggcagacttcaggcagaccctcagcgctttcctcatgggat
tcgccagctagctaattatgttcacagcaaaggactgaagctagggattt
atgcagatgttggaaataaaacctgcgcaggcttccctgggagttttgga
tactacgacattgatgcccagacctttgctgactggggagtagatctgct
aaaatttgatggttgttactgtgacagtttggaaaataggcagatggtta
taagcacatgtccttggccctgaataggactggcagaagcattgtgtact
cctgtgagtggcctctttatatgtggccctttcaaaagcccaattataca
gaaatccgacagtactgcaatcactggcgaaattttgctgacattgatga
ttcctggaaaagtataaagagtatcttggactggacatcttttaaccagg
agagaattgttgatgttgctggaccagggggttggaatgacccagatatg
ttagtgattggcaactttggcctcagctggaatcagcaagtaactcagat
ggccctctgggctatcatggctgctcctttattcatgtctaatgacctcc
gacacatcagccctcaagccaaagctctccttcaggataaggacgtaatt
gccatcaatcaggaccccttgggcaagcaagggtaccagcttagacaggg
agacaactttgaagtgtgggaacgacctctctcaggcttagcctgggctg
tagctatgataaaccggcaggagattggtggacctcgctcttataccatc
gcagttgcttccctgggtaaaggagtggcctgtaatcctgcctgcttcat
cacacagctcctccctgtgaaaaggaagctagggttctatgaatggactt
caaggttaagaagtcacataaatcccacaggcactgttttgcttcagcta
gaaaatacaatgcagatgtcattaaaagacttactttaa
glucosidase_beta_acid(GBA):
NM_000157 (SEQ ID NO: 106):
atggagttttcaagtccttccagagaggaatgtcccaagcctttgagtag
ggtaagcatcatggctggcagcctcacaggattgcttctacttcaggcag
tgtcgtgggcatcaggtgcccgcccctgcatccctaaaagcttcggctac
agctcggtggtgtgtgtctgcaatgccacatactgtgactcttttgaccc
cccgacctttcctgcccttggtaccttcagccgctatgagagtacacgca
gtgggcgacggatggagctgagtatggggcccatccaggctaatcacacg
ggcacaggcctgctactgaccctgcagccagaacagaagttccagaaagt
gaagggatttggaggggccatgacagatgctgctgctctcaacatccttg
ccctgtcaccccctgcccaaaatttgctacttaaatcgtacttctctgaa
gaaggaatcggatataacatcatccgggtacccatggccagctgtgactt
ctccatccgcacctacacctatgcagacacccctgatgatttccagttgc
acaacttcagcctcccagaggaagataccaagctcaagatacccctgatt
caccgagccctgcagttggcccagcgtcccgtttcactccttgccagccc
ctggacatcacccacttggctcaagaccaatggagcggtgaatgggaagg
ggtcactcaagggacagcccggagacatctaccaccagacctgggccaga
tactttgtgaagttcctggatgcctatgctgagcacaagttacagttctg
ggcagtgacagctgaaaatgagccttctgctgggctgttgagtggatacc
ccttccagtgcctgggcttcacccctgaacatcagcgagacttcattgcc
cgtgacctaggtcctaccctcgccaacagtactcaccacaatgtccgcct
actcatgctggatgaccaacgcttgctgctgccccactgggcaaaggtgg
tactgacagacccagaagcagctaaatatgttcatggcattgctgtacat
tggtacctggactttctggctccagccaaagccaccctaggggagacaca
ccgcctgttccccaacaccatgctctttgcctcagaggcctgtgtgggct
ccaagttctgggagcagagtgtgcggctaggctcctgggatcgagggatg
cagtacagccacagcatcatcacgaacctcctgtaccatgtggtcggctg
gaccgactggaaccttgccctgaaccccgaaggaggacccaattgggtgc
gtaactttgtcgacagtcccatcattgtagacatcaccaaggacacgttt
tacaaacagcccatgttctaccaccttggccacttcagcaagttcattcc
tgagggctcccagagagtggggctggttgccagtcagaagaacgacctgg
acgcagtggcactgatgcatcccgatggctctgctgttgtggtcgtgcta
aaccgctcctctaaggatgtgcctcttaccatcaaggatcctgctgtggg
cttcctggagacaatctcacctggctactccattcacacctacctgtggc
gtcgccagtga; NM_008094.
von_Hippel_Lindau_tumor_suppressor(VHL):
NM_000551 (SEQ ID NO: 107):
atgccccggagggcggagaactgggacgaggccgaggtaggcgcggagga
ggcaggcgtcgaagagtacggccctgaagaagacggcggggaggagtcgg
gcgccgaggagtccggcccggaagagtccggcccggaggaactgggcgcc
gaggaggagatggaggccgggcggccgcggcccgtgctgcgctcggtgaa
ctcgcgcgagccctcccaggtcatcttctgcaatcgcagtccgcgcgtcg
tgctgcccgtatggctcaacttcgacggcgagccgcagccctacccaacg
ctgccgcctggcacgggccgccgcatccacagctaccgaggtcacctttg
gctcttcagagatgcagggacacacgatgggcttctggttaaccaaactg
aattatttgtgccatctctcaatgttgacggacagcctatttttgccaat
atcacactgccagtgtatactctgaaagagcgatgcctccaggttgtccg
gagcctagtcaagcctgagaattacaggagactggacatcgtcaggtcgc
tctacgaagatctggaagaccacccaaatgtgcagaaagacctggagcgg
ctgacacaggagcgcattgcacatcaacggatgggagattga.
Beta_globin(HBB): NM_000518 (SEQ ID NO: 108):
atggtgcatctgactcctgaggagaagtctgccgttactgccctgtgggg
caaggtgaacgtggatgaagttggtggtgaggccctgggcaggctgctgg
tggtctacccttggacccagaggttctttgagtcctttggggatctgtcc
actcctgatgctgttatgggcaaccctaaggtgaaggctcatggcaagaa
agtgctcggtgcctttagtgatggcctggctcacctggacaacctcaagg
gcacctttgccacactgagtgagctgcactgtgacaagctgcacgtggat
cctgagaacttcaggctcctgggcaacgtgctggtctgtgtgctggccca
tcactttggcaaagaattcaccccaccagtgcaggctgcctatcagaaag
tggtggctggtgtggctaatgccctggcccacaagtatcactaa.
PARK2: NM_013988 (SEQ ID NO: 109):
atgatagtgtttgtcaggttcaactccagccatggtttcccagtggaggt
cgattctgacaccagcatcttccagctcaaggaggtggttgctaagcgac
agggggttccggctgaccagttgcgtgtgattttcgcagggaaggagctg
aggaatgactggactgtgcaggaatttttctttaaatgtggagcacaccc
cacctctgacaaggaaacatcagtagctttgcacctgatcgcaacaaata
gtcggaacatcacttgcattacgtgcacagacgtcaggagccccgtcctg
gttttccagtgcaactcccgccacgtgatttgcttagactgtttccactt
atactgtgtgacaagactcaatgatcggcagtttgttcacgaccctcaac
ttggctactccctgccttgtgtggctggctgtcccaactccttgattaaa
gagctccatcacttcaggattctgggagaagagcagtacaaccggtacca
gcagtatggtgcagaggagtgtgtcctgcagatggggggcgtgttatgcc
cccgccctggctgtggagcggggctgctgccggagcctgaccagaggaaa
gtcacctgcgaagggggcaatggcctgggctgtgggtttgccttctgccg
ggaatgtaaagaagcgtaccatgaaggggagtgcagtgccgtatttgaag
cctcaggaacaactactcaggcctacagagtcgatgaaagagccgccgag
caggctcgagggaagcagcctccaaagaaaccatcaagaaaaccaccaag
ccctgtccccgctgccatgtaccagtggaaaaaaatggaggctgcatgca
catgaagtgtccgcagccccagtgcaggctcgagtggtgctggaactgtg
gctgcgagtggaaccgcgtctgcatgggggaccactggttcgacgtgt
ag; NM_004562; NM_020093.

The contents of all parenthetically cited publications and the following United States patents, are noted and incorporated by reference in their entireties: U.S. Pat. Nos. 7,211,247, 5,677,139, 6,432,711 and 5,453,357, U.S. Pat. No. 05,593,875, U.S. Pat. No. 05,783,566, U.S. Pat. No. 5,928,944, U.S. Pat. No. 05,910,488, U.S. Pat. No. 05,824,547,

1-135. (canceled)

136. A method of cellular reprogramming for the interconversion of a differentiated or somatic cell of a first cellular phenotype to a second, multipotent, pluripotent, self-renewing or differentiating phenotype, the method comprising:
growing said cell in a growth medium containing one or more cytokines selected from EGF, IL-7, oncostatin, CNTF, soluble gp130, bFGF, steel factor, LIF, cardiotrophin, OSM, IL6, hyper IL6, a cytokine having LIF activity, and other growth enhancing cytokines, effective that the selected cells grow at a first growth rate;
contacting said cell in a contacting step comprising contacting said cell with a polypeptide, or with ribonucleic acids or deoxyribonucleic acids encoding one or more polypeptides, wherein said one or more of said polypeptides promotes desirable differentiation;
genetically-modifying said cell with a nucleotide sequence, polypeptide, or nucleic acid encoding a polypeptide;
incubating said cell in a differentiation medium comprising at least one differentiation agent conducive to promote differentiation of said cell into a desired differentiating cell;
assessing said cell according to morphology, expression of cellular markers, transgenic markers, antibiotic markers, or fluorescent markers; and
transplanting said cell to an individual or to a patient in need thereof.

137. A method of cellular reprogramming for the interconversion of a differentiated or somatic cell of a first cellular phenotype to a second, multipotent, pluripotent, self-renewing or differentiating phenotype, the method comprising:
contacting a nucleated cell in a first contacting step with a first polypeptide or vector encoding a first polypeptide in a first contacting step, said first contacting step comprising contacting said cell with said first polypeptide, or with one or more ribonucleic acids or deoxyribonucleic acids encoding said first polypeptide, wherein said first polypeptide is Oct3/4, Sox2, LIN28 or Nanog;
further contacting said cell in a second contacting step with one or more second polypeptides, or with ribonucleic acids or deoxyribonucleic acids encoding one or more second polypeptides, wherein said one or more second polypeptide is selected from Numb, Notch, FGF4, HOXB4, LIF, LIFR, CNTF, cardiotrophin, OSM, OSM-R, IL6, IL6R, hyper IL6, IL-11, gp130, stat3, c-myc, and a polypeptide with LIF activity;
growing said cell in a growth medium containing one or more cytokines selected from EGF, IL-7, oncostatin, CNTF, soluble gp130, bFGF, steel factor, LIF, cardiotrophin, OSM, IL6, hyper IL6, a cytokine having LIF activity, and other growth enhancing cytokines, effective that said cell grows at a first growth rate;
contacting said cell in a third contacting step comprising contacting said cell with a third polypeptide, or with ribonucleic acids or deoxyribonucleic acids encoding one or more third polypeptides, wherein said one or more third polypeptide promotes desirable differentiation;
genetically-modifying said cell with a nucleotide sequence, polypeptide, or nucleic acid encoding a polypeptide;
incubating said cell in a differentiation medium comprising at least one differentiation agent conducive to promote differentiation of said cell into a desired differentiating cell;
assessing said cell according to morphology, expression of cellular markers, transgenic markers, antibiotic markers, or fluorescent markers; and
transplanting said cell to an individual or to a patient in need thereof.

138. The method of claim 137, wherein said first contacting step comprises contacting said cell with a vector encoding said first polypeptide, wherein said vector does not integrate into the genome of said cell, said vector comprising a nucleic acid sequence coding for only one first polypeptide, wherein said only one first polypeptide is selected from Oct3/4, Sox2, LIN28 or Nanog.

139. The method of claim 137, wherein said cell incubated in said differentiation medium grows at a second growth rate in said differentiation medium, wherein said second growth rate is a reduced growth rate as compared with said first growth rate.

140. The method of claim 137, wherein said second contacting step comprises contacting said cell with a vector encoding said second polypeptide, wherein said vector does not integrate into the genome of said cell, said vector comprising a nucleic acid sequence coding for one or more second polypeptide, wherein said second polypeptide is selected from Numb, Notch, HoxB4, FGF4, LIF, LIFR, cardiotrophin, oncostatin, oncostatin receptor, IL6, hyper IL6, gp130, stat3, c-myc, CNTF, and a polypeptide with LIF activity.

141. The method of claim 137 where the cell is genetically-modified by exposure to a vector comprising a synthetic oligonucleotide decoy sequence and synthetic oligonucleotides directed against a HIV co-receptor, wherein said vector is capable of retarding HIV-1 and/or HIV-2 infection.

142. The method of claim 141, wherein said vector comprises an HIV-2 RRE decoy sequence and an HIV-2 TAR decoy sequence, wherein said vector is capable of retarding HIV-1 and/or HIV-2 infection.

143. The method of claim 141, wherein said synthetic oligonucleotides are selected from siRNA, miRNA and shRNA.

144. The method of claim 142, wherein said vector is a viral vector, and wherein the synthetic oligonucleotides comprise miRNA sequences directed against CXCR4 and directed against CCR5.

145. The method of claim 137 where the nucleated cell is selected from the group of stem/progenitor cells and somatic cell types consisting of autologous cells, histocompatible cells, reprogrammed cells, induced multipotent cells, induced pluripotent cells, cells derived from the bone marrow, the peripheral blood, placental blood, amniotic fluid, umbilical cord blood, banked or cryopreserved sources, skin, adipose tissue, and non-human embryos, hematopoietic cells, spermatogonia, primordial germ cells, leukocytes, lymphocytes, epithelial cells, buccal cheek cells, genetically-modified cells, cells genetically-modified not to express CCR5, and cells genetically-modified not to express CXCR4.

146. The method of claim 137, wherein said second contacting step does not comprise contact with a second polypeptide.

147. The method of claim 137, wherein said second contacting step comprises contacting said cell with a vector encoding said second polypeptide, wherein said vector does not integrate into the genome of said cell, said vector comprising a nucleic acid sequence coding for one or more second polypeptides, wherein said second polypeptide is selected from Numb, Notch, FGF4, HOXB4, LIF, LIFR, CNTF, cardiotrophin, OSM, OSM-R, IL6, IL6R, hyper IL6, IL-11, gp130, stat3, c-myc, and a polypeptide with LIF activity.

148. The method of claim 139, further comprising a fourth contacting step, wherein said fourth contacting step comprises contacting said cell with a fourth polypeptide, or contacting said cell with a vector comprising a nucleic acid sequence encoding a fourth polypeptide or antisense RNA, wherein said fourth polypeptide promotes differentiation, and wherein the fourth polypeptide is selected from a short Numb isoform, Numblike, MyoD, myogenin, Myocardin, Ifrd1, Myf 5, Myf 6, Mef2, Gata 4, Gata 5, and Gata 6, Sox9, CREB-binding polypeptide, Runx2, HNF-1, HNF-3, HNF-4, HNF-6, Nurr1, REN, Neurogenin1, Neurogenin2, Neurogenin3, Mash 1 (Ascl 1), Phox2a, Phox2b, dHand, Gata3, Shh, FGF8, Lmx1b, Nk×2.2, Pet1, Lbx1, Rnx, PITX2, D1x2, D1x5, REN, Ngn2, Ptx-3, Gata2, REST4, Foxa2, Sox17, HLXB9, Runx1/AML, Pdx1, OLIG1, OLIG2, NOV(CCN3), and Zfp488, or said vector comprises antisense Hes1 RNA.

149. The method of claim 137, wherein said first contacting step comprises contacting said cell with a vector, wherein said vector does not integrate into the genome of said cell.

150. The method of claim 137 wherein said first contacting step comprises contacting the cell with a nucleic acid that does not integrate into the genome of the cell and which encodes a first polypeptide, or contacting the cell with at first polypeptide, wherein said first polypeptide is selected from Nanog, Oct3/4 or Sox2; and further contacting said cell with a second polypeptide selected from HoxB4, Numb, Notch, c-myc, and polypeptides having LIF activity; wherein the cell is genetically-modified using a vector comprising a HIV decoy synthetic oligonucleotide sequence and a synthetic oligonucleotide directed against a HIV co-receptor; and further contacting the cell with a nucleic acid encoding NOV(CCN3) or Runx1/AML1, or with a polypeptide that is NOV(CCN3) or Runx1/AML1.

151. The method of claim 148, comprising contacting a cancerous or dysplastic cell with a polypeptide comprising numblike or a short numb isoform, or with a vector comprising a nucleic acid sequence encoding a polypeptide, wherein said polypeptide is numblike.

152. The method of claim 137, wherein said first polypeptide is Oct3/4, and further contacting said cell with a further polypeptide, or with a nucleic acid encoding a further polypeptide, wherein said further polypeptide is Sox2.

153. The method of claim 139, wherein said cells grown at a reduced growth rate are grown in an environment selected from de-cellularized cadaveric tissue, a two-dimensional scaffold engineered to replicate tissue structure or an organ, a three-dimensional scaffold engineered to replicate tissue structure or an organ, and an ink-jet style cartridge for subsequent placement of said cell in a desired position utilizing ink-jet printer technology.

154. The method of claim 137, further comprising isolating selected cells, wherein selected cells are isolated from the growth or differentiation medium after achieving a desired cell number and a desired state of differentiation.

155. A cell treated according to the method of claim 137.

156. A vector for use in the method of claim 137, wherein said vector is capable of retarding HIV-1 and/or HIV-2 infection that includes decoy, synthetic oligonucleotide sequence(s) and synthetic oligonucleotides directed against HIV co-receptor(s), where the synthetic oligonucleotides include oligonucleotides selected from the group siRNA, miRNA and shRNA.

157. The method of claim 154, further comprising administering said isolated selected cells to a subject in need of such cells.

158. A method of ameliorating infection in a patient comprising:
a) providing a vector of claim 156,
b) contacting a cell with said vector according to the method of claim 1 to provide a multipotent, pluripotent, self-renewing, differentiating or disease-resistant cell, wherein said contacted cell is selected from a nucleated cell, an umbilical cord blood cell, a hematopoietic cell, a peripheral blood cell, a bone marrow cell, a spermatogonial cell, a primordial germ cell, a CD4+ cell, a macrophage, a T cell, a CD34+ stem/progenitor cell, and/or a cell capable of further differentiation in vitro or in vivo into an HIV target cell, and
c) introducing the multipotent, pluripotent, self-renewing, differentiating or disease-resistant cell into the patient's circulation, bone marrow, organ, nervous system or venticular system.

159. The method of claim 137 wherein said differentiation medium comprises an agent selected from the group consisting of retinoic acid, Neurotrophin 3 (NT3), nerve growth factor (NGF), glial cell-line derived growth factor (GDNF), interferon γ (IFN-γ), hexamethylene bis acrylamide, dimethylsulfoxide, fetal bovine serum (FBS), normal bovine serum (NBS), vascular endothelial growth factor (VEGF), thrombopoietin, a colony stimulating factor, M-CSF (CSF-1), GM-CSF, IL-7, and cardiomyocyte conditioned medium.

160. The method of claim 159, wherein the cell is contacted in a first contacting step with one first polypeptide, or with one or more nucleic acids encoding one first polypeptide, wherein said one first polypeptide is Oct3/4, and incubating the cell in a differentiation medium comprising one or more agents selected from the group consisting of retinoic acid, Neurotrophin 3 (NT3), LIF, nerve growth factor (NGF), glial cell-line derived growth factor (GDNF), interferon γ (IFN-γ), hexamethylene bis acrylamide, dimethylsulfoxide, fetal bovine serum (FBS), normal bovine serum (NBS), vascular endothelial growth factor (VEGF), a colony stimulating factor, thrombopoietin, M-CSF (CSF-1), GM-CSF, IL-7, and cardiomyocyte conditioned medium.

161. The method of claim 137 wherein a contacting step comprises transfection with therapeutic vectors comprising a synthetic oligonucleotide or a nucleic acid sequence encoding a protein selected from telomerase, aspartoacyclase, hexosaminidase A (HEXA), hexosaminidase B (HEXB), HPRT, Huntingtin, galactosidase alpha (HLA), glucosidase beta acid (GBA), von Hippel Lindau tumor suppressor (VHL), Beta globin (HBB), NPC1, and PARK2.

162. The method of claim 137, wherein one or more contacting step comprises use of one or more of electroporation, a nanocapsule, a nanovault, and a liposome, or comprises avoiding retroviral/lentiviral integration or other random alteration of the genomes of said selected cells.

163. The method of claim 137 wherein selected cells are assessed according to expression of nanog, tubulin III, a reporter gene, a transgenic marker, a marker gene encoded by a transgene expressing vector, an antibiotic resistance gene, a fluorescent protein gene, or a reporter gene under the control of a cell type specific promoter.

164. The method of claim 137 wherein the contacting of said cells with a protein or nucleotide sequence in vivo causes an amelioration of hypoplastic, dysplastic, or cancerous behaviour in said cells.

165. The method of claim 137 wherein a candidate polypeptide, nucleic acid or agent is present in order to assess its ability to induce a desired cell behavior.
